(19) United States
(12) Patent Application Publication
(10) Pub. No.: US 2025/0295639 A1
(43) Pub. Date: Sep. 25, 2025

(54) METHODS FOR TREATING HEART DISEASES

(71) Applicant: Cytokinetics, Incorporated, South San Francisco, CA (US)

(72) Inventors: Fady MALIK, Burlingame, CA (US); Stuart KUPFER, Highland Park, IL (US); Stephen B. HEITNER, Portland, OR (US); Daniel Louis JACOBY, Madison, CT (US); Lixin MENG, Cupertino, CA (US)

(21) Appl. No.: 19/002,411

(22) Filed: Dec. 26, 2024

Related U.S. Application Data
(60) Provisional application No. 63/567,935, filed on Mar. 20, 2024.
(60) Provisional application No. 63/568,801, filed on Mar. 22, 2024.
(60) Provisional application No. 63/572,074, filed on Mar. 29, 2024.
(60) Provisional application No. 63/636,352, filed on Apr. 19, 2024.
(60) Provisional application No. 63/650,810, filed on May 22, 2024.
(60) Provisional application No. 63/656,504, filed on Jun. 5, 2024.
(60) Provisional application No. 63/665,947, filed on Jun. 28, 2024.
(60) Provisional application No. 63/672,627, filed on Jul. 17, 2024.
(60) Provisional application No. 63/681,620, filed on Aug. 9, 2024.
(60) Provisional application No. 63/690,691, filed on Sep. 4, 2024.
(60) Provisional application No. 63/697,315, filed on Sep. 20, 2024.

(51) Int. Cl. A61K 31/4245; A61K 9/20; A61K 45/06; A61P 9/00

(52) U.S. Cl. CPC A61K 31/4245; A61K 9/2018; A61K 9/2054; A61K 45/06; A61P 9/00

(57) ABSTRACT

Provided herein are methods of treating patients by administering aficamten, or a pharmaceutically acceptable salt thereof, including in combination with beta blockers. The treatment methods include improving heart rate reserve, peak metabolic equivalents (METs), exercise duration, peak oxygen uptake (pVO₂) as well as other parameters related to exercise capacity.

### CROSS REFERENCE TO RELATED APPLICATIONS

This application claims priority to U.S. Provisional Patent Application No. 63/567,935, filed Mar. 20, 2024, U.S. Provisional Patent Application No. 63/568,801, filed Mar. 22, 2024, U.S. Provisional Patent Application No. 63/572,074, filed Mar. 29, 2024, U.S. Provisional Patent Application No. 63/636,352, filed Apr. 19, 2024, U.S. Provisional Patent Application No. 63/650,810, filed May 22, 2024, U.S. Provisional Patent Application No. 63/656,504, filed Jun. 5, 2024, U.S. Provisional Patent Application No. 63/665,947, filed Jun. 28, 2024, U.S. Provisional Patent Application No. 63/672,627, filed Jul. 17, 2024, U.S. Provisional Patent Application No. 63/681,620, filed Aug. 9, 2024, U.S. Provisional Patent Application No. 63/690,691, filed Sep. 4, 2024, and U.S. Provisional Patent Application No. 63/697,315, filed Sep. 20, 2024, the contents of which are hereby incorporated herein by reference in their entireties for all purposes.

### FIELD

The disclosure herein relates to the treatment of heart diseases, and compounds and compositions that may be used for treating heart diseases.

### BACKGROUND

A cardinal clinical feature common to numerous heart diseases is exercise intolerance. The evaluation of patients with HCM during the physiologic stress of exercise with a cardiopulmonary exercise test (CPET) can ascertain the extent to which these cardiospecific limitations impair exercise performance and their reversibility. CPET enables an objective and reproducible assessment of all stages of exercise performance. Peak oxygen uptake (pVO₂) and other exercise physiology metrics measured by CPET have already been shown to predict clinical events in certain heart diseases (Masri A, Pierson L M, Smedira N G, et al. Predictors of long-term outcomes in patients with hypertrophic cardiomyopathy undergoing cardiopulmonary stress testing and echocardiography. Am Heart J. 2015; 169 (5): 684-692.e1.).

The cardiac sarcomere is composed of a network of contractile and structural proteins that regulate cardiac muscle function. The components of the cardiac sarcomere present targets for the treatment of various cardiac diseases and conditions, for example by increasing contractility or facilitating complete relaxation to modulate systolic and diastolic function, respectively. The force and speed of cardiac muscle contraction is a major determinant of organ function and is modulated by the cyclical interactions of actin and myosin. Regulation of actin and myosin binding is determined by a network of myofilament regulatory proteins and the level of intracellular Ca²⁺. The troponin complex and tropomyosin are thin filament proteins which govern the availability of actin binding sites, and the essential and regulatory light chains, and myosin binding protein C modulate the position and mechanical properties of myosin.

Abnormalities in the cardiac sarcomere have been identified as the driving cause for a variety of cardiac diseases and conditions, such as hypertrophic cardiomyopathy (HCM) and heart failure with preserved ejection fraction (HFpEF). Mutations in the proteins of the sarcomere cause disease by rendering the cardiac muscle either ‘hyper’ or ‘hypo’ contractile. Modulators of the cardiac sarcomere can be used to rebalance contractility and stop or reverse the course of disease.

Current agents that target the cardiac sarcomere, such as inotropes (drugs that increase the contractile ability of the heart) are poorly selective for cardiac tissue, which leads to recognized adverse effects that limit their use. These adverse effects include cell damage caused by an increased rate of energy expenditure, exacerbation of relaxation abnormalities, and potential arrhythmogenic side effects that may result from increased cytosolic Ca++ and cyclic AMP concentrations in the inotropically stimulated myocardium. Given the limitations of current agents, new approaches are needed to improve cardiac function in HCM and HFpEF.

Cardiac myosin inhibitors (CMIs) have been developed as a therapeutic option for patients with oHCM by targeting the underlying etiopathology of the disease. They act by directly reducing excessive actin-myosin cross bridges at the level of the sarcomere and mitigate cardiac hypercontractility. Mavacamten has shown efficacy by improving peak oxygen uptake (pVO₂) and other CPET parameters (Cui H, Schaff H V., Olson T P, et al. Cardiopulmonary Exercise Test in Patients with Obstructive Hypertrophic Cardiomyopathy. J Thorac Cardiovasc Surg. May 2022.). Aficamten is a second generation CMI.

Patients suffering from heart diseases often receive multiple therapies. It is important to consider if and how new drugs may be co-administered with existing therapies. For example, while beta blockers have been shown to reduce symptom burden and improve hemodynamics, (Dybro, A, Rasmussen, T, Nielsen, R. et al. Randomized Trial of Metoprolol in Patients With Obstructive Hypertrophic Cardiomyopathy. J Am Coll Cardiol. 2021 December, 78 (25) 2505-2517), they can be associated with poor tolerability and are known to reduce heart rate in patients and therefore, parameters affected by heart rate are expected to be affected by beta blocker use (e.g., peak VO₂, METs, exercise time). Notably, when used in combination with mavacamten, beta blockers were shown to affect various clinical parameters negatively such as pVO₂, METs and exercise time (European Journal of Heart Failure (2023), 25, 260-270). There is a need for treatments that can improve the life of heart disease patients and can be effectively combined with beta blockers.

### BRIEF SUMMARY

Methods and compositions for improving a heart rate dependent CPET (cardiopulmonary exercise testing) parameter in a patient in need thereof, comprising administering to the patient a therapeutically effective amount of aficamten, or a pharmaceutically acceptable salt thereof, and wherein the patient is concurrently undergoing a treatment with a beta blocker, are provided therein.

Methods and compositions for improving a heart rate dependent CPET (cardiopulmonary exercise testing) parameter in a patient in need thereof, comprising administering to the patient a therapeutically effective amount of aficamten, or a pharmaceutically acceptable salt thereof, and wherein the patient was administered a beta blocker within at least 1 week, at least 2 weeks, at least 3 weeks, or at least 4 weeks prior to administration of the therapeutically effective amount of aficamten, or the pharmaceutically acceptable salt thereof, are provided therein.

Methods and compositions for improving a heart rate dependent CPET (cardiopulmonary exercise testing) parameter in a patient in need thereof, comprising administering to the patient a therapeutically effective amount of aficamten, or a pharmaceutically acceptable salt thereof, resulting in an improvement in the heart rate dependent CPET (cardiopulmonary exercise testing) parameter; wherein the patient is concurrently undergoing a treatment with a beta blocker; and wherein the improvement in the heart rate dependent CPET (cardiopulmonary exercise testing) parameter is not substantially diminished by the treatment with the beta blocker, are provided therein.

Methods and compositions for improving a heart rate dependent CPET (cardiopulmonary exercise testing) parameter in a patient in need thereof, comprising administering to the patient a therapeutically effective amount of aficamten, or a pharmaceutically acceptable salt thereof, and wherein the patient has been undergoing a treatment with a beta blocker prior to administration of aficamten, or the pharmaceutically acceptable salt thereof, and the treatment with the beta blocker remains unchanged concurrently to the administration of aficamten, or the pharmaceutically acceptable salt thereof, are provided therein.

Methods and compositions for improving a heart rate dependent CPET (cardiopulmonary exercise testing) parameter in a patient in need thereof, comprising administering to the patient a therapeutically effective amount of aficamten, or a pharmaceutically acceptable salt thereof, and wherein treatment with a beta blocker is not contraindicated for the patient, are provided therein.

Methods and compositions for improving a heart rate dependent CPET (cardiopulmonary exercise testing) parameter in a patient in need thereof, comprising administering to the patient a beta blocker, or a pharmaceutically acceptable salt thereof, and wherein the patient is concurrently undergoing treatment with aficamten, or a pharmaceutically acceptable salt thereof, are provided therein.

Methods and compositions for improving a heart rate dependent CPET (cardiopulmonary exercise testing) parameter in a patient in need thereof, comprising administering to the patient a therapeutically effective amount of aficamten, or a pharmaceutically acceptable salt thereof, and a beta blocker, are provided therein.

Methods and compositions for treating a patient in need thereof, comprising administering to the patient a therapeutically effective amount of aficamten, or a pharmaceutically acceptable salt thereof, and a beta blocker, wherein (a) the co-administration of the beta blocker with aficamten, or a pharmaceutically acceptable salt thereof, does not cause chronotropic incompetence in the patient, and/or (b) the co-administration of the beta blocker with aficamten, or a pharmaceutically acceptable salt thereof, does not impact the ability of the patient's heart to increase its rate commensurate with increased activity or demand, and/or (c) the patient's heart maintains its ability to increase its rate commensurate with increased activity or demand compared to administration of aficamten, or a pharmaceutically acceptable salt thereof, without a beta blocker, are provided therein.

Methods and compositions for improving heart rate reserve in a patient in need thereof, comprising administering to the patient a therapeutically effective amount of aficamten, or a pharmaceutically acceptable salt thereof, and wherein the patient is concurrently undergoing a treatment with a beta blocker, are provided therein.

Methods and compositions for improving heart rate reserve in a patient in need thereof, comprising administering to the patient a therapeutically effective amount of aficamten, or a pharmaceutically acceptable salt thereof, and wherein the patient was administered a beta blocker within at least 1 week, at least 2 weeks, at least 3 weeks, or at least 4 weeks prior to administration of the therapeutically effective amount of aficamten, or the pharmaceutically acceptable salt thereof, are provided therein.

Methods and compositions for improving heart rate reserve in a patient in need thereof, comprising administering to the patient a therapeutically effective amount of aficamten, or a pharmaceutically acceptable salt thereof, resulting in an improvement in heart rate reserve; wherein the patient is concurrently undergoing a treatment with a beta blocker; and wherein the improvement in heart rate reserve is not substantially diminished by the treatment with the beta blocker, are provided therein.

Methods and compositions for improving heart rate reserve in a patient in need thereof, comprising administering to the patient a therapeutically effective amount of aficamten, or a pharmaceutically acceptable salt thereof, wherein the patient has been undergoing a treatment with a beta blocker prior to administration of aficamten, or the pharmaceutically acceptable salt thereof, and the treatment with the beta blocker remains unchanged concurrently to the administration of aficamten, or the pharmaceutically acceptable salt thereof.

Methods and compositions for improving heart rate reserve in a patient in need thereof, comprising administering to the patient a therapeutically effective amount of aficamten, or a pharmaceutically acceptable salt thereof, and wherein the patient has been undergoing a treatment with a beta blocker prior to administration of aficamten, or the pharmaceutically acceptable salt thereof, and the treatment with the beta blocker is not changed for at least 1 week, at least 2 weeks, at least 3 weeks, or at least 4 weeks during the concurrent administration of aficamten, or the pharmaceutically acceptable salt thereof, are provided therein.

Methods and compositions for improving heart rate reserve in a patient in need thereof, comprising administering to the patient a therapeutically effective amount of aficamten, or a pharmaceutically acceptable salt thereof, and wherein treatment with a beta blocker is not contraindicated for the patient, are provided therein.

Methods and compositions for improving heart rate reserve in a patient in need thereof, comprising administering to the patient a beta blocker, or a pharmaceutically acceptable salt thereof, and wherein the patient is concurrently undergoing a treatment with aficamten, or a pharmaceutically acceptable salt thereof, are provided therein.

Methods and compositions for improving heart rate reserve in a patient in need thereof, comprising administering to the patient a therapeutically effective amount of aficamten, or a pharmaceutically acceptable salt thereof, and a beta blocker, are provided therein.

Methods and compositions for improving peak metabolic equivalents (METs) in a patient in need thereof, comprising administering to the patient a therapeutically effective amount of aficamten, or a pharmaceutically acceptable salt thereof, and wherein the patient is concurrently undergoing a treatment with a beta blocker, are provided therein.

Methods and compositions for improving peak metabolic equivalents (METs) in a patient in need thereof, comprising administering to the patient a therapeutically effective amount of aficamten, or a pharmaceutically acceptable salt thereof, and wherein the patient was administered a beta blocker within at least 1 week, at least 2 weeks, at least 3 weeks, or at least 4 weeks prior to administration of the therapeutically effective amount of aficamten, or the pharmaceutically acceptable salt thereof, are provided therein.

Methods and compositions for improving peak metabolic equivalents (METs) in a patient in need thereof, comprising administering to the patient a therapeutically effective amount of aficamten, or a pharmaceutically acceptable salt thereof, resulting in an improvement in peak metabolic equivalents (METs); wherein the patient is concurrently undergoing a treatment with a beta blocker; and wherein the improvement in peak metabolic equivalents (METs) is not substantially diminished by the treatment with the beta blocker, are provided therein.

Methods and compositions for improving peak metabolic equivalents (METs) in a patient in need thereof, comprising administering to the patient a therapeutically effective amount of aficamten, or a pharmaceutically acceptable salt thereof, wherein the patient has been undergoing a treatment with a beta blocker prior to administration of aficamten, or the pharmaceutically acceptable salt thereof, and the treatment with the beta blocker remains unchanged concurrently to the administration of aficamten, or the pharmaceutically acceptable salt thereof.

Methods and compositions for improving peak metabolic equivalents (METs) in a patient in need thereof, comprising administering to the patient a therapeutically effective amount of aficamten, or a pharmaceutically acceptable salt thereof, and wherein the patient has been undergoing a treatment with a beta blocker prior to administration of aficamten, or the pharmaceutically acceptable salt thereof, and the treatment with the beta blocker is not changed for at least 1 week, at least 2 weeks, at least 3 weeks, or at least 4 weeks during the concurrent administration of aficamten, or the pharmaceutically acceptable salt thereof, are provided therein.

Methods and compositions for improving peak metabolic equivalents (METs) in a patient in need thereof, comprising administering to the patient a therapeutically effective amount of aficamten, or a pharmaceutically acceptable salt thereof, and wherein treatment with a beta blocker is not contraindicated for the patient, are provided therein.

Methods and compositions for improving peak metabolic equivalents (METs) in a patient in need thereof, comprising administering to the patient a beta blocker, or a pharmaceutically acceptable salt thereof, and wherein the patient is concurrently undergoing a treatment with aficamten, or a pharmaceutically acceptable salt thereof, are provided therein.

Methods and compositions for improving peak metabolic equivalents (METs) in a patient in need thereof, comprising administering to the patient a therapeutically effective amount of aficamten, or a pharmaceutically acceptable salt thereof, and a beta blocker, are provided therein.

Methods and compositions for improving exercise duration in a patient in need thereof, comprising administering to the patient a therapeutically effective amount of aficamten, or a pharmaceutically acceptable salt thereof, and wherein the patient is concurrently undergoing a treatment with a beta blocker, are provided therein.

Methods and compositions for improving exercise duration in a patient in need thereof, comprising administering to the patient a therapeutically effective amount of aficamten, or a pharmaceutically acceptable salt thereof, and wherein the patient was administered a beta blocker within at least 1 week, at least 2 weeks, at least 3 weeks, or at least 4 weeks prior to administration of the therapeutically effective amount of aficamten, or the pharmaceutically acceptable salt thereof, are provided therein.

Methods and compositions for improving exercise duration in a patient in need thereof, comprising administering to the patient a therapeutically effective amount of aficamten, or a pharmaceutically acceptable salt thereof, resulting in an improvement in exercise duration; wherein the patient is concurrently undergoing a treatment with a beta blocker; and wherein the improvement in exercise duration is not substantially diminished by the treatment with the beta blocker, are provided therein.

Methods and compositions for improving exercise duration in a patient in need thereof, comprising administering to the patient a therapeutically effective amount of aficamten, or a pharmaceutically acceptable salt thereof, wherein the patient has been undergoing a treatment with a beta blocker prior to administration of aficamten, or the pharmaceutically acceptable salt thereof, and the treatment with the beta blocker remains unchanged concurrently to the administration of aficamten, or the pharmaceutically acceptable salt thereof.

Methods and compositions for improving exercise duration in a patient in need thereof, comprising administering to the patient a therapeutically effective amount of aficamten, or a pharmaceutically acceptable salt thereof, and wherein the patient has been undergoing a treatment with a beta blocker prior to administration of aficamten, or the pharmaceutically acceptable salt thereof, and the treatment with the beta blocker is not changed for at least 1 week, at least 2 weeks, at least 3 weeks, or at least 4 weeks during the concurrent administration of aficamten, or the pharmaceutically acceptable salt thereof, are provided therein.

Methods and compositions for improving exercise duration in a patient in need thereof, comprising administering to the patient a therapeutically effective amount of aficamten, or a pharmaceutically acceptable salt thereof, and wherein treatment with a beta blocker is not contraindicated for the patient, are provided therein.

Methods and compositions for improving exercise duration in a patient in need thereof, comprising administering to the patient a beta blocker, or a pharmaceutically acceptable salt thereof, and wherein the patient is concurrently undergoing a treatment with aficamten, or a pharmaceutically acceptable salt thereof, are provided therein.

Methods and compositions for improving exercise duration in a patient in need thereof, comprising administering to the patient a therapeutically effective amount of aficamten, or a pharmaceutically acceptable salt thereof, and a beta blocker, are provided therein.

Methods and compositions for improving one or more of integrated 2 component Z-score metric, pVO₂, peak workload, peak METs, peak circulatory power, exercise duration, peak respiratory exchange ratio, heart rate reserve, peak heart rate, O₂ pulse at peak exercise, peak proportionate pulse pressure, proportionate pulse pressure at rest, ventilatory efficiency pre-anaerobic threshold, ventilatory efficiency throughout exercise, ventilatory power, ventilatory anaerobic threshold, and acrobic efficiency in a patient in need thereof, comprising administering to the patient a therapeutically effective amount of aficamten, or a pharmaceutically acceptable salt thereof, and wherein the patient is concurrently undergoing a treatment with a beta blocker, are provided therein.

Methods and compositions for improving one or more of integrated 2 component Z-score metric, pVO₂, peak workload, peak METs, peak circulatory power, exercise duration, peak respiratory exchange ratio, heart rate reserve, peak heart rate, O₂ pulse at peak exercise, peak proportionate pulse pressure, proportionate pulse pressure at rest, ventilatory efficiency pre-anaerobic threshold, ventilatory efficiency throughout exercise, ventilatory power, ventilatory anaerobic threshold, and aerobic efficiency in a patient in need thereof, comprising administering to the patient a therapeutically effective amount of aficamten, or a pharmaceutically acceptable salt thereof, and wherein the patient was administered a beta blocker within at least 1 week, at least 2 weeks, at least 3 weeks, or at least 4 weeks prior to administration of the therapeutically effective amount of aficamten, or the pharmaceutically acceptable salt thereof, are provided therein.

Methods and compositions for improving one or more of integrated 2 component Z-score metric, pVO₂, peak workload, peak METs, peak circulatory power, exercise duration, peak respiratory exchange ratio, heart rate reserve, peak heart rate, O₂ pulse at peak exercise, peak proportionate pulse pressure, proportionate pulse pressure at rest, ventilatory efficiency pre-anaerobic threshold, ventilatory efficiency throughout exercise, ventilatory power, ventilatory anaerobic threshold, and aerobic efficiency in a patient in need thereof, comprising administering to the patient a therapeutically effective amount of aficamten, or a pharmaceutically acceptable salt thereof, resulting in an improvement in one or more of integrated 2 component Z-score metric, pVO₂, peak workload, peak METs, peak circulatory power, exercise duration, peak respiratory exchange ratio, heart rate reserve, peak heart rate, O₂ pulse at peak exercise, peak proportionate pulse pressure, proportionate pulse pressure at rest, ventilatory efficiency pre-anaerobic threshold, ventilatory efficiency throughout exercise, ventilatory power, ventilatory anaerobic threshold, and aerobic efficiency; wherein the patient is concurrently undergoing a treatment with a beta blocker; and wherein the improvement in one or more of integrated 2 component Z-score metric, pVO₂, peak workload, peak METs, peak circulatory power, exercise duration, peak respiratory exchange ratio, heart rate reserve, peak heart rate, O₂ pulse at peak exercise, peak proportionate pulse pressure, proportionate pulse pressure at rest, ventilatory efficiency pre-anaerobic threshold, ventilatory efficiency throughout exercise, ventilatory power, ventilatory anaerobic threshold, and aerobic efficiency is not substantially diminished by the treatment with the beta blocker, are provided therein.

Methods and compositions for improving one or more of integrated 2 component Z-score metric, pVO₂, peak workload, peak METs, peak circulatory power, exercise duration, peak respiratory exchange ratio, heart rate reserve, peak heart rate, O₂ pulse at peak exercise, peak proportionate pulse pressure, proportionate pulse pressure at rest, ventilatory efficiency pre-anaerobic threshold, ventilatory efficiency throughout exercise, ventilatory power, ventilatory anaerobic threshold, and acrobic efficiency in a patient in need thereof, comprising administering to the patient a therapeutically effective amount of aficamten, or a pharmaceutically acceptable salt thereof, wherein the patient has been undergoing a treatment with a beta blocker prior to administration of aficamten, or the pharmaceutically acceptable salt thereof, and the treatment with the beta blocker remains unchanged concurrently to the administration of aficamten, or the pharmaceutically acceptable salt thereof.

Methods and compositions for improving one or more of integrated 2 component Z-score metric, pVO₂, peak workload, peak METs, peak circulatory power, exercise duration, peak respiratory exchange ratio, heart rate reserve, peak heart rate, O₂ pulse at peak exercise, peak proportionate pulse pressure, proportionate pulse pressure at rest, ventilatory efficiency pre-anaerobic threshold, ventilatory efficiency throughout exercise, ventilatory power, ventilatory anaerobic threshold, and aerobic efficiency in a patient in need thereof, comprising administering to the patient a therapeutically effective amount of aficamten, or a pharmaceutically acceptable salt thereof, and wherein the patient has been undergoing a treatment with a beta blocker prior to administration of aficamten, or the pharmaceutically acceptable salt thereof, and the treatment with the beta blocker is not changed for at least 1 week, at least 2 weeks, at least 3 weeks, or at least 4 weeks during the concurrent administration of aficamten, or the pharmaceutically acceptable salt thereof, are provided therein.

Methods and compositions for improving one or more of integrated 2 component Z-score metric, pVO₂, peak workload, peak METs, peak circulatory power, exercise duration, peak respiratory exchange ratio, heart rate reserve, peak heart rate, O₂ pulse at peak exercise, peak proportionate pulse pressure, proportionate pulse pressure at rest, ventilatory efficiency pre-anaerobic threshold, ventilatory efficiency throughout exercise, ventilatory power, ventilatory anaerobic threshold, and acrobic efficiency in a patient in need thereof, comprising administering to the patient a therapeutically effective amount of aficamten, or a pharmaceutically acceptable salt thereof, and wherein treatment with a beta blocker is not contraindicated for the patient, are provided therein.

Methods and compositions for improving one or more of integrated 2 component Z-score metric, pVO₂, peak workload, peak METs, peak circulatory power, exercise duration, peak respiratory exchange ratio, heart rate reserve, peak heart rate, O₂ pulse at peak exercise, peak proportionate pulse pressure, proportionate pulse pressure at rest, ventilatory efficiency pre-anaerobic threshold, ventilatory efficiency throughout exercise, ventilatory power, ventilatory anaerobic threshold, and aerobic efficiency in a patient in need thereof, comprising administering to the patient a beta blocker, or a pharmaceutically acceptable salt thereof, and wherein the patient is concurrently undergoing a treatment with aficamten, or a pharmaceutically acceptable salt thereof, are provided therein.

Methods and compositions for improving one or more of integrated 2 component Z-score metric, pVO₂, peak workload, peak METs, peak circulatory power, exercise duration, peak respiratory exchange ratio, heart rate reserve, peak heart rate, O₂ pulse at peak exercise, peak proportionate pulse pressure, proportionate pulse pressure at rest, ventilatory efficiency pre-anaerobic threshold, ventilatory efficiency throughout exercise, ventilatory power, ventilatory anaerobic threshold, and aerobic efficiency, in a patient in need thereof, comprising administering to the patient a therapeutically effective amount of aficamten, or a pharmaceutically acceptable salt thereof, and a beta blocker, are provided therein.

Methods and compositions for improving peak oxygen uptake (pVO₂) in a patient in need thereof, comprising administering to the patient a therapeutically effective amount of aficamten, or a pharmaceutically acceptable salt thereof, and wherein the patient is concurrently undergoing a treatment with a beta blocker, are provided therein.

Methods and compositions for improving peak oxygen uptake (pVO₂) in a patient in need thereof, comprising administering to the patient a therapeutically effective amount of aficamten, or a pharmaceutically acceptable salt thereof, and wherein the patient was administered a beta blocker within at least 1 week, at least 2 weeks, at least 3 weeks, or at least 4 weeks prior to administration of the therapeutically effective amount of aficamten, or the pharmaceutically acceptable salt thereof, are provided therein.

Methods and compositions for improving peak oxygen uptake (pVO₂) in a patient in need thereof, comprising administering to the patient a therapeutically effective amount of aficamten, or a pharmaceutically acceptable salt thereof, resulting in an improvement in peak oxygen uptake (pVO₂); wherein the patient is concurrently undergoing a treatment with a beta blocker; and wherein the improvement in peak oxygen uptake (pVO₂) is not substantially diminished by the treatment with the beta blocker, are provided therein.

Methods and compositions for improving peak oxygen uptake (pVO₂) in a patient in need thereof, comprising administering to the patient a therapeutically effective amount of aficamten, or a pharmaceutically acceptable salt thereof, wherein the patient has been undergoing a treatment with a beta blocker prior to administration of aficamten, or the pharmaceutically acceptable salt thereof, and the treatment with the beta blocker remains unchanged concurrently to the administration of aficamten, or the pharmaceutically acceptable salt thereof.

Methods and compositions for improving peak oxygen uptake (pVO₂) in a patient in need thereof, comprising administering to the patient a therapeutically effective amount of aficamten, or a pharmaceutically acceptable salt thereof, and wherein the patient has been undergoing a treatment with a beta blocker prior to administration of aficamten, or the pharmaceutically acceptable salt thereof, and the treatment with the beta blocker is not changed for at least 1 week, at least 2 weeks, at least 3 weeks, or at least 4 weeks during the concurrent administration of aficamten, or the pharmaceutically acceptable salt thereof, are provided therein.

Methods and compositions for improving peak oxygen uptake (pVO₂) in a patient in need thereof, comprising administering to the patient a therapeutically effective amount of aficamten, or a pharmaceutically acceptable salt thereof, and wherein treatment with a beta blocker is not contraindicated for the patient, are provided therein.

Methods and compositions for improving peak oxygen uptake (pVO₂) in a patient in need thereof, comprising administering to the patient a beta blocker, or a pharmaceutically acceptable salt thereof, and wherein the patient is concurrently undergoing a treatment with aficamten, or a pharmaceutically acceptable salt thereof, are provided therein.

Methods and compositions for improving peak oxygen uptake (pVO₂) in a patient in need thereof, comprising administering to the patient a therapeutically effective amount of aficamten, or a pharmaceutically acceptable salt thereof, and a beta blocker, are provided therein.

Methods and compositions for improving heart rate reserve in a patient in need thereof, comprising administering to the patient a therapeutically effective amount of aficamten, or a pharmaceutically acceptable salt thereof, are provided therein.

Methods and compositions for improving peak metabolic equivalents (METs) in a patient in need thereof, comprising administering to the patient a therapeutically effective amount of aficamten, or a pharmaceutically acceptable salt thereof, are provided therein.

Methods and compositions for improving exercise duration in a patient in need thereof, comprising administering to the patient a therapeutically effective amount of aficamten, or a pharmaceutically acceptable salt thereof, are provided therein.

Methods and compositions for improving one or more of integrated 2 component Z-score metric, pVO₂, peak workload, peak METs, peak circulatory power, exercise duration, peak respiratory exchange ratio, heart rate reserve, peak heart rate, O₂ pulse at peak exercise, peak proportionate pulse pressure, proportionate pulse pressure at rest, ventilatory efficiency pre-anaerobic threshold, ventilatory efficiency throughout exercise, ventilatory power, ventilatory anaerobic threshold, and aerobic efficiency, in a patient in need thereof, comprising administering to the patient a therapeutically effective amount of aficamten, or a pharmaceutically acceptable salt thereof, are provided therein.

Methods and compositions for improving peak oxygen uptake (pVO₂) in a patient in need thereof, comprising administering to the patient a therapeutically effective amount of aficamten, or a pharmaceutically acceptable salt thereof, are provided therein.

In some implementations of the methods disclosed herein, the patient receives an additional medical therapy.

In some implementations of the methods disclosed herein, the patient has a heart disease. In some embodiments, the heart disease is hypertrophic cardiomyopathy (HCM). In some embodiments, the HCM is obstructive or nonobstructive or is associated with a sarcomeric and/or non-sarcomeric mutation. In some embodiments, the heart disease is heart failure with preserved ejection fraction (HFpEF). In some embodiments, the heart disease is selected from the group consisting of diastolic dysfunction, primary or secondary restrictive cardiomyopathy, myocardial infarction, angina pectoris, left ventricular outflow tract obstruction, hypertensive heart disease, congenital heart disease, cardiac ischemia, coronary heart disease, diabetic heart disease, congestive heart failure, right heart failure, cardiorenal syndrome, and infiltrative cardiomyopathy. In some embodiments, the heart disease is or is related to one or more conditions selected from the group consisting of cardiac senescence, diastolic dysfunction due to aging, left ventricular hypertrophy and concentric left ventricular remodeling.

Methods and compositions for accelerating oxygen uptake recovery (VO₂Rec) in a patient in need thereof, comprising administering to the patient a therapeutically effective amount of aficamten or a pharmaceutically acceptable salt thereof, are provided therein.

Methods and compositions for reducing or resolving left ventricular hypertrophy (LVH) in a patient in need thereof, comprising administering to the patient a therapeutically effective amount of aficamten or a pharmaceutically acceptable salt thereof, are provided therein.

Methods and compositions for decreasing maximal wall thickness (MWT) in a patient in need thereof, comprising administering to the patient a therapeutically effective amount of aficamten or a pharmaceutically acceptable salt thereof, are provided therein.

Methods and compositions for treating a disease or condition in a patient in need thereof, comprising administering to the patient a therapeutically effective amount of aficamten, or a pharmaceutically acceptable salt thereof, and optionally a beta blocker;
wherein the disease or condition is selected from the group consisting of:
   (a) hypertrophic cardiomyopathy (HCM);
   (b) a heart disease selected from the group consisting of diastolic dysfunction, primary or secondary restrictive cardiomyopathy, myocardial infarction and angina pectoris, left ventricular outflow tract obstruction, hypertensive heart disease, congenital heart disease, cardiac ischemia, coronary heart disease, diabetic heart disease, congestive heart failure, right heart failure, cardiorenal syndrome, and infiltrative cardiomyopathy; and
   (c) a heart disease that is or is related to one or more conditions selected from the group consisting of cardiac senescence, diastolic dysfunction due to aging, left ventricular hypertrophy and concentric left ventricular remodeling;
and
wherein the method improves one or more parameter in the patient wherein each of the one or more parameter is selected from the group consisting of:
   (1) heart rate reserve;
   (2) peak metabolic equivalents (METs);
   (3) exercise duration;
   (4) peak oxygen uptake (pVO₂); and
   (5) one or more of integrated 2 component Z-score metric, peak workload, peak circulatory power, peak respiratory exchange ratio, peak heart rate, O₂ pulse at peak exercise, peak proportionate pulse pressure, proportionate pulse pressure at rest, ventilatory efficiency pre-anaerobic threshold, ventilatory efficiency throughout exercise, ventilatory power, ventilatory anaerobic threshold, and aerobic efficiency in the patient.

In some implementations of the methods disclosed herein, the therapeutically effective amount of aficamten, or a pharmaceutically acceptable salt thereof, is selected by titrating a daily dose of aficamten, or a pharmaceutically acceptable salt thereof, administered to the patient.

In some implementations of the methods disclosed herein, aficamten, or a pharmaceutically acceptable salt thereof is administered at a daily dose of about 5 mg to about 20 mg.

In some implementations of the methods disclosed herein, aficamten, or a pharmaceutically acceptable salt thereof, is administered once daily.

In some implementations of the methods disclosed herein, the methods comprise administering aficamten, or a pharmaceutically acceptable salt thereof, for at least about 2, at least about 3, at least about 4, at least about 5, at least about 6, at least about 7, at least about 8, at least about 9, at least about 10, at least about 11, at least about 12, at least about 16, at least about 20, at least about 24, at least about 28, at least about 32, at least about 36, at least about 40, at least about 44, or at least about 48 weeks.

In some implementations of the methods disclosed herein, the methods result in an improvement for at least about 2, at least about 3, at least about 4, at least about 5, at least about 6, at least about 7, at least about 8, at least about 9, at least about 10, at least about 11, at least about 12, at least about 16, at least about 20, at least about 24, at least about 28, at least about 32, at least about 36, at least about 40, at least about 44, or at least about 48 weeks after beginning administering aficamten, or a pharmaceutically acceptable salt thereof.

In some implementations of the methods disclosed herein, aficamten or pharmaceutically acceptable salt thereof is administered as a tablet.

In some implementations of the methods disclosed herein, aficamten or pharmaceutically acceptable salt thereof comprises one or more of polymorphic Form I, Form II, Form III, Form IV, Form V, and Form VI of aficamten.

### BRIEF DESCRIPTION OF THE DRAWINGS

FIG. 1 shows the design of an exemplary Phase 3 clinical trial for aficamten (Example 8).

FIG. 2 shows responder analyses of categorical change in peak oxygen uptake with aficamten versus placebo at 24 weeks.

FIGS. 3A, 3B, and 3C show clinical response measures in patients with obstructive HCM from baseline to week 24. FIG. 3A shows the percentage of patients having a complete hemodynamic response, defined as rest outflow gradient <30 mmHg and Valsalva outflow gradient <50 mmHg. FIG. 3B shows the percentage of patients having a symptomatic response of ≥1 improvement in NYHA class and/or ≥10 increase KCCQ-CSS. FIG. 3C shows the percentage of patients with ≥50% reduction in NT-proBNP relative to baseline.

FIG. 4 shows the percentage of patients that achieved 0, 1, 2, 3, or 4 clinical efficacy outcomes at the end of treatment.

FIG. 5 shows a schematic description of the global efficacy of aficamten in treating oHCM.

FIGS. 6A and 6B demonstrate the treatment benefit of aficamten across subgroups for the KCCQ-OS and SAQ-SS, respectively.

### DETAILED DESCRIPTION

Described herein is a cardiac myosin inhibitor aficamten and various methods.

Aficamten is a small molecule cardiac myosin inhibitor having the structure shown below.

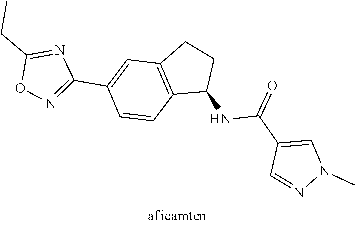 [SMILES: CCc1nc(-c2ccc3c(c2)CC[C@H]3NC(=O)c2cnn(C)c2)no1]

The chemical name of aficamten is (R)—N-(5-(5-ethyl-1,2,4-oxadiazol-3-yl)-2,3-dihydro-1H-inden-1-yl)-1-methyl-1H-pyrazole-4-carboxamide. Aficamten is also referred to as CK-3773274 or CK-274. The small molecule inhibitor may be, for example, orally administered to a patient for the treatment of obstructive hypertrophic cardiomyopathy.

Aficamten has been described in WO 2019/144041 and U.S. Pat. No. 10,836,755, the disclosures of which are incorporated by reference herein. Aficamten, or a pharmaceutically acceptable salt thereof, may be obtained following the methods described therein. Aficamten used in the disclosed methods can be present as a pharmaceutically acceptable salt, solvate, hydrate, polymorph, or combination thereof, and can be formulated into any suitable pharmaceutical formulation. Aficamten can also be present in its free base form. Polymorphs of aficamten have been described in WO 2021/011807 and U.S. Patent Publication US-2022-0315571-A1, the disclosures of which are incorporated by reference herein. Formulations of aficamten have been described in WO 2021/011808 and U.S. Patent Publication US-2022-0265612-A1, the disclosures of which are incorporated by reference herein. Aficamten was designed to reduce the hypercontractility that is associated with hypertrophic cardiomyopathy (HCM). Without being bound by theory, in preclinical models, aficamten reduces myocardial contractility by binding directly to cardiac myosin at a distinct and selective allosteric binding site, thereby preventing myosin from entering a force producing state. Aficamten reduces the number of active actin-myosin cross bridges during each cardiac cycle and consequently reduces myocardial contractility. This mechanism of action may be therapeutically effective in conditions characterized by excessive hypercontractility, such as HCM (e.g., obstructive HCM, also referred to as oHCM). Aficamten has been the object of several clinical trials and has been further described in WO 2023/288324 and in U.S. Patent Publication US-2023-0058927-A1, WO 2024/020468 and U.S. Patent Publication US-2024-0091203-A1, and WO 2024/031016 and U.S. Patent Publication US-2024-0115554-A1, the disclosures of which are incorporated by reference herein.

### Definitions

As used in the present specification, the following words and phrases are generally intended to have the meanings as set forth below, except to the extent that the context in which they are used indicates otherwise.

Throughout this application, unless the context indicates otherwise, references to aficamten include amorphous forms thereof or polymorphs thereof, including any one of polymorphic Forms I, II, III, IV, V, or VI as described herein, or a mixture thereof.

Reference to “about” a value or parameter herein includes (and describes) that value or parameter per se, and any value or parameter 5% above or 5% below said parameter. For example, description to “about X” includes description of “X” and “X+/−5%”. For example, a daily dose of “about 5 mg” includes “5 mg+/−5%”, which includes dose of 4.75 mg, 5.25 mg, or any amount therebetween.

“NYHA classification” or “NYHA class” refers to the New York Heart Association functional classification of heart failure symptoms. Descriptions of each of NYHA classes I, II, III, and IV can be found in “Classes of Heart Failure”, American Heart Association, https://www.heart.org/en/health-topics/heart-failure/what-is-heart-failure/classes-of-heart-failure, adapted from: 1) Dolgin M, Association N Y H, Fox A C, Gorlin R, Levin R I, New York Heart Association. Criteria Committee. “Nomenclature and criteria for diagnosis of diseases of the heart and great vessels”. 9th ed. Boston, MA: Lippincott Williams and Wilkins; Mar. 1, 1994; and 2) Criteria Committee, New York Heart Association, Inc. Diseases of the Heart and Blood Vessels. Nomenclature and Criteria for diagnosis, 6th edition Boston, Little, Brown and Co. 1964, p 114. Briefly, NYHA class I indicates that the patient has no limitation in ordinary physical activity (e.g., shortness of breath when walking, climbing stairs). NYHA class II indicates that the patient has mild symptoms (e.g., mild shortness of breath and/or angina) and slight limitation during ordinary activity. NYHA class III indicates that the patient has in activity due to symptoms, even during less-than-ordinary activity (e.g., walking short distances [20-100 m]); comfortable only at rest. NYHA class IV indicates that the patient has severe limitations, experiences symptoms even while at rest; mostly bedbound participants.

The term “pharmaceutically acceptable salt” refers to a salt of any of the compounds herein that are known to be non-toxic and are commonly used in the pharmaceutical literature. In some embodiments, the pharmaceutically acceptable salt of a compound retains the biological effectiveness of the compounds described herein and are not biologically or otherwise undesirable. Examples of pharmaceutically acceptable salts can be found in Berge et al., Pharmaceutical Salts, J. Pharmaceutical Sciences, January 1977, 66 (1), 1-19. Pharmaceutically acceptable acid addition salts can be formed with inorganic acids and organic acids. Inorganic acids from which salts can be derived include, for example, hydrochloric acid, hydrobromic acid, sulfuric acid, nitric acid, and phosphoric acid. Organic acids from which salts can be derived include, for example, acetic acid, propionic acid, glycolic acid, pyruvic acid, lactic acid, oxalic acid, malic acid, maleic acid, malonic acid, succinic acid, fumaric acid, tartaric acid, citric acid, benzoic acid, cinnamic acid, mandelic acid, methanesulfonic acid, ethanesulfonic acid, 2-hydroxyethylsulfonic acid, p-toluenesulfonic acid, stearic acid, and salicylic acid.

Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, and aluminum. Organic bases from which salts can be derived include, for example, primary, secondary, and tertiary amines; substituted amines including naturally occurring substituted amines; cyclic amines; and basic ion exchange resins. Examples of organic bases include isopropylamine, trimethylamine, diethylamine, triethylamine, tripropylamine, and ethanolamine. In some embodiments, the pharmaceutically acceptable base addition salt is selected from ammonium, potassium, sodium, calcium, and magnesium salts.

If the compound described herein is obtained as an acid addition salt, the free base can be obtained by basifying a solution of the acid salt. Conversely, if the compound is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds (see, e.g., Berge et al., Pharmaceutical Salts, J. Pharmaceutical Sciences, January 1977, 66 (1), 1-19). Those skilled in the art will recognize various synthetic methodologies that may be used to prepare pharmaceutically acceptable addition salts.

The term “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in pharmaceutical compositions is contemplated. Supplementary active ingredients can also be incorporated into the pharmaceutical compositions.

The terms “patient,” “individual,” and “subject” refer to an animal, such as a mammal. Mammals include, for example, mice, rats, dogs, cats, pigs, sheep, horses, cows, and humans. In some embodiments, the patient or subject is a human, for example a human that has been or will be the object of treatment, observation, or experiment. The compounds, compositions and methods described herein can be useful in both human therapy and veterinary applications.

The term “therapeutically effective amount” or “effective amount” refers to that amount of a compound disclosed and/or described herein that is sufficient to affect treatment, as defined herein, when administered to a patient in need of such treatment. A therapeutically effective amount of a compound may be an amount sufficient to treat a disease responsive to modulation of the cardiac sarcomere. The therapeutically effective amount will vary depending upon, for example, the subject and disease condition being treated, the weight and age of the subject, the severity of the disease condition, the particular compound, the dosing regimen to be followed, timing of administration, the manner of administration, all of which can readily be determined by one of ordinary skill in the art. The therapeutically effective amount may be ascertained experimentally, for example by assaying blood concentration of the chemical entity, or theoretically, by calculating bioavailability.

“Treatment” (and related terms, such as “treat”, “treated”, “treating”) includes one or more of: inhibiting a disease or disorder; slowing or arresting the development of clinical symptoms of a disease or disorder; and/or relieving a disease or disorder (i.e., causing relief from or regression of clinical symptoms). The term covers both complete and partial reduction or prevention of the condition or disorder, and complete or partial reduction of clinical symptoms of a disease or disorder. Thus, compounds described and/or disclosed herein may prevent an existing disease or disorder from worsening, assist in the management of the disease or disorder, or reduce or eliminate the disease or disorder.

Reference to any dose amount of a compound or pharmaceutically acceptable salt thereof described herein (e.g., 5 mg, 10 mg, 20 mg, etc. of aficamten) refers to the amount (i.e., equivalent mass) of said compound without any salt.

The term “chronotropic incompetence” refers to a patient's inability to increase its heart rate commensurate with increased activity or demand.

The term “substantially diminished by the treatment with the beta blocker” is understood to mean that the magnitude of the improvement of the clinical parameter is reduced in a statistically significant manner when observing a clinical population (e.g. in a phase 3 clinical trial) comprising patients undergoing a beta blocker treatment and patients not undergoing a beta blocker treatment and comparing the magnitude of the improvement of the clinical parameter in the two subpopulations.

### Improvement of Cardiac Parameters and Treatment of Various Diseases

As further described herein a therapeutically effective amount of aficamten, or a pharmaceutically acceptable salt thereof, may be administered to a patient for improving a heart rate dependent CPET (cardiopulmonary exercise testing) parameter in a patient in need thereof, wherein the patient is concurrently undergoing a treatment with a beta blocker.

As further described herein a therapeutically effective amount of aficamten, or a pharmaceutically acceptable salt thereof, may be administered to a patient for improving a heart rate dependent CPET (cardiopulmonary exercise testing) parameter in a patient in need thereof, wherein the patient was administered a beta blocker within at least 1 week, at least 2 weeks, at least 3 weeks, or at least 4 weeks prior to administration of the therapeutically effective amount of aficamten, or the pharmaceutically acceptable salt thereof. In some embodiments, the patient was administered a beta blocker within at least 1 day, at least 2 days, at least 3 days, at least 4 days, at least 5 days, at least 6 days, at least 1 week, at least 8 days, at least 9 days, at least 10 days, at least 11 days, at least 12 days, at least 13 days, at least 2 weeks, at least 15 days, at least 16 days, at least 17 days, at least 18 days, at least 19 days, at least 20 days, at least 3 weeks, at least 22 days, at least 23 days, at least 24 days, at least 25 days, at least 26 days, at least 27 days, at least 4 weeks, at least 1 month, at least 5 weeks, at least 6 weeks, at least 7 weeks, at least 8 weeks, at least 2 months, at least 9 weeks, at least 10 weeks, at least 11 weeks, at least 12 weeks, at least 3 months, at least 16 weeks, at least 4 months, at least 20 weeks, at least 5 months, at least 24 weeks, at least 6 months, at least 28 weeks, at least 7 months, at least 32 weeks, at least 8 months, at least 36 weeks, at least 9 months, at least 40 weeks, at least 10 months, at least 44 weeks, at least 11 months, at least 48 weeks, or at least 12 months prior to administration of the therapeutically effective amount of aficamten, or the pharmaceutically acceptable salt thereof. In some embodiments, the patient continues receiving the beta blocker after the administration of aficamten, or the pharmaceutically acceptable salt thereof, started.

As further described herein a therapeutically effective amount of aficamten, or a pharmaceutically acceptable salt thereof, may be administered to a patient for improving a heart rate dependent CPET (cardiopulmonary exercise testing) parameter in a patient in need thereof, resulting in an improvement in the heart rate dependent CPET (cardiopulmonary exercise testing) parameter; wherein the patient is concurrently undergoing a treatment with a beta blocker; and wherein the improvement in the heart rate dependent CPET (cardiopulmonary exercise testing) parameter is not substantially diminished by the treatment with the beta blocker.

As further described herein a therapeutically effective amount of aficamten, or a pharmaceutically acceptable salt thereof, may be administered to a patient for improving a heart rate dependent CPET (cardiopulmonary exercise testing) parameter in a patient in need thereof, wherein the patient has been undergoing a treatment with a beta blocker prior to administration of aficamten, or the pharmaceutically acceptable salt thereof, and the treatment with the beta blocker remains unchanged concurrently to the administration of aficamten, or the pharmaceutically acceptable salt thereof. In some embodiments, the treatment with the beta blocker is not changed for at least 1 day, at least 2 days, at least 3 days, at least 4 days, at least 5 days, at least 6 days, at least 1 week, at least 8 days, at least 9 days, at least 10 days, at least 11 days, at least 12 days, at least 13 days, at least 2 weeks, at least 15 days, at least 16 days, at least 17 days, at least 18 days, at least 19 days, at least 20 days, at least 3 weeks, at least 22 days, at least 23 days, at least 24 days, at least 25 days, at least 26 days, at least 27 days, at least 4 weeks, at least 1 month, at least 5 weeks, at least 6 weeks, at least 7 weeks, at least 8 weeks, at least 2 months, at least 9 weeks, at least 10 weeks, at least 11 weeks, at least 12 weeks, at least 3 months, at least 16 weeks, at least 4 months, at least 20 weeks, at least 5 months, at least 24 weeks, at least 6 months, at least 28 weeks, at least 7 months, at least 32 weeks, at least 8 months, at least 36 weeks, at least 9 months, at least 40 weeks, at least 10 months, at least 44 weeks, at least 11 months, at least 48 weeks, or at least 12 months during the concurrent administration of the therapeutically effective amount of aficamten, or the pharmaceutically acceptable salt thereof. In some embodiments, the treatment with the beta blocker is not changed for at least 1 day, at least 2 days, at least 3 days, at least 4 days, at least 5 days, at least 6 days, at least 1 week, at least 8 days, at least 9 days, at least 10 days, at least 11 days, at least 12 days, at least 13 days, at least 2 weeks, at least 15 days, at least 16 days, at least 17 days, at least 18 days, at least 19 days, at least 20 days, at least 3 weeks, at least 22 days, at least 23 days, at least 24 days, at least 25 days, at least 26 days, at least 27 days, at least 4 weeks, at least 1 month, at least 5 weeks, at least 6 weeks, at least 7 weeks, at least 8 weeks, at least 2 months, at least 9 weeks, at least 10 weeks, at least 11 weeks, at least 12 weeks, at least 3 months, at least 16 weeks, at least 4 months, at least 20 weeks, at least 5 months, at least 24 weeks, at least 6 months, at least 28 weeks, at least 7 months, at least 32 weeks, at least 8 months, at least 36 weeks, at least 9 months, at least 40 weeks, at least 10 months, at least 44 weeks, at least 11 months, at least 48 weeks, or at least 12 months following administration of the therapeutically effective amount of aficamten, or the pharmaceutically acceptable salt thereof.

As further described herein a therapeutically effective amount of aficamten, or a pharmaceutically acceptable salt thereof, may be administered to a patient for improving a heart rate dependent CPET (cardiopulmonary exercise testing) parameter in a patient in need thereof, wherein treatment with a beta blocker is not contraindicated for the patient.

As further described herein a therapeutically effective amount of a beta blocker, or a pharmaceutically acceptable salt thereof, may be administered to a patient for improving a heart rate dependent CPET (cardiopulmonary exercise testing) parameter in a patient in need thereof, wherein the patient is concurrently undergoing a treatment with aficamten, or a pharmaceutically acceptable salt thereof.

As further described herein a therapeutically effective amount of aficamten, or a pharmaceutically acceptable salt thereof, and a beta blocker may be administered to a patient for improving a heart rate dependent CPET (cardiopulmonary exercise testing) parameter in a patient in need thereof.

As further described herein a therapeutically effective amount of aficamten, or a pharmaceutically acceptable salt thereof, optionally in combination with a beta blocker may be administered to a patient for improving a heart rate dependent CPET (cardiopulmonary exercise testing) parameter in a patient in need thereof.

As further described herein a therapeutically effective amount of aficamten, or a pharmaceutically acceptable salt thereof, may be administered to a patient for treating a patient in need thereof, wherein (a) the co-administration of the beta blocker with aficamten, or a pharmaceutically acceptable salt thereof, does not cause chronotropic incompetence in the patient, and/or (b) the co-administration of the beta blocker with aficamten, or a pharmaceutically acceptable salt thereof, does not impact the ability of the patient's heart to increase its rate commensurate with increased activity or demand, and/or (c) the patient's heart maintains its ability to increase its rate commensurate with increased activity or demand compared to administration of aficamten, or a pharmaceutically acceptable salt thereof, without a beta blocker.

As further described herein a therapeutically effective amount of aficamten, or a pharmaceutically acceptable salt thereof, may be administered to a patient for improving heart rate reserve in a patient in need thereof, wherein the patient is concurrently undergoing a treatment with a beta blocker.

As further described herein a therapeutically effective amount of aficamten, or a pharmaceutically acceptable salt thereof, may be administered to a patient for improving heart rate reserve in a patient in need thereof, wherein the patient was administered a beta blocker within at least 1 week, at least 2 weeks, at least 3 weeks, or at least 4 weeks prior to administration of the therapeutically effective amount of aficamten, or the pharmaceutically acceptable salt thereof. In some embodiments, the patient was administered a beta blocker within at least 1 day, at least 2 days, at least 3 days, at least 4 days, at least 5 days, at least 6 days, at least 1 week, at least 8 days, at least 9 days, at least 10 days, at least 11 days, at least 12 days, at least 13 days, at least 2 weeks, at least 15 days, at least 16 days, at least 17 days, at least 18 days, at least 19 days, at least 20 days, at least 3 weeks, at least 22 days, at least 23 days, at least 24 days, at least 25 days, at least 26 days, at least 27 days, at least 4 weeks, at least 1 month, at least 5 weeks, at least 6 weeks, at least 7 weeks, at least 8 weeks, at least 2 months, at least 9 weeks, at least 10 weeks, at least 11 weeks, at least 12 weeks, at least 3 months, at least 16 weeks, at least 4 months, at least 20 weeks, at least 5 months, at least 24 weeks, at least 6 months, at least 28 weeks, at least 7 months, at least 32 weeks, at least 8 months, at least 36 weeks, at least 9 months, at least 40 weeks, at least 10 months, at least 44 weeks, at least 11 months, at least 48 weeks, or at least 12 months prior to administration of the therapeutically effective amount of aficamten, or the pharmaceutically acceptable salt thereof. In some embodiments, the patient continues receiving the beta blocker after the administration of aficamten, or the pharmaceutically acceptable salt thereof, started.

As further described herein a therapeutically effective amount of aficamten, or a pharmaceutically acceptable salt thereof, may be administered to a patient for improving heart rate reserve in a patient in need thereof, resulting in an improvement in heart rate reserve; wherein the patient is concurrently undergoing a treatment with a beta blocker; and wherein the improvement in heart rate reserve is not substantially diminished by the treatment with the beta blocker.

As further described herein a therapeutically effective amount of aficamten, or a pharmaceutically acceptable salt thereof, may be administered to a patient for improving heart rate reserve in a patient in need thereof, wherein the patient has been undergoing a treatment with a beta blocker prior to administration of aficamten, or the pharmaceutically acceptable salt thereof, and the treatment with the beta blocker remains unchanged concurrently to the administration of aficamten, or the pharmaceutically acceptable salt thereof. In some embodiments, the treatment with the beta blocker is not changed for at least 1 day, at least 2 days, at least 3 days, at least 4 days, at least 5 days, at least 6 days, at least 1 week, at least 8 days, at least 9 days, at least 10 days, at least 11 days, at least 12 days, at least 13 days, at least 2 weeks, at least 15 days, at least 16 days, at least 17 days, at least 18 days, at least 19 days, at least 20 days, at least 3 weeks, at least 22 days, at least 23 days, at least 24 days, at least 25 days, at least 26 days, at least 27 days, at least 4 weeks, at least 1 month, at least 5 weeks, at least 6 weeks, at least 7 weeks, at least 8 weeks, at least 2 months, at least 9 weeks, at least 10 weeks, at least 11 weeks, at least 12 weeks, at least 3 months, at least 16 weeks, at least 4 months, at least 20 weeks, at least 5 months, at least 24 weeks, at least 6 months, at least 28 weeks, at least 7 months, at least 32 weeks, at least 8 months, at least 36 weeks, at least 9 months, at least 40 weeks, at least 10 months, at least 44 weeks, at least 11 months, at least 48 weeks, or at least 12 months during the concurrent administration of the therapeutically effective amount of aficamten, or the pharmaceutically acceptable salt thereof. In some embodiments, the treatment with the beta blocker is not changed for at least 1 day, at least 2 days, at least 3 days, at least 4 days, at least 5 days, at least 6 days, at least 1 week, at least 8 days, at least 9 days, at least 10 days, at least 11 days, at least 12 days, at least 13 days, at least 2 weeks, at least 15 days, at least 16 days, at least 17 days, at least 18 days, at least 19 days, at least 20 days, at least 3 weeks, at least 22 days, at least 23 days, at least 24 days, at least 25 days, at least 26 days, at least 27 days, at least 4 weeks, at least 1 month, at least 5 weeks, at least 6 weeks, at least 7 weeks, at least 8 weeks, at least 2 months, at least 9 weeks, at least 10 weeks, at least 11 weeks, at least 12 weeks, at least 3 months, at least 16 weeks, at least 4 months, at least 20 weeks, at least 5 months, at least 24 weeks, at least 6 months, at least 28 weeks, at least 7 months, at least 32 weeks, at least 8 months, at least 36 weeks, at least 9 months, at least 40 weeks, at least 10 months, at least 44 weeks, at least 11 months, at least 48 weeks, or at least 12 months following administration of the therapeutically effective amount of aficamten, or the pharmaceutically acceptable salt thereof.

As further described herein a therapeutically effective amount of aficamten, or a pharmaceutically acceptable salt thereof, may be administered to a patient for improving heart rate reserve in a patient in need thereof, wherein the patient has been undergoing a treatment with a beta blocker prior to administration of aficamten, or the pharmaceutically acceptable salt thereof, and the treatment with the beta blocker is not changed for at least 1 week, at least 2 weeks, at least 3 weeks, or at least 4 weeks during the concurrent administration of aficamten, or the pharmaceutically acceptable salt thereof. In some embodiments, the treatment with the beta blocker is not changed for at least 1 day, at least 2 days, at least 3 days, at least 4 days, at least 5 days, at least 6 days, at least 1 week, at least 8 days, at least 9 days, at least 10 days, at least 11 days, at least 12 days, at least 13 days, at least 2 weeks, at least 15 days, at least 16 days, at least 17 days, at least 18 days, at least 19 days, at least 20 days, at least 3 weeks, at least 22 days, at least 23 days, at least 24 days, at least 25 days, at least 26 days, at least 27 days, at least 4 weeks, at least 1 month, at least 5 weeks, at least 6 weeks, at least 7 weeks, at least 8 weeks, at least 2 months, at least 9 weeks, at least 10 weeks, at least 11 weeks, at least 12 weeks, at least 3 months, at least 16 weeks, at least 4 months, at least 20 weeks, at least 5 months, at least 24 weeks, at least 6 months, at least 28 weeks, at least 7 months, at least 32 weeks, at least 8 months, at least 36 weeks, at least 9 months, at least 40 weeks, at least 10 months, at least 44 weeks, at least 11 months, at least 48 weeks, or at least 12 months during the concurrent administration of the therapeutically effective amount of aficamten, or the pharmaceutically acceptable salt thereof.

As further described herein a therapeutically effective amount of aficamten, or a pharmaceutically acceptable salt thereof, may be administered to a patient for improving heart rate reserve in a patient in need thereof, wherein treatment with a beta blocker is not contraindicated for the patient.

As further described herein a therapeutically effective amount of a beta blocker, or a pharmaceutically acceptable salt thereof, may be administered to a patient for improving heart rate reserve in a patient in need thereof, wherein the patient is concurrently undergoing a treatment with aficamten, or a pharmaceutically acceptable salt thereof.

As further described herein a therapeutically effective amount of aficamten, or a pharmaceutically acceptable salt thereof, and a beta blocker may be administered to a patient for improving heart rate reserve in a patient in need thereof.

As further described herein a therapeutically effective amount of aficamten, or a pharmaceutically acceptable salt thereof, optionally in combination with a beta blocker may be administered to a patient for improving heart rate reserve in a patient in need thereof.

As further described herein a therapeutically effective amount of aficamten, or a pharmaceutically acceptable salt thereof, may be administered to a patient for improving peak metabolic equivalents (METs) in a patient in need thereof, wherein the patient is concurrently undergoing a treatment with a beta blocker.

As further described herein a therapeutically effective amount of aficamten, or a pharmaceutically acceptable salt thereof, may be administered to a patient for improving peak metabolic equivalents (METs) in a patient in need thereof, wherein the patient was administered a beta blocker within at least 1 week, at least 2 weeks, at least 3 weeks, or at least 4 weeks prior to administration of the therapeutically effective amount of aficamten, or the pharmaceutically acceptable salt thereof. In some embodiments, the patient was administered a beta blocker within at least 1 day, at least 2 days, at least 3 days, at least 4 days, at least 5 days, at least 6 days, at least 1 week, at least 8 days, at least 9 days, at least 10 days, at least 11 days, at least 12 days, at least 13 days, at least 2 weeks, at least 15 days, at least 16 days, at least 17 days, at least 18 days, at least 19 days, at least 20 days, at least 3 weeks, at least 22 days, at least 23 days, at least 24 days, at least 25 days, at least 26 days, at least 27 days, at least 4 weeks, at least 1 month, at least 5 weeks, at least 6 weeks, at least 7 weeks, at least 8 weeks, at least 2 months, at least 9 weeks, at least 10 weeks, at least 11 weeks, at least 12 weeks, at least 3 months, at least 16 weeks, at least 4 months, at least 20 weeks, at least 5 months, at least 24 weeks, at least 6 months, at least 28 weeks, at least 7 months, at least 32 weeks, at least 8 months, at least 36 weeks, at least 9 months, at least 40 weeks, at least 10 months, at least 44 weeks, at least 11 months, at least 48 weeks, or at least 12 months prior to administration of the therapeutically effective amount of aficamten, or the pharmaceutically acceptable salt thereof. In some embodiments, the patient continues receiving the beta blocker after the administration of aficamten, or the pharmaceutically acceptable salt thereof, started.

As further described herein a therapeutically effective amount of aficamten, or a pharmaceutically acceptable salt thereof, may be administered to a patient for improving peak metabolic equivalents (METs) in a patient in need thereof, resulting in an improvement in peak metabolic equivalents (METs); wherein the patient is concurrently undergoing a treatment with a beta blocker; and wherein the improvement in peak metabolic equivalents (METs) is not substantially diminished by the treatment with the beta blocker.

As further described herein a therapeutically effective amount of aficamten, or a pharmaceutically acceptable salt thereof, may be administered to a patient for improving peak metabolic equivalents (METs) in a patient in need thereof, wherein the patient has been undergoing a treatment with a beta blocker prior to administration of aficamten, or the pharmaceutically acceptable salt thereof, and the treatment with the beta blocker remains unchanged concurrently to the administration of aficamten, or the pharmaceutically acceptable salt thereof. In some embodiments, the treatment with the beta blocker is not changed for at least 1 day, at least 2 days, at least 3 days, at least 4 days, at least 5 days, at least 6 days, at least 1 week, at least 8 days, at least 9 days, at least 10 days, at least 11 days, at least 12 days, at least 13 days, at least 2 weeks, at least 15 days, at least 16 days, at least 17 days, at least 18 days, at least 19 days, at least 20 days, at least 3 weeks, at least 22 days, at least 23 days, at least 24 days, at least 25 days, at least 26 days, at least 27 days, at least 4 weeks, at least 1 month, at least 5 weeks, at least 6 weeks, at least 7 weeks, at least 8 weeks, at least 2 months, at least 9 weeks, at least 10 weeks, at least 11 weeks, at least 12 weeks, at least 3 months, at least 16 weeks, at least 4 months, at least 20 weeks, at least 5 months, at least 24 weeks, at least 6 months, at least 28 weeks, at least 7 months, at least 32 weeks, at least 8 months, at least 36 weeks, at least 9 months, at least 40 weeks, at least 10 months, at least 44 weeks, at least 11 months, at least 48 weeks, or at least 12 months during the concurrent administration of the therapeutically effective amount of aficamten, or the pharmaceutically acceptable salt thereof. In some embodiments, the treatment with the beta blocker is not changed for at least 1 day, at least 2 days, at least 3 days, at least 4 days, at least 5 days, at least 6 days, at least 1 week, at least 8 days, at least 9 days, at least 10 days, at least 11 days, at least 12 days, at least 13 days, at least 2 weeks, at least 15 days, at least 16 days, at least 17 days, at least 18 days, at least 19 days, at least 20 days, at least 3 weeks, at least 22 days, at least 23 days, at least 24 days, at least 25 days, at least 26 days, at least 27 days, at least 4 weeks, at least 1 month, at least 5 weeks, at least 6 weeks, at least 7 weeks, at least 8 weeks, at least 2 months, at least 9 weeks, at least 10 weeks, at least 11 weeks, at least 12 weeks, at least 3 months, at least 16 weeks, at least 4 months, at least 20 weeks, at least 5 months, at least 24 weeks, at least 6 months, at least 28 weeks, at least 7 months, at least 32 weeks, at least 8 months, at least 36 weeks, at least 9 months, at least 40 weeks, at least 10 months, at least 44 weeks, at least 11 months, at least 48 weeks, or at least 12 months following administration of the therapeutically effective amount of aficamten, or the pharmaceutically acceptable salt thereof.

As further described herein a therapeutically effective amount of aficamten, or a pharmaceutically acceptable salt thereof, may be administered to a patient for improving peak metabolic equivalents (METs) in a patient in need thereof, wherein the patient has been undergoing a treatment with a beta blocker prior to administration of aficamten, or the pharmaceutically acceptable salt thereof, and the treatment with the beta blocker is not changed for at least 1 week, at least 2 weeks, at least 3 weeks, or at least 4 weeks during the concurrent administration of aficamten, or the pharmaceutically acceptable salt thereof. In some embodiments, the treatment with the beta blocker is not changed for at least 1 day, at least 2 days, at least 3 days, at least 4 days, at least 5 days, at least 6 days, at least 1 week, at least 8 days, at least 9 days, at least 10 days, at least 11 days, at least 12 days, at least 13 days, at least 2 weeks, at least 15 days, at least 16 days, at least 17 days, at least 18 days, at least 19 days, at least 20 days, at least 3 weeks, at least 22 days, at least 23 days, at least 24 days, at least 25 days, at least 26 days, at least 27 days, at least 4 weeks, at least 1 month, at least 5 weeks, at least 6 weeks, at least 7 weeks, at least 8 weeks, at least 2 months, at least 9 weeks, at least 10 weeks, at least 11 weeks, at least 12 weeks, at least 3 months, at least 16 weeks, at least 4 months, at least 20 weeks, at least 5 months, at least 24 weeks, at least 6 months, at least 28 weeks, at least 7 months, at least 32 weeks, at least 8 months, at least 36 weeks, at least 9 months, at least 40 weeks, at least 10 months, at least 44 weeks, at least 11 months, at least 48 weeks, or at least 12 months during the concurrent administration of the therapeutically effective amount of aficamten, or the pharmaceutically acceptable salt thereof.

As further described herein a therapeutically effective amount of aficamten, or a pharmaceutically acceptable salt thereof, may be administered to a patient for improving peak metabolic equivalents (METs) in a patient in need thereof, wherein treatment with a beta blocker is not contraindicated for the patient.

As further described herein a therapeutically effective amount of a beta blocker, or a pharmaceutically acceptable salt thereof, may be administered to a patient for improving peak metabolic equivalents (METs) in a patient in need thereof, wherein the patient is concurrently undergoing a treatment with aficamten, or a pharmaceutically acceptable salt thereof.

As further described herein a therapeutically effective amount of aficamten, or a pharmaceutically acceptable salt thereof, and a beta blocker may be administered to a patient for improving peak metabolic equivalents (METs) in a patient in need thereof.

As further described herein a therapeutically effective amount of aficamten, or a pharmaceutically acceptable salt thereof, optionally in combination with a beta blocker may be administered to a patient for improving peak metabolic equivalents (METs) in a patient in need thereof.

As further described herein a therapeutically effective amount of aficamten, or a pharmaceutically acceptable salt thereof, may be administered to a patient for improving exercise duration in a patient in need thereof, wherein the patient is concurrently undergoing a treatment with a beta blocker.

As further described herein a therapeutically effective amount of aficamten, or a pharmaceutically acceptable salt thereof, may be administered to a patient for improving exercise duration in a patient in need thereof, wherein the patient was administered a beta blocker within at least 1 week, at least 2 weeks, at least 3 weeks, or at least 4 weeks prior to administration of the therapeutically effective amount of aficamten, or the pharmaceutically acceptable salt thereof. In some embodiments, the patient was administered a beta blocker within at least 1 day, at least 2 days, at least 3 days, at least 4 days, at least 5 days, at least 6 days, at least 1 week, at least 8 days, at least 9 days, at least 10 days, at least 11 days, at least 12 days, at least 13 days, at least 2 weeks, at least 15 days, at least 16 days, at least 17 days, at least 18 days, at least 19 days, at least 20 days, at least 3 weeks, at least 22 days, at least 23 days, at least 24 days, at least 25 days, at least 26 days, at least 27 days, at least 4 weeks, at least 1 month, at least 5 weeks, at least 6 weeks, at least 7 weeks, at least 8 weeks, at least 2 months, at least 9 weeks, at least 10 weeks, at least 11 weeks, at least 12 weeks, at least 3 months, at least 16 weeks, at least 4 months, at least 20 weeks, at least 5 months, at least 24 weeks, at least 6 months, at least 28 weeks, at least 7 months, at least 32 weeks, at least 8 months, at least 36 weeks, at least 9 months, at least 40 weeks, at least 10 months, at least 44 weeks, at least 11 months, at least 48 weeks, or at least 12 months prior to administration of the therapeutically effective amount of aficamten, or the pharmaceutically acceptable salt thereof. In some embodiments, the patient continues receiving the beta blocker after the administration of aficamten, or the pharmaceutically acceptable salt thereof, started.

As further described herein a therapeutically effective amount of aficamten, or a pharmaceutically acceptable salt thereof, may be administered to a patient for improving exercise duration in a patient in need thereof, resulting in an improvement in exercise duration; wherein the patient is concurrently undergoing a treatment with a beta blocker; and wherein the improvement in exercise duration is not substantially diminished by the treatment with the beta blocker.

As further described herein a therapeutically effective amount of aficamten, or a pharmaceutically acceptable salt thereof, may be administered to a patient for improving exercise duration in a patient in need thereof, wherein the patient has been undergoing a treatment with a beta blocker prior to administration of aficamten, or the pharmaceutically acceptable salt thereof, and the treatment with the beta blocker remains unchanged concurrently to the administration of aficamten, or the pharmaceutically acceptable salt thereof. In some embodiments, the treatment with the beta blocker is not changed for at least 1 day, at least 2 days, at least 3 days, at least 4 days, at least 5 days, at least 6 days, at least 1 week, at least 8 days, at least 9 days, at least 10 days, at least 11 days, at least 12 days, at least 13 days, at least 2 weeks, at least 15 days, at least 16 days, at least 17 days, at least 18 days, at least 19 days, at least 20 days, at least 3 weeks, at least 22 days, at least 23 days, at least 24 days, at least 25 days, at least 26 days, at least 27 days, at least 4 weeks, at least 1 month, at least 5 weeks, at least 6 weeks, at least 7 weeks, at least 8 weeks, at least 2 months, at least 9 weeks, at least 10 weeks, at least 11 weeks, at least 12 weeks, at least 3 months, at least 16 weeks, at least 4 months, at least 20 weeks, at least 5 months, at least 24 weeks, at least 6 months, at least 28 weeks, at least 7 months, at least 32 weeks, at least 8 months, at least 36 weeks, at least 9 months, at least 40 weeks, at least 10 months, at least 44 weeks, at least 11 months, at least 48 weeks, or at least 12 months during the concurrent administration of the therapeutically effective amount of aficamten, or the pharmaceutically acceptable salt thereof. In some embodiments, the treatment with the beta blocker is not changed for at least 1 day, at least 2 days, at least 3 days, at least 4 days, at least 5 days, at least 6 days, at least 1 week, at least 8 days, at least 9 days, at least 10 days, at least 11 days, at least 12 days, at least 13 days, at least 2 weeks, at least 15 days, at least 16 days, at least 17 days, at least 18 days, at least 19 days, at least 20 days, at least 3 weeks, at least 22 days, at least 23 days, at least 24 days, at least 25 days, at least 26 days, at least 27 days, at least 4 weeks, at least 1 month, at least 5 weeks, at least 6 weeks, at least 7 weeks, at least 8 weeks, at least 2 months, at least 9 weeks, at least 10 weeks, at least 11 weeks, at least 12 weeks, at least 3 months, at least 16 weeks, at least 4 months, at least 20 weeks, at least 5 months, at least 24 weeks, at least 6 months, at least 28 weeks, at least 7 months, at least 32 weeks, at least 8 months, at least 36 weeks, at least 9 months, at least 40 weeks, at least 10 months, at least 44 weeks, at least 11 months, at least 48 weeks, or at least 12 months following administration of the therapeutically effective amount of aficamten, or the pharmaceutically acceptable salt thereof.

As further described herein a therapeutically effective amount of aficamten, or a pharmaceutically acceptable salt thereof, may be administered to a patient for improving exercise duration in a patient in need thereof, wherein the patient has been undergoing a treatment with a beta blocker prior to administration of aficamten, or the pharmaceutically acceptable salt thereof, and the treatment with the beta blocker is not changed for at least 1 week, at least 2 weeks, at least 3 weeks, or at least 4 weeks during the concurrent administration of aficamten, or the pharmaceutically acceptable salt thereof. In some embodiments, the treatment with the beta blocker is not changed for at least 1 day, at least 2 days, at least 3 days, at least 4 days, at least 5 days, at least 6 days, at least 1 week, at least 8 days, at least 9 days, at least 10 days, at least 11 days, at least 12 days, at least 13 days, at least 2 weeks, at least 15 days, at least 16 days, at least 17 days, at least 18 days, at least 19 days, at least 20 days, at least 3 weeks, at least 22 days, at least 23 days, at least 24 days, at least 25 days, at least 26 days, at least 27 days, at least 4 weeks, at least 1 month, at least 5 weeks, at least 6 weeks, at least 7 weeks, at least 8 weeks, at least 2 months, at least 9 weeks, at least 10 weeks, at least 11 weeks, at least 12 weeks, at least 3 months, at least 16 weeks, at least 4 months, at least 20 weeks, at least 5 months, at least 24 weeks, at least 6 months, at least 28 weeks, at least 7 months, at least 32 weeks, at least 8 months, at least 36 weeks, at least 9 months, at least 40 weeks, at least 10 months, at least 44 weeks, at least 11 months, at least 48 weeks, or at least 12 months during the concurrent administration of the therapeutically effective amount of aficamten, or the pharmaceutically acceptable salt thereof.

As further described herein a therapeutically effective amount of aficamten, or a pharmaceutically acceptable salt thereof, may be administered to a patient for improving exercise duration in a patient in need thereof, wherein treatment with a beta blocker is not contraindicated for the patient.

As further described herein a therapeutically effective amount of a beta blocker, or a pharmaceutically acceptable salt thereof, may be administered to a patient for improving exercise duration in a patient in need thereof, wherein the patient is concurrently undergoing a treatment with aficamten, or a pharmaceutically acceptable salt thereof.

As further described herein a therapeutically effective amount of aficamten, or a pharmaceutically acceptable salt thereof, and a beta blocker may be administered to a patient for improving exercise duration in a patient in need thereof.

As further described herein a therapeutically effective amount of aficamten, or a pharmaceutically acceptable salt thereof, optionally in combination with a beta blocker may be administered to a patient for improving exercise duration in a patient in need thereof.

As further described herein a therapeutically effective amount of aficamten, or a pharmaceutically acceptable salt thereof, may be administered to a patient for improving one or more of integrated 2 component Z-score metric, pVO₂, peak workload, peak METs, peak circulatory power, exercise duration, peak respiratory exchange ratio, heart rate reserve, peak heart rate, O₂ pulse at peak exercise, peak proportionate pulse pressure, proportionate pulse pressure at rest, ventilatory efficiency pre-anaerobic threshold, ventilatory efficiency throughout exercise, ventilatory power, ventilatory anaerobic threshold, and acrobic efficiency in a patient in need thereof, wherein the patient is concurrently undergoing a treatment with a beta blocker.

As further described herein a therapeutically effective amount of aficamten, or a pharmaceutically acceptable salt thereof, may be administered to a patient for improving one or more of integrated 2 component Z-score metric, pVO₂, peak workload, peak METs, peak circulatory power, exercise duration, peak respiratory exchange ratio, heart rate reserve, peak heart rate, O₂ pulse at peak exercise, peak proportionate pulse pressure, proportionate pulse pressure at rest, ventilatory efficiency pre-anaerobic threshold, ventilatory efficiency throughout exercise, ventilatory power, ventilatory anaerobic threshold, and aerobic efficiency in a patient in need thereof, wherein the patient was administered a beta blocker within at least 1 week, at least 2 weeks, at least 3 weeks, or at least 4 weeks prior to administration of the therapeutically effective amount of aficamten, or the pharmaceutically acceptable salt thereof. In some embodiments, the patient was administered a beta blocker within at least 1 day, at least 2 days, at least 3 days, at least 4 days, at least 5 days, at least 6 days, at least 1 week, at least 8 days, at least 9 days, at least 10 days, at least 11 days, at least 12 days, at least 13 days, at least 2 weeks, at least 15 days, at least 16 days, at least 17 days, at least 18 days, at least 19 days, at least 20 days, at least 3 weeks, at least 22 days, at least 23 days, at least 24 days, at least 25 days, at least 26 days, at least 27 days, at least 4 weeks, at least 1 month, at least 5 weeks, at least 6 weeks, at least 7 weeks, at least 8 weeks, at least 2 months, at least 9 weeks, at least 10 weeks, at least 11 weeks, at least 12 weeks, at least 3 months, at least 16 weeks, at least 4 months, at least 20 weeks, at least 5 months, at least 24 weeks, at least 6 months, at least 28 weeks, at least 7 months, at least 32 weeks, at least 8 months, at least 36 weeks, at least 9 months, at least 40 weeks, at least 10 months, at least 44 weeks, at least 11 months, at least 48 weeks, or at least 12 months prior to administration of the therapeutically effective amount of aficamten, or the pharmaceutically acceptable salt thereof. In some embodiments, the patient continues receiving the beta blocker after the administration of aficamten, or the pharmaceutically acceptable salt thereof, started.

As further described herein a therapeutically effective amount of aficamten, or a pharmaceutically acceptable salt thereof, may be administered to a patient for improving one or more of integrated 2 component Z-score metric, pVO₂, peak workload, peak METs, peak circulatory power, exercise duration, peak respiratory exchange ratio, heart rate reserve, peak heart rate, O₂ pulse at peak exercise, peak proportionate pulse pressure, proportionate pulse pressure at rest, ventilatory efficiency pre-anaerobic threshold, ventilatory efficiency throughout exercise, ventilatory power, ventilatory anaerobic threshold, and acrobic efficiency in a patient in need thereof, resulting in an improvement in one or more of integrated 2 component Z-score metric, pVO₂, peak workload, peak METs, peak circulatory power, exercise duration, peak respiratory exchange ratio, heart rate reserve, peak heart rate, O₂ pulse at peak exercise, peak proportionate pulse pressure, proportionate pulse pressure at rest, ventilatory efficiency pre-anaerobic threshold, ventilatory efficiency throughout exercise, ventilatory power, ventilatory anaerobic threshold, and aerobic efficiency; wherein the patient is concurrently undergoing a treatment with a beta blocker; and wherein the improvement in one or more of integrated 2 component Z-score metric, pVO₂, peak workload, peak METs, peak circulatory power, exercise duration, peak respiratory exchange ratio, heart rate reserve, peak heart rate, O₂ pulse at peak exercise, peak proportionate pulse pressure, proportionate pulse pressure at rest, ventilatory efficiency pre-anaerobic threshold, ventilatory efficiency throughout exercise, ventilatory power, ventilatory anaerobic threshold, and acrobic efficiency is not substantially diminished by the treatment with the beta blocker.

As further described herein a therapeutically effective amount of aficamten, or a pharmaceutically acceptable salt thereof, may be administered to a patient for improving one or more of integrated 2 component Z-score metric, pVO₂, peak workload, peak METs, peak circulatory power, exercise duration, peak respiratory exchange ratio, heart rate reserve, peak heart rate, O₂ pulse at peak exercise, peak proportionate pulse pressure, proportionate pulse pressure at rest, ventilatory efficiency pre-anaerobic threshold, ventilatory efficiency throughout exercise, ventilatory power, ventilatory anaerobic threshold, and aerobic efficiency in a patient in need thereof, wherein the patient has been undergoing a treatment with a beta blocker prior to administration of aficamten, or the pharmaceutically acceptable salt thereof, and the treatment with the beta blocker remains unchanged concurrently to the administration of aficamten, or the pharmaceutically acceptable salt thereof. In some embodiments, the treatment with the beta blocker is not changed for at least 1 day, at least 2 days, at least 3 days, at least 4 days, at least 5 days, at least 6 days, at least 1 week, at least 8 days, at least 9 days, at least 10 days, at least 11 days, at least 12 days, at least 13 days, at least 2 weeks, at least 15 days, at least 16 days, at least 17 days, at least 18 days, at least 19 days, at least 20 days, at least 3 weeks, at least 22 days, at least 23 days, at least 24 days, at least 25 days, at least 26 days, at least 27 days, at least 4 weeks, at least 1 month, at least 5 weeks, at least 6 weeks, at least 7 weeks, at least 8 weeks, at least 2 months, at least 9 weeks, at least 10 weeks, at least 11 weeks, at least 12 weeks, at least 3 months, at least 16 weeks, at least 4 months, at least 20 weeks, at least 5 months, at least 24 weeks, at least 6 months, at least 28 weeks, at least 7 months, at least 32 weeks, at least 8 months, at least 36 weeks, at least 9 months, at least 40 weeks, at least 10 months, at least 44 weeks, at least 11 months, at least 48 weeks, or at least 12 months during the concurrent administration of the therapeutically effective amount of aficamten, or the pharmaceutically acceptable salt thereof. In some embodiments, the treatment with the beta blocker is not changed for at least 1 day, at least 2 days, at least 3 days, at least 4 days, at least 5 days, at least 6 days, at least 1 week, at least 8 days, at least 9 days, at least 10 days, at least 11 days, at least 12 days, at least 13 days, at least 2 weeks, at least 15 days, at least 16 days, at least 17 days, at least 18 days, at least 19 days, at least 20 days, at least 3 weeks, at least 22 days, at least 23 days, at least 24 days, at least 25 days, at least 26 days, at least 27 days, at least 4 weeks, at least 1 month, at least 5 weeks, at least 6 weeks, at least 7 weeks, at least 8 weeks, at least 2 months, at least 9 weeks, at least 10 weeks, at least 11 weeks, at least 12 weeks, at least 3 months, at least 16 weeks, at least 4 months, at least 20 weeks, at least 5 months, at least 24 weeks, at least 6 months, at least 28 weeks, at least 7 months, at least 32 weeks, at least 8 months, at least 36 weeks, at least 9 months, at least 40 weeks, at least 10 months, at least 44 weeks, at least 11 months, at least 48 weeks, or at least 12 months following administration of the therapeutically effective amount of aficamten, or the pharmaceutically acceptable salt thereof.

As further described herein a therapeutically effective amount of aficamten, or a pharmaceutically acceptable salt thereof, may be administered to a patient for improving one or more of integrated 2 component Z-score metric, pVO₂, peak workload, peak METs, peak circulatory power, exercise duration, peak respiratory exchange ratio, heart rate reserve, peak heart rate, O₂ pulse at peak exercise, peak proportionate pulse pressure, proportionate pulse pressure at rest, ventilatory efficiency pre-anaerobic threshold, ventilatory efficiency throughout exercise, ventilatory power, ventilatory anaerobic threshold, and aerobic efficiency in a patient in need thereof, wherein the patient has been undergoing a treatment with a beta blocker prior to administration of aficamten, or the pharmaceutically acceptable salt thereof, and the treatment with the beta blocker is not changed for at least 1 week, at least 2 weeks, at least 3 weeks, or at least 4 weeks during the concurrent administration of aficamten, or the pharmaceutically acceptable salt thereof. In some embodiments, the treatment with the beta blocker is not changed for at least 1 day, at least 2 days, at least 3 days, at least 4 days, at least 5 days, at least 6 days, at least 1 week, at least 8 days, at least 9 days, at least 10 days, at least 11 days, at least 12 days, at least 13 days, at least 2 weeks, at least 15 days, at least 16 days, at least 17 days, at least 18 days, at least 19 days, at least 20 days, at least 3 weeks, at least 22 days, at least 23 days, at least 24 days, at least 25 days, at least 26 days, at least 27 days, at least 4 weeks, at least 1 month, at least 5 weeks, at least 6 weeks, at least 7 weeks, at least 8 weeks, at least 2 months, at least 9 weeks, at least 10 weeks, at least 11 weeks, at least 12 weeks, at least 3 months, at least 16 weeks, at least 4 months, at least 20 weeks, at least 5 months, at least 24 weeks, at least 6 months, at least 28 weeks, at least 7 months, at least 32 weeks, at least 8 months, at least 36 weeks, at least 9 months, at least 40 weeks, at least 10 months, at least 44 weeks, at least 11 months, at least 48 weeks, or at least 12 months during the concurrent administration of the therapeutically effective amount of aficamten, or the pharmaceutically acceptable salt thereof.

As further described herein a therapeutically effective amount of aficamten, or a pharmaceutically acceptable salt thereof, may be administered to a patient for improving one or more of integrated 2 component Z-score metric, pVO₂, peak workload, peak METs, peak circulatory power, exercise duration, peak respiratory exchange ratio, heart rate reserve, peak heart rate, O₂ pulse at peak exercise, peak proportionate pulse pressure, proportionate pulse pressure at rest, ventilatory efficiency pre-anaerobic threshold, ventilatory efficiency throughout exercise, ventilatory power, ventilatory anaerobic threshold, and aerobic efficiency in a patient in need thereof, wherein treatment with a beta blocker is not contraindicated for the patient.

As further described herein a therapeutically effective amount of a beta blocker, or a pharmaceutically acceptable salt thereof, may be administered to a patient for improving one or more of integrated 2 component Z-score metric, pVO₂, peak workload, peak METs, peak circulatory power, exercise duration, peak respiratory exchange ratio, heart rate reserve, peak heart rate, O₂ pulse at peak exercise, peak proportionate pulse pressure, proportionate pulse pressure at rest, ventilatory efficiency pre-anaerobic threshold, ventilatory efficiency throughout exercise, ventilatory power, ventilatory anaerobic threshold, and aerobic efficiency in a patient in need thereof, wherein the patient is concurrently undergoing a treatment with aficamten, or a pharmaceutically acceptable salt thereof.

As further described herein a therapeutically effective amount of aficamten, or a pharmaceutically acceptable salt thereof, and a beta blocker may be administered to a patient for improving one or more of integrated 2 component Z-score metric, pVO₂, peak workload, peak METs, peak circulatory power, exercise duration, peak respiratory exchange ratio, heart rate reserve, peak heart rate, O₂ pulse at peak exercise, peak proportionate pulse pressure, proportionate pulse pressure at rest, ventilatory efficiency pre-anaerobic threshold, ventilatory efficiency throughout exercise, ventilatory power, ventilatory anaerobic threshold, and aerobic efficiency in a patient in need thereof.

As further described herein a therapeutically effective amount of aficamten, or a pharmaceutically acceptable salt thereof, optionally in combination with a beta blocker may be administered to a patient for improving one or more of integrated 2 component Z-score metric, pVO₂, peak workload, peak METs, peak circulatory power, exercise duration, peak respiratory exchange ratio, heart rate reserve, peak heart rate, O₂ pulse at peak exercise, peak proportionate pulse pressure, proportionate pulse pressure at rest, ventilatory efficiency pre-anaerobic threshold, ventilatory efficiency throughout exercise, ventilatory power, ventilatory anaerobic threshold, and acrobic efficiency, in a patient in need thereof.

As further described herein a therapeutically effective amount of aficamten, or a pharmaceutically acceptable salt thereof, may be administered to a patient for improving peak oxygen uptake (pVO₂) in a patient in need thereof, wherein the patient is concurrently undergoing a treatment with a beta blocker.

As further described herein a therapeutically effective amount of aficamten, or a pharmaceutically acceptable salt thereof, may be administered to a patient for improving peak oxygen uptake (pVO₂) in a patient in need thereof, wherein the patient was administered a beta blocker within at least 1 week, at least 2 weeks, at least 3 weeks, or at least 4 weeks prior to administration of the therapeutically effective amount of aficamten, or the pharmaceutically acceptable salt thereof. In some embodiments, the patient was administered a beta blocker within at least 1 day, at least 2 days, at least 3 days, at least 4 days, at least 5 days, at least 6 days, at least 1 week, at least 8 days, at least 9 days, at least 10 days, at least 11 days, at least 12 days, at least 13 days, at least 2 weeks, at least 15 days, at least 16 days, at least 17 days, at least 18 days, at least 19 days, at least 20 days, at least 3 weeks, at least 22 days, at least 23 days, at least 24 days, at least 25 days, at least 26 days, at least 27 days, at least 4 weeks, at least 1 month, at least 5 weeks, at least 6 weeks, at least 7 weeks, at least 8 weeks, at least 2 months, at least 9 weeks, at least 10 weeks, at least 11 weeks, at least 12 weeks, at least 3 months, at least 16 weeks, at least 4 months, at least 20 weeks, at least 5 months, at least 24 weeks, at least 6 months, at least 28 weeks, at least 7 months, at least 32 weeks, at least 8 months, at least 36 weeks, at least 9 months, at least 40 weeks, at least 10 months, at least 44 weeks, at least 11 months, at least 48 weeks, or at least 12 months prior to administration of the therapeutically effective amount of aficamten, or the pharmaceutically acceptable salt thereof. In some embodiments, the patient continues receiving the beta blocker after the administration of aficamten, or the pharmaceutically acceptable salt thereof, started.

As further described herein a therapeutically effective amount of aficamten, or a pharmaceutically acceptable salt thereof, may be administered to a patient for improving peak oxygen uptake (pVO₂) in a patient in need thereof, resulting in an improvement in peak oxygen uptake (pVO₂); wherein the patient is concurrently undergoing a treatment with a beta blocker; and wherein the improvement in peak oxygen uptake (pVO₂) is not substantially diminished by the treatment with the beta blocker.

As further described herein a therapeutically effective amount of aficamten, or a pharmaceutically acceptable salt thereof, may be administered to a patient for improving peak oxygen uptake (pVO₂) in a patient in need thereof, wherein the patient has been undergoing a treatment with a beta blocker prior to administration of aficamten, or the pharmaceutically acceptable salt thereof, and the treatment with the beta blocker remains unchanged concurrently to the administration of aficamten, or the pharmaceutically acceptable salt thereof. In some embodiments, the treatment with the beta blocker is not changed for at least 1 day, at least 2 days, at least 3 days, at least 4 days, at least 5 days, at least 6 days, at least 1 week, at least 8 days, at least 9 days, at least 10 days, at least 11 days, at least 12 days, at least 13 days, at least 2 weeks, at least 15 days, at least 16 days, at least 17 days, at least 18 days, at least 19 days, at least 20 days, at least 3 weeks, at least 22 days, at least 23 days, at least 24 days, at least 25 days, at least 26 days, at least 27 days, at least 4 weeks, at least 1 month, at least 5 weeks, at least 6 weeks, at least 7 weeks, at least 8 weeks, at least 2 months, at least 9 weeks, at least 10 weeks, at least 11 weeks, at least 12 weeks, at least 3 months, at least 16 weeks, at least 4 months, at least 20 weeks, at least 5 months, at least 24 weeks, at least 6 months, at least 28 weeks, at least 7 months, at least 32 weeks, at least 8 months, at least 36 weeks, at least 9 months, at least 40 weeks, at least 10 months, at least 44 weeks, at least 11 months, at least 48 weeks, or at least 12 months during the concurrent administration of the therapeutically effective amount of aficamten, or the pharmaceutically acceptable salt thereof. In some embodiments, the treatment with the beta blocker is not changed for at least 1 day, at least 2 days, at least 3 days, at least 4 days, at least 5 days, at least 6 days, at least 1 week, at least 8 days, at least 9 days, at least 10 days, at least 11 days, at least 12 days, at least 13 days, at least 2 weeks, at least 15 days, at least 16 days, at least 17 days, at least 18 days, at least 19 days, at least 20 days, at least 3 weeks, at least 22 days, at least 23 days, at least 24 days, at least 25 days, at least 26 days, at least 27 days, at least 4 weeks, at least 1 month, at least 5 weeks, at least 6 weeks, at least 7 weeks, at least 8 weeks, at least 2 months, at least 9 weeks, at least 10 weeks, at least 11 weeks, at least 12 weeks, at least 3 months, at least 16 weeks, at least 4 months, at least 20 weeks, at least 5 months, at least 24 weeks, at least 6 months, at least 28 weeks, at least 7 months, at least 32 weeks, at least 8 months, at least 36 weeks, at least 9 months, at least 40 weeks, at least 10 months, at least 44 weeks, at least 11 months, at least 48 weeks, or at least 12 months following administration of the therapeutically effective amount of aficamten, or the pharmaceutically acceptable salt thereof.

As further described herein a therapeutically effective amount of aficamten, or a pharmaceutically acceptable salt thereof, may be administered to a patient for improving peak oxygen uptake (pVO₂) in a patient in need thereof, wherein the patient has been undergoing a treatment with a beta blocker prior to administration of aficamten, or the pharmaceutically acceptable salt thereof, and the treatment with the beta blocker is not changed for at least 1 week, at least 2 weeks, at least 3 weeks, or at least 4 weeks during the concurrent administration of aficamten, or the pharmaceutically acceptable salt thereof. In some embodiments, the treatment with the beta blocker is not changed for at least 1 day, at least 2 days, at least 3 days, at least 4 days, at least 5 days, at least 6 days, at least 1 week, at least 8 days, at least 9 days, at least 10 days, at least 11 days, at least 12 days, at least 13 days, at least 2 weeks, at least 15 days, at least 16 days, at least 17 days, at least 18 days, at least 19 days, at least 20 days, at least 3 weeks, at least 22 days, at least 23 days, at least 24 days, at least 25 days, at least 26 days, at least 27 days, at least 4 weeks, at least 1 month, at least 5 weeks, at least 6 weeks, at least 7 weeks, at least 8 weeks, at least 2 months, at least 9 weeks, at least 10 weeks, at least 11 weeks, at least 12 weeks, at least 3 months, at least 16 weeks, at least 4 months, at least 20 weeks, at least 5 months, at least 24 weeks, at least 6 months, at least 28 weeks, at least 7 months, at least 32 weeks, at least 8 months, at least 36 weeks, at least 9 months, at least 40 weeks, at least 10 months, at least 44 weeks, at least 11 months, at least 48 weeks, or at least 12 months during the concurrent administration of the therapeutically effective amount of aficamten, or the pharmaceutically acceptable salt thereof.

As further described herein a therapeutically effective amount of aficamten, or a pharmaceutically acceptable salt thereof, may be administered to a patient for improving peak oxygen uptake (pVO₂) in a patient in need thereof, wherein treatment with a beta blocker is not contraindicated for the patient.

As further described herein a therapeutically effective amount of a beta blocker, or a pharmaceutically acceptable salt thereof, may be administered to a patient for improving peak oxygen uptake (pVO₂) in a patient in need thereof, wherein the patient is concurrently undergoing a treatment with aficamten, or a pharmaceutically acceptable salt thereof.

As further described herein a therapeutically effective amount of aficamten, or a pharmaceutically acceptable salt thereof, and a beta blocker may be administered to a patient for improving peak oxygen uptake (pVO₂) in a patient in need thereof.

As further described herein a therapeutically effective amount of aficamten, or a pharmaceutically acceptable salt thereof, optionally in combination with a beta blocker may be administered to a patient for improving peak oxygen uptake (pVO₂) in a patient in need thereof.

As further described herein a therapeutically effective amount of aficamten, or a pharmaceutically acceptable salt thereof, and a beta blocker may be administered to a patient for improving heart rate reserve in a patient in need thereof.

As further described herein a therapeutically effective amount of aficamten, or a pharmaceutically acceptable salt thereof, and a beta blocker may be administered to a patient for improving peak metabolic equivalents (METs) in a patient in need thereof.

As further described herein a therapeutically effective amount of aficamten, or a pharmaceutically acceptable salt thereof, and a beta blocker may be administered to a patient for improving exercise duration in a patient in need thereof.

As further described herein a therapeutically effective amount of aficamten, or a pharmaceutically acceptable salt thereof, and a beta blocker may be administered to a patient for improving one or more of integrated 2 component Z-score metric, pVO₂, peak workload, peak METs, peak circulatory power, exercise duration, peak respiratory exchange ratio, heart rate reserve, peak heart rate, O₂ pulse at peak exercise, peak proportionate pulse pressure, proportionate pulse pressure at rest, ventilatory efficiency pre-anaerobic threshold, ventilatory efficiency throughout exercise, ventilatory power, ventilatory anaerobic threshold, and aerobic efficiency, in a patient in need thereof.

As further described herein a therapeutically effective amount of aficamten, or a pharmaceutically acceptable salt thereof, and a beta blocker may be administered to a patient for improving peak oxygen uptake (pVO₂) in a patient in need thereof.

As further described herein a therapeutically effective amount of aficamten, or a pharmaceutically acceptable salt thereof, may be administered to a patient for improving heart rate reserve in a patient in need thereof, comprising administering to the patient a therapeutically effective amount of aficamten, or a pharmaceutically acceptable salt thereof, and a beta blocker, are provided therein.

As further described herein a therapeutically effective amount of aficamten, or a pharmaceutically acceptable salt thereof, may be administered to a patient for improving peak metabolic equivalents (METs) in a patient in need thereof, comprising administering to the patient a therapeutically effective amount of aficamten, or a pharmaceutically acceptable salt thereof, and a beta blocker, are provided therein.

As further described herein a therapeutically effective amount of aficamten, or a pharmaceutically acceptable salt thereof, may be administered to a patient for improving exercise duration in a patient in need thereof, comprising administering to the patient a therapeutically effective amount of aficamten, or a pharmaceutically acceptable salt thereof, and a beta blocker, are provided therein.

As further described herein a therapeutically effective amount of aficamten, or a pharmaceutically acceptable salt thereof, may be administered to a patient for improving one or more of integrated 2 component Z-score metric, pVO₂, peak workload, peak METs, peak circulatory power, exercise duration, peak respiratory exchange ratio, heart rate reserve, peak heart rate, O₂ pulse at peak exercise, peak proportionate pulse pressure, proportionate pulse pressure at rest, ventilatory efficiency pre-anaerobic threshold, ventilatory efficiency throughout exercise, ventilatory power, ventilatory anaerobic threshold, and aerobic efficiency, in a patient in need thereof, comprising administering to the patient a therapeutically effective amount of aficamten, or a pharmaceutically acceptable salt thereof, and a beta blocker, are provided therein.

As further described herein a therapeutically effective amount of aficamten, or a pharmaceutically acceptable salt thereof, may be administered to a patient for improving peak oxygen uptake (pVO₂) in a patient in need thereof, comprising administering to the patient a therapeutically effective amount of aficamten, or a pharmaceutically acceptable salt thereof, and a beta blocker, are provided therein.

Aficamten, or a pharmaceutically acceptable salt thereof, may be administered at a constant dose level, aficamten, or a pharmaceutically acceptable salt thereof, may be administered at a titrated dose level. For example, the dose of aficamten, or a pharmaceutically acceptable salt thereof, may be adjusted depending on the patient's response to the drug. That is, the dose of the aficamten, or a pharmaceutically acceptable salt thereof, may be periodically increased, decreased, or maintained depending on the measurement of a drug response, such as one or more of a left ventricular outflow tract pressure gradient (LVOT-G), biplane left ventricular ejection fraction (LVEF), and/or post-Valsalva LVOT-G measurements.

In some embodiments, the patient has a heart disease. In some embodiments, the heart disease is hypertrophic cardiomyopathy (HCM). In some embodiments, the HCM is obstructive or nonobstructive or is associated with a sarcomeric and/or non-sarcomeric mutation. In some embodiments, the heart disease is heart failure with preserved ejection fraction (HFpEF). In some embodiments, the heart disease is selected from the group consisting of diastolic dysfunction, primary or secondary restrictive cardiomyopathy, myocardial infarction and angina pectoris, left ventricular outflow tract obstruction, hypertensive heart disease, congenital heart disease, cardiac ischemia, coronary heart disease, diabetic heart disease, congestive heart failure, right heart failure, cardiorenal syndrome, and infiltrative cardiomyopathy. In some embodiments, the heart disease is or is related to one or more conditions selected from the group consisting of cardiac senescence, diastolic dysfunction due to aging, left ventricular hypertrophy and concentric left ventricular remodeling.

In some embodiments, the patient has a disease or condition associated with HCM. In some embodiments, the disease or condition associated with HCM selected from the group consisting of Fabry's Disease, Danon Disease, mitochondrial cardiomyopathies, and Noonan Syndrome. In some embodiments, the patient has a disease or condition selected from the group consisting of Fabry's Disease, Danon Disease, mitochondrial cardiomyopathies, and Noonan Syndrome.

In some embodiments, the patient has a disease or condition that is associated with secondary left ventricular wall thickening. In some embodiments, the disease or condition associated with secondary left ventricular wall thickening is selected from the group consisting of hypertension, valvular heart diseases (aortic stenosis, Mitral valve regurgitation), metabolic syndromes (diabetes, obesity), end stage renal disease, scleroderma, sleep apnea, amyloidosis, Fabry's disease, Friedreich Ataxia, Danon disease, Noonan syndrome, and Pompe disease. In some embodiments, the patient has a disease or condition selected from the group consisting of hypertension, valvular heart diseases (aortic stenosis, Mitral valve regurgitation), metabolic syndromes (diabetes, obesity), end stage renal disease, scleroderma, sleep apnea, amyloidosis, Fabry's disease, Friedreich Ataxia, Danon disease, Noonan syndrome, and Pompe disease.

In some embodiments, the patient has a disease or condition that is associated with small left ventricular cavity, cavity obliteration, hyperdynamic left ventricular contraction, myocardial ischemia, or cardiac fibrosis.

In some embodiments, the patient has a disease or condition selected from muscular dystrophies and glycogen storage diseases.

As further described herein a therapeutically effective amount of aficamten, or a pharmaceutically acceptable salt thereof, optionally in combination with a beta blocker may be administered to a patient for treating a disease or condition in a patient in need thereof, comprising administering to the patient a therapeutically effective amount of aficamten, or a pharmaceutically acceptable salt thereof, and optionally a beta blocker;
wherein the disease or condition is selected from the group consisting of:
   (a) hypertrophic cardiomyopathy (HCM);
   (b) a heart disease selected from the group consisting of diastolic dysfunction, primary or secondary restrictive cardiomyopathy, myocardial infarction and angina pectoris, left ventricular outflow tract obstruction, hypertensive heart disease, congenital heart disease, cardiac ischemia, coronary heart disease, diabetic heart disease, congestive heart failure, right heart failure, cardiorenal syndrome, and infiltrative cardiomyopathy;
   (c) a heart disease that is or is related to one or more conditions selected from the group consisting of cardiac senescence, diastolic dysfunction due to aging, left ventricular hypertrophy and concentric left ventricular remodeling;
   (d) a disease or condition associated with HCM;
   (e) a disease or condition that is associated with secondary left ventricular wall thickening;
   (f) a disease or condition that is associated with small left ventricular cavity, cavity obliteration, hyperdynamic left ventricular contraction, myocardial ischemia, or cardiac fibrosis; and
   (g) a disease or condition selected from muscular dystrophies and glycogen storage diseases;
and
wherein the method improves one or more parameter in the patient wherein each of the one or more parameter is selected from the group consisting of:
   (1) heart rate reserve;
   (2) peak metabolic equivalents (METs);
   (3) exercise duration;
   (4) peak oxygen uptake (pVO₂); and
   (5) one or more of integrated 2 component Z-score metric, peak workload, peak circulatory power, peak respiratory exchange ratio, O₂ pulse at peak exercise, peak proportionate pulse pressure, proportionate pulse pressure at rest, ventilatory efficiency pre-anaerobic threshold, ventilatory efficiency throughout exercise, ventilatory power, ventilatory anaerobic threshold, and aerobic efficiency in the patient.

As further described herein a therapeutically effective amount of aficamten, or a pharmaceutically acceptable salt thereof, optionally in combination with a beta blocker may be administered to a patient for treating a disease or condition in a patient in need thereof, comprising administering to the patient a therapeutically effective amount of aficamten, or a pharmaceutically acceptable salt thereof, and optionally a beta blocker;
wherein the disease or condition is selected from the group consisting of:
   (a) hypertrophic cardiomyopathy (HCM);
   (b) a heart disease selected from the group consisting of diastolic dysfunction, primary or secondary restrictive cardiomyopathy, myocardial infarction and angina pectoris, left ventricular outflow tract obstruction, hypertensive heart disease, congenital heart disease, cardiac ischemia, coronary heart disease, diabetic heart disease, congestive heart failure, right heart failure, cardiorenal syndrome, and infiltrative cardiomyopathy; and
   (c) a heart disease that is or is related to one or more conditions selected from the group consisting of cardiac senescence, diastolic dysfunction due to aging, left ventricular hypertrophy and concentric left ventricular remodeling;
and
wherein the method improves one or more parameter in the patient wherein each of the one or more parameter is selected from the group consisting of:
   (1) heart rate reserve;
   (2) peak metabolic equivalents (METs);
   (3) exercise duration;
   (4) peak oxygen uptake (pVO₂); and
   (5) one or more of integrated 2 component Z-score metric, peak workload, peak circulatory power, peak respiratory exchange ratio, peak heart rate, O₂ pulse at peak exercise, peak proportionate pulse pressure, proportionate pulse pressure at rest, ventilatory efficiency pre-anaerobic threshold, ventilatory efficiency throughout exercise, ventilatory power, ventilatory anaerobic threshold, and aerobic efficiency in the patient.

In some implementations of the methods disclosed herein, the therapeutically effective amount of aficamten, or a pharmaceutically acceptable salt thereof, is selected by titrating a daily dose of aficamten, or a pharmaceutically acceptable salt thereof, administered to the patient.

In some implementations of the methods disclosed herein, aficamten, or a pharmaceutically acceptable salt thereof is administered at a daily dose of about 5 mg to about 20 mg.

In some implementations of the methods disclosed herein, aficamten, or a pharmaceutically acceptable salt thereof, is administered once daily.

In some implementations of the methods disclosed herein, the methods comprise administering aficamten, or a pharmaceutically acceptable salt thereof, for at least about 2 weeks, at least about 3 weeks, at least about 4 weeks, at least about 1 month weeks, at least about 5 weeks, at least about 6 weeks, at least about 7 weeks, at least about 8 weeks, at least about 2 months, about 9 weeks, at least about 10 weeks, at least about 11 weeks, at least about 12 weeks, at least 3 months, about 16 weeks, at least about 4 months, about 20 weeks, at least 5 months, about 24 weeks, at least 6 months, about 28 weeks, at least about 7 months, about 32 weeks, at least about 8 months, 36 weeks, at least about 9 months, at least 40 weeks, at least about 10 months, at least about 44 weeks, at least about 11 months, at least about 48 weeks, or at least about 12 months. In some embodiments, the methods comprise administering aficamten, or a pharmaceutically acceptable salt thereof, for at least about 2, at least about 3, at least about 4, at least about 5, at least about 6, at least about 7, at least about 8, at least about 9, at least about 10, at least about 11, at least about 12, at least about 16, at least about 20, at least about 24, at least about 28, at least about 32, at least about 36, at least about 40, at least about 44, or at least about 48 weeks.

In some implementations of the methods disclosed herein, the methods result in an improvement for at least about 2 weeks, at least about 3 weeks, at least about 4 weeks, at least about 1 month weeks, at least about 5 weeks, at least about 6 weeks, at least about 7 weeks, at least about 8 weeks, at least about 2 months, about 9 weeks, at least about 10 weeks, at least about 11 weeks, at least about 12 weeks, at least 3 months, about 16 weeks, at least about 4 months, about 20 weeks, at least 5 months, about 24 weeks, at least 6 months, about 28 weeks, at least about 7 months, about 32 weeks, at least about 8 months, 36 weeks, at least about 9 months, at least 40 weeks, at least about 10 months, at least about 44 weeks, at least about 11 months, at least about 48 weeks, or at least about 12 months after beginning administering aficamten, or a pharmaceutically acceptable salt thereof. In some embodiments, the methods result in an improvement for at least about 2, at least about 3, at least about 4, at least about 5, at least about 6, at least about 7, at least about 8, at least about 9, at least about 10, at least about 11, at least about 12, at least about 16, at least about 20, at least about 24, at least about 28, at least about 32, at least about 36, at least about 40, at least about 44, or at least about 48 weeks after beginning administering aficamten, or a pharmaceutically acceptable salt thereof.

In some implementations of the methods disclosed herein, aficamten or pharmaceutically acceptable salt thereof is administered as a tablet.

In some implementations of the methods disclosed herein, aficamten or pharmaceutically acceptable salt thereof comprises one or more of polymorphic Form I, Form II, Form III, Form IV, Form V, and Form VI of aficamten.

### Heart Rate Reserve

In some implementations of the methods disclosed herein, heart rate reserve is improved by at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, at least 50, at least 55, at least 60, at least 65, at least 70, at least 75, at least 80, at least 85, at least 90, at least 95, or at least 100 beats/minute (bpm) compared to the patient's heart rate reserve prior to administration of aficamten, or the pharmaceutically acceptable salt thereof. In some embodiments, heart rate reserve is improved by at least 1, at least 2, at least 3, at least 4, at least 5, at least 6, at least 7, at least 8, at least 9, at least 10, at least 11, at least 12, at least 13, at least 14, at least 15, at least 20, at least 25, at least 30, at least 35, at least 40, at least 45, at least 50, at least 60, at least 70, at least 80, at least 90, or at least 100 beats/minute (bpm) compared to the patient's heart rate reserve prior to administration of aficamten, or the pharmaceutically acceptable salt thereof.

In some embodiments, heart rate reserve is improved by between 1 and 100 beats/minute (bpm), between 2 and 100 beats/minute (bpm), between 3 and 100 beats/minute (bpm), between 4 and 100 beats/minute (bpm), between 5 and 100 beats/minute (bpm), between 6 and 100 beats/minute (bpm), between 7 and 100 beats/minute (bpm), between 8 and 100 beats/minute (bpm), between 9 and 100 beats/minute (bpm), between 1 and 8 beats/minute (bpm), between 2 and 8 beats/minute (bpm), between 3 and 8 beats/minute (bpm), between 4 and 10 beats/minute (bpm), between 10 and 100 beats/minute (bpm), between 11 and 100 beats/minute (bpm), between 12 and 100 beats/minute (bpm), between 13 and 100 beats/minute (bpm), between 14 and 100 beats/minute (bpm), between 15 and 100 beats/minute (bpm), between 20 and 100 beats/minute (bpm), between 25 and 100 beats/minute (bpm), between 30 and 100 beats/minute (bpm), between 35 and 100 beats/minute (bpm), between 40 and 100 beats/minute (bpm), between 45 and 100 beats/minute (bpm), between 50 and 100 beats/minute (bpm), between 60 and 100 beats/minute (bpm), between 70 and 100 beats/minute (bpm), between 80 and 100 beats/minute (bpm), or between 90 and 100 beats/minute (bpm) compared to the patient's heart rate reserve prior to administration of aficamten, or the pharmaceutically acceptable salt thereof. In some embodiments, heart rate reserve is improved by between 1 and 20 beats/minute (bpm), between 2 and 20 beats/minute (bpm), between 3 and 20 beats/minute (bpm), between 4 and 20 beats/minute (bpm), between 5 and 20 beats/minute (bpm), between 6 and 20 beats/minute (bpm), between 7 and 20 beats/minute (bpm), between 8 and 20 beats/minute (bpm), between 9 and 20 beats/minute (bpm), between 10 and 20 beats/minute (bpm), between 11 and 20 beats/minute (bpm), between 12 and 20 beats/minute (bpm), between 13 and 20 beats/minute (bpm), between 14 and 20 beats/minute (bpm), or between 15 and 20 beats/minute (bpm) compared to the patient's heart rate reserve prior to administration of aficamten, or the pharmaceutically acceptable salt thereof.

### Peak Heart Rate

In some implementations of the methods disclosed herein, peak heart rate is improved by at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, at least 50, at least 55, at least 60, at least 65, at least 70, at least 75, at least 80, at least 85, at least 90, at least 95, or at least 100 beats/minute (bpm) compared to the patient's peak heart rate prior to administration of aficamten, or the pharmaceutically acceptable salt thereof. In some embodiments, peak heart rate is improved by at least 1, at least 2, at least 3, at least 4, at least 5, at least 6, at least 7, at least 8, at least 9, at least 10, at least 11, at least 12, at least 13, at least 14, at least 15, at least 20, at least 25, at least 30, at least 35, at least 40, at least 45, at least 50, at least 60, at least 70, at least 80, at least 90, or at least 100 beats/minute (bpm) compared to the patient's peak heart rate prior to administration of aficamten, or the pharmaceutically acceptable salt thereof.

In some embodiments, peak heart rate is improved by between 1 and 100 beats/minute (bpm), between 2 and 100 beats/minute (bpm), between 3 and 100 beats/minute (bpm), between 4 and 100 beats/minute (bpm), between 5 and 100 beats/minute (bpm), between 6 and 100 beats/minute (bpm), between 7 and 100 beats/minute (bpm), between 8 and 100 beats/minute (bpm), between 9 and 100 beats/minute (bpm), between 1 and 8 beats/minute (bpm), between 2 and 8 beats/minute (bpm), between 3 and 8 beats/minute (bpm), between 4 and 10 beats/minute (bpm), between 10 and 100 beats/minute (bpm), between 11 and 100 beats/minute (bpm), between 12 and 100 beats/minute (bpm), between 13 and 100 beats/minute (bpm), between 14 and 100 beats/minute (bpm), between 15 and 100 beats/minute (bpm), between 20 and 100 beats/minute (bpm), between 25 and 100 beats/minute (bpm), between 30 and 100 beats/minute (bpm), between 35 and 100 beats/minute (bpm), between 40 and 100 beats/minute (bpm), between 45 and 100 beats/minute (bpm), between 50 and 100 beats/minute (bpm), between 60 and 100 beats/minute (bpm), between 70 and 100 beats/minute (bpm), between 80 and 100 beats/minute (bpm), or between 90 and 100 beats/minute (bpm) compared to the patient's peak heart rate prior to administration of aficamten, or the pharmaceutically acceptable salt thereof. In some embodiments, peak heart rate is improved by between 1 and 20 beats/minute (bpm), between 2 and 20 beats/minute (bpm), between 3 and 20 beats/minute (bpm), between 4 and 20 beats/minute (bpm), between 5 and 20 beats/minute (bpm), between 6 and 20 beats/minute (bpm), between 7 and 20 beats/minute (bpm), between 8 and 20 beats/minute (bpm), between 9 and 20 beats/minute (bpm), between 10 and 20 beats/minute (bpm), between 11 and 20 beats/minute (bpm), between 12 and 20 beats/minute (bpm), between 13 and 20 beats/minute (bpm), between 14 and 20 beats/minute (bpm), or between 15 and 20 beats/minute (bpm) compared to the patient's peak heart rate prior to administration of aficamten, or the pharmaceutically acceptable salt thereof.

### Metabolic Equivalents (METs)

In some implementations of the methods disclosed herein, peak METs are improved by at least 0.1, at least 0.2, at least 0.3, at least 0.4, at least 0.5, at least 0.6, at least 0.7, at least 0.8, at least 0.9, at least 1.0, at least 1.1, at least 1.2, at least 1.3, at least 1.4, at least 1.5, at least 1.6, at least 1.7, at least 1.8, at least 1.9, at least 2.0, at least 2.1, at least 2.2, at least 2.3, at least 2.4, at least 2.5, at least 2.6, at least 2.7, at least 2.8, at least 2.9, at least 3.0, at least 4.0, at least 5.0, at least 6.0, at least 7.0, at least 8.0, at least 9.0, at least 10, at least 15, at least 20, at least 25, or at least 30 mL/kg/min compared to the patient's peak METs prior to administration of aficamten, or the pharmaceutically acceptable salt thereof. In some embodiments, peak METs are improved by between 0.1 and 30, between 0.2 and 30, between 0.3 and 30, between 0.4 and 30, between 0.5 and 30, between 0.6 and 30, between 0.7 and 30, between 0.8 and 30, between 0.9 and 30, between 1.0 and 30, between 1.1 and 30, between 1.2 and 30, between 1.3 and 30, between 1.4 and 30, between 1.5 and 30, between 1.6 and 30, between 1.7 and 30, between 1.8 and 30, between 1.9 and 30, between 2.0 and 30, between 2.1 and 30, between 2.2 and 30, between 2.3 and 30, between 2.4 and 30, between 2.5 and 30, between 2.6 and 30, between 2.7 and 30, between 2.8 and 30, between 2.9 and 30, between 3.0 and 30, between 4.0 and 30, between 5.0 and 30, between 6.0 and 30, between 7.0 and 30, between 8.0 and 30, between 9.0 and 30, between 10 and 30, between 15 and 30, between 20 and 30, or between 25 and 30 mL/kg/min compared to the patient's peak METs prior to administration of aficamten, or the pharmaceutically acceptable salt thereof. In some embodiments, peak METs are improved by between 0.1 and 5.0, between 0.1 and 1.0, between 0.1 and 0.6, between 0.1 and 0.7, between 0.2 and 5.0, between 0.2 and 1.0, between 0.2 and 0.7, between 0.3 and 5, between 0.3 and 0.7, between 0.4 and 5.0, between 0.5 and 5.0, between 0.6 and 5.0, between 0.7 and 5.0, between 0.8 and 5.0, between 0.9 and 5.0, between 1.0 and 5.0, between 1.1 and 5.0, between 1.2 and 5.0, between 1.3 and 5.0, between 1.4 and 5.0, between 1.5 and 5.0, between 1.6 and 5.0, between 1.7 and 5.0, between 1.8 and 5.0, between 1.9 and 5.0, between 2.0 and 5.0, between 2.1 and 5.0, between 2.2 and 5.0, between 2.3 and 5.0, between 2.4 and 5.0, between 2.5 and 5.0, between 2.6 and 5.0, between 2.7 and 5.0, between 2.8 and 5.0, between 2.9 and 5.0, between 3.0 and 5.0, or between 4.0 and 5.0 mL/kg/min compared to the patient's peak METs prior to administration of aficamten, or the pharmaceutically acceptable salt thereof.

### Exercise Duration

In some implementations of the methods disclosed herein, exercise duration is improved by at least 0.1, at least 0.2, at least 0.3, at least 0.4, at least 0.5, at least 0.6, at least 0.7, at least 0.8, at least 0.9, at least 1.0, at least 1.1, at least 1.2, at least 1.3, at least 1.4, at least 1.5, at least 1.6, at least 1.7, at least 1.8, at least 1.9, at least 2.0, at least 2.1, at least 2.2, at least 2.3, at least 2.4, at least 2.5, at least 2.6, at least 2.7, at least 2.8, at least 2.9, at least 3.0, at least 4.0, at least 5.0, at least 6.0, at least 7.0, at least 8.0, at least 9.0, at least 10, at least 15, at least 20, at least 25, at least 30, at least 35, at least 40, at least 45, at least 50, at least 55, or at least 60 min compared to the patient's exercise duration prior to administration of aficamten, or the pharmaceutically acceptable salt thereof. In some embodiments, exercise duration is improved by at least 0.1, at least 0.2, at least 0.3, at least 0.4, at least 0.5, at least 0.6, at least 0.7, at least 0.8, at least 0.9, at least 1.0, at least 1.1, at least 1.2, at least 1.3, at least 1.4, at least 1.5, at least 1.6, at least 1.7, at least 1.8, at least 1.9, at least 2.0, at least 2.1, at least 2.2, at least 2.3, at least 2.4, at least 2.5, at least 2.6, at least 2.7, at least 2.8, at least 2.9, at least 3.0, at least 4.0, at least 5.0, at least 6.0, at least 7.0, at least 8.0, at least 9.0, at least 10, or at least 15 min compared to the patient's exercise duration prior to administration of aficamten, or the pharmaceutically acceptable salt thereof. In some embodiments, exercise duration is improved by between 0.1 and 60, between 0.2 and 60, between 0.3 and 60, between 0.4 and 60, between 0.5 and 60, between 0.6 and 60, between 0.7 and 60, between 0.8 and 60, between 0.9 and 60, between 1.0 and 60, between 1.1 and 60, between 1.2 and 60, between 1.3 and 60, between 1.4 and 60, between 1.5 and 60, between 1.6 and 60, between 1.7 and 60, between 1.8 and 60, between 1.9 and 60, between 2.0 and 60, between 2.1 and 60, between 2.2 and 60, between 2.3 and 60, between 2.4 and 60, between 2.5 and 60, between 2.6 and 60, between 2.7 and 60, between 2.8 and 60, between 2.9 and 60, between 3.0 and 60, between 4.0 and 60, between 5.0 and 60, between 6.0 and 60, between 7.0 and 60, between 8.0 and 60, between 9.0 and 60, between 10 and 60, between 15 and 60, between 20 and 60, between 25 and 60, between 30 and 60, between 35 and 60, between 40 and 60, between 45 and 60, between 50 and 60, between 55 and 60 min compared to the patient's exercise duration prior to administration of aficamten, or the pharmaceutically acceptable salt thereof. In some embodiments, exercise duration is improved by between 0.1 and 10, between 0.2 and 10, between 0.3 and 10, between 0.4 and 10, between 0.5 and 10, between 0.6 and 10, between 0.7 and 10, between 0.8 and 10, between 0.9 and 10, between 1.0 and 10, between 1.0 and 2.0, between 0.8 and 1.4, between 0.6 and 1.5, between 1.1 and 10, between 1.2 and 10, between 1.3 and 10, between 1.4 and 10, between 1.5 and 10, between 1.6 and 10, between 1.7 and 10, between 1.8 and 10, between 1.9 and 10, between 2.0 and 10, between 2.1 and 10, between 2.2 and 10, between 2.3 and 10, between 2.4 and 10, between 2.5 and 10, between 2.6 and 10, between 2.7 and 10, between 2.8 and 10, between 2.9 and 10, between 3.0 and 10, between 4.0 and 10, between 5.0 and 10, between 6.0 and 10, between 7.0 and 10, between 8.0 and 10, or between 9.0 and 10 min compared to the patient's exercise duration prior to administration of aficamten, or the pharmaceutically acceptable salt thereof.

Peak Oxygen Uptake (pVO₂)

In some implementations of the methods disclosed herein, pVO₂ is improved by at least 0.1, at least 0.2, at least 0.3, at least 0.4, at least 0.5, at least 0.6, at least 0.7, at least 0.8, at least 0.9, at least 1.0, at least 1.1, at least 1.2, at least 1.3, at least 1.4, at least 1.5, at least 1.6, at least 1.7, at least 1.8, at least 1.9, at least 2.0, at least 2.1, at least 2.2, at least 2.3, at least 2.4, at least 2.5, at least 2.6, at least 2.7, at least 2.8, at least 2.9, at least 3.0, at least 4.0, at least 5.0, at least 6.0, at least 7.0, at least 8.0, at least 9.0, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30 mL/kg/min compared to the patient's pVO₂ prior to administration of aficamten, or the pharmaceutically acceptable salt thereof. In some embodiments, pVO₂ is improved by at least 0.1, at least 0.2, at least 0.3, at least 0.4, at least 0.5, at least 0.6, at least 0.7, at least 0.8, at least 0.9, at least 1.0, at least 1.1, at least 1.2, at least 1.3, at least 1.4, at least 1.5, at least 1.6, at least 1.7, at least 1.8, at least 1.9, at least 2.0, at least 2.1, at least 2.2, at least 2.3, at least 2.4, at least 2.5, at least 2.6, at least 2.7, at least 2.8, at least 2.9, at least 3.0, at least 4.0, at least 5.0, at least 6.0, at least 7.0, at least 8.0, at least 9.0, at least 10, at least 15, at least 20, at least 25, or at least 30 mL/kg/min compared to the patient's pVO₂ prior to administration of aficamten, or the pharmaceutically acceptable salt thereof. In some embodiments, pVO₂ is improved by between 0.1 and 30, between at least 0.2 and 30, between 0.3 and 30, between 0.4 and 30, between 0.5 and 30, between 0.6 and 30, between 0.7 and 30, between 0.8 and 30, between 0.9 and 30, between 1.0 and 30, between 1.1 and 30, between 1.2 and 30, between 1.3 and 30, between 1.4 and 30, between 1.5 and 30, between 1.6 and 30, between 1.7 and 30, between 1.7 and 2.0, between 1.75 and 2.0, between 1.6 and 2.0, between 1.5 and 2.0, between 1.5 and 2.0, between 1.8 and 2.0, between 1.8 and 30, between 1.9 and 30, between 2.0 and 30, between 2.1 and 30, between 2.2 and 30, between 2.3 and 30, between 2.4 and 30, between 2.5 and 30, between 2.6 and 30, between 2.7 and 30, between 2.8 and 30, between 2.9 and 30, between 3.0 and 30, between 4.0 and 30, between 5.0 and 30, between 6.0 and 30, between 7.0 and 30, between 8.0 and 30, between 9.0 and 30, between 10 and 30, between 15 and 30, between 20 and 30, between 25 and 30 mL/kg/min compared to the patient's pVO₂ prior to administration of aficamten, or the pharmaceutically acceptable salt thereof. In some embodiments, pVO₂ is improved by between 0.1 and 10, between at least 0.2 and 10, between 0.3 and 10, between 0.4 and 10, between 0.5 and 10, between 0.6 and 10, between 0.7 and 10, between 0.8 and 10, between 0.9 and 10, between 1.0 and 10, between 1.1 and 10, between 1.2 and 10, between 1.3 and 10, between 1.4 and 10, between 1.5 and 10, between 1.6 and 10, between 1.7 and 10, between 1.8 and 10, between 1.9 and 10, between 2.0 and 10, between 2.1 and 10, between 2.2 and 10, between 2.3 and 10, between 2.4 and 10, between 2.5 and 10, between 2.6 and 10, between 2.7 and 10, between 2.8 and 10, between 2.9 and 10, between 3.0 and 10, between 4.0 and 10, between 5.0 and 10, between 6.0 and 10, between 7.0 and 10, between 8.0 and 10, or between 9.0 and 10 mL/kg/min compared to the patient's pVO₂ prior to administration of aficamten, or the pharmaceutically acceptable salt thereof.

### Beta Blockers

In some implementations of the methods disclosed herein, aficamten, or the pharmaceutically acceptable salt thereof is administered with a beta blocker. In some embodiments, the beta blocker is selected from the group consisting of a non-selective beta blocker, a β₁-selective beta blocker, a β₂-selective beta blocker, and a β₃-selective beta blocker. In some embodiments, the beta blocker is selected from the group consisting of acebutolol, atenolol, metoprolol, nadolol, nebivolol, propranolol, betaxolol, bisoprolol, celiprolol, esmolol, bucindolol, carteolol, carvedilol, labetalol, nadolol, oxprenolol, penbutolol, pindolol, sotalol, timolol, and butaxamine.

In some embodiments, the beta blocker is administered once daily. In some embodiments, the beta blocker is administered twice daily.

In some embodiments, aficamten, or the pharmaceutically acceptable salt thereof, and the beta blocker are administered concurrently. In some embodiments, aficamten, or the pharmaceutically acceptable salt thereof, and the beta blocker are administered sequentially.

### Dose-Titration

In some implementations of the methods disclosed herein, the therapeutically effective amount of aficamten, or a pharmaceutically acceptable salt thereof, is selected by titrating a daily dose of aficamten, or a pharmaceutically acceptable salt thereof, administered to the patient. Dose titration methods are described in further details herein. Dose titration methods are described in WO 2023/288324, U.S. Patent Publication No. US-2023-0058927-A1, WO 2024/020468 and U.S. Patent Publication US-2024-0091203-A1, and WO 2024/031016 and U.S. Patent Publication US-2024-0115554-A1, the disclosures of which are incorporated by reference herein.

### Oxygen Uptake Recovery (VO₂Rec)

Methods and compositions for accelerating oxygen uptake recovery (VO₂Rec) in a patient in need thereof, comprising administering to the patient a therapeutically effective amount of aficamten or a pharmaceutically acceptable salt thereof, are provided therein. In some embodiments, the patient has a heart disease. In some embodiments, the heart disease is hypertrophic cardiomyopathy (HCM). In some embodiments, the heart disease is oHCM. In some embodiments, the heart disease is nHCM. In some embodiments, the heart disease is heart failure with preserved ejection fraction.

### Left Ventricular Hypertrophy (LVH)

Methods and compositions for reducing or resolving left ventricular hypertrophy (LVH) in a patient in need thereof, comprising administering to the patient a therapeutically effective amount of aficamten or a pharmaceutically acceptable salt thereof, are provided therein. In some embodiments, the patient has a heart disease. In some embodiments, the heart disease is hypertrophic cardiomyopathy (HCM). In some embodiments, the heart disease is oHCM. In some embodiments, the heart disease is nHCM. In some embodiments, the heart disease is heart failure with preserved ejection fraction. In some embodiments, the LVH is LVH with strain pattern. In some embodiments, the LVH is LVH with strain pattern on electrocardiogram.

### Maximal Wall Thickness (MWT)

Methods and compositions for decreasing maximal wall thickness (MWT) in a patient in need thereof, comprising administering to the patient a therapeutically effective amount of aficamten or a pharmaceutically acceptable salt thereof, are provided therein. In some embodiments, the patient has a heart disease. In some embodiments, the heart disease is hypertrophic cardiomyopathy (HCM). In some embodiments, the heart disease is oHCM. In some embodiments, the heart disease is nHCM. In some embodiments, the heart disease is heart failure with preserved ejection fraction.

### Chest Pain

Methods and compositions for reducing chest pain in a patient in need thereof, comprising administering to the patient a therapeutically effective amount of aficamten or a pharmaceutically acceptable salt thereof, are provided therein. In some embodiments, the patient has a heart disease. In some embodiments, the heart disease is hypertrophic cardiomyopathy (HCM). In some embodiments, the heart disease is oHCM. In some embodiments, the heart disease is nHCM. In some embodiments, the heart disease is heart failure with preserved ejection fraction.

### Additional Parameters

Methods and compositions for treating a patient in need thereof, comprising administering to the patient a therapeutically effective amount of aficamten or a pharmaceutically acceptable salt thereof, wherein the method improves one or more parameter in the patient wherein each of the one or more parameter is selected from the group consisting of: improvement in mitral valve SAM, improvement in peak A velocity, complete resolution of mitral valve SAM, increase in LV end systolic volume index, increase in LV end systolic volume, increase in Lateral e′ velocity, increase in Septal e′ velocity, increase in LV end systolic dimension, increase in absolute LV GLS (left ventricular global longitudinal strain), decrease in LA volume index, decrease in wall thickness, decrease in maximum wall thickness, decrease in interventricular septal wall thickness, decrease in interventricular septal wall, decrease in inferolateral wall thickness, decrease in LV mass index, decrease in LV global circumferential strain, decrease in TAPSE, decrease in RV s′ velocity, decrease in LA width, decrease in lateral E/e′, decrease in septal E/e′, decrease in peak E wave velocity, and decrease in LV GCS (left ventricular global circumferential strain), are provided therein. In some embodiments, the patient has a heart disease. In some embodiments, the heart disease is hypertrophic cardiomyopathy (HCM). In some embodiments, the HCM is obstructive or nonobstructive or is associated with a sarcomeric and/or non-sarcomeric mutation. In some embodiments, the heart disease is oHCM. In some embodiments, the heart disease is nHCM. In some embodiments, the heart disease is heart failure with preserved ejection fraction.

### Treatment of Hypertrophic Cardiomyopathy

As further described herein a therapeutically effective amount of aficamten, or a pharmaceutically acceptable salt thereof, may be administered to a patient for the treatment of hypertrophic cardiomyopathy. Aficamten, or a pharmaceutically acceptable salt thereof, may be administered at a constant dose level. aficamten, or a pharmaceutically acceptable salt thereof, may be administered at a titrated dose level. For example, the dose of aficamten, or a pharmaceutically acceptable salt thereof, may be adjusted depending on the patient's response to the drug. That is, the dose of the aficamten, or a pharmaceutically acceptable salt thereof, may be periodically increased, decreased, or maintained depending on the measurement of a drug response, such as one or more of a left ventricular outflow tract pressure gradient (LVOT-G), biplane left ventricular ejection fraction (LVEF), and/or post-Valsalva LVOT-G measurements. Aficamten, or a pharmaceutically acceptable salt thereof, is administered at a

therapeutically effective dosage, e.g., a dosage sufficient to provide treatment for the disease state. For a human, the daily dose may be between about 1 mg and about 50 mg. For example, the daily dose may be about 5 mg, about 10 mg, about 15 mg, about 20 mg, or about 30 mg, or any amount therebetween. A daily dose is the total amount administered in a day. A daily dose may be, but is not limited to be, administered each day, every other day, each week, every 2 weeks, every month, or at a varied interval. In some embodiments, the daily dose is administered for a period ranging from a single day to the life of the subject. In some embodiments, the daily dose is administered once a day. In some embodiments, the daily dose is administered in multiple divided doses, such as in 2, 3, or 4 divided doses. In some embodiments, the daily dose is administered in 2 divided doses.

In one example, a patient is treated for hypertrophic cardiomyopathy by administering to the patient a daily dose of about 5 mg to about 30 mg of aficamten, or a pharmaceutically acceptable salt thereof. In one example, a patient is treated for hypertrophic cardiomyopathy by administering to the patient a daily dose of about 5 mg aficamten, or a pharmaceutically acceptable salt thereof. In one example, a patient is treated for hypertrophic cardiomyopathy by administering to the patient a daily dose of about 10 mg aficamten, or a pharmaceutically acceptable salt thereof. In one example, a patient is treated for hypertrophic cardiomyopathy by administering to the patient a daily dose of about 15 mg aficamten, or a pharmaceutically acceptable salt thereof. In one example, a patient is treated for hypertrophic cardiomyopathy by administering to the patient a daily dose of about 20 mg aficamten, or a pharmaceutically acceptable salt thereof. In one example, a patient is treated for hypertrophic cardiomyopathy by administering to the patient a daily dose of about 25 mg aficamten, or a pharmaceutically acceptable salt thereof. In one example, a patient is treated for hypertrophic cardiomyopathy by administering to the patient a daily dose of about 30 mg aficamten, or a pharmaceutically acceptable salt thereof. In some embodiments of any of the foregoing, the treatment for hypertrophic cardiomyopathy further comprises administering disopyramide to the patient.

In some embodiments, provided are methods for the treatment for hypertrophic cardiomyopathy comprising administration of aficamten, or a pharmaceutically acceptable salt thereof, or a pharmaceutical composition containing aficamten, or a pharmaceutically acceptable salt thereof, in combination with a second therapeutic agent, wherein the second therapeutic agent is disopyramide. In some embodiments, the hypertrophic cardiomyopathy is obstructive hypertrophic cardiomyopathy and/or treatment refractory hypertrophic cardiomyopathy. In some embodiments, provided are methods for the treatment for obstructive hypertrophic cardiomyopathy, treatment refractory hypertrophic cardiomyopathy, or treatment refractive obstructive hypertrophic cardiomyopathy, comprising administration of aficamten, or a pharmaceutically acceptable salt thereof, or a pharmaceutical composition containing aficamten, or a pharmaceutically acceptable salt thereof, in combination with a second therapeutic agent, wherein the second therapeutic agent is disopyramide. In some embodiments, the combination of aficamten, or a pharmaceutically acceptable salt thereof, and disopyramide comprises simultaneous administration of aficamten, or a pharmaceutically acceptable salt thereof, and disopyramide. In some embodiments, the combination of aficamten, or a pharmaceutically acceptable salt thereof, and disopyramide comprises sequential administration of aficamten, or a pharmaceutically acceptable salt thereof, and disopyramide. In some embodiments, the combination of aficamten, or a pharmaceutically acceptable salt thereof, and disopyramide comprises administration of aficamten, or a pharmaceutically acceptable salt thereof, to a patient already undergoing treatment with disopyramide.

During the course of treatment for hypertrophic cardiomyopathy, the dose of aficamten, or a pharmaceutically acceptable salt thereof, administered to the patient may be titrated, for example by increasing, decreasing, or maintaining the dose. Titration may occur once during treatment, or may be performed iteratively separated by a period of time. For example, in some implementations, the dose of aficamten, or a pharmaceutically acceptable salt thereof, is titrated two or more times (e.g., 3, 4, 5 or more) during the course of treatment. In some embodiments, a new daily dose amount is administered to the patient at a constant amount for about 1 week to about 8 weeks (or about 2 weeks to about 6 weeks, or about 4 weeks) before the daily dose amount is titrated. In some embodiments, a new daily dose amount is administered to the patient at a constant amount for about 2 weeks before being titrated. For example, a first daily dose may be administered to the patient for about 2 weeks before a first titration, wherein the daily dose amount is increased, decreased, or maintained. The second titration may then occur approximately 2 weeks after the first titration. Titration of the dose allows the dose to be personalized to the patient's response to the drug, thus maximizing the potential treatment effect for patients.

Titration of the dose can be based on one or more of a left ventricular outflow tract pressure gradient (LVOT-G), biplane left ventricular ejection fraction (LVEF), and/or post-Valsalva LVOT-G measured in the patient. The measurement or measurements may be determined, for example, using an echocardiogram. The echocardiogram is taken following administration of the daily dose, for example about 1 hour to about 3 hours following administration of the dose. In some embodiments, the echocardiogram is taken about 2 hours following administration of the daily dose.

In some embodiments, an initial daily dose of about 5 mg, about 10 mg, about 15 mg, about 20 mg, or about 30 mg aficamten, or a pharmaceutically acceptable salt thereof, or any amount therebetween, is administered to the patient. After a period of time (e.g., about 2 weeks), resting LVOT-G, biplane LVEF, and/or post-Valsalva LVOT-G is measured, for example by echocardiography, following the administration of the dose (for example, about 1-3 hours, or about 2 hours following the administration of the dose). If the resting LVOT-G at or above a predetermined resting LVOT-G threshold (e.g., about 25 mmHg or more, about 30 mmHg or more, or about 35 mmHg or more) and the biplane LVEF is at or above a predetermined biplane LVEF threshold (e.g., about 40% or more, about 45% or more, about 50% or more, about 55% or more, or about 60% or more), the daily dose is increased. Alternatively, if the resting LVOT-G is not at or above the predetermined resting LVOT-G threshold, the dose may still be increased if the post-Valsalva LVOT-G is at or above a predetermined post-Valsalva LVOT-G threshold (e.g., about 40 mmHg or more, about 45 mmHg or more, about 50 mmHg or more, about 55 mmHg or more, or about 60 mmHg or more) and the biplane LVEF is at or above the predetermined biplane LVEF threshold. The dose may be maintained if the biplane LVEF is at or above the predetermined threshold, but the resting LVOT-G is below the resting LVOT-G threshold and the post-Valsalva LVOT-G is below the post-Valsalva LVOT-G threshold. The dose may be decreased or terminated if the biplane LVEF is below the biplane LVEF threshold. For example, the dose may be decreased if the biplane LVEF is below the biplane LVEF threshold and the current dose is not the lowest (e.g., first) dose. The dose may be terminated if the biplane LVEF is below the biplane LVEF threshold and the current dose is the lowest (e.g., first) dose. In some embodiments, the resting LVOT-G threshold is about 30 mmHg, the biplane LVEF threshold is about 50%, and the post-Valsalva LVOT-G threshold is about 50 mmHg. In some embodiments, titration of the dose of aficamten, or a pharmaceutically acceptable salt thereof, comprises maintaining the dose at the current dose; increasing the dose by about 1 mg, about 2 mg, about 3 mg, about 4 mg, about 5 mg, or about 10 mg, or any amount therebetween; decreasing the dose by about 1 mg, about 2 mg, about 3 mg, about 4 mg, about 5 mg, or about 10 mg, or any amount therebetween; or terminating administration. In some embodiments, titration of the dose comprises maintaining the dose at the current dose; increasing the dose by about 1 mg, about 2 mg, about 3 mg, about 4 mg, about 5 mg, or about 10 mg, or any amount therebetween; or decreasing the dose by about 1 mg, about 2 mg, about 3 mg, about 4 mg, about 5 mg, or about 10 mg, or any amount therebetween.

After a period of time (e.g., about 2 weeks) of the patient being administered the first titrated dose, the dose may be again titrated (i.e., increased, decreased, or maintained) based on the resting LVOT-G, biplane LVEF, and/or post-Valsalva LVOT-G of the patient, for example using the same threshold parameters as discussed above. An exemplary dose titration schedule includes: administration of a first titrated dose for about 2 weeks, about 3 weeks, about 4 weeks, about 5 weeks, about 6 weeks, about 8 weeks, about 10 weeks, or about 12 weeks, or any amount of time therebetween, followed by dose titration based on the resting LVOT-G, biplane LVEF, and/or post-Valsalva LVOT-G of the patient, for example using the same threshold parameters as discussed above. Further iterations of administration and dose titration may be conducted accordingly.

In some embodiments, the method results in a reduction of the resting left ventricular outflow tract pressure gradient (LVOT-G) to less than a particular value in the patient. The reduction in resting LVOT-G to less than the particular value may occur within ten weeks of initiating treatment with Compound 1, or a pharmaceutically acceptable salt thereof. The reduction in resting LVOT-G to less than the particular value may occur within one week, two weeks, three weeks, four weeks, one month, five weeks, six week, seven weeks, eight weeks, two months, nine weeks, or ten weeks of initiating treatment with Compound 1, or a pharmaceutically acceptable salt thereof. In some embodiments, reduction in resting LVOT-G is sustained for at least 10 weeks of treatment. In some embodiments, the reduction in resting LVOT-G occurs within two to six weeks of the end of a dose titration. In some embodiments, the reduction in resting LVOT-G peaks within two to six weeks of the end of a dose titration. In some embodiments, the particular resting LVOT-G value is: 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, or 25 mmHg.

In some embodiments, the method results in a reduction of the post-Valsalva left ventricular outflow tract pressure gradient (LVOT-G) to less than a particular value in the patient. The reduction in post-Valsalva LVOT-G to less than the particular value may occur within one week, two weeks, three weeks, four weeks, one month, five weeks, six week, seven weeks, eight weeks, two months, nine weeks, or ten weeks of initiating treatment with Compound 1, or a pharmaceutically acceptable salt thereof. The reduction in post-Valsalva LVOT-G may be sustained for at least 10 weeks of treatment. In some embodiments, the reduction in post-Valsalva LVOT-G occurs within two to six weeks of the end of dose titration. In some embodiments, the reduction in post-Valsalva LVOT-G peaks within two to six weeks of the end of dose titration. In some embodiments, the particular post-Valsalva LVOT-G value is: 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, or 30 mmHg.

Provided herein is a method of reducing resting left ventricular outflow tract pressure gradient (LVOT-G) to less than a particular value in a patient with obstructive hypertrophic cardiomyopathy (oHCM) comprises administering to the patient a therapeutically effective amount of Compound 1, or a pharmaceutically acceptable salt thereof. The reduction in resting LVOT-G to less than the particular value may occur within one week, two weeks, three weeks, four weeks, one month, five weeks, six week, seven weeks, eight weeks, two months, nine weeks, or ten weeks of initiating treatment with Compound 1, or a pharmaceutically acceptable salt thereof. The reduction in resting LVOT-G to less than the particular value may occur within two weeks of initiating treatment with Compound 1, or a pharmaceutically acceptable salt thereof. In some implementations, reduction in resting LVOT-G is sustained for at least 10 weeks of treatment. In some embodiments, the reduction in resting LVOT-G occurs within two to six weeks of the end of a dose titration. In some embodiments, the reduction in resting LVOT-G peaks within two to six weeks of the end of a dose titration. In some embodiments, the particular resting LVOT-G value is: 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, or 25 mmHg.

Provided herein is a method of reducing post-Valsalva left ventricular outflow tract pressure gradient (LVOT-G) to less than a particular value in a patient with obstructive hypertrophic cardiomyopathy (oHCM) which comprises administering to the patient a therapeutically effective amount of Compound 1, or a pharmaceutically acceptable salt thereof. The reduction in post-Valsalva LVOT-G to less than the particular value may occur within one week, two weeks, three weeks, four weeks, one month, five weeks, six week, seven weeks, eight weeks, two months, nine weeks, or ten weeks of initiating treatment with Compound 1, or a pharmaceutically acceptable salt thereof. The reduction in post-Valsalva LVOT-G may be sustained for at least 10 weeks of treatment. In some embodiments, the reduction in post-Valsalva LVOT-G occurs within two to six weeks of the end of dose titration. In some embodiments, the reduction in post-Valsalva LVOT-G peaks within two to six weeks of the end of dose titration. In some embodiments, the particular post-Valsalva LVOT-G value is: 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, or 30 mmHg.

Provided herein is a method of reducing resting left ventricular outflow tract pressure gradient (LVOT-G) to less than a particular value and reducing post-Valsalva left ventricular outflow tract pressure gradient (LVOT-G) to less than a particular value in a patient with obstructive hypertrophic cardiomyopathy (oHCM) which comprises administering to the patient a therapeutically effective amount of Compound 1, or a pharmaceutically acceptable salt thereof. The reduction in resting LVOT-G to less than the particular value and the reduction in post-Valsalva LVOT-G to less than the particular value may occur within one week, two weeks, three weeks, four weeks, one month, five weeks, six week, seven weeks, eight weeks, two months, nine weeks, or ten weeks of initiating treatment with Compound 1, or a pharmaceutically acceptable salt thereof. The reduction in resting LVOT-G to less than the particular value and the reduction in post-Valsalva LVOT-G to less than the particular value may occur within two weeks of initiating treatment with Compound 1, or a pharmaceutically acceptable salt thereof. In some implementations, reduction in resting LVOT-G and reduction in post-Valsalva LVOT-G is sustained for at least 10 weeks of treatment. In some embodiments, both the reduction in resting LVOT-G and the reduction in post-Valsalva LVOT-G may occur within two to six weeks of the end of a dose titration. In some embodiments, both the reduction in resting LVOT-G and the reduction in post-Valsalva LVOT-G peak within two to six weeks of the end of a dose titration. In some embodiments, the particular resting LVOT-G value is: 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, or 25 mmHg. In some embodiments, the particular post-Valsalva LVOT-G value is: 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, or 30 mmHg. In some embodiments, the particular resting LVOT-G value is 30 mm Hg, and the particular post-Valsalva LVOT-G value is 50 mmHg.

Provided herein is a method of treating obstructive hypertrophic cardiomyopathy (oHCM) in a patient eligible for septal reduction therapy (SRT), which comprises administering to the patient a therapeutically effective amount of Compound 1, or a pharmaceutically acceptable salt thereof. Also provided herein is a method of treating oHCM in a patient in need of SRT, which comprises administering to the patient a therapeutically effective amount of Compound 1, or a pharmaceutically acceptable salt thereof, wherein the method precludes the need for SRT in the patient. In some embodiments, the SRT is myectomy. In some embodiments, the SRT is alcohol septal ablation.

Further provided herein is a method of treating obstructive hypertrophic cardiomyopathy (oHCM) in a patient with heart failure symptoms, comprising administering to the patient a therapeutically effective amount of Compound 1, or a pharmaceutically acceptable salt thereof, wherein the method results in a reduction of heart failure symptoms as assessed by NYHA classification. In some embodiments of the foregoing, the methods improve heart failure symptoms by at least one NYHA class in the patient, for instance, by one or two NYHA class(es). In some embodiments of the foregoing, the methods convert patients from NYHA class III to class II or class I. In some embodiments of the foregoing, the methods convert patients from NYHA class III to class II. In some embodiments of the foregoing, the methods convert patients from NYHA class III to class I. In some embodiments of the foregoing, the methods convert patients from NYHA class II to class I. In some embodiments of the foregoing, reduction of heart failure symptoms occurs within ten weeks of initiating treatment with Compound 1, or a pharmaceutically acceptable salt thereof.

In some embodiments, titration of the daily dose of aficamten, or a pharmaceutically acceptable salt thereof, is based on the results of an echocardiogram that includes a biplane LVEF and a post-Valsalva LVOT-G. For example, based on the results of the biplane LVEF and/or post-Valsalva LVOT-G, the daily dose of aficamten, or a pharmaceutically acceptable salt thereof, may be increased, maintained, or decreased (or terminated, for example if the subject is already receiving the lowest (e.g., first) daily dose). For example, based on the results of the biplane LVEF and/or post-Valsalva LVOT-G, the daily dose of aficamten, or a pharmaceutically acceptable salt thereof, may be increased, maintained, or decreased (or terminated, for example if the subject is already receiving the first daily dose). A first daily dose of aficamten, or a pharmaceutically acceptable salt thereof, is administered to the patient for a first time period (e.g., about two weeks). A second daily dose for the subject, or termination of administration of aficamten, or a pharmaceutically acceptable salt thereof, or the pharmaceutically acceptable salt thereof, is then selected based on a biplane LVEF and post-Valsalva LVOT-G for the patient acquired after the first time period. The administration of aficamten, or a pharmaceutically acceptable salt thereof, or the pharmaceutically acceptable salt thereof may be terminated if the biplane LVEF of the echocardiogram is below a first predetermined biplane LVEF threshold (e.g., 50%). The administration of aficamten, or a pharmaceutically acceptable salt thereof, or the pharmaceutically acceptable salt thereof may be terminated if the biplane LVEF of the echocardiogram is below a first predetermined biplane LVEF threshold (e.g., 50%) and the patient is already receiving the lowest (e.g., first) daily dose. If the biplane LVEF of the echocardiogram is below a first predetermined biplane LVEF threshold (e.g., 50%) and the patient is not already receiving the lowest daily dose, then the daily dose may be decreased (i.e., the second daily dose is lesser than the first daily dose). If the biplane LVEF is at or above the first predetermined biplane threshold and below a second predetermined biplane LVEF threshold (e.g., 55%), or if the biplane LVVEF is at or above the second predetermined biplane LVEF threshold and the post-Valsalva LVOT-G is below a predetermined post-Valsalva LVOT-G threshold (e.g., 30 mmHg), then the daily dose is maintained (i.e., the second daily dose is the same as the first daily dose). If the biplane LVEF is at or above the second predetermined biplane threshold and the post-Valsalva LVOT-G is above the predetermined post-Valsalva LVOT-G threshold, the daily dose may be increased (i.e., the second daily dose is greater than the first daily dose).

The second daily dose may be administered to the patient for a second time period (e.g., about two weeks) before again being titrated based on the results of a second echocardiogram that includes a biplane LVEF and a post-Valsalva LVOT-G for the patient acquired after the second time period. For example, a third daily dose, or termination of the administration, may be selected or the administration terminated based on the second echocardiogram and the second daily dose. If the second daily dose is the same as (or lower than) the first daily dose and the biplane LVEF of the second echocardiogram is below the first predetermined biplane LVEF threshold, the administration may be terminated. If the second daily dose is higher than the first daily dose and the biplane LVEF of the second echocardiogram is below the first predetermined biplane LVEF threshold, the third daily dose may be decreased relative to the second daily dose, for example to the amount of the first daily dose. If the second daily dose is the same as (or lower than) the first daily dose (e.g., if the second daily dose is the lowest dose) and the biplane LVEF of the second echocardiogram is below the first predetermined biplane LVEF threshold, the administration may be terminated. If the second daily dose is higher than the lowest (e.g., first) daily dose and the biplane LVEF of the second echocardiogram is below the first predetermined biplane LVEF threshold, the third daily dose may be decreased relative to the second daily dose, for example to the amount of the first daily dose. If the biplane LVEF is at or above the first predetermined biplane LVEF threshold and below the second predetermined biplane LVEF threshold, or if the biplane LVEF is at or above the second predetermined biplane LVEF threshold and the post-Valsalva LVOT-G is below the predetermined post-Valsalva LVOT-G threshold, the daily dose may be maintained (i.e., the third daily dose is the same as the second daily dose). The third daily dose may be increased relative to the second daily dose if the biplane LVEF of the second echocardiogram is above the second predetermined biplane LVEF threshold and the post-Valsalva LVOT-G of the second echocardiogram is at or above the predetermined post-Valsalva LVOT-G threshold. The third daily dose is then administered to the patient for a third time period (e.g., two weeks).

Titration of the daily dose may be repeated for additional rounds, if desired, to select a fourth daily dose of aficamten, or a pharmaceutically acceptable salt thereof or termination of administration. For example, a third echocardiogram comprising a biplane LVEF and a post-Valsalva LVOT-G may be acquired for the patient after the third time period, and a fourth daily dose of aficamten, or a pharmaceutically acceptable salt thereof, may be selected based on the third echocardiogram and the third daily dose. If the biplane LVEF of the third echocardiogram is below the first predetermined biplane LVEF threshold and the third daily dose is the same as (or lower than) the first daily dose, the administration of aficamten, or a pharmaceutically acceptable salt thereof, may be terminated. If the third daily dose is higher than the first daily dose, and the biplane LVEF of the third echocardiogram is below the first predetermined biplane LVEF threshold, then the fourth daily dose is decreased relative to the third daily dose. If the biplane LVEF of the third echocardiogram is below the first predetermined biplane LVEF threshold and the third daily dose is the same as (or lower than) the first daily dose (e.g., if the third daily dose is the lowest dose), the administration of aficamten, or a pharmaceutically acceptable salt thereof, may be terminated. If the third daily dose is higher than the lowest (e.g., first) daily dose, and the biplane LVEF of the third echocardiogram is below the first predetermined biplane LVEF threshold, then the fourth daily dose is decreased relative to the third daily dose. If the biplane LVEF of the third echocardiogram is at or above the predetermined biplane LVEF threshold and below the second predetermined biplane LVEF threshold, or the biplane LVEF of the third echocardiogram is at or above the second predetermined biplane LVEF threshold and the post-Valsalva LVOT-G of the third echocardiogram is below the predetermined post-Valsalva LVOT-G threshold, then the fourth daily dose may be the same as the third daily dose. If the biplane LVEF of the third echocardiogram is above the second predetermined biplane LVEF threshold and the post-Valsalva LVOT-G of the third echocardiogram is at or above the predetermined post-Valsalva LVOT-G threshold, then the fourth daily dose may be increased relative to the third daily dose.

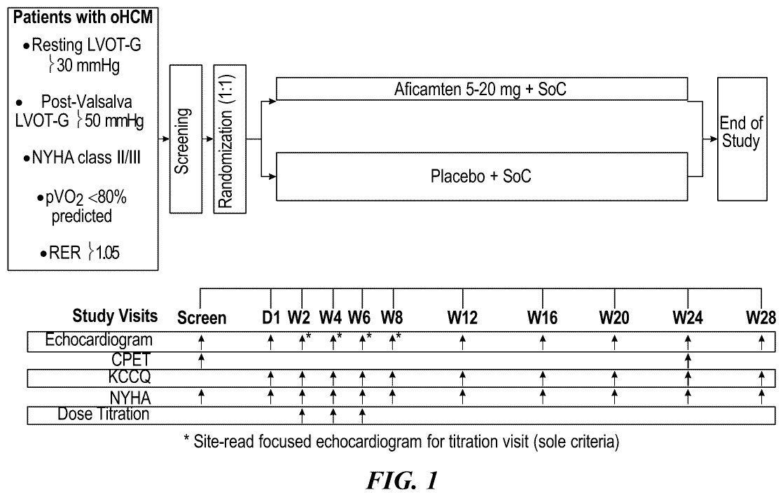

FIG. 1 of WO 2024/031016 and U.S. Patent Publication US-2024-0115554-A1 illustrates and exemplary method for treating obstructive hypertrophic cardiomyopathy (oHCM) in a patient that included titrating the daily dose of aficamten (Compound 1 in FIG. 1 of WO 2024/031016 and U.S. Patent Publication US-2024-0115554-A1), or a pharmaceutically acceptable salt thereof. The exemplary method shown in FIG. 1 of WO 2024/031016 and U.S. Patent Publication US-2024-0115554-A1 provides four daily dose levels, with a first daily dose level being the lowest daily dose level, but may be readily modified to include additional or fewer dose levels. The exemplary method shown in FIG. 1 of WO 2024/031016 and U.S. Patent Publication US-2024-0115554-A1 may be further modified such that the first daily dose level is not the lowest daily dose level. At 102, the first daily dose level (e.g., about 5 mg) of aficamten, or a pharmaceutically acceptable salt thereof, is administered to a patient. After a first time period, the daily dose level is increased or maintained, or the administration is terminated, at 104. The selection may be based on a first echocardiogram acquired for the patient after the first time period. Termination of administration 106 may be selected if the biplane LVEF of the first echocardiogram is below a predetermined biplane LVEF threshold (e.g., 50%), wherein the no further dose of aficamten, or a pharmaceutically acceptable salt thereof, or pharmaceutically acceptable salt thereof is administered to the patient. Maintenance of the first daily dose level (e.g., about 5 mg) may be selected when either of the following conditions are met on the first echocardiogram: (1) the biplane LVEF is at or above the predetermined biplane LVEF threshold (e.g., 50%) and below a second predetermined biplane LVEF threshold (e.g., 55%); or (2) the biplane LVEF is at or above the second predetermined biplane LVEF threshold (e.g., 55%) and the post-Valsalva LVOT-G of the first echocardiogram is below a predetermined post-Valsalva LVOT-G threshold (e.g., 30 mmHg). Alternatively, maintenance of the first daily dose level (e.g., about 5 mg) may be selected when the biplane LVEF of the first echocardiogram is at or above the predetermined biplane LVEF threshold (e.g., 50%), the resting LVOT-G of the first echocardiogram is below a predetermined resting LVOT-G threshold (e.g., 30 mmHg), and the post-Valsalva LVOT-G of the first echocardiogram is below a predetermined post-Valsalva LVOT-G threshold (e.g., 50 mmHg). If maintenance is selected, the first daily dose level of aficamten, or a pharmaceutically acceptable salt thereof, is administered to the patient at 102 for a second time period, and, optionally, the daily dose may be re-titrated at 104 after a second time period. The daily dose level may be increased to a second daily dose level (e.g., 10 mg) when either of the following conditions are met on the first echocardiogram: (1) the biplane LVEF is at or above the predetermined biplane LVEF threshold (e.g., 50%) and below a second predetermined biplane LVEF threshold (e.g., 55%); or (2) the biplane LVEF is at or above the second predetermined biplane LVEF threshold (e.g., 55%) and the post-Valsalva LVOT-G of the first echocardiogram is below a second predetermined post-Valsalva LVOT-G threshold (e.g., 30 mmHg). Alternatively, the daily dose level may be increased to a second daily dose level (e.g., 10 mg) when either of the following conditions are met on the first echocardiogram: (1) the biplane LVEF is at or above the predetermined biplane LVEF threshold (e.g., 50%) and the resting LVOT-G is at or above the predetermined resting LVOT-G threshold (e.g., 30 mmHg), or (2) the biplane LVEF is at or above the predetermined biplane LVEF threshold (e.g., 50%), the resting LVOT-G is below the predetermined resting LVOT-G threshold (e.g., 30 mmHg), and the post-Valsalva LVOT-G is at or above the predetermined post-Valsalva LVOT-G threshold (e.g., 50 mmHg). If an increase in the daily dose level is selected, the second daily dose level of aficamten, or a pharmaceutically acceptable salt thereof, is administered to the patient for the second time period at 108.

If the second daily dose level of aficamten, or a pharmaceutically acceptable salt thereof, (e.g., 10 mg) is administered to the patient at 108, the daily dose may be re-titrated (i.e., select an increase, decrease, or maintenance of the daily dose) based on an echocardiogram at 110. The daily dose may be decreased to the first daily dose level (e.g., from 10 mg to 5 mg) if the biplane LVEF of the echocardiogram is below a predetermined biplane LVEF threshold (e.g., 50%). If the daily dose is decreased to the first daily dose level, the first daily dose level is administered to the patient at 102. Maintenance of the second daily dose level (e.g., about 10 mg) may be selected when either of the following conditions are met on the echocardiogram: (1) the biplane LVEF is at or above the predetermined biplane LVEF threshold (e.g., 50%) and below a second predetermined biplane LVEF threshold (e.g., 55%); or (2) the biplane LVEF is at or above the second predetermined biplane LVEF threshold (e.g., 55%) and the post-Valsalva LVOT-G of the echocardiogram is below a predetermined post-Valsalva LVOT-G threshold (e.g., 30 mmHg). Alternatively, maintenance of the second daily dose level (e.g., about 10 mg) may be selected when the biplane LVEF of the echocardiogram is at or above the predetermined biplane LVEF threshold (e.g., 50%), the resting LVOT-G of the echocardiogram is below a predetermined resting LVOT-G threshold (e.g., 30 mmHg), and the post-Valsalva LVOT-G of the second echocardiogram is below a predetermined post-Valsalva LVOT-G threshold (e.g., 50 mmHg). If maintenance is selected, the second daily dose level of aficamten, or a pharmaceutically acceptable salt thereof, is administered to the patient at 108 for a further time period, and, optionally, the daily dose may be re-titrated at 110 after said time period. The daily dose level may be increased to a third daily dose level (e.g., 15 mg) when either of the following conditions are met on the echocardiogram: (1) the biplane LVEF is at or above the predetermined biplane LVEF threshold (e.g., 50%) and below a second predetermined biplane LVEF threshold (e.g., 55%); or (2) the biplane LVEF is at or above the second predetermined biplane LVEF threshold (e.g., 55%) and the post-Valsalva LVOT-G of the echocardiogram is below a second predetermined post-Valsalva LVOT-G threshold (e.g., 30 mmHg). Alternatively, the daily dose level may be increased to a third daily dose level (e.g., 15 mg) when either of the following conditions are met on the echocardiogram: (1) the biplane LVEF is at or above the predetermined biplane LVEF threshold (e.g., 50%) and the resting LVOT-G is at or above the predetermined resting LVOT-G threshold (e.g., 30 mmHg), or (2) the biplane LVEF is at or above the predetermined biplane LVEF threshold (e.g., 50%), the resting LVOT-G is below the predetermined resting LVOT-G threshold (e.g., 30 mmHg), and the post-Valsalva LVOT-G is at or above the predetermined post-Valsalva LVOT-G threshold (e.g., 50 mmHg). If an increase in the daily dose level is selected, the second daily dose level of aficamten, or a pharmaceutically acceptable salt thereof, is administered to the patient for the time period at 112.

If the third daily dose level of aficamten, or a pharmaceutically acceptable salt thereof, (e.g., 10 mg) is administered to the patient at 112, the daily dose may be re-titrated (i.e., select an increase, decrease, or maintenance of the daily dose) based on an echocardiogram at 114. The daily dose may be decreased to the second daily dose level (e.g., from 15 mg to 10 mg) if the biplane LVEF of the echocardiogram is below a predetermined biplane LVEF threshold (e.g., 50%). If the daily dose is decreased to the second daily dose level, the second daily dose level is administered to the patient at 108. Maintenance of the third daily dose level (e.g., about 15 mg) may be selected when either of the following conditions are met on the echocardiogram: (1) the biplane LVEF is at or above the predetermined biplane LVEF threshold (e.g., 50%) and below a second predetermined biplane LVEF threshold (e.g., 55%); or (2) the biplane LVEF is at or above the second predetermined biplane LVEF threshold (e.g., 55%) and the post-Valsalva LVOT-G of the echocardiogram is below a predetermined post-Valsalva LVOT-G threshold (e.g., 30 mmHg). Alternatively, maintenance of the third daily dose level (e.g., about 15 mg) may be selected when the biplane LVEF of the echocardiogram is at or above the predetermined biplane LVEF threshold (e.g., 50%), the resting LVOT-G of the echocardiogram is below a predetermined resting LVOT-G threshold (e.g., 30 mmHg), and the post-Valsalva LVOT-G of the echocardiogram is below a predetermined post-Valsalva LVOT-G threshold (e.g., 50 mmHg). If maintenance is selected, the third daily dose level of aficamten, or a pharmaceutically acceptable salt thereof, is administered to the patient at 112 for a further time period, and, optionally, the daily dose may be re-titrated at 114 after said time period. The daily dose level may be increased to a fourth daily dose level (e.g., 20 mg) when either of the following conditions are met on the echocardiogram: (1) the biplane LVEF is at or above the predetermined biplane LVEF threshold (e.g., 50%) and below a second predetermined biplane LVEF threshold (e.g., 55%); or (2) the biplane LVEF is at or above the second predetermined biplane LVEF threshold (e.g., 55%) and the post-Valsalva LVOT-G of the echocardiogram is below a second predetermined post-Valsalva LVOT-G threshold (e.g., 30 mmHg). Alternatively, the daily dose level may be increased to a fourth daily dose level (e.g., 20 mg) when either of the following conditions are met on the echocardiogram: (1) the biplane LVEF is at or above the predetermined biplane LVEF threshold (e.g., 50%) and the resting LVOT-G is at or above the predetermined resting LVOT-G threshold (e.g., 30 mmHg), or (2) the biplane LVEF is at or above the predetermined biplane LVEF threshold (e.g., 50%), the resting LVOT-G is below the predetermined resting LVOT-G threshold (e.g., 30 mmHg), and the post-Valsalva LVOT-G is at or above the predetermined post-Valsalva LVOT-G threshold (e.g., 50 mmHg). If an increase in the daily dose level is selected, the fourth daily dose level of aficamten, or a pharmaceutically acceptable salt thereof, is administered to the patient for the second time period at 116.

In the exemplary method shown in FIG. 1 of WO 2024/031016 and U.S. Patent Publication US-2024-0115554-A1, the first daily dose level is the minimum dose, and therefore may not be further decreased. Nevertheless, in other embodiments, the first daily dose level may be other than the minimum dose, and therefore may be decreased to a lower dose level (e.g., from 10 mg to 5 mg) if the biplane LVEF of the echocardiogram is below a predetermined biplane LVEF threshold (e.g., 50%).

In the exemplary method shown in FIG. 1 of WO 2024/031016 and U.S. Patent Publication US-2024-0115554-A1, the fourth daily dose level is the maximum dose, and therefore may not be further increased. Nevertheless, in other embodiments, additional dose levels may be available and the daily dose may be further increased at 118. The method shown in FIG. 1 of WO 2024/031016 and U.S. Patent Publication US-2024-0115554-A1, at 118 a selection is made to maintain the fourth daily dose level or decrease the daily dose level based on an echocardiogram. The daily dose may be decreased to the third daily dose level (e.g., from 20 mg to 15 mg) if the biplane LVEF of the echocardiogram is below a predetermined biplane LVEF threshold (e.g., 50%). If the daily dose is decreased to the third daily dose level, the second daily dose level is administered to the patient at 112. Maintenance of the third daily dose level (e.g., about 20 mg) may be selected when either of the following conditions are met on the echocardiogram: (1) the biplane LVEF is at or above the predetermined biplane LVEF threshold (e.g., 50%) and below a second predetermined biplane LVEF threshold (e.g., 55%); or (2) the biplane LVEF is at or above the second predetermined biplane LVEF threshold (e.g., 55%) and the post-Valsalva LVOT-G of the echocardiogram is below a predetermined post-Valsalva LVOT-G threshold (e.g., 30 mmHg). Alternatively, maintenance of the third daily dose level (e.g., about 15 mg) may be selected when the biplane LVEF of the echocardiogram is at or above the predetermined biplane LVEF threshold (e.g., 50%), the resting LVOT-G of the echocardiogram is below a predetermined resting LVOT-G threshold (e.g., 30 mmHg), and the post-Valsalva LVOT-G of the echocardiogram is below a predetermined post-Valsalva LVOT-G threshold (e.g., 50 mmHg). If maintenance is selected, the third daily dose level of aficamten, or a pharmaceutically acceptable salt thereof, is administered to the patient at 116 for a further time period, and, optionally, the daily dose may be re-titrated at 118 after said time period.

Exemplary daily dose increases include an increase from about 5 mg to about 10 mg aficamten, or a pharmaceutically acceptable salt thereof, about 10 mg to about 15 mg aficamten, or a pharmaceutically acceptable salt thereof, about 10 mg to about 20 mg aficamten, or a pharmaceutically acceptable salt thereof, or about 20 mg to about 30 mg. Other dose increases may be readily envisioned, for instance, increasing a given initial daily dose by about 1 mg, about 2 mg, about 3 mg, about 4 mg, about 5 mg, or about 10 mg, or any amount therebetween. Exemplary daily dose decreases include a decrease from about 30 mg to about 20 mg, about 20 mg to about 10 mg, about 15 mg to about 10 mg, or about 10 mg to about 5 mg. Other dose decreases may be readily envisioned, for instance, decreasing a given initial daily dose by about 1 mg, about 2 mg, about 3 mg, about 4 mg, about 5 mg, or about 10 mg, or any amount therebetween.

Exemplary embodiments of the methods described herein comprise administering a first daily dose (e.g., a first daily dose of between about 1 mg and about 20 mg, such as 1 mg, 2 mg, 3 mg, 4 mg, 5 mg, 6 mg, 7 mg, 8 mg, 9 mg, 10 mg, 15 mg, or 20 mg) or any amount therebetween) of aficamten, or a pharmaceutically acceptable salt thereof, for a first period (e.g., about one week, two weeks, three weeks, four weeks, five weeks, six weeks, seven weeks, eight weeks, nine weeks, ten weeks, twelve weeks, or any length of time therebetween), followed by maintaining the daily dose, decreasing the daily dose (e.g., decreasing the daily dose by between about 1 mg and about 10 mg, such as decreasing the daily dose by 1 mg, 2 mg, 3 mg, 4 mg, 5 mg 6 mg, 7 mg, 8 mg, 9 mg, or 10 mg, or any amount therebetween), increasing the daily dose (e.g., increasing the daily dose by between about 1 mg and about 10 mg, such as increasing the daily dose by 1 mg, 2 mg, 3 mg, 4 mg, 5 mg 6 mg, 7 mg, 8 mg, 9 mg, or 10 mg, or any amount therebetween), or terminating administration based on the resting LVOT-G, biplane LVEF, and/or post-Valsalva LVOT-G of the patient to arrive at a second daily dose. Another exemplary embodiment of the methods described herein comprises administering a first daily dose of aficamten, or a pharmaceutically acceptable salt thereof, for about two weeks, followed by maintaining the daily dose, decreasing the daily dose by about 5 mg, increasing the daily dose by about 5 mg, or terminating administration based on the resting LVOT-G, biplane LVEF, and/or post-Valsalva LVOT-G of the patient to arrive at a second daily dose. Another exemplary embodiment of the methods described herein comprises administering a first daily dose of aficamten, or a pharmaceutically acceptable salt thereof, for about three weeks, followed by maintaining the daily dose, decreasing the daily dose by about 5 mg, increasing the daily dose by about 5 mg, or terminating administration based on the resting LVOT-G, biplane LVEF, and/or post-Valsalva LVOT-G of the patient to arrive at a second daily dose. Another exemplary embodiment of the methods described herein comprises administering a first daily dose of aficamten, or a pharmaceutically acceptable salt thereof, for about two weeks, followed by maintaining the daily dose, decreasing the daily dose by about 10 mg, increasing the daily dose by about 10 mg, or terminating administration based on the resting LVOT-G, biplane LVEF, and/or post-Valsalva LVOT-G of the patient to arrive at a second daily dose. Another exemplary embodiment of the methods described herein comprises administering a first daily dose of aficamten, or a pharmaceutically acceptable salt thereof, for about three weeks, followed by maintaining the daily dose, decreasing the daily dose by about 10 mg, increasing the daily dose by about 10 mg, or terminating administration based on the resting LVOT-G, biplane LVEF, and/or post-Valsalva LVOT-G of the patient to arrive at a second daily dose. Another exemplary embodiment of the methods described herein comprises administering a first daily dose of aficamten, or a pharmaceutically acceptable salt thereof, for between about 2 and about 12 weeks, followed by maintaining the daily dose, decreasing the daily dose by about 10 mg, increasing the daily dose by about 10 mg, or terminating administration based on the resting LVOT-G, biplane LVEF, and/or post-Valsalva LVOT-G of the patient to arrive at a second daily dose.

Treatment for hypertrophic cardiomyopathy can result in improved exercise capacity and/or relieve symptoms in patients with hyperdynamic ventricular contraction resulting from hypertrophic cardiomyopathy. In some embodiments, the method includes administering a therapeutically effective daily dose of aficamten, or a pharmaceutically acceptable salt thereof, to an individual with hypertrophic cardiomyopathy, thereby improving the exercise capacity of the individual. In some embodiments, the method includes administering a therapeutically effective daily dose of aficamten, or a pharmaceutically acceptable salt thereof, to an individual with hypertrophic cardiomyopathy, thereby relieving one or more symptoms of hyperdynamic ventricular contraction. In some embodiments, the method includes administering a therapeutically effective daily dose of aficamten, or a pharmaceutically acceptable salt thereof, in combination with disopyramide to an individual with hypertrophic cardiomyopathy, thereby improving the exercise capacity of the individual. In some embodiments, the method includes administering a therapeutically effective daily dose of aficamten, or a pharmaceutically acceptable salt thereof, in combination with disopyramide to an individual with hypertrophic cardiomyopathy, thereby relieving one or more symptoms of hyperdynamic ventricular contraction.

In some embodiments, a therapeutically effective amount of aficamten, or a pharmaceutically acceptable salt thereof, is administered to a patient with hypertrophic cardiomyopathy, thereby decreasing resting LVOT-G in the patient. In some embodiments, the patient has a baseline resting LVOT-G of about 30 mmHg or more, about 40 mmHg or more, or about 50 mmHg or more. In response to administration of the therapeutically effective amount of aficamten, or a pharmaceutically acceptable salt thereof, the resting LVOT-G can decrease to less than 30 mmHg, for example to about 25 mmHg or less, about 20 mmHg or less, or about 15 mmHg or less. In some embodiments, in response to administration of the therapeutically effective amount of aficamten, or a pharmaceutically acceptable salt thereof, the resting LVOT-G decreases by about 10 mmHg or more, about 15 mmHg or more, about 20 mmHg or more, about 25 mmHg or more, about 30 mmHg or more, or about 35 mmHg or more. In some embodiments, in response to administration of the therapeutically effective amount of aficamten, or a pharmaceutically acceptable salt thereof, the resting LVOT-G decreases by about 10 mmHg to about 40 mmHg. The decrease in resting LVOT-G may occur after about 1 week, after about 2 weeks, after about 3 weeks, after about 4 weeks, after about 5 weeks, after about 6 weeks, after about 8 weeks, or after about 10 weeks of daily dose administration.

In some embodiments, a therapeutically effective amount of aficamten, or a pharmaceutically acceptable salt thereof, is administered to a patient with hypertrophic cardiomyopathy, thereby decreasing post-Valsalva LVOT-G in the patient. In some embodiments, a therapeutically effective amount of aficamten, or a pharmaceutically acceptable salt thereof, is administered in combination with disopyramide to a patient with hypertrophic cardiomyopathy, thereby decreasing post-Valsalva LVOT-G in the patient. In some embodiments, the patient has a baseline post-Valsalva LVOT-G of about 30 mmHg or more, about 40 mmHg or more, 50 mmHg or more, about 60 mmHg or more, or about 70 mmHg or more. In response to administration of the therapeutically effective amount of aficamten, or a pharmaceutically acceptable salt thereof, the post-Valsalva LVOT-G can decrease to less than 50 mmHg, for example to about 45 mmHg or less, about 40 mmHg or less, about 35 mmHg or less, or about 30 mmHg or less. In some embodiments, in response to administration of the therapeutically effective amount of aficamten, or a pharmaceutically acceptable salt thereof, the post-Valsalva LVOT-G decreases by about 10 mmHg or more, about 15 mmHg or more, about 20 mmHg or more, about 25 mmHg or more, about 30 mmHg or more, or about 35 mmHg or more. In some embodiments, in response to administration of the therapeutically effective amount of aficamten, or a pharmaceutically acceptable salt thereof, the post-Valsalva LVOT-G decreases by about 10 mmHg to about 40 mmHg. The decrease in post-Valsalva LVOT-G may occur after about 1 week, after about 2 weeks, after about 3 weeks, after about 4 weeks, after about 5 weeks, after about 6 weeks, after about 8 weeks, or after about 10 weeks of daily dose administration.

In some embodiments, a therapeutically effective amount of aficamten, or a pharmaceutically acceptable salt thereof, is administered to a patient with hypertrophic cardiomyopathy to treat the hypertrophic cardiomyopathy, wherein the biplane LVEF is maintained at or above 50%. In some embodiments, a therapeutically effective amount of aficamten, or a pharmaceutically acceptable salt thereof, is administered in combination with disopyramide to a patient with hypertrophic cardiomyopathy to treat the hypertrophic cardiomyopathy, wherein the biplane LVEF is maintained at or above 50%. In some embodiments, in response to administration of the therapeutically effective amount of aficamten, or a pharmaceutically acceptable salt thereof, the biplane LVEF decreases by less than about 20%, less than about 15%, less than about 10%, or less than about 5%. The maintenance interval of the biplane LVEF may be about 1 week or longer, about 2 weeks or longer, about 3 weeks or longer, about 4 weeks or longer, about 5 weeks or longer, about 6 weeks or longer, about 8 weeks or longer, or about 10 weeks or longer of daily dose administration.

### Treatment of Non-Obstructive Hypertrophic Cardiomyopathy

Described herein is a cardiac myosin inhibitor (CK-3773274, also referred to as aficamten, or a pharmaceutically acceptable salt thereof, or aficamten) and various methods, e.g., for treating non-obstructive hypertrophic cardiomyopathy or hypertrophic cardiomyopathy with mid-ventricular obstruction, using the cardiac myosin inhibitor. Treatment methods may include adjusting a dose, for example to increase, decrease or maintain a dose, based on the results of one or more biplane left ventricular ejection fraction (LVEF) measurements. These measurements may be taken, for example, using an echocardiogram.

As further described herein a therapeutically effective amount of aficamten, or a pharmaceutically acceptable salt thereof, may be administered to a patient for the treatment of non-obstructive hypertrophic cardiomyopathy. Aficamten, or a pharmaceutically acceptable salt thereof, may be administered at a constant dose level. Aficamten, or a pharmaceutically acceptable salt thereof, may be administered at a titrated dose level. For example, the dose of aficamten, or a pharmaceutically acceptable salt thereof, may be adjusted depending on the patient's response to the drug. That is, the dose of the aficamten, or a pharmaceutically acceptable salt thereof, may be periodically increased, decreased, maintained, or suspended depending on the measurement of a drug response, such as one or more of a left ventricular biplane left ventricular ejection fraction (LVEF) measurements.

Aficamten, or a pharmaceutically acceptable salt thereof, is administered at a therapeutically effective dosage, e.g., a dosage sufficient to provide treatment for the disease state. For a human, the daily dose may be between about 1 mg and about 50 mg. For example, the daily dose may be about 5 mg, about 10 mg, about 15 mg, about 20 mg, or about 30 mg, or any amount therebetween. A daily dose is the total amount administered in a day. A daily dose may be, but is not limited to be, administered each day, every other day, each week, every 2 weeks, every month, or at a varied interval. In some embodiments, the daily dose is administered for a period ranging from a single day to the life of the subject. In some embodiments, the daily dose is administered once a day. In some embodiments, the daily dose is administered in multiple divided doses, such as in 2, 3, or 4 divided doses. In some embodiments, the daily dose is administered in 2 divided doses.

In one example, a patient is treated for non-obstructive hypertrophic cardiomyopathy by administering to the patient a daily dose of about 5 mg to about 15 mg of aficamten, or a pharmaceutically acceptable salt thereof. In one example, a patient is treated for non-obstructive hypertrophic cardiomyopathy by administering to the patient a daily dose of about 5 mg aficamten, or a pharmaceutically acceptable salt thereof. In one example, a patient is treated for non-obstructive hypertrophic cardiomyopathy by administering to the patient a daily dose of about 10 mg aficamten, or a pharmaceutically acceptable salt thereof. In one example, a patient is treated for non-obstructive hypertrophic cardiomyopathy by administering to the patient a daily dose of about 15 mg aficamten, or a pharmaceutically acceptable salt thereof. In one example, a patient is treated for non-obstructive hypertrophic cardiomyopathy by administering to the patient a daily dose of about 20 mg aficamten, or a pharmaceutically acceptable salt thereof.

In some embodiments, provided are methods for the treatment for non-obstructive hypertrophic cardiomyopathy comprising administration of aficamten, or a pharmaceutically acceptable salt thereof, or a pharmaceutical composition containing aficamten, or a pharmaceutically acceptable salt thereof. In some embodiments, the non-obstructive hypertrophic cardiomyopathy is treatment refractory non-obstructive hypertrophic cardiomyopathy. In some embodiments, provided are methods for the treatment for non-obstructive hypertrophic cardiomyopathy, treatment refractory hypertrophic cardiomyopathy, or treatment refractive non-obstructive hypertrophic cardiomyopathy, comprising administration of aficamten, or a pharmaceutically acceptable salt thereof, or a pharmaceutical composition containing aficamten, or a pharmaceutically acceptable salt thereof.

During the course of treatment for non-obstructive hypertrophic cardiomyopathy, the dose of aficamten, or a pharmaceutically acceptable salt thereof, administered to the patient may be titrated, for example by increasing, decreasing, maintaining, or suspending the dose. Titration may occur once during treatment, or may be performed iteratively separated by a period of time. For example, in some implementations, the dose of aficamten, or a pharmaceutically acceptable salt thereof, is titrated two or more times (e.g., 3, 4, 5 or more) during the course of treatment. In some embodiments, a new daily dose amount is administered to the patient at a constant amount for about 1 week to about 8 weeks (or about 2 weeks to about 6 weeks, or about 4 weeks) before the daily dose amount is titrated. In some embodiments, a new daily dose amount is administered to the patient at a constant amount for about 2 weeks before being titrated. For example, a first daily dose may be administered to the patient for about 2 weeks before a first titration, wherein the daily dose amount is increased, decreased, or maintained. The second titration may then occur approximately 2 weeks after the first titration. Titration of the dose allows the dose to be personalized to the patient's response to the drug, thus maximizing the potential treatment effect for patients.

Titration of the dose can be based on biplane left ventricular ejection fraction (LVEF measured in the patient. The measurement or measurements may be determined, for example, using an echocardiogram. The echocardiogram may be taken following administration of the daily dose, for example about 1 hour to about 3 hours following administration of the dose. In some embodiments, the echocardiogram is taken about 2 hours following administration of the daily dose.

In some embodiments, an initial daily dose of about 5 mg, about 10 mg, about 15 mg, about 20 mg, or about 30 mg aficamten, or a pharmaceutically acceptable salt thereof, or any amount therebetween, is administered to the patient. After a period of time (e.g., about 2 weeks), biplane LVEF is measured, for example by echocardiography, following the administration of the dose (for example, about 1-3 hours, or about 2 hours following the administration of the dose). If the biplane LVEF is at or above a predetermined biplane LVEF threshold (e.g., about 40% or more, about 45% or more, about 50% or more, about 55% or more, or about 60% or more), the daily dose is increased. In some embodiments, the daily dose is increased if the biplane LVEF is at or above a predetermined biplane LVEF threshold of about 55%. In some embodiments, the daily dose is increased if the biplane LVEF is at or above a predetermined biplane LVEF threshold of about 60%. The dose may be decreased or terminated if the biplane LVEF is below the biplane LVEF threshold. In some embodiments, the biplane LVEF threshold is about 50%.

After a period of time (e.g., about 2 weeks) of the patient being administered the first titrated dose, the dose may be again titrated (i.e., increased, decreased, or maintained) or suspended based on the biplane LVEF of the patient, for example using the same threshold parameters as discussed above.

Provided herein is a method of treating non-obstructive hypertrophic cardiomyopathy (nHCM) in a patient eligible for septal reduction therapy (SRT), which comprises administering to the patient a therapeutically effective amount of aficamten, or a pharmaceutically acceptable salt thereof. Also provided herein is a method of treating nHCM in a patient in need of SRT, which comprises administering to the patient a therapeutically effective amount of aficamten, or a pharmaceutically acceptable salt thereof, wherein the method precludes the need for SRT in the patient. In some embodiments, the SRT is myectomy. In some embodiments, the SRT is alcohol septal ablation.

Further provided herein is a method of treating non-obstructive hypertrophic cardiomyopathy (nHCM) in a patient with heart failure symptoms, comprising administering to the patient a therapeutically effective amount of aficamten, or a pharmaceutically acceptable salt thereof, wherein the method results in a reduction of heart failure symptoms as assessed by NYHA classification. In some embodiments of the foregoing, the methods improve heart failure symptoms by at least one NYHA class in the patient, for instance, by one or two NYHA class(es). In some embodiments of the foregoing, the methods convert patients from NYHA class III to class II or class I. In some embodiments of the foregoing, the methods convert patients from NYHA class III to class II. In some embodiments of the foregoing, the methods convert patients from NYHA class III to class I. In some embodiments of the foregoing, the methods convert patients from NYHA class II to class I. In some embodiments of the foregoing, reduction of heart failure symptoms occurs within ten weeks of initiating treatment with aficamten, or a pharmaceutically acceptable salt thereof.

In some embodiments, titration of the daily dose of aficamten, or a pharmaceutically acceptable salt thereof, is based on the results of an echocardiogram that includes a biplane LVEF. For example, based on the results of the biplane LVEF, the daily dose of aficamten, or a pharmaceutically acceptable salt thereof, may be increased, maintained, or decreased (or terminated, for example if the subject is already receiving the lowest (e.g., first) daily dose). A first daily dose of aficamten, or a pharmaceutically acceptable salt thereof, is administered to the patient for a first time period (e.g., about two weeks). A second daily dose for the subject, or termination of administration of aficamten, or a pharmaceutically acceptable salt thereof, or the pharmaceutically acceptable salt thereof, is then selected based on a biplane LVEF for the patient acquired after the first time period. The administration of aficamten, or a pharmaceutically acceptable salt thereof, or the pharmaceutically acceptable salt thereof may be terminated if the biplane LVEF of the echocardiogram is below a first predetermined biplane LVEF threshold (e.g., 50%) and the patient is already receiving the lowest (e.g., first) daily dose. If the biplane LVEF of the echocardiogram is below a first predetermined biplane LVEF threshold (e.g., 50%) and the patient is not already receiving the lowest daily dose, then the daily dose may be decreased (i.e., the second daily dose is lesser than the first daily dose). If the biplane LVEF is at or above the first predetermined biplane threshold and below a second predetermined biplane LVEF threshold (e.g., 55%), then the daily dose is maintained (i.e., the second daily dose is the same as the first daily dose). If the biplane LVEF is at or above the second predetermined biplane threshold, the daily dose may be increased (i.e., the second daily dose is greater than the first daily dose). In some embodiments, the second predetermined biplane LVEF threshold is 60%.

The second daily dose may be administered to the patient for a second time period (e.g., about two weeks) before again being titrated based on the results of a second echocardiogram that includes a biplane LVEF for the patient acquired after the second time period. (The second time period may be, for example, about 2 weeks, about 10 weeks, about 12 weeks, about 6 months, about 1 year, about 2 years, about 3 years, about 4 years, or about 5 years, or indefinite.) For example, a third daily dose, or termination of the administration, may be selected or the administration terminated based on the second echocardiogram and the second daily dose. If the second daily dose is the same as (or lower than) the first daily dose (e.g., if the second daily dose is the lowest dose) and the biplane LVEF of the second echocardiogram is below the first predetermined biplane LVEF threshold, the administration may be terminated. If the second daily dose is higher than the first daily dose and the biplane LVEF of the second echocardiogram is below the first predetermined biplane LVEF threshold, the third daily dose may be decreased relative to the second daily dose, for example to the amount of the first daily dose. If the biplane LVEF is at or above the first predetermined biplane LVEF threshold and below the second predetermined biplane LVEF threshold, the daily dose may be maintained (i.e., the third daily dose is the same as the second daily dose). The third daily dose may be increased relative to the second daily dose if the biplane LVEF of the second echocardiogram is above the second predetermined biplane LVEF threshold. The third daily dose is then administered to the patient for a third time period (e.g., two weeks). The third time period may be, for example, about 2 weeks, about 10 weeks, about 12 weeks, about 6 months, about 1 year, about 2 years, about 3 years, about 4 years, or about 5 years, or indefinite.

Titration of the daily dose may be repeated for additional rounds, if desired, to select a fourth daily dose of aficamten, or a pharmaceutically acceptable salt thereof or termination of administration. For example, a third echocardiogram comprising a biplane LVEF may be acquired for the patient after the third time period, and a fourth daily dose of aficamten, or a pharmaceutically acceptable salt thereof, may be selected based on the third echocardiogram and the third daily dose. If the biplane LVEF of the third echocardiogram is below the first predetermined biplane LVEF threshold and the third daily dose is the same as (or lower than) the first daily dose, the administration of aficamten, or a pharmaceutically acceptable salt thereof, may be terminated. If the third daily dose is higher than the first daily dose, and the biplane LVEF of the third echocardiogram is below the first predetermined biplane LVEF threshold, then the fourth daily dose is decreased relative to the third daily dose. If the biplane LVEF of the third echocardiogram is below the first predetermined biplane LVEF threshold and the third daily dose is the same as (or lower than) the first daily dose (e.g., if the third daily dose is the lowest dose), the administration of aficamten, or a pharmaceutically acceptable salt thereof, may be terminated. If the third daily dose is higher than the lowest (e.g., first) daily dose, and the biplane LVEF of the third echocardiogram is below the first predetermined biplane LVEF threshold, then the fourth daily dose is decreased relative to the third daily dose. If the biplane LVEF of the third echocardiogram is at or above the predetermined biplane LVEF threshold, then the fourth daily dose may be the same as the third daily dose. If the biplane LVEF is at or above the first predetermined biplane LVEF threshold and below the second predetermined biplane LVEF threshold, the daily dose may be maintained (i.e., the fourth daily dose is the same as the third daily dose). The fourth daily dose may be increased relative to the third daily dose if the biplane LVEF of the second echocardiogram is above the second predetermined biplane LVEF threshold. The fourth daily dose is then administered to the patient for a fourth time period (e.g., two weeks). The fourth time period may be, for example, about 2 weeks, about 10 weeks, about 12 weeks, about 6 months, about 1 year, about 2 years, about 3 years, about 4 years, or about 5 years, or indefinite.

FIG. 1 of WO 2024/020468 and U.S. Patent Publication US-2024-0091203-A1 illustrates and exemplary method for treating non-obstructive hypertrophic cardiomyopathy (nHCM) in a patient that includes titrating the daily dose of aficamten (Compound 1 of WO 2024/020468 and U.S. Patent Publication US-2024-0091203-A1), or a pharmaceutically acceptable salt thereof. The exemplary method shown in FIG. 1 of WO 2024/020468 and U.S. Patent Publication US-2024-0091203-A1 provides three daily dose levels, with a first daily dose level being the lowest daily dose level, but may be readily modified to include additional or fewer dose levels, or may be further modified such that the first daily dose level is not the lowest daily dose level. At 102, the first daily dose level (e.g., about 5 mg) of aficamten, or a pharmaceutically acceptable salt thereof, is administered to a patient. After a first time period, the daily dose level is increased or maintained, or the administration is terminated, at 104. The selection may be based on a first echocardiogram acquired for the patient after the first time period. Termination of administration 106 may be selected if the biplane LVEF of the first echocardiogram is below a predetermined biplane LVEF threshold (e.g., 50%), wherein the no further dose of aficamten, or a pharmaceutically acceptable salt thereof, or pharmaceutically acceptable salt thereof is administered to the patient. Maintenance of the first daily dose level (e.g., about 5 mg) may be selected when the biplane LVEF is at or above the predetermined biplane LVEF threshold (e.g., 50%) and below a second predetermined biplane LVEF threshold (e.g., 55%). If maintenance is selected, the first daily dose level of aficamten, or a pharmaceutically acceptable salt thereof, is administered to the patient at 102 for a second time period, and, optionally, the daily dose may be re-titrated at 104 after the second time period. The daily dose level may be increased to a second daily dose level (e.g., 10 mg) when the biplane LVEF is at or above the second predetermined biplane LVEF threshold (e.g., 55%). If an increase in the daily dose level is selected, the second daily dose level of aficamten, or a pharmaceutically acceptable salt thereof, is administered to the patient for the second time period at 108.

If the second daily dose level of aficamten, or a pharmaceutically acceptable salt thereof, (e.g., 10 mg) is administered to the patient at 108, the daily dose may be re-titrated (i.e., select an increase, decrease, or maintenance of the daily dose) based on an echocardiogram at 110. The daily dose may be decreased to the first daily dose level (e.g., from 10 mg to 5 mg) if the biplane LVEF of the echocardiogram is below a predetermined biplane LVEF threshold (e.g., 50%). If the daily dose is decreased to the first daily dose level, the first daily dose level is administered to the patient at 102. Maintenance of the second daily dose level (e.g., about 10 mg) may be selected when the biplane LVEF is at or above the predetermined biplane LVEF threshold (e.g., 50%) and below a second predetermined biplane LVEF threshold (e.g., 55%). If maintenance is selected, the second daily dose level of aficamten, or a pharmaceutically acceptable salt thereof, is administered to the patient at 108 for a further time period, and, optionally, the daily dose may be re-titrated at 110 after said time period. The daily dose level may be increased to a third daily dose level (e.g., 15 mg) when the biplane LVEF is at or above the second predetermined biplane LVEF threshold (e.g., 55%). If an increase in the daily dose level is selected, the second daily dose level of aficamten, or a pharmaceutically acceptable salt thereof, is administered to the patient for the time period at 112.

If the third daily dose level of aficamten, or a pharmaceutically acceptable salt thereof, (e.g., 10 mg) is administered to the patient at 112, the daily dose may be re-titrated (i.e., select a decrease or maintenance of the daily dose) based on an echocardiogram at 114. The daily dose may be decreased to the second daily dose level (e.g., from 15 mg to 10 mg) if the biplane LVEF of the echocardiogram is below a predetermined biplane LVEF threshold (e.g., 50%). If the daily dose is decreased to the second daily dose level, the second daily dose level is administered to the patient at 108. Maintenance of the third daily dose level (e.g., about 15 mg) may be selected when the biplane LVEF is at or above the predetermined biplane LVEF threshold (e.g., 50%). If maintenance is selected, the third daily dose level of aficamten, or a pharmaceutically acceptable salt thereof, is administered to the patient at 112 for a further time period, and, optionally, the daily dose may be re-titrated at 114 after said time period.

In the exemplary method shown in FIG. 1 of WO 2024/020468 and U.S. Patent Publication US-2024-0091203-A1, the first daily dose level is the minimum dose, and therefore may not be further decreased. Nevertheless, in other embodiments, the first daily dose level may be other than the minimum dose, and therefore may be decreased to a lower dose level (e.g., from 10 mg to 5 mg) if the biplane LVEF of the echocardiogram is below a predetermined biplane LVEF threshold (e.g., 50%).

In the exemplary method shown in FIG. 1 of WO 2024/020468 and U.S. Patent Publication US-2024-0091203-A1, the third daily dose level is the maximum dose, and therefore may not be further increased. Nevertheless, in other embodiments, additional dose levels may be available and the daily dose may be further increased at 114. The method shown in FIG. 1 of WO 2024/020468 and U.S. Patent Publication US-2024-0091203-A1, at 114 a selection is made to maintain the third daily dose level or decrease the daily dose level based on an echocardiogram. The daily dose may be decreased to the second daily dose level (e.g., from 15 mg to 10 mg) if the biplane LVEF of the echocardiogram is below a predetermined biplane LVEF threshold (e.g., 50%). If the daily dose is decreased to the second daily dose level, the second daily dose level is administered to the patient at 108. Maintenance of the third daily dose level (e.g., about 15 mg) may be selected when the biplane LVEF is at or above the predetermined biplane LVEF threshold (e.g., 50%). If maintenance is selected, the third daily dose level of aficamten, or a pharmaceutically acceptable salt thereof, is administered to the patient at 112 for a further time period, and, optionally, the daily dose may be re-titrated at 114 after said time period. In embodiments wherein additional dose levels may be available and the daily dose may be further increased at 114, the daily dose may be increased to a fourth daily dose level (e.g., about 20 mg) if the biplane LVEF is at or above the second predetermined biplane LVEF threshold (e.g., 60%).

Exemplary daily dose increases include an increase from about 5 mg to about 10 mg aficamten, or a pharmaceutically acceptable salt thereof, or about 10 mg to about 15 mg aficamten, or a pharmaceutically acceptable salt thereof. Other exemplary daily dose increases include an increase from about 5 mg to about 10 mg aficamten, or a pharmaceutically acceptable salt thereof, from about 10 mg to about 15 mg aficamten, or a pharmaceutically acceptable salt thereof, or from about 15 mg to about 20 mg aficamten, or a pharmaceutically acceptable salt thereof. Other dose increases may be readily envisioned, for instance, increasing a given initial daily dose by about 1 mg, about 2 mg, about 3 mg, about 4 mg, about 5 mg, or about 10 mg, or any amount therebetween. Exemplary daily dose decreases include a decrease from about 15 mg to about 10 mg, or about 10 mg to about 5 mg. Other exemplary daily dose decreases include a decrease from about 20 mg to about 15 mg, from about 15 mg to about 10 mg, or from about 10 mg to about 5 mg. Other dose decreases may be readily envisioned, for instance, decreasing a given initial daily dose by about 1 mg, about 2 mg, about 3 mg, about 4 mg, about 5 mg, or about 10 mg, or any amount therebetween.

Exemplary embodiments of the methods described herein comprise administering a first daily dose or Dose 1 (e.g., a first daily dose of between about 1 mg and about 20 mg, such as 1 mg, 2 mg, 3 mg, 4 mg, 5 mg, 6 mg, 7 mg, 8 mg, 9 mg, 10 mg, 15 mg, or 20 mg) or any amount therebetween) of aficamten, or a pharmaceutically acceptable salt thereof, for a first period (e.g., about one week, two weeks, three weeks, four weeks, five weeks, six weeks, seven weeks, eight weeks, nine weeks, ten weeks, twelve weeks, or any length of time therebetween), followed by maintaining the daily dose, decreasing the daily dose (e.g., decreasing the daily dose by between about 1 mg and about 10 mg, such as decreasing the daily dose by 1 mg, 2 mg, 3 mg, 4 mg, 5 mg 6 mg, 7 mg, 8 mg, 9 mg, or 10 mg, or any amount therebetween), increasing the daily dose (e.g., increasing the daily dose by between about 1 mg and about 10 mg, such as increasing the daily dose by 1 mg, 2 mg, 3 mg, 4 mg, 5 mg 6 mg, 7 mg, 8 mg, 9 mg, or 10 mg, or any amount therebetween), or terminating administration based on the biplane LVEF of the patient to arrive at a second daily dose. Another exemplary embodiment of the methods described herein comprises administering a first daily dose of aficamten, or a pharmaceutically acceptable salt thereof, for about two weeks, followed by maintaining the daily dose, decreasing the daily dose by about 5 mg, increasing the daily dose by about 5 mg, or terminating administration based on the biplane LVEF of the patient to arrive at a second daily dose. Another exemplary embodiment of the methods described herein comprises administering a first daily dose of aficamten, or a pharmaceutically acceptable salt thereof, for about three weeks, followed by maintaining the daily dose, decreasing the daily dose by about 5 mg, increasing the daily dose by about 5 mg, or terminating administration based on the biplane LVEF of the patient to arrive at a second daily dose. Another exemplary embodiment of the methods described herein comprises administering a first daily dose of aficamten, or a pharmaceutically acceptable salt thereof, for about two weeks, followed by maintaining the daily dose, decreasing the daily dose by about 10 mg, increasing the daily dose by about 10 mg, or terminating administration based on the biplane LVEF of the patient to arrive at a second daily dose. Another exemplary embodiment of the methods described herein comprises administering a first daily dose of aficamten, or a pharmaceutically acceptable salt thereof, for about three weeks, followed by maintaining the daily dose, decreasing the daily dose by about 10 mg, increasing the daily dose by about 10 mg, or terminating administration based on the biplane LVEF of the patient to arrive at a second daily dose. Another exemplary embodiment of the methods described herein comprises administering a first daily dose of aficamten, or a pharmaceutically acceptable salt thereof, for between about 2 and about 12 weeks, followed by maintaining the daily dose, decreasing the daily dose by about 10 mg, increasing the daily dose by about 10 mg, or terminating administration based on the biplane LVEF of the patient to arrive at a second daily dose.

Treatment for non-obstructive hypertrophic cardiomyopathy can result in improved exercise capacity and/or relieve symptoms in patients with hyperdynamic ventricular contraction resulting from non-obstructive hypertrophic cardiomyopathy. In some embodiments, the method includes administering a therapeutically effective daily dose of aficamten, or a pharmaceutically acceptable salt thereof, to an individual with non-obstructive hypertrophic cardiomyopathy, thereby improving the exercise capacity of the individual. In some embodiments, exercise capacity can be measured by one or more cardiopulmonary exercise testing (CPET) parameters, or a composite of one or more CPET parameters. In some embodiments, exercise capacity can be measured by peak oxygen uptake (pVO₂) (maximal exercise capacity) and/or minute ventilation/carbon dioxide production (VE/VCO₂) slope (sub-maximal exercise capacity). In some embodiments, the method includes administering a therapeutically effective daily dose of aficamten, or a pharmaceutically acceptable salt thereof, to an individual with non-obstructive hypertrophic cardiomyopathy, thereby resulting in a change in pVO₂ of ≥1.0 mL/kg/min from baseline, and ≥1 class improvement in NYHA Functional Class in the individual.). In some embodiments, the method includes administering a therapeutically effective daily dose of aficamten, or a pharmaceutically acceptable salt thereof, to an individual with non-obstructive hypertrophic cardiomyopathy, thereby resulting in a change in pVO₂ of ≥2.0 mL/kg/min from baseline, and no worsening in NYHA Functional Class. In some embodiments, the method includes administering a therapeutically effective daily dose of aficamten, or a pharmaceutically acceptable salt thereof, to an individual with non-obstructive hypertrophic cardiomyopathy, thereby relieving one or more symptoms of hyperdynamic ventricular contraction.

In some embodiments, a therapeutically effective amount of aficamten, or a pharmaceutically acceptable salt thereof, is administered to a patient with non-obstructive hypertrophic cardiomyopathy to treat the non-obstructive hypertrophic cardiomyopathy, wherein the biplane LVEF is maintained at or above 50%. In some embodiments, in response to administration of the therapeutically effective amount of aficamten, or a pharmaceutically acceptable salt thereof, the biplane LVEF decreases by less than about 20%, less than about 15%, less than about 10%, or less than about 5%. The maintenance interval of the biplane LVEF may be about 1 week or longer, about 2 weeks or longer, about 3 weeks or longer, about 4 weeks or longer, about 5 weeks or longer, about 6 weeks or longer, about 8 weeks or longer, or about 10 weeks or longer of daily dose administration.

In some embodiments, a therapeutically effective amount of aficamten, or a pharmaceutically acceptable salt thereof, is administered to a patient with non-obstructive hypertrophic cardiomyopathy, thereby decreasing a left ventricle mass index (LVMI) for the patient. In response to administration of the therapeutically effective amount of aficamten, or a pharmaceutically acceptable salt thereof, the LVMI can decrease by about 1 g/m² or more, by about 1.5 g/m² or more, by about 2 g/m² or more, by about 2.5 g/m² or more, by about 3 g/m² or more, by about 3.5 g/m² or more, or by about 4 g/m² or more. In some embodiments, in response to administration of the therapeutically effective amount of aficamten, or a pharmaceutically acceptable salt thereof, the LVMI decreases by about 1 g/m² to about 10 g/m² mmHg, such as by about 1 g/m² to about 6 g/m², or about 2 g/m² to about 5 g/m². The decrease in LVMI may occur after about 1 week, after about 2 weeks, after about 3 weeks, after about 4 weeks, after about 5 weeks, after about 6 weeks, after about 8 weeks, or after about 10 weeks of daily dose administration.

In some embodiments, a therapeutically effective amount of aficamten, or a pharmaceutically acceptable salt thereof, is administered to a patient with non-obstructive hypertrophic cardiomyopathy, thereby decreasing a left arterial volume index (LAVI) for the patient. In response to administration of the therapeutically effective amount of aficamten, or a pharmaceutically acceptable salt thereof, the LAVI can decrease by about 0.5 mL/m2 or more, by about 1 mL/m2 or more, by about 1.5 mL/m2 or more, by about 2 mL/m2 or more, or by about 2.5 mL/m2 or more. In some embodiments, in response to administration of the therapeutically effective amount of aficamten, or a pharmaceutically acceptable salt thereof, the LAVI decreases by about 0.5 mL/m2 to about 5 mL/m2 mmHg, such as by about 0.5 ml/m2 to about 4 g/m², or about 1 mL/m2 to about 3 mL/m2. The decrease in LAVI may occur after about 1 week, after about 2 weeks, after about 3 weeks, after about 4 weeks, after about 5 weeks, after about 6 weeks, after about 8 weeks, or after about 10 weeks of daily dose administration.

In some embodiments, a therapeutically effective amount of aficamten, or a pharmaceutically acceptable salt thereof, is administered to a patient with non-obstructive hypertrophic cardiomyopathy, thereby decreasing an e′ value for the patient. In response to administration of the therapeutically effective amount of aficamten, or a pharmaceutically acceptable salt thereof, the e′ value can decrease by about 0.1 cm/s or more, by about 0.15 cm/s or more, by about 0.2 cm/s or more, or by about 0.25 cm/s or more. In some embodiments, in response to administration of the therapeutically effective amount of aficamten, or a pharmaceutically acceptable salt thereof, the e′ value decreases by about 0.05 cm/s to about 0.3 cm/s, such as by about 0.1 cm/s to about 0.25 cm/s, or about 0.15 cm/s to about 0.25 cm/s. The decrease in e′ value may occur after about 1 week, after about 2 weeks, after about 3 weeks, after about 4 weeks, after about 5 weeks, after about 6 weeks, after about 8 weeks, or after about 10 weeks of daily dose administration.

In some embodiments, a therapeutically effective amount of aficamten, or a pharmaceutically acceptable salt thereof, is administered to a patient with non-obstructive hypertrophic cardiomyopathy, thereby decreasing a lateral E/e′ ratio for the patient. In response to administration of the therapeutically effective amount of aficamten, or a pharmaceutically acceptable salt thereof, the lateral E/e′ ratio can decrease by about 0.5 or more, by 1 or more, by about 1.2 or more, by about 1.5 or more, or by about 1.8 or more. In some embodiments, in response to administration of the therapeutically effective amount of aficamten, or a pharmaceutically acceptable salt thereof, the lateral E/e′ ratio decreases by about 0.5 to about 2, such as by about 1 to about 1.8, or about 1.5 to about 1.8. The decrease in lateral E/e′ ratio may occur after about 1 week, after about 2 weeks, after about 3 weeks, after about 4 weeks, after about 5 weeks, after about 6 weeks, after about 8 weeks, or after about 10 weeks of daily dose administration.

In some embodiments, a therapeutically effective amount of aficamten, or a pharmaceutically acceptable salt thereof, is administered to a patient with non-obstructive hypertrophic cardiomyopathy, thereby decreasing a likelihood of systolic anterior motion (SAM) of the mitral valve leaflet for the patient.

In some embodiments, a therapeutically effective amount of aficamten, or a pharmaceutically acceptable salt thereof, is administered to a patient with non-obstructive hypertrophic cardiomyopathy, thereby decreasing a level of brain natriuretic peptide or N-terminal prohormone of brain natriuretic peptide (NT-proBNP) in the patient. The decrease in a level of brain natriuretic peptide or N-terminal prohormone of brain natriuretic peptide (NT-proBNP) in the patient may occur after about 1 week, after about 2 weeks, after about 3 weeks, after about 4 weeks, after about 5 weeks, after about 6 weeks, after about 8 weeks, or after about 10 weeks of daily dose administration.

In some embodiments, a therapeutically effective amount of aficamten, or a pharmaceutically acceptable salt thereof, is administered to a patient with non-obstructive hypertrophic cardiomyopathy, thereby decreasing a level of cardiac troponin I. The decrease in a level of cardiac troponin I in the patient may occur after about 1 week, after about 2 weeks, after about 3 weeks, after about 4 weeks, after about 5 weeks, after about 6 weeks, after about 8 weeks, or after about 10 weeks of daily dose administration.

In some embodiments of any of the foregoing, the patient with non-obstructive hypertrophic cardiomyopathy is classified as NYHA class III when administration with aficamten, or a pharmaceutically acceptable salt thereof, is initiated. In some embodiments, the patient with non-obstructive hypertrophic cardiomyopathy is classified as NYHA class II when administration with aficamten, or a pharmaceutically acceptable salt thereof, is initiated.

In some embodiments of any of the methods disclosed herein, the methods result in a reduction in the patient's NYHA classification by one or more classes, and/or a decrease in NT-proBNP, and/or a decrease in the level of troponin I (e.g., cardiac troponin I). In some embodiments, the methods result in a reduction in the patient's NYHA classification by one or more classes, or a decrease in NT-proBNP, or a decrease in the level of troponin I (e.g., cardiac troponin I). In some embodiments, the methods result in a reduction in the patient's NYHA classification by one or more classes, and a decrease in NT-proBNP. In some embodiments, the methods result in a reduction in the patient's NYHA classification by one or more classes, and a decrease in the level of troponin I (e.g., cardiac troponin I). In some embodiments, the methods result in a decrease in NT-proBNP and a decrease in the level of troponin I (e.g., cardiac troponin I). In some embodiments, the methods result in a reduction in the patient's NYHA classification by one or more classes, a decrease in NT-proBNP, and a decrease in the level of troponin I (e.g., cardiac troponin I).

In some embodiments of any of the foregoing, administration of aficamten, or a pharmaceutically acceptable salt thereof, to a patient with non-obstructive hypertrophic cardiomyopathy further results in decreased left ventricular wall stress and/or decreased myocardial injury in the patient. The decrease in left ventricular wall stress and/or the decrease in myocardial injury in the patient may occur after about 1 week, after about 2 weeks, after about 3 weeks, after about 4 weeks, after about 5 weeks, after about 6 weeks, after about 8 weeks, or after about 10 weeks of daily dose administration.

In some embodiments of any of the foregoing, administration of aficamten, or a pharmaceutically acceptable salt thereof, to a patient with non-obstructive hypertrophic cardiomyopathy results in an improvement in patient health status. In some embodiments of any of the foregoing, administration of aficamten, or a pharmaceutically acceptable salt thereof, to a patient with hypertrophic cardiomyopathy results in an improvement in patient health status as determined by changes in the Short Form 36 physical function sub-scale (SF-36-PFS). In some embodiments of any of the foregoing, administration of aficamten, or a pharmaceutically acceptable salt thereof, to a patient with hypertrophic cardiomyopathy results in an improvement in patient health status as determined by changes in the Kansas City Cardiomyopathy Questionnaire-Clinical Summary Score (KCCQ-CSS). In some embodiments, administration of aficamten, or a pharmaceutically acceptable salt thereof, to a patient with hypertrophic cardiomyopathy results in improvement in KCCQ-CSS by 1 point, 2 points, 3 points, 4 points, 5 points, or more than 5 points. In some embodiments of any of the foregoing, administration of aficamten, or a pharmaceutically acceptable salt thereof, to a patient with hypertrophic cardiomyopathy results in an improvement in patient health status and health-related quality of life as measured by PRO questionnaire, as determined by changes from responses to the EuroQol 5-dimension 5-level instrument (EQ-5D-5L). In some embodiments of any of the foregoing, administration of aficamten, or a pharmaceutically acceptable salt thereof, to a patient with hypertrophic cardiomyopathy results in an improvement in patient health status as determined by changes in Seattle Angina Questionnaire-Angina Frequency (SAQ-AF). In some embodiments of any of the foregoing, administration of aficamten, or a pharmaceutically acceptable salt thereof, to a patient with hypertrophic cardiomyopathy results in an improvement in patient health status as determined by changes in Seattle Angina Questionnaire-7 (SAQ-7).

In some embodiments, administration with aficamten, or a pharmaceutically acceptable salt thereof, to a patient with non-obstructive hypertrophic cardiomyopathy results in a sustained effect for at least 10 weeks, 12 weeks, 6 months, 1 year, 2 years, 3 years, 4 years, or 5 years. In some embodiments, administration with aficamten, or a pharmaceutically acceptable salt thereof, to a patient with non-obstructive hypertrophic cardiomyopathy results in a sustained effect for at least 6 months. In some embodiments, administration with aficamten, or a pharmaceutically acceptable salt thereof, to a patient with hypertrophic cardiomyopathy results in a sustained effect for at least 1 year. In some embodiments, administration with aficamten, or a pharmaceutically acceptable salt thereof, to a patient with non-obstructive hypertrophic cardiomyopathy results in a sustained effect for at least 5 years.

Methods and compositions for accelerating oxygen uptake recovery (VO₂Rec) in a patient in need thereof, comprising administering to the patient a therapeutically effective amount of aficamten or a pharmaceutically acceptable salt thereof, are provided therein. In some embodiments, the patient has a heart disease. In some embodiments, the heart disease is hypertrophic cardiomyopathy (HCM). In some embodiments, the heart disease is nHCM.

Methods and compositions for reducing or resolving left ventricular hypertrophy (LVH) in a patient in need thereof, comprising administering to the patient a therapeutically effective amount of aficamten or a pharmaceutically acceptable salt thereof, are provided therein. In some embodiments, the patient has a heart disease. In some embodiments, the heart disease is hypertrophic cardiomyopathy (HCM). In some embodiments, the heart disease is nHCM. In some embodiments, the LVH is LVH with strain pattern. In some embodiments, the LVH is LVH with strain pattern on electrocardiogram.

Methods and compositions for decreasing maximal wall thickness (MWT) in a patient in need thereof, comprising administering to the patient a therapeutically effective amount of aficamten or a pharmaceutically acceptable salt thereof, are provided therein. In some embodiments, the patient has a heart disease. In some embodiments, the heart disease is hypertrophic cardiomyopathy (HCM). In some embodiments, the heart disease is nHCM.

Methods and compositions for reducing chest pain in a patient in need thereof, comprising administering to the patient a therapeutically effective amount of aficamten or a pharmaceutically acceptable salt thereof, are provided therein. In some embodiments, the patient has a heart disease. In some embodiments, the heart disease is hypertrophic cardiomyopathy (HCM). In some embodiments, the heart disease is nHCM.

Treatment of Hypertrophic Cardiomyopathy with Mid-Ventricular Obstruction

In some embodiments of any of the foregoing methods, the non-obstructive HCM is HCM with mid-ventricular obstruction (MVO). For instance, in some embodiments, the methods disclosed herein comprise administering a therapeutically effective amount of aficamten, or a pharmaceutically acceptable salt thereof, to a patient for the treatment of hypertrophic cardiomyopathy with mid-ventricular obstruction (MVO), as described herein for the treatment of non-obstructive hypertrophic cardiomyopathy. For instance, in some embodiments, the patient in need thereof has HCM with mid-ventricular obstruction. It is to be understood that the term “mid-ventricular obstruction” (MVO) may be used interchangeably with “mid-cavitary obstruction” in the embodiments disclosed herein.

### Treatment of Obstructive Hypertrophic Cardiomyopathy

As further described herein a therapeutically effective amount of aficamten may be administered to a patient for the treatment of obstructive hypertrophic cardiomyopathy. Aficamten may be administered at a constant dose level. Aficamten may be administered at a titrated dose level. For example, the dose of aficamten may be adjusted depending on the patient's response to the drug. That is, the dose of the aficamten may be periodically increased, decreased, maintained, or terminated depending on the measurement of a drug response, such as one or more of a biplane left ventricular ejection fraction (LVEF) and post-Valsalva LVOT-G measurement.

Results of a recent clinical trial (see Example 1) demonstrated that treatment with aficamten for 10 weeks resulted in substantial and statistically significant reductions from baseline compared to placebo in the average resting left ventricular outflow tract pressure gradient (LVOT-G) (p=0.0003, p=0.0004, Cohort 1 and Cohort 2, respectively) and the average post-Valsalva LVOT-G (p=0.001, p<0.0001, Cohort 1 and Cohort 2, respectively). The majority of patients treated with aficamten (78.6% in Cohort 1 and 92.9% in Cohort 2) achieved the target goal of treatment, defined as resting gradient <30 mmHg and post-Valsalva gradient <50 mmHg at Week 10 compared to placebo (7.7%). Reductions in LVOT-G occurred within two weeks of initiating treatment with aficamten, peaked within two to six weeks of the end of dose titration, and were sustained until the end of treatment at 10 weeks. The observed reductions in LVOT-G were dose dependent, with patients achieving greater reductions of LVOT-G with increasing doses of aficamten.

Treatment with aficamten in the clinical trial was well tolerated. Overall, the incidence of adverse events was similar between treatment arms. No serious adverse events were attributed to aficamten and no treatment interruptions occurred on aficamten. No new cases of atrial fibrillation were reported by the investigators. In this dose-range finding trial, one patient experienced a transient decrease in left ventricular ejection fraction (LVEF) that required dose adjustment but not dose interruption. LVEF returned towards baseline within two weeks after the end of treatment in both cohorts, confirming the reversibility of effect with aficamten as was similarly observed in healthy participants in the Phase 1 study of aficamten.

Aficamten is administered at a therapeutically effective dosage, e.g., a dosage sufficient to provide treatment for the disease state. For a human, the daily dose may be between about 1 mg and about 50 mg. For example, the daily dose may be about 5 mg, about 10 mg, about 15 mg, about 20 mg, or any amount therebetween. A daily dose is the total amount administered in a day. A daily dose may be, but is not limited to be, administered each day, every other day, each week, every 2 weeks, every month, or at a varied interval. In some embodiments, the daily dose is administered for a period ranging from a single day to the life of the subject. In some embodiments, the daily dose is administered once a day. In some embodiments, the daily dose is administered in multiple divided doses, such as in 2, 3, or 4 divided doses. In some embodiments, the daily dose is administered in 2 divided doses.

In one example, a patient is treated for oHCM by administering to the patient a daily dose of about 5 mg to about 20 mg of aficamten. In one example, a patient is treated for obstructive hypertrophic cardiomyopathy by administering to the patient a daily dose of about 5 mg aficamten. In one example, a patient is treated for obstructive hypertrophic cardiomyopathy by administering to the patient a daily dose of about 10 mg aficamten. In one example, a patient is treated for obstructive hypertrophic cardiomyopathy by administering to the patient a daily dose of about 15 mg aficamten. In one example, a patient is treated for obstructive hypertrophic cardiomyopathy by administering to the patient a daily dose of about 20 mg aficamten.

In some embodiments, provided are methods for the treatment of oHCM comprising administration of aficamten, or a pharmaceutically acceptable salt thereof, or a pharmaceutical composition containing aficamten, or a pharmaceutically acceptable salt thereof, as a monotherapy, wherein the patient has been recently diagnosed with oHCM, the patient is treatment naïve, or the patient has chronic oHCM. In some embodiments, provided are methods for the treatment of oHCM comprising administration of aficamten, or a pharmaceutically acceptable salt thereof, or a pharmaceutical composition containing aficamten, or a pharmaceutically acceptable salt thereof, as a monotherapy, wherein patient has chronic oHCM and has previously received SOC medical therapy for oHCM prior to administering aficamten, or a pharmaceutically acceptable salt thereof. In some embodiments, provided are methods for the treatment of oHCM comprising administration of aficamten, or a pharmaceutically acceptable salt thereof, or a pharmaceutical composition containing aficamten, or a pharmaceutically acceptable salt thereof, as a monotherapy, wherein patient has chronic oHCM and has previously received SOC medical therapy for oHCM prior to administering aficamten, or a pharmaceutically acceptable salt thereof, and has discontinued the SOC medical therapy to oHCM prior to administering aficamten, or a pharmaceutically acceptable salt thereof.

In some embodiments, the patient has LVEF≥60% and resting LVOT-G≥30 mm Hg prior to administering aficamten, or a pharmaceutically acceptable salt thereof. In some embodiments, the patient has LVEF≥60% and post-Valsalva LVOT-G≥50 mmHg prior to administering aficamten. In some embodiments, the patient has LVEF≥60%, resting LVOT-G≥30 mm Hg, and post-Valsalva LVOT-G≥50 mmHg prior to administering aficamten. In some embodiments, the patient has LVEF≥60%, resting LVOT-G≥30 mm Hg, and NYHA Class II or Class III prior to administering aficamten, or a pharmaceutically acceptable salt thereof. In some embodiments, the patient has LVEF≥60%, post-Valsalva LVOT-G≥50 mmHg, and NYHA Class II or Class III prior to administering aficamten. In some embodiments, the patient has LVEF≥60%, resting LVOT-G≥30 mm Hg, post-Valsalva LVOT-G≥50 mmHg, and NYHA Class II or Class III prior to administering aficamten.

During the course of treatment for oHCM, the dose of aficamten administered to the patient may be titrated, for example by increasing, decreasing, or maintaining the dose. Titration may occur once during treatment, or may be performed iteratively separated by a period of time. For example, in some implementations, the dose of aficamten is titrated two or more times (e.g., 3, 4, 5 or more) during the course of treatment. In some embodiments, a new daily dose amount is administered to the patient at a constant amount for about 1 week to about 8 weeks (or about 2 weeks to about 6 weeks, or about 4 weeks) before the daily dose amount is titrated. In some embodiments, a new daily dose amount is administered to the patient at a constant amount for about 2 weeks before being titrated. For example, a first daily dose may be administered to the patient for about 2 weeks before a first titration, wherein the daily dose amount is increased, decreased, or maintained. The second titration may then occur approximately 2 weeks after the first titration. Titration of the dose allows the dose to be personalized to the patient's response to the drug, thus maximizing the potential treatment effect for patients.

Titration of the dose can be based on one or more of a biplane left ventricular ejection fraction (LVEF) and post-Valsalva LVOT-G measured in the patient. The measurement or measurements may be determined, for example, using an echocardiogram. The echocardiogram is taken following administration of the daily dose, for example about 1 hour to about 3 hours following administration of the dose. In some embodiments, the echocardiogram is taken about 2 hours following administration of the daily dose.

In some embodiments, an initial daily dose of about 5 mg, about 10 mg, about 15 mg, or about 20 mg aficamten, or any amount therebetween, is administered to the patient. After a period of time (e.g., about 2 weeks), biplane LVEF and/or post-Valsalva LVOT-G is measured, for example by echocardiography, following the administration of the dose (for example, about 1-3 hours, or about 2 hours following the administration of the dose). The dose may be decreased or terminated if the biplane LVEF is below a predetermined biplane LVEF threshold (e.g., lower than 50%). For example, the dose may be decreased if the biplane LVEF is below the biplane LVEF threshold and the current dose is not the lowest (e.g., first) dose. The dose may be terminated if the biplane LVEF is below the biplane LVEF threshold and the current dose is the lowest (e.g., first) dose. The dose may be maintained if the biplane LVEF is at or above the predetermined biplane LVEF threshold, and below a second predetermined biplane LVEF threshold (e.g., 50%≤LVEF<55%), or the dose may be maintained if the biplane LVEF is at or above the second predetermined biplane LVEF threshold (e.g., 55% or higher) and the post-Valsalva LVOT-G is below the predetermined LVOT-G threshold (e.g., less than 30%). If the post-Valsalva LVOT-G is at or above a predetermined LVOT-G threshold (e.g., about 30 mmHg or more) and the biplane LVEF is at or above a second predetermined biplane LVEF threshold (e.g., about 55% or more), the daily dose is increased. In some embodiments, the biplane LVEF threshold is about 50%, and the post-Valsalva LVOT-G threshold is about 30 mmHg. If the LVEF is <40% at any time, administration of the daily dose is temporarily interrupted. In some embodiments, titration of the dose of aficamten comprises maintaining the dose at the current dose; increasing the dose by about 1 mg, about 2 mg, about 3 mg, about 4 mg, about 5 mg, or about 10 mg, or any amount therebetween; decreasing the dose by about 1 mg, about 2 mg, about 3 mg, about 4 mg, about 5 mg, or about 10 mg, or any amount therebetween; or terminating administration. In some embodiments, titration of the dose comprises maintaining the dose at the current dose; increasing the dose by about 1 mg, about 2 mg, about 3 mg, about 4 mg, about 5 mg, or about 10 mg, or any amount therebetween; or decreasing the dose by about 1 mg, about 2 mg, about 3 mg, about 4 mg, about 5 mg, or about 10 mg, or any amount therebetween.

After a period of time (e.g., about 2 weeks) of the patient being administered the first titrated dose, the dose may be again titrated (i.e., increased, decreased, or maintained) based on the biplane LVEF and/or post-Valsalva LVOT-G of the patient, for example using the same threshold parameters as discussed above. An exemplary dose titration schedule includes: administration of a first titrated dose for about 2 weeks, about 3 weeks, about 4 weeks, about 5 weeks, about 6 weeks, about 8 weeks, about 10 weeks, or about 12 weeks, or any amount of time therebetween, followed by dose titration based on the biplane LVEF and/or post-Valsalva LVOT-G of the patient, for example using the same threshold parameters as discussed above. Further iterations of administration and dose titration may be conducted accordingly.

Provided herein is a method of reducing post-Valsalva LVOT-G to less than a particular value in a patient with symptomatic obstructive hypertrophic cardiomyopathy (oHCM) which comprises administering to the patient a therapeutically effective amount of aficamten, or a pharmaceutically acceptable salt thereof, wherein the patient has been recently diagnosed with oHCM, the patient is treatment naïve, or the patient has chronic oHCM. The reduction in post-Valsalva LVOT-G to less than the particular value may occur within one week, two weeks, three weeks, four weeks, one month, five weeks, six week, seven weeks, eight weeks, two months, nine weeks, or ten weeks of initiating treatment with aficamten, or a pharmaceutically acceptable salt thereof. The reduction in post-Valsalva LVOT-G may be sustained for at least 10 weeks of treatment. In some embodiments, the reduction in post-Valsalva LVOT-G occurs within two to six weeks of the end of dose titration. In some embodiments, the reduction in post-Valsalva LVOT-G peaks within two to six weeks of the end of dose titration. In some embodiments, the particular post-Valsalva LVOT-G value is: 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, or 30 mmHg.

Further provided herein is a method of treating obstructive hypertrophic cardiomyopathy (oHCM) in a patient with heart failure symptoms, wherein the patient has been recently diagnosed with oHCM, the patient is treatment naïve, or the patient has chronic oHCM, comprising administering to the patient a therapeutically effective amount of aficamten, or a pharmaceutically acceptable salt thereof, wherein the method results in a reduction of heart failure symptoms as assessed by NYHA classification. In some embodiments of the foregoing, the methods improve heart failure symptoms by at least one NYHA class in the patient, for instance, by one or two NYHA class(es). In some embodiments of the foregoing, the methods convert patients from NYHA class III to class II or class I. In some embodiments of the foregoing, the methods convert patients from NYHA class III to class II. In some embodiments of the foregoing, the methods convert patients from NYHA class III to class I. In some embodiments of the foregoing, the methods convert patients from NYHA class II to class I. In some embodiments of the foregoing, reduction of heart failure symptoms occurs within ten weeks of initiating treatment with aficamten, or a pharmaceutically acceptable salt thereof.

FIG. 1 of WO 2024/031016 and U.S. Patent Publication US-2024-0115554-A1 illustrates and exemplary method for treating obstructive hypertrophic cardiomyopathy (oHCM) in a patient, wherein the patient has been recently diagnosed with oHCM (e.g., within the past 12 months), the patient is treatment naïve for oHCM, or the patient has previously received standard of care medical therapy for oHCM and has discontinued said therapy prior to administering aficamten, that includes titrating the daily dose of aficamten, or a pharmaceutically acceptable salt thereof. The exemplary method shown in FIG. 1 of WO 2024/031016 and U.S. Patent Publication US-2024-0115554-A1 provides four daily dose levels (e.g., about 5 mg, 10 mg, 15 mg, and 20 mg), with a first daily dose level being the lowest daily dose level (e.g., about 5 mg), but may be readily modified to include additional or fewer dose levels. The exemplary method shown in FIG. 1 of WO 2024/031016 and U.S. Patent Publication US-2024-0115554-A1 may be further modified such that the first daily dose level is not the lowest daily dose level. At 102, the first daily dose level (e.g., about 5 mg) of aficamten (Compound 1 of WO 2024/031016 and U.S. Patent Publication US-2024-0115554-A1), or a pharmaceutically acceptable salt thereof, is administered to a patient. After a first time period, the daily dose level is increased or maintained, or the administration is terminated, at 104. The selection may be based on a first echocardiogram acquired for the patient after the first time period. Termination of administration 106 may be selected if the biplane LVEF of the first echocardiogram is below a predetermined biplane LVEF threshold (e.g., 50%), wherein the no further dose of aficamten or pharmaceutically acceptable salt thereof is administered to the patient. Maintenance of the first daily dose level (e.g., about 5 mg) may be selected when either of the following conditions are met on the first echocardiogram: (1) the biplane LVEF is at or above the predetermined biplane LVEF threshold (e.g., 50%) and below a second predetermined biplane LVEF threshold (e.g., 55%); or (2) the biplane LVEF is at or above the second predetermined biplane LVEF threshold (e.g., 55%) and the post-Valsalva LVOT-G of the first echocardiogram is below a predetermined post-Valsalva LVOT-G threshold (e.g., 30 mmHg). If maintenance is selected, the first daily dose level of aficamten, or a pharmaceutically acceptable salt thereof, is administered to the patient at 102 for a second time period, and, optionally, the daily dose may be re-titrated at 104 after a second time period. The daily dose level may be increased to a second daily dose level (e.g., 10 mg) when either of the following conditions are met on the first echocardiogram: (1) the biplane LVEF is at or above the predetermined biplane LVEF threshold (e.g., 50%) and below a second predetermined biplane LVEF threshold (e.g., 55%); or (2) the biplane LVEF is at or above the second predetermined biplane LVEF threshold (e.g., 55%) and the post-Valsalva LVOT-G of the first echocardiogram is above a predetermined post-Valsalva LVOT-G threshold (e.g., 30 mmHg). If an increase in the daily dose level is selected, the second daily dose level of aficamten, or a pharmaceutically acceptable salt thereof, is administered to the patient for the second time period at 108.

If the second daily dose level of aficamten, or a pharmaceutically acceptable salt thereof, (e.g., 10 mg) is administered to the patient at 108, the daily dose may be re-titrated (i.e., select an increase, decrease, or maintenance of the daily dose) based on an echocardiogram at 110. The daily dose may be decreased to the first daily dose level (e.g., from about 10 mg to about 5 mg) if the biplane LVEF of the echocardiogram is below a predetermined biplane LVEF threshold (e.g., 50%). If the daily dose is decreased to the first daily dose level, the first daily dose level (e.g., about 5 mg) is administered to the patient at 102. Maintenance of the second daily dose level (e.g., about 10 mg) may be selected when either of the following conditions are met on the echocardiogram: (1) the biplane LVEF is at or above the predetermined biplane LVEF threshold (e.g., 50%) and below a second predetermined biplane LVEF threshold (e.g., 55%); or (2) the biplane LVEF is at or above the second predetermined biplane LVEF threshold (e.g., 55%) and the post-Valsalva LVOT-G of the echocardiogram is below a predetermined post-Valsalva LVOT-G threshold (e.g., 30 mmHg). If maintenance is selected, the second daily dose level of aficamten, or a pharmaceutically acceptable salt thereof, is administered to the patient at 108 for a further time period, and, optionally, the daily dose may be re-titrated at 110 after said time period. The daily dose level may be increased to a third daily dose level (e.g., 15 mg) when the biplane LVEF is at or above the second predetermined biplane LVEF threshold (e.g., 55%) and the post-Valsalva LVOT-G of the echocardiogram is above a predetermined post-Valsalva LVOT-G threshold (e.g., 30 mmHg). If an increase in the daily dose level is selected, the second daily dose level of aficamten, or a pharmaceutically acceptable salt thereof, is administered to the patient for the time period at 112.

If the third daily dose level of aficamten, or a pharmaceutically acceptable salt thereof, (e.g., 10 mg) is administered to the patient at 112, the daily dose may be re-titrated (i.e., select an increase, decrease, or maintenance of the daily dose) based on an echocardiogram at 114. The daily dose may be decreased to the second daily dose level (e.g., from 15 mg to 10 mg) if the biplane LVEF of the echocardiogram is below a predetermined biplane LVEF threshold (e.g., 50%). If the daily dose is decreased to the second daily dose level, the second daily dose level is administered to the patient at 108. Maintenance of the third daily dose level (e.g., about 15 mg) may be selected when either of the following conditions are met on the echocardiogram: (1) the biplane LVEF is at or above the predetermined biplane LVEF threshold (e.g., 50%) and below a second predetermined biplane LVEF threshold (e.g., 55%); or (2) the biplane LVEF is at or above the second predetermined biplane LVEF threshold (e.g., 55%) and the post-Valsalva LVOT-G of the echocardiogram is below a predetermined post-Valsalva LVOT-G threshold (e.g., 30 mmHg). If maintenance is selected, the third daily dose level of aficamten, or a pharmaceutically acceptable salt thereof, is administered to the patient at 112 for a further time period, and, optionally, the daily dose may be re-titrated at 114 after said time period. The daily dose level may be increased to a fourth daily dose level (e.g., 20 mg) when the biplane LVEF is at or above the second predetermined biplane LVEF threshold (e.g., 55%) and the post-Valsalva LVOT-G of the echocardiogram is above a second predetermined post-Valsalva LVOT-G threshold (e.g., 30 mmHg). If an increase in the daily dose level is selected, the fourth daily dose level of aficamten, or a pharmaceutically acceptable salt thereof, is administered to the patient for the second time period at 116.

In the exemplary method shown in FIG. 1 of WO 2024/031016 and U.S. Patent Publication US-2024-0115554-A1, the first daily dose level is the minimum dose, and therefore may not be further decreased. Nevertheless, in other embodiments, the first daily dose level may be other than the minimum dose, and therefore may be decreased to a lower dose level (e.g., from 10 mg to 5 mg) if the biplane LVEF of the echocardiogram is below a predetermined biplane LVEF threshold (e.g., 50%).

In the exemplary method shown in FIG. 1 of WO 2024/031016 and U.S. Patent Publication US-2024-0115554-A1, the fourth daily dose level is the maximum dose, and therefore may not be further increased. Nevertheless, in other embodiments, additional dose levels may be available and the daily dose may be further increased at 118. The method shown in FIG. 1 of WO 2024/031016 and U.S. Patent Publication US-2024-0115554-A1, at 118 a selection is made to maintain the fourth daily dose level or decrease the daily dose level based on an echocardiogram. The daily dose may be decreased to the third daily dose level (e.g., from 20 mg to 15 mg) if the biplane LVEF of the echocardiogram is below a predetermined biplane LVEF threshold (e.g., 50%). If the daily dose is decreased to the third daily dose level, the second daily dose level is administered to the patient at 112. Maintenance of the third daily dose level (e.g., about 15 mg) may be selected when either of the following conditions are met on the echocardiogram: (1) the biplane LVEF is at or above the predetermined biplane LVEF threshold (e.g., 50%) and below a second predetermined biplane LVEF threshold (e.g., 55%); or (2) the biplane LVEF is at or above the second predetermined biplane LVEF threshold (e.g., 55%) and the post-Valsalva LVOT-G of the echocardiogram is below a predetermined post-Valsalva LVOT-G threshold (e.g., 30 mmHg). If maintenance is selected, the third daily dose level of aficamten, or a pharmaceutically acceptable salt thereof, is administered to the patient at 116 for a further time period, and, optionally, the daily dose may be re-titrated at 118 after said time period.

Exemplary daily dose increases include an increase from about 5 mg to about 10 mg aficamten, about 10 mg to about 15 mg aficamten, or about 10 mg to about 20 mg aficamten. Other dose increases may be readily envisioned, for instance, increasing a given initial daily dose by about 1 mg, about 2 mg, about 3 mg, about 4 mg, about 5 mg, or about 10 mg, or any amount therebetween. Exemplary daily dose decreases include a decrease from about 20 mg to about 10 mg, about 15 mg to about 10 mg, or about 10 mg to about 5 mg. Other dose decreases may be readily envisioned, for instance, decreasing a given initial daily dose by about 1 mg, about 2 mg, about 3 mg, about 4 mg, about 5 mg, or about 10 mg, or any amount therebetween.

Exemplary embodiments of the methods described herein comprise administering a first daily dose (e.g., a first daily dose of between about 1 mg and about 20 mg, such as 1 mg, 2 mg, 3 mg, 4 mg, 5 mg, 6 mg, 7 mg, 8 mg, 9 mg, 10 mg, 15 mg, or 20 mg) or any amount therebetween) of aficamten, or a pharmaceutically acceptable salt thereof, for a first period (e.g., about one week, two weeks, three weeks, four weeks, five weeks, six weeks, seven weeks, eight weeks, nine weeks, ten weeks, twelve weeks, or any length of time therebetween), followed by maintaining the daily dose, decreasing the daily dose (e.g., decreasing the daily dose by between about 1 mg and about 10 mg, such as decreasing the daily dose by 1 mg, 2 mg, 3 mg, 4 mg, 5 mg 6 mg, 7 mg, 8 mg, 9 mg, or 10 mg, or any amount therebetween), increasing the daily dose (e.g., increasing the daily dose by between about 1 mg and about 10 mg, such as increasing the daily dose by 1 mg, 2 mg, 3 mg, 4 mg, 5 mg 6 mg, 7 mg, 8 mg, 9 mg, or 10 mg, or any amount therebetween), or terminating administration based on the biplane LVEF and/or post-Valsalva LVOT-G of the patient to arrive at a second daily dose. Another exemplary embodiment of the methods described herein comprises administering a first daily dose of aficamten, or a pharmaceutically acceptable salt thereof, for about two weeks, followed by maintaining the daily dose, decreasing the daily dose by about 5 mg, increasing the daily dose by about 5 mg, or terminating administration based on the biplane LVEF and/or post-Valsalva LVOT-G of the patient to arrive at a second daily dose. Another exemplary embodiment of the methods described herein comprises administering a first daily dose of aficamten, or a pharmaceutically acceptable salt thereof, for about three weeks, followed by maintaining the daily dose, decreasing the daily dose by about 5 mg, increasing the daily dose by about 5 mg, or terminating administration based on the resting LVOT-G, biplane LVEF, and/or post-Valsalva LVOT-G of the patient to arrive at a second daily dose. Another exemplary embodiment of the methods described herein comprises administering a first daily dose of aficamten, or a pharmaceutically acceptable salt thereof, for about two weeks, followed by maintaining the daily dose, decreasing the daily dose by about 10 mg, increasing the daily dose by about 10 mg, or terminating administration based on the biplane LVEF and/or post-Valsalva LVOT-G of the patient to arrive at a second daily dose. Another exemplary embodiment of the methods described herein comprises administering a first daily dose of aficamten, or a pharmaceutically acceptable salt thereof, for about three weeks, followed by maintaining the daily dose, decreasing the daily dose by about 10 mg, increasing the daily dose by about 10 mg, or terminating administration based on the biplane LVEF and/or post-Valsalva LVOT-G of the patient to arrive at a second daily dose. Another exemplary embodiment of the methods described herein comprises administering a first daily dose of aficamten, or a pharmaceutically acceptable salt thereof, for between about 2 and about 12 weeks, followed by maintaining the daily dose, decreasing the daily dose by about 10 mg, increasing the daily dose by about 10 mg, or terminating administration based on the biplane LVEF and/or post-Valsalva LVOT-G of the patient to arrive at a second daily dose.

Treatment for oHCM can result in improved exercise capacity and/or relieve symptoms in patients with hyperdynamic ventricular contraction resulting from obstructive hypertrophic cardiomyopathy. The methods disclosed herein may be useful for treating patients who have been recently diagnosed, are treatment naïve, or have chronic oHCM.

In some embodiments, a therapeutically effective amount of aficamten, or a pharmaceutically acceptable salt thereof, is administered to a patient with obstructive hypertrophic cardiomyopathy, thereby decreasing post-Valsalva LVOT-G in the patient, wherein the patient has been recently diagnosed with oHCM (e.g., within the past 12 months), the patient is treatment naïve for oHCM, or the patient has previously received standard of care medical therapy for oHCM and has discontinued said therapy prior to administering aficamten. In some embodiments, the patient has a baseline post-Valsalva LVOT-G of about 30 mmHg or more, about 40 mmHg or more, 50 mmHg or more, about 60 mmHg or more, or about 70 mmHg or more. In response to administration of the therapeutically effective amount of aficamten, or a pharmaceutically acceptable salt thereof, the post-Valsalva LVOT-G can decrease to less than 50 mmHg, for example to about 45 mmHg or less, about 40 mmHg or less, about 35 mmHg or less, or about 30 mmHg or less. In some embodiments, in response to administration of the therapeutically effective amount of aficamten, or a pharmaceutically acceptable salt thereof, the post-Valsalva LVOT-G decreases by about 10 mmHg or more, about 15 mmHg or more, about 20 mmHg or more, about 25 mmHg or more, about 30 mmHg or more, or about 35 mmHg or more. In some embodiments, in response to administration of the therapeutically effective amount of aficamten, or a pharmaceutically acceptable salt thereof, the post-Valsalva LVOT-G decreases by about 10 mmHg to about 40 mmHg. The decrease in post-Valsalva LVOT-G may occur after about 1 week, after about 2 weeks, after about 3 weeks, after about 4 weeks, after about 5 weeks, after about 6 weeks, after about 8 weeks, or after about 10 weeks of daily dose administration.

In some embodiments, a therapeutically effective amount of aficamten, or a pharmaceutically acceptable salt thereof, is administered to a patient with obstructive hypertrophic cardiomyopathy, thereby decreasing a left ventricle mass index (LVMI) for the patient, wherein the patient has been recently diagnosed with oHCM (e.g., within the past 12 months), the patient is treatment naïve for oHCM, or the patient has previously received standard of care medical therapy for oHCM and has discontinued said therapy prior to administering aficamten. In response to administration of the therapeutically effective amount of aficamten, or a pharmaceutically acceptable salt thereof, the LVMI can decrease by about 1 g/m² or more, by about 1.5 g/m² or more, by about 2 g/m² or more, by about 2.5 g/m² or more, by about 3 g/m² or more, by about 3.5 g/m² or more, or by about 4 g/m² or more. In some embodiments, in response to administration of the therapeutically effective amount of aficamten, or a pharmaceutically acceptable salt thereof, the LVMI decreases by about 1 g/m² to about 10 g/m², such as by about 1 g/m2 to about 6 g/m², or about 2 g/m² to about 5 g/m². The decrease in LVMI may occur after about 1 week, after about 2 weeks, after about 3 weeks, after about 4 weeks, after about 5 weeks, after about 6 weeks, after about 8 weeks, or after about 10 weeks of daily dose administration.

In some embodiments, a therapeutically effective amount of aficamten, or a pharmaceutically acceptable salt thereof, is administered to a patient with obstructive hypertrophic cardiomyopathy, thereby decreasing a left arterial volume index (LAVI) for the patient, wherein the patient has been recently diagnosed with oHCM (e.g., within the past 12 months), the patient is treatment naïve for oHCM, or the patient has previously received standard of care medical therapy for oHCM and has discontinued said therapy prior to administering aficamten. In response to administration of the therapeutically effective amount of aficamten, or a pharmaceutically acceptable salt thereof, the LAVI can decrease by about 0.5 mL/m2 or more, by about 1 mL/m2 or more, by about 1.5 mL/m2 or more, by about 2 mL/m2 or more, or by about 2.5 mL/m2 or more. In some embodiments, in response to administration of the therapeutically effective amount of aficamten, or a pharmaceutically acceptable salt thereof, the LAVI decreases by about 0.5 mL/m2 to about 5 mL/m2 mmHg, such as by about 0.5 mL/m2 to about 4 g/m², or about 1 mL/m2 to about 3 mL/m2. The decrease in LAVI may occur after about 1 week, after about 2 weeks, after about 3 weeks, after about 4 weeks, after about 5 weeks, after about 6 weeks, after about 8 weeks, or after about 10 weeks of daily dose administration.

In some embodiments, a therapeutically effective amount of aficamten, or a pharmaceutically acceptable salt thereof, is administered to a patient with obstructive hypertrophic cardiomyopathy, thereby decreasing an e′ value for the patient, wherein the patient has been recently diagnosed with oHCM (e.g., within the past 12 months), the patient is treatment naïve for oHCM, or the patient has previously received standard of care medical therapy for oHCM and has discontinued said therapy prior to administering aficamten. In response to administration of the therapeutically effective amount of aficamten, or a pharmaceutically acceptable salt thereof, the e′ value can decrease by about 0.1 cm/s or more, by about 0.15 cm/s or more, by about 0.2 cm/s or more, or by about 0.25 cm/s or more. In some embodiments, in response to administration of the therapeutically effective amount of aficamten, or a pharmaceutically acceptable salt thereof, the e′ value decreases by about 0.05 cm/s to about 0.3 cm/s, such as by about 0.1 cm/s to about 0.25 cm/s, or about 0.15 cm/s to about 0.25 cm/s. The decrease in e′ value may occur after about 1 week, after about 2 weeks, after about 3 weeks, after about 4 weeks, after about 5 weeks, after about 6 weeks, after about 8 weeks, or after about 10 weeks of daily dose administration.

In some embodiments, a therapeutically effective amount of aficamten, or a pharmaceutically acceptable salt thereof, is administered to a patient with obstructive hypertrophic cardiomyopathy, thereby decreasing a lateral E/e′ ratio for the patient, wherein the patient has been recently diagnosed with oHCM (e.g., within the past 12 months), the patient is treatment naïve for oHCM, or the patient has previously received standard of care medical therapy for oHCM and has discontinued said therapy prior to administering aficamten. In response to administration of the therapeutically effective amount of aficamten, or a pharmaceutically acceptable salt thereof, the lateral E/e′ ratio can decrease by about 0.5 or more, by 1 or more, by about 1.2 or more, by about 1.5 or more, or by about 1.8 or more. In some embodiments, in response to administration of the therapeutically effective amount of aficamten, or a pharmaceutically acceptable salt thereof, the lateral E/e′ ratio decreases by about 0.5 to about 2, such as by about 1 to about 1.8, or about 1.5 to about 1.8. The decrease in lateral E/e′ ratio may occur after about 1 week, after about 2 weeks, after about 3 weeks, after about 4 weeks, after about 5 weeks, after about 6 weeks, after about 8 weeks, or after about 10 weeks of daily dose administration.

In some embodiments, a therapeutically effective amount of aficamten, or a pharmaceutically acceptable salt thereof, is administered to a patient with obstructive hypertrophic cardiomyopathy, thereby decreasing a level of brain natriuretic peptide or N-terminal prohormone of brain natriuretic peptide (NT-proBNP) in the patient, wherein the patient has been recently diagnosed with oHCM (e.g., within the past 12 months), the patient is treatment naïve for oHCM, or the patient has previously received standard of care medical therapy for oHCM and has discontinued said therapy prior to administering aficamten. The decrease in a level of brain natriuretic peptide or N-terminal prohormone of brain natriuretic peptide (NT-proBNP) in the patient may occur after about 1 week, after about 2 weeks, after about 3 weeks, after about 4 weeks, after about 5 weeks, after about 6 weeks, after about 8 weeks, or after about 10 weeks of daily dose administration.

In some embodiments, a therapeutically effective amount of aficamten, or a pharmaceutically acceptable salt thereof, is administered to a patient with obstructive hypertrophic cardiomyopathy, thereby decreasing a level of cardiac troponin I, wherein the patient has been recently diagnosed with oHCM (e.g., within the past 12 months), the patient is treatment naïve for oHCM, or the patient has previously received standard of care medical therapy for oHCM and has discontinued said therapy prior to administering aficamten. The decrease in a level of cardiac troponin I in the patient may occur after about 1 week, after about 2 weeks, after about 3 weeks, after about 4 weeks, after about 5 weeks, after about 6 weeks, after about 8 weeks, or after about 10 weeks of daily dose administration.

In some embodiments of any of the foregoing, the patient with obstructive hypertrophic cardiomyopathy is classified as NYHA class III when administration with aficamten, or a pharmaceutically acceptable salt thereof, is initiated, wherein the patient has been recently diagnosed with oHCM (e.g., within the past 12 months), the patient is treatment naïve for oHCM, or the patient has previously received standard of care medical therapy for oHCM and has discontinued said therapy prior to administering aficamten. In some embodiments, the patient with obstructive hypertrophic cardiomyopathy is classified as NYHA class II when administration with aficamten, or a pharmaceutically acceptable salt thereof, is initiated.

In some embodiments of any of the foregoing, administration of aficamten, or a pharmaceutically acceptable salt thereof, to a patient with obstructive hypertrophic cardiomyopathy results in an improvement in exercise capacity in the patient, wherein the patient has been recently diagnosed with oHCM (e.g., within the past 12 months), the patient is treatment naïve for oHCM, or the patient has previously received standard of care medical therapy for oHCM and has discontinued said therapy prior to administering aficamten. In some embodiments of any of the foregoing, administration of aficamten, or a pharmaceutically acceptable salt thereof, to a patient with obstructive hypertrophic cardiomyopathy results in an improvement in exercise capacity in the patient, for example as measured by change in peak oxygen uptake (pVO₂) or change in peak oxygen uptake (pVO₂) by cardiopulmonary exercise testing (CPET).

In some embodiments of any of the foregoing, administration of aficamten, or a pharmaceutically acceptable salt thereof, to a patient with obstructive hypertrophic cardiomyopathy results in an improvement in total workload during CPET, wherein the patient has been recently diagnosed with oHCM (e.g., within the past 12 months), the patient is treatment naïve for oHCM, or the patient has previously received standard of care medical therapy for oHCM and has discontinued said therapy prior to administering aficamten.

In some embodiments of any of the foregoing, administration of aficamten, or a pharmaceutically acceptable salt thereof, to a patient with obstructive hypertrophic cardiomyopathy results in an improvement in other CPET parameters, including but not limited to one or more of: (1) ventilator efficiency (VE/VCO₂ slope); (2) circulatory power (VO₂× systolic BP); and (3) ventilator anaerobic threshold (VAT), wherein the patient has been recently diagnosed with oHCM (e.g., within the past 12 months), the patient is treatment naïve for oHCM, or the patient has previously received standard of care medical therapy for oHCM and has discontinued said therapy prior to administering aficamten.

In some embodiments of any of the foregoing, administration of aficamten, or a pharmaceutically acceptable salt thereof, to a patient with obstructive hypertrophic cardiomyopathy results in an improvement in patient health status, wherein the patient has been recently diagnosed with oHCM (e.g., within the past 12 months), the patient is treatment naïve for oHCM, or the patient has previously received standard of care medical therapy for oHCM and has discontinued said therapy prior to administering aficamten. In some embodiments of any of the foregoing, administration of aficamten, or a pharmaceutically acceptable salt thereof, to a patient with obstructive hypertrophic cardiomyopathy results in an improvement in patient health status as determined by changes in the Kansas City Cardiomyopathy Questionnaire-Overall Summary Score (KCCQ-OSS). In some embodiments of any of the foregoing, administration of aficamten, or a pharmaceutically acceptable salt thereof, to a patient with obstructive hypertrophic cardiomyopathy results in an improvement in patient health status as determined by changes in the Kansas City Cardiomyopathy Questionnaire-Clinical Summary Score (KCCQ-CSS). In some embodiments of any of the foregoing, administration of aficamten, or a pharmaceutically acceptable salt thereof, to a patient with obstructive hypertrophic cardiomyopathy results in an improvement in patient health status as determined by changes in the Kansas City Cardiomyopathy Questionnaire-Total Symptom Score (KCCQ-TSS). In some embodiments of any of the foregoing, administration of aficamten, or a pharmaceutically acceptable salt thereof, to a patient with obstructive hypertrophic cardiomyopathy results in an improvement in patient health status as determined by changes in the Kansas City Cardiomyopathy Questionnaire-Physical Limitation Score (KCCQ-PLS). In some embodiments of any of the foregoing, administration of aficamten, or a pharmaceutically acceptable salt thereof, to a patient with obstructive hypertrophic cardiomyopathy results in an improvement in patient health status as determined by changes in the Kansas City Cardiomyopathy Questionnaire-Social Limitation Score (KCCQ-SLS). In some embodiments of any of the foregoing, administration of aficamten, or a pharmaceutically acceptable salt thereof, to a patient with obstructive hypertrophic cardiomyopathy results in an improvement in patient health status as determined by changes in the Kansas City Cardiomyopathy Questionnaire quality of live (KCCQ-QoL). In some embodiments, administration of aficamten, or a pharmaceutically acceptable salt thereof, to a patient with obstructive hypertrophic cardiomyopathy results in improvement in one or more KCCQ domain scores (such as KCCQ-OSS, KCCQ-CSS, KCCQ-TSS, KCCQ-PLS, KCCQ-SLS, or KCCQ-QoL) by at least about 5 points, at least about 10 points, or at least about 20 points. In some embodiments, administration of aficamten, or a pharmaceutically acceptable salt thereof, to a patient with obstructive hypertrophic cardiomyopathy results in improvement in one or more KCCQ score (such as KCCQ-OSS, KCCQ-CSS, KCCQ-TSS, KCCQ-PLS, KCCQ-SLS, or KCCQ-QoL) by between about 5 points and less than 10 points, between about 10 points and less than 20 points, or by at least 20 points. In some such embodiments, the improvement in the one or more KCCQ domain scores is an improvement in KCCQ-OSS. In some embodiments, the improvement in the one or more KCCQ domain scores is sustained for about 6 months. In some embodiments, administration of aficamten, or a pharmaceutically acceptable salt thereof, to a patient with obstructive hypertrophic cardiomyopathy results in improvement in KCCQ-CSS by 1 point, 2 points, 3 points, 4 points, 5 points, or more than 5 points. In some embodiments of any of the foregoing, administration of aficamten, or a pharmaceutically acceptable salt thereof, to a patient with obstructive hypertrophic cardiomyopathy results in an improvement in patient health status and health-related quality of life as measured by PRO questionnaire, as determined by changes from responses to the EuroQol 5-dimension 5-level instrument (EQ-5D-5L). In some embodiments administration with aficamten, or a pharmaceutically acceptable salt thereof, results in an improvement in one or more HCM symptoms. In some embodiments, improvement in one or more HCM symptoms comprises reduced severity and/or frequency of chest pain, dizziness, shortness of breath, fainting during physical activity, tiredness, low energy, or limitation in physical activity.

Combinations of the foregoing results are also contemplated. For example, in some embodiments of any of the foregoing, administration of aficamten, or a pharmaceutically acceptable salt thereof, to a patient with obstructive hypertrophic cardiomyopathy, wherein the patient has been recently diagnosed with oHCM (e.g., within the past 12 months), the patient is treatment naïve for oHCM, or the patient has previously received standard of care medical therapy for oHCM and has discontinued said therapy prior to administering aficamten, results in an improvement in exercise capacity and functional class, for instance, as determined by (1) change from baseline of ≥1.5 mL/kg/min in pVO₂ and >1 class improvement in NYHA Functional Class; or (2) change from baseline of ≥3.0 mL/kg/min in pVO₂ and no worsening of NYHA Functional Class. In some embodiments of any of the foregoing, administration of aficamten, or a pharmaceutically acceptable salt thereof, to a patient with obstructive hypertrophic cardiomyopathy results in resting LVOT-G<30 mmHg, post-Valsalva LVOT-G<50 mmHg, and NYHA Functional Class I in the patient. In some embodiments of any of the foregoing, administration of aficamten, or a pharmaceutically acceptable salt thereof, to a patient with obstructive hypertrophic cardiomyopathy results in resting LVOT-G<30 mmHg, post-Valsalva LVOT-G<50 mmHg, and ≥1 class improvement in NYHA Functional Class in the patient.

In some embodiments, the methods disclosed herein result in one or more effects selected from the group consisting of: reduction in resting LVOT-G to less than 30 mmHg; reduction in post-Valsalva LVOT-G to less than 50 mmHg; improvement in mitral regurgitation; improvement in cardiac relaxation; beneficial cardiac remodeling; reverse cardiac remodeling; beneficial cardiac structural remodeling; beneficial cardiac functional remodeling; reversal of adverse cardiac remodeling; reduction in mean left ventricular mass index (LVMI); improvement in left ventricular (LV) filling pressures; reduction in left atrial volume index (LAVI); reduction in the categorical assessment of systolic anterior motion of the mitral valve leaflet; reduction in systolic anterior motion of the mitral valve leaflet; reduction in the frequency of eccentric mitral regurgitation; reduction in mitral regurgitation; reduction in lateral E/e′; reduction in lateral E/E; reduction in brain natriuretic peptide (BNP) levels; reduction in N-terminal prohormone of brain natriuretic peptide (NT-proBNP) levels; reduction in cardiac troponin I levels; decreased left ventricular wall stress; decreased myocardial injury; and reduction in heart failure symptoms, for example, reduction in NYHA classification.

Methods and compositions for accelerating oxygen uptake recovery (VO₂Rec) in a patient in need thereof, comprising administering to the patient a therapeutically effective amount of aficamten or a pharmaceutically acceptable salt thereof, are provided therein. In some embodiments, the patient has a heart disease. In some embodiments, the heart disease is hypertrophic cardiomyopathy (HCM). In some embodiments, the heart disease is oHCM.

Methods and compositions for reducing or resolving left ventricular hypertrophy (LVH) in a patient in need thereof, comprising administering to the patient a therapeutically effective amount of aficamten or a pharmaceutically acceptable salt thereof, are provided therein. In some embodiments, the patient has a heart disease. In some embodiments, the heart disease is hypertrophic cardiomyopathy (HCM). In some embodiments, the heart disease is oHCM. In some embodiments, the LVH is LVH with strain pattern. In some embodiments, the LVH is LVH with strain pattern on electrocardiogram.

Methods and compositions for decreasing maximal wall thickness (MWT) in a patient in need thereof, comprising administering to the patient a therapeutically effective amount of aficamten or a pharmaceutically acceptable salt thereof, are provided therein. In some embodiments, the patient has a heart disease. In some embodiments, the heart disease is hypertrophic cardiomyopathy (HCM). In some embodiments, the heart disease is oHCM.

Methods and compositions for reducing chest pain in a patient in need thereof, comprising administering to the patient a therapeutically effective amount of aficamten or a pharmaceutically acceptable salt thereof, are provided therein. In some embodiments, the patient has a heart disease. In some embodiments, the heart disease is hypertrophic cardiomyopathy (HCM). In some embodiments, the heart disease is oHCM.

Methods and compositions for treating oCHM in a patient in need thereof, comprising administering to the patient a therapeutically effective amount of aficamten or a pharmaceutically acceptable salt thereof, wherein the patient had a SAQ-SS score of less than 50 prior to beginning administration of aficamten, are provided therein. In some embodiments, the patient has been recently diagnosed with oHCM (e.g., within the past 12 months), the patient is treatment naïve for oHCM, or the patient has previously received standard of care medical therapy for oHCM and has discontinued said therapy prior to administering aficamten.

### Additional Parameters

In some embodiments of the method described herein, the method improves one or more parameter in the patient wherein each of the one or more parameter is selected from the group consisting of: improvement in mitral valve SAM, improvement in peak A velocity, complete resolution of mitral valve SAM, increase in LV end systolic volume index, increase in LV end systolic volume, increase in Lateral e′ velocity, increase in Septal e′ velocity, increase in LV end systolic dimension, increase in absolute LV GLS (left ventricular global longitudinal strain), decrease in LA volume index, decrease in wall thickness, decrease in maximum wall thickness, decrease in interventricular septal wall thickness, decrease in interventricular septal wall, decrease in inferolateral wall thickness, decrease in LV mass index, decrease in LV global circumferential strain, decrease in TAPSE, decrease in RV s′ velocity, decrease in LA width, decrease in lateral E/e′, decrease in septal E/e′, decrease in peak E wave velocity, and decrease in LV GCS (left ventricular global circumferential strain), are provided therein. In some embodiments, the patient has a heart disease. In some embodiments, the heart disease is hypertrophic cardiomyopathy (HCM). In some embodiments, the HCM is obstructive or nonobstructive or is associated with a sarcomeric and/or non-sarcomeric mutation. In some embodiments, the heart disease is oHCM. In some embodiments, the heart disease is nHCM. In some embodiments, the heart disease is heart failure with preserved ejection fraction.

### Administration Routes and Dosage Forms

Administration of the compounds and compositions disclosed and/or described herein can be via any accepted mode of administration for therapeutic agents including, but not limited to, oral, sublingual, subcutaneous, parenteral, intravenous, intranasal, topical, transdermal, intraperitoneal, intramuscular, intrapulmonary, vaginal, rectal, or intraocular administration. In some embodiments, the compound or composition is administered orally or intravenously. In some embodiments, the compound or composition disclosed and/or described herein is administered orally. In some embodiments, the compound or composition disclosed and/or described herein is administered by injection. In some embodiments, the compound or composition disclosed and/or described herein is administered intranasally. In some embodiments, the compound or composition disclosed and/or described herein is administered transdermally.

Pharmaceutically acceptable compositions include solid, semi-solid, liquid and aerosol dosage forms, such as tablet, capsule, powder, liquid, suspension, suppository, and aerosol forms. The compounds disclosed and/or described herein can also be administered in sustained or controlled release dosage forms (e.g., controlled/sustained release pill, depot injection, osmotic pump, or transdermal (including electrotransport) patch forms) for prolonged timed, and/or pulsed administration at a predetermined rate. In some embodiments, the compositions are provided in unit dosage forms suitable for single administration of a precise dose.

Aficamten can be administered either alone or in combination with one or more conventional pharmaceutical carriers or excipients (e.g., mannitol, lactose, starch, magnesium stearate, sodium saccharine, talcum, cellulose, sodium croscarmellose, glucose, gelatin, sucrose, magnesium carbonate). If desired, the pharmaceutical composition can also contain minor amounts of nontoxic auxiliary substances such as wetting agents, emulsifying agents, solubilizing agents, pH buffering agents and the like (e.g., sodium acetate, sodium citrate, cyclodextrin derivatives, sorbitan monolaurate, triethanolamine acetate, triethanolamine oleate). Generally, depending on the intended mode of administration, the pharmaceutical composition will contain about 0.005% to 95%, or about 0.5% to 50%, by weight of a compound disclosed and/or described herein. Actual methods of preparing such dosage forms are known, or will be apparent, to those skilled in this art; for example, see Remington's Pharmaceutical Sciences, Mack Publishing Company, Easton, Pennsylvania. Suitable formulations of aficamten are disclosed in WO 2021/011808, which is incorporated by reference herein in its entirety.

In some embodiments, aficamten is provided in a formulation comprising: (i) aficamten or a pharmaceutically acceptable salt thereof; (ii) a filler; (iii) a binder; (iv) a disintegrant; (v) a surfactant; and (vi) a lubricant. In some embodiments, aficamten is provided in a formulation comprising: (i) aficamten; (ii) a filler; (iii) a binder; (iv) a disintegrant; (v) a surfactant; and (vi) a lubricant. In some embodiments, the filler is selected from the group consisting of powdered cellulose, microcrystalline cellulose, silicified microcrystalline cellulose, kaolin, corn starch, maize starch, starch derivatives, pregelatinized starch, calcium phosphate, calcium hydrogen phosphate, dicalcium phosphate, tricalcium phosphate, compressible sugar, sugar alcohol, mannitol, sorbitol, maltitol, xylitol, lactitol, lactose, dextrose, maltose, sucrose, glucose, fructose, saccharose, raffinose, dextrates, trehalose, maltodextrins, and mixtures of any of the foregoing.

In some embodiments, the binder is selected from the group consisting of arabic gum, acacia gum, alginate, alginic acid, corn starch, copolyvidone, polyvinylpyrrolidone, gelatin, glyceryl behenate, hydroxyethylcellulose, hydroxypropyl cellulose, carboxymethylcellulose, carboxymethylcellulose calcium, carboxymethylcellulose sodium, methylcellulose, hypromellose, lactose, polyvinyl alcohol, povidone, polyethylene oxide, polyacrylates, potato starch, pregelatinized starch, sodium alginate, sodium starch, sodium carboxy methyl cellulose, starch, and mixtures of any of the foregoing.

In some embodiments, the disintegrant is selected from the group consisting of alginic acid, croscarmellose sodium, cellulose, carboxymethylcellulose calcium, carboxymethylcellulose sodium, microcrystalline cellulose, crospovidone, sodium starch glycolate, low-substituted hydroxypropyl cellulose, polacrillin potassium, pregelatinized starch, partially hydrolyzed starch, sodium carboxymethyl starch, starch, sodium alginate, sodium carboxy methyl cellulose, and mixtures of any of the foregoing.

In some embodiments, the surfactant is selected from the group consisting of cetylpyridine chloride, heptadecaethylene oxycetanol, lecithins, polyoxyethylene stearate, nonoxynol 9, nonoxynol 10, octoxynol 9, sorbitan fatty acid esters, Span 20, Span 40, Span 60, Span 80, Span 85, polysorbates, polysorbate 20, polysorbate 21, polysorbate 40, polysorbate 60, polysorbate 61, polysorbate 65, polysorbate 80, sodium salts of fatty alcohol sulfates, sodium lauryl sulfate, sodium salts of sulfosuccinates, sodium dioctylsulfosuccinate, partial esters of fatty acids with alcohols, glycerine monostearate, glyceryl monooleate, ethers of fatty alcohols with polyoxyethylene, esters of fatty acids with polyoxyethylene, copolymers of ethylenoxide and propylenoxide (Pluronic®), benzalkonium chloride, ethoxylated triglycerides, and mixtures of any of the foregoing.

In some embodiments, the lubricant is selected from the group consisting of hydrogenated castor oil, magnesium stearate, glyceryl monostearate, calcium stearate, glyceryl behenate, glycerol distearate, glyceryl dipalmitostearate, behenoyl polyoxyl-8 glycerides, sodium stearyl fumarate, stearic acid, talc, zinc stearate, mineral oil, polyethylene glycol, polaxamer, sodium lauryl sulfate, and mixtures of any of the foregoing.

In some embodiments, the formulation comprises: (i) about 1% by weight to about 80% by weight of the aficamten or a pharmaceutically acceptable salt thereof; (ii) about 15% by weight to about 90% by weight of the filler; (iii) about 0.1% by weight to about 10% by weight of the binder; (iv) about 1% by weight to about 10% by weight of the disintegrant; (v) about 0.1% by weight to about 10% by weight of the surfactant; and (vi) about 0.1% by weight to about 10% by weight of the lubricant.

In some embodiments, the formulation comprises: (i) about 1% by weight to about 50% by weight of the aficamten or pharmaceutically acceptable salt thereof; (ii) about 40% by weight to about 80% by weight of the filler; (iii) about 0.5% by weight to about 5% by weight of the binder; (iv) about 2% by weight to about 8% by weight of the disintegrant; (v) about 0.5% by weight to about 5% by weight of the surfactant; and (vi) about 0.5% by weight to about 5% by weight of the lubricant.

In some embodiments, the formulation comprises: (i) about 10% by weight to about 30% by weight of the aficamten, or pharmaceutically acceptable salt thereof; (ii) about 60% by weight to about 80% by weight of the filler; (iii) about 1% by weight to about 3% by weight of the binder; (iv) about 4% by weight to about 6% by weight of the disintegrant; (v) about 1% by weight to about 3% by weight of the surfactant; and (vi) about 0.5% by weight to about 1.5% by weight of the lubricant.

In some embodiments, the formulation comprises: (i) about 1% by weight to about 10% by weight of the aficamten, or pharmaceutically acceptable salt thereof; (ii) about 70% by weight to about 90% by weight of the filler; (iii) about 1% by weight to about 3% by weight of the binder; (iv) about 4% by weight to about 6% by weight of the disintegrant; (v) about 1% by weight to about 3% by weight of the surfactant; and (vi) about 0.5% by weight to about 1.5% by weight of the lubricant.

In some embodiments, the formulation comprises: (i) about 5% by weight of the aficamten, or pharmaceutically acceptable salt thereof; (ii) about 85% by weight of the filler; (iii) about 2% by weight of the binder; (iv) about 5% by weight of the disintegrant; (v) about 2% by weight of the surfactant; and (vi) about 1% by weight of the lubricant.

In some embodiments, the formulation comprises: (i) about 10% by weight of the aficamten, or pharmaceutically acceptable salt thereof; (ii) about 80% by weight of the filler; (iii) about 2% by weight of the binder; (iv) about 5% by weight of the disintegrant; (v) about 2% by weight of the surfactant; and (vi) about 1% by weight of the lubricant.

In some embodiments, the formulation comprises: (i) about 1% by weight to about 50% by weight of aficamten, or pharmaceutically acceptable salt thereof; (ii-1) about 10% by weight to about 60% by weight of mannitol; (ii-2) about 5% by weight to about 45% by weight of microcrystalline cellulose; (iii) about 0.1% by weight to about 10% by weight of hydroxypropyl cellulose; (iv) about 1% by weight to about 10% by weight of croscarmellose sodium; (v) about 0.1% by weight to about 10% by weight of sodium lauryl sulfate; and (vi) about 0.1% by weight to about 10% by weight of magnesium stearate.

In some embodiments, the formulation comprises: (i) about 10% by weight to about 30% by weight of the aficamten, or pharmaceutically acceptable salt thereof; (ii-1) about 40% by weight to about 50% by weight of mannitol; (ii-2) about 20% by weight to about 30% by weight of microcrystalline cellulose; (iii) about 1% by weight to about 3% by weight of hydroxypropyl cellulose; (iv) about 4% by weight to about 6% by weight of croscarmellose sodium; (v) about 1% by weight to about 3% by weight of sodium lauryl sulfate; and (vi) about 0.5% by weight to about 1.5% by weight of magnesium stearate.

In some embodiments, the formulation comprises: (i) about 1% by weight to about 10% by weight of aficamten, or pharmaceutically acceptable salt thereof; (ii-1) about 50% by weight to about 60% by weight of mannitol; (ii-2) about 25% by weight to about 35% by weight of microcrystalline cellulose; (iii) about 1% by weight to about 3% by weight of hydroxypropyl cellulose; (iv) about 4% by weight to about 6% by weight of croscarmellose sodium; (v) about 1% by weight to about 3% by weight of sodium lauryl sulfate; and (vi) about 0.5% by weight to about 1.5% by weight of the magnesium stearate.

In some embodiments, the formulation comprises: (i) about 20% by weight of the aficamten, or pharmaceutically acceptable salt thereof; (ii-1) about 44% by weight of mannitol; (ii-2) about 26% by weight of microcrystalline cellulose; (iii) about 2% by weight of hydroxypropyl cellulose; (iv) about 5% by weight of croscarmellose sodium; (v) about 2% by weight of sodium lauryl sulfate; and (vi) about 1% by weight of magnesium stearate.

In some embodiments, the formulation comprises: (i) about 10% by weight of the aficamten, or pharmaceutically acceptable salt thereof; (ii-1) about 50% by weight of mannitol; (ii-2) about 30% by weight of microcrystalline cellulose; (iii) about 2% by weight of hydroxypropyl cellulose; (iv) about 5% by weight of croscarmellose sodium; (v) about 2% by weight of sodium lauryl sulfate; and (vi) about 1% by weight of magnesium stearate.

In some embodiments, the formulation comprises: (i) about 5% by weight of the aficamten, or pharmaceutically acceptable salt thereof; (ii-1) about 54% by weight of mannitol; (ii-2) about 31% by weight of microcrystalline cellulose; (iii) about 2% by weight of hydroxypropyl cellulose; (iv) about 5% by weight of croscarmellose sodium; (v) about 2% by weight of sodium lauryl sulfate; and (vi) about 1% by weight of the magnesium stearate.

In some embodiments, the aforementioned formulations are in the form of a tablet. In some embodiments, the tablet further comprises a coating (e.g., a film coating as described elsewhere herein). In such embodiments, the weight percentages herein are provided with respect to the core tablet, not including the weight of the coating.

In some embodiments, aficamten or pharmaceutical composition containing aficamten will take the form of a pill or tablet and thus the composition may contain, along with a compounds disclosed and/or described herein, one or more of a diluent (e.g., lactose, sucrose, dicalcium phosphate), a lubricant (e.g., magnesium stearate), and/or a binder (e.g., starch, gum acacia, polyvinylpyrrolidine, gelatin, cellulose, cellulose derivatives). Other solid dosage forms include a powder, marume, solution or suspension (e.g., in propylene carbonate, vegetable oils or triglycerides) encapsulated in a gelatin capsule.

In some embodiments, aficamten is provided in a tablet comprising: (i) a core having a total core weight comprising: (a) an intra-granular component comprising: (a-i) aficamten, or pharmaceutically acceptable salt thereof; (a-ii) an intra-granular filler; (a-iii) an intra-granular binder; (a-iv) an intra-granular disintegrant; and (a-v) an intra-granular surfactant; and (b) an extra-granular component comprising: (b-i) an extra-granular filler; (b-ii) an extra-granular disintegrant; and (b-iii) an extra-granular lubricant; and optionally (ii) a coating layer comprising a coating agent.

In some embodiments, the intra-granular filler is selected from the group consisting of powdered cellulose, microcrystalline cellulose, silicified microcrystalline cellulose, kaolin, corn starch, maize starch, starch derivatives, pregelatinized starch, calcium phosphate, calcium hydrogen phosphate, dicalcium phosphate, tricalcium phosphate, compressible sugar, sugar alcohol, mannitol, sorbitol, maltitol, xylitol, lactitol, lactose, dextrose, maltose, sucrose, glucose, fructose, saccharose, raffinose, dextrates, trehalose, maltodextrines, and mixtures of any of the foregoing.

In some embodiments, the intra-granular binder is selected from the group consisting of arabic gum, acacia gum, alginate, alginic acid, corn starch, copolyvidone, polyvinylpyrrolidone, gelatin, glyceryl behenate, hydroxyethylcellulose, hydroxypropyl cellulose, carboxymethylcellulose, carboxymethylcellulose calcium, carboxymethylcellulose sodium, methylcellulose, hypromellose, lactose, polyvinyl alcohol, povidone, polyethylene oxide, polyacrylates, potato starch, pregelatinized starch, sodium alginate, sodium starch, sodium carboxy methyl cellulose, starch, and mixtures of any of the foregoing.

In some embodiments, the intra-granular disintegrant is selected from the group consisting of alginic acid, croscarmellose sodium, cellulose, carboxymethylcellulose calcium, carboxymethylcellulose sodium, microcrystalline cellulose, crospovidone, sodium starch glycolate, low-substituted hydroxypropyl cellulose, polacrillin potassium, pregelatinized starch, partially hydrolyzed starch, sodium carboxymethyl starch, starch, sodium alginate, sodium carboxy methyl cellulose, and mixtures of any of the foregoing.

In some embodiments, the intra-granular surfactant is selected from the group consisting of cetylpyridine chloride, heptadecaethylene oxycetanol, lecithins, polyoxyethylene stearate, nonoxynol 9, nonoxynol 10, octoxynol 9, sorbitan fatty acid esters, Span 20, Span 40, Span 60, Span 80, Span 85, polysorbates, polysorbate 20, polysorbate 21, polysorbate 40, polysorbate 60, polysorbate 61, polysorbate 65, polysorbate 80, sodium salts of fatty alcohol sulfates, sodium lauryl sulfate, sodium salts of sulfosuccinates, sodium dioctylsulfosuccinate, partial esters of fatty acids with alcohols, glycerine monostearate, glyceryl monooleate, ethers of fatty alcohols with polyoxyethylene, esters of fatty acids with polyoxyethylene, copolymers of ethylenoxide and propylenoxide (Pluronic®), benzalkonium chloride, ethoxylated triglycerides, and mixtures of any of the foregoing.

In some embodiments, the extra-granular filler is selected from the group consisting of powdered cellulose, microcrystalline cellulose, silicified microcrystalline cellulose, kaolin, corn starch, maize starch, starch derivatives, pregelatinized starch, calcium phosphate, calcium hydrogen phosphate, dicalcium phosphate, tricalcium phosphate, compressible sugar, sugar alcohol, mannitol, sorbitol, maltitol, xylitol, lactitol, lactose, dextrose, maltose, sucrose, glucose, fructose, saccharose, raffinose, dextrates, trehalose, maltodextrines, and mixtures of any of the foregoing.

In some embodiments, the extra-granular disintegrant is selected from the group consisting of alginic acid, croscarmellose sodium, cellulose, carboxymethylcellulose calcium, carboxymethylcellulose sodium, microcrystalline cellulose, crospovidone, sodium starch glycolate, low-substituted hydroxypropyl cellulose, polacrillin potassium, pregelatinized starch, partially hydrolyzed starch, sodium carboxymethyl starch, starch, sodium alginate, sodium carboxy methyl cellulose, and mixtures of any of the foregoing.

In some embodiments, the extra-granular lubricant is selected from the group consisting of hydrogenated castor oil, magnesium stearate, glyceryl monostearate, calcium stearate, glyceryl behenate, glycerol distearate, glyceryl dipalmitostearate, behenoyl polyoxyl-8 glycerides, sodium stearyl fumarate, stearic acid, talc, zinc stearate, mineral oil, polyethylene glycol, polaxamer, sodium lauryl sulfate, and mixtures of any of the foregoing.

In some embodiments, the core comprises: (a) an intra-granular component comprising: (a-i) about 1% to about 80% of the total core weight of the aficamten, or pharmaceutically acceptable salt thereof; (a-ii) about 10% to about 80% of the total core weight of the intra-granular filler; (a-iii) about 0.1% to about 10% of the total core weight of the intra-granular binder; (a-iv) about 0.1% to about 5% of the total core weight of the intra-granular disintegrant; and (a-v) about 0.1% to about 5% of the total core weight of the intra-granular surfactant; and (b) an extra-granular component comprising: (b-i) about 5% to about 15% of the total core weight of the extra-granular filler; (b-ii) about 0.1% to about 5% of the total core weight of the extra-granular disintegrant; and (b-iii) about 0.1% to about 5% of the total core weight of the extra-granular lubricant. In some embodiments, the total core weight is about 50 mg, about 70 mg, about 100 mg, about 150 mg, about 200 mg, or about 400 mg. In some embodiments, the total core weight is about 50 mg, about 100 mg, about 150 mg, or about 200 mg. In some embodiments, the total core weight is about 50 mg. In some embodiments, the total core weight is about 70 mg. In some embodiments, the total core weight is about 100 mg. In some embodiments, the total core weight is about 150 mg. In some embodiments, the total core weight is about 200 mg. In some embodiments, the total core weight is about 400 mg.

In some embodiments, the core comprises: (a) an intra-granular component comprising: (a-i) about 1% to about 80% of the total core weight of the aficamten, or pharmaceutically acceptable salt thereof; (a-ii) about 10% to about 80% of the total core weight of the intra-granular filler; (a-iii) about 0.1% to about 10% of the total core weight of the intra-granular binder; (a-iv) about 0.1% to about 5% of the total core weight of the intra-granular disintegrant; and (a-v) about 0.1% to about 5% of the total core weight of the intra-granular surfactant; and (b) an extra-granular component comprising: (b-i) about 5% to about 15% of the total core weight of the extra-granular filler; (b-ii) about 0.1% to about 5% of the total core weight of the extra-granular disintegrant; and (b-iii) about 0.1% to about 5% of the total core weight of the extra-granular lubricant. In some embodiments, the total core weight is about 50 mg, about 70 mg, about 100 mg, about 150 mg, about 200 mg, or about 400 mg. In some embodiments, the total core weight is about 50 mg, about 100 mg, about 150 mg, or about 200 mg. In some embodiments, the total core weight is about 50 mg. In some embodiments, the total core weight is about 70 mg. In some embodiments, the total core weight is about 100 mg. In some embodiments, the total core weight is about 150 mg. In some embodiments, the total core weight is about 200 mg. In some embodiments, the total core weight is about 400 mg.

In some embodiments, the core comprises: (a) an intra-granular component comprising: (a-i) about 1% to about 50% of the total core weight of the aficamten, or pharmaceutically acceptable salt thereof; (a-ii) about 40% to about 80% of the total core weight of the intra-granular filler; (a-iii) about 1% to about 5% of the total core weight of the intra-granular binder; (a-iv) about 1% to about 5% of the total core weight of the intra-granular disintegrant; and (a-v) about 1% to about 5% of the total core weight of the intra-granular surfactant; and (b) an extra-granular component comprising: (b-i) about 5% to about 15% of the total core weight of the extra-granular filler; (b-ii) about 1% to about 5% of the total core weight of the extra-granular disintegrant; and (b-iii) about 0.1% to about 2% of the total core weight of the extra-granular lubricant. In some embodiments, the total core weight is about 50 mg, about 70 mg, about 100 mg, about 150 mg, about 200 mg, or about 400 mg. In some embodiments, the total core weight is about 50 mg, about 100 mg, about 150 mg, or about 200 mg. In some embodiments, the total core weight is about 50 mg. In some embodiments, the total core weight is about 70 mg. In some embodiments, the total core weight is about 100 mg. In some embodiments, the total core weight is about 150 mg. In some embodiments, the total core weight is about 200 mg. In some embodiments, the total core weight is about 400 mg.

In some embodiments, the core comprises: (a) an intra-granular component comprising: (a-i) about 10% to about 30% of the total core weight of the aficamten, or pharmaceutically acceptable salt thereof; (a-ii) about 50% to about 70% of the total core weight of the intra-granular filler; (a-iii) about 1% to about 3% of the total core weight of the intra-granular binder; (a-iv) about 2% to about 4% of the total core weight of the intra-granular disintegrant; and (a-v) about 1% to about 3% of the total core weight of the intra-granular surfactant; and (b) an extra-granular component comprising: (b-i) about 5% to about 15% of the total core weight of the an extra-granular filler; (b-ii) about 1% to about 3% of the total core weight of the extra-granular disintegrant; and (b-iii) about 0.1% to about 1.5% of the total core weight of the extra-granular lubricant. In some embodiments, the total core weight is about 50 mg, about 70 mg, about 100 mg, about 150 mg, about 200 mg, or about 400 mg. In some embodiments, the total core weight is about 50 mg, about 100 mg, about 150 mg, or about 200 mg. In some embodiments, the total core weight is about 50 mg. In some embodiments, the total core weight is about 70 mg. In some embodiments, the total core weight is about 100 mg. In some embodiments, the total core weight is about 150 mg. In some embodiments, the total core weight is about 200 mg. In some embodiments, the total core weight is about 400 mg.

In some embodiments, the core comprises: (a) an intra-granular component comprising: (a-i) about 1% to about 10% of the total core weight of the aficamten, or pharmaceutically acceptable salt thereof; (a-ii) about 60% to about 80% of the total core weight of the intra-granular filler; (a-iii) about 1% to about 3% of the total core weight of the intra-granular binder; (a-iv) about 2% to about 4% of the total core weight of the intra-granular disintegrant; and (a-v) about 1% to about 3% of the total core weight of the intra-granular surfactant; and (b) an extra-granular component comprising: (b-i) about 5% to about 15% of the total core weight of the an extra-granular filler; (b-ii) about 1% to about 3% of the total core weight of the extra-granular disintegrant; and (b-iii) about 0.1% to about 1.5% of the total core weight of the extra-granular lubricant. In some embodiments, the total core weight is about 50 mg, about 70 mg, about 100 mg, about 150 mg, about 200 mg, or about 400 mg. In some embodiments, the total core weight is about 50 mg, about 100 mg, about 150 mg, or about 200 mg. In some embodiments, the total core weight is about 50 mg. In some embodiments, the total core weight is about 70 mg. In some embodiments, the total core weight is about 100 mg. In some embodiments, the total core weight is about 150 mg. In some embodiments, the total core weight is about 200 mg. In some embodiments, the total core weight is about 400 mg.

In some embodiments, the core comprises: (a) an intra-granular component comprising: (a-i) about 5% of the total core weight of the aficamten, or pharmaceutically acceptable salt thereof; (a-ii) about 74% of the total core weight of the intra-granular filler; (a-iii) about 2% of the total core weight of the intra-granular binder; (a-iv) about 3% of the total core weight of the intra-granular disintegrant; and (a-v) about 2% of the total core weight of the intra-granular surfactant; and (b) an extra-granular component comprising: (b-i) about 11% of the total core weight of the an extra-granular filler; (b-ii) about 2% of the total core weight of the extra-granular disintegrant; and (b-iii) about 1% of the total core weight of the extra-granular lubricant. In some embodiments, the total core weight is about 50 mg, about 70 mg, about 100 mg, about 150 mg, about 200 mg, or about 400 mg. In some embodiments, the total core weight is about 50 mg, about 100 mg, about 150 mg, or about 200 mg. In some embodiments, the total core weight is about 50 mg. In some embodiments, the total core weight is about 70 mg. In some embodiments, the total core weight is about 100 mg. In some embodiments, the total core weight is about 150 mg. In some embodiments, the total core weight is about 200 mg. In some embodiments, the total core weight is about 400 mg.

In some embodiments, the core comprises: (a) an intra-granular component comprising: (a-i) about 10% of the total core weight of the aficamten, or pharmaceutically acceptable salt thereof; (a-ii) about 69% of the total core weight of the intra-granular filler; (a-iii) about 2% of the total core weight of the intra-granular binder; (a-iv) about 3% of the total core weight of the intra-granular disintegrant; and (a-v) about 2% of the total core weight of the intra-granular surfactant; and (b) an extra-granular component comprising: (b-i) about 11% of the total core weight of the an extra-granular filler; (b-ii) about 2% of the total core weight of the extra-granular disintegrant; and (b-iii) about 1% of the total core weight of the extra-granular lubricant. In some embodiments, the total core weight is about 50 mg, about 70 mg, about 100 mg, about 150 mg, about 200 mg, or about 400 mg. In some embodiments, the total core weight is about 50 mg, about 100 mg, about 150 mg, or about 200 mg. In some embodiments, the total core weight is about 50 mg. In some embodiments, the total core weight is about 70 mg. In some embodiments, the total core weight is about 100 mg. In some embodiments, the total core weight is about 150 mg. In some embodiments, the total core weight is about 200 mg. In some embodiments, the total core weight is about 400 mg.

In some embodiments, the core comprises: (a) an intra-granular component comprising: (a-i) about 20% of the total core weight of the aficamten, or pharmaceutically acceptable salt thereof; (a-ii) about 59% of the total core weight of the intra-granular filler; (a-iii) about 2% of the total core weight of the intra-granular binder; (a-iv) about 3% of the total core weight of the intra-granular disintegrant; and (a-v) about 2% of the total core weight of the intra-granular surfactant; and (b) an extra-granular component comprising: (b-i) about 11% of the total core weight of the an extra-granular filler; (b-ii) about 2% of the total core weight of the extra-granular disintegrant; and (b-iii) about 1% of the total core weight of the extra-granular lubricant. In some embodiments, the total core weight is about 50 mg, about 70 mg, about 100 mg, about 150 mg, about 200 mg, or about 400 mg. In some embodiments, the total core weight is about 50 mg, about 100 mg, about 150 mg, or about 200 mg. In some embodiments, the total core weight is about 50 mg. In some embodiments, the total core weight is about 70 mg. In some embodiments, the total core weight is about 100 mg. In some embodiments, the total core weight is about 150 mg. In some embodiments, the total core weight is about 200 mg. In some embodiments, the total core weight is about 400 mg.

In some embodiments, the core comprises: (a) an intra-granular component comprising: (a-i) about 1% to about 50% of the total core weight of the aficamten, or pharmaceutically acceptable salt thereof; (a-ii-1) about 40% to about 60% of the total core weight of mannitol; (a-ii-2) about 10% to about 30% of the total core weight of microcrystalline cellulose; (a-iii) about 1% to about 5% of the total core weight of hydroxypropyl cellulose; (a-iv) about 1% to about 5% of the total core weight of croscarmellose sodium; and (a-v) about 1% to about 5% of the total core weight of sodium lauryl sulfate; and (b) an extra-granular component comprising: (b-i) about 5% to about 15% of the total core weight of microcrystalline cellulose; (b-ii) about 1% to about 5% of the total core weight of croscarmellose sodium; and (b-iii) about 0.1% to about 2% of the total core weight of extra-granular lubricant. In some embodiments, the extra-granular lubricant is magnesium stearate. In some embodiments, the total core weight is about 50 mg, about 70 mg, about 100 mg, about 150 mg, about 200 mg, or about 400 mg. In some embodiments, the total core weight is about 50 mg, about 100 mg, about 150 mg, or about 200 mg. In some embodiments, the total core weight is about 50 mg. In some embodiments, the total core weight is about 70 mg. In some embodiments, the total core weight is about 100 mg. In some embodiments, the total core weight is about 150 mg. In some embodiments, the total core weight is about 200 mg. In some embodiments, the total core weight is about 400 mg.

In some embodiments, the core comprises: (a) an intra-granular component comprising: (a-i) about 10% to about 30% of the total core weight of the aficamten, or pharmaceutically acceptable salt thereof; (a-ii-1) about 40% to about 50% of the total core weight of mannitol; (a-ii-2) about 10% to about 20% of the total core weight of microcrystalline cellulose; (a-iii) about 1% to about 3% of the total core weight of hydroxypropyl cellulose; (a-iv) about 2% to about 4% of the total core weight of croscarmellose sodium; and (a-v) about 1% to about 3% of the total core weight of sodium lauryl sulfate; and (b) an extra-granular component comprising: (b-i) about 5% to about 15% of the total core weight of microcrystalline cellulose; (b-ii) about 1% to about 3% of the total core weight of croscarmellose sodium; and (b-iii) about 0.1% to about 1.5% of the total core weight of extra-granular lubricant. In some embodiments, the extra-granular lubricant is magnesium stearate. In some embodiments, the total core weight is about 50 mg, about 70 mg, about 100 mg, about 150 mg, about 200 mg, or about 400 mg. In some embodiments, the total core weight is about 50 mg, about 100 mg, about 150 mg, or about 200 mg. In some embodiments, the total core weight is about 50 mg. In some embodiments, the total core weight is about 70 mg. In some embodiments, the total core weight is about 100 mg. In some embodiments, the total core weight is about 150 mg. In some embodiments, the total core weight is about 200 mg. In some embodiments, the total core weight is about 400 mg.

In some embodiments, the core comprises: (a) an intra-granular component comprising: (a-i) about 1% by weight to about 10% of the total core weight of the aficamten, or pharmaceutically acceptable salt thereof; (a-ii-1) about 50% to about 60% of the total core weight of mannitol; (a-ii-2) about 15% to about 25% of the total core weight of microcrystalline cellulose; (a-iii) about 1% to about 3% of the total core weight of hydroxypropyl cellulose; (a-iv) about 2% to about 4% of the total core weight of croscarmellose sodium; and (a-v) about 1% to about 3% of the total core weight of sodium lauryl sulfate; and (b) an extra-granular component comprising: (b-i) about 5% to about 15% of the total core weight of microcrystalline cellulose; (b-ii) about 1% to about 3% of the total core weight of croscarmellose sodium; and (b-iii) about 0.1% to about 1.5% of the total core weight of extra-granular lubricant. In some embodiments, the extra-granular lubricant is magnesium stearate. In some embodiments, the total core weight is about 50 mg, about 70 mg, about 100 mg, about 150 mg, about 200 mg, or about 400 mg. In some embodiments, the total core weight is about 50 mg, about 100 mg, about 150 mg, or about 200 mg. In some embodiments, the total core weight is about 50 mg. In some embodiments, the total core weight is about 70 mg. In some embodiments, the total core weight is about 100 mg. In some embodiments, the total core weight is about 150 mg. In some embodiments, the total core weight is about 200 mg. In some embodiments, the total core weight is about 400 mg.

In some embodiments, the core comprises: (a) an intra-granular component comprising: (a-i) about 20% of the total core weight of the aficamten, or pharmaceutically acceptable salt thereof; (a-ii-1) about 44% of the total core weight of mannitol; (a-ii-2) about 15% of the total core weight of microcrystalline cellulose; (a-iii) about 2% of the total core weight of hydroxypropyl cellulose; (a-iv) about 3% of the total core weight of croscarmellose sodium; and (a-v) about 2% of the total core weight of sodium lauryl sulfate; and (b) an extra-granular component comprising: (b-i) about 11% of the total core weight of microcrystalline cellulose; (b-ii) about 2% of the total core weight of croscarmellose sodium; and (b-iii) about 1% of the total core weight of extra-granular lubricant. In some embodiments, the extra-granular lubricant is magnesium stearate. In some embodiments, the total core weight is about 50 mg, about 70 mg, about 100 mg, about 150 mg, about 200 mg, or about 400 mg. In some embodiments, the total core weight is about 50 mg, about 100 mg, about 150 mg, or about 200 mg. In some embodiments, the total core weight is about 50 mg. In some embodiments, the total core weight is about 70 mg. In some embodiments, the total core weight is about 100 mg. In some embodiments, the total core weight is about 150 mg. In some embodiments, the total core weight is about 200 mg. In some embodiments, the total core weight is about 400 mg.

In some embodiments, the core comprises: (a) an intra-granular component comprising: (a-i) about 10% of the total core weight of the aficamten, or pharmaceutically acceptable salt thereof; (a-ii-1) about 50% of the total core weight of mannitol; (a-ii-2) about 19% of the total core weight of microcrystalline cellulose; (a-iii) about 2% of the total core weight of hydroxypropyl cellulose; (a-iv) about 3% of the total core weight of croscarmellose sodium; and (a-v) about 2% of the total core weight of sodium lauryl sulfate; and (b) an extra-granular component comprising: (b-i) about 11% of the total core weight of microcrystalline cellulose; (b-ii) about 2% of the total core weight of croscarmellose sodium; and (b-iii) about 1% of the total core weight of extra-granular lubricant. In some embodiments, the extra-granular lubricant is magnesium stearate. In some embodiments, the total core weight is about 50 mg, about 70 mg, about 100 mg, about 150 mg, about 200 mg, or about 400 mg. In some embodiments, the total core weight is about 50 mg, about 100 mg, about 150 mg, or about 200 mg. In some embodiments, the total core weight is about 50 mg. In some embodiments, the total core weight is about 70 mg. In some embodiments, the total core weight is about 100 mg. In some embodiments, the total core weight is about 150 mg. In some embodiments, the total core weight is about 200 mg. In some embodiments, the total core weight is about 400 mg.

In some embodiments, the core comprises: (a) an intra-granular component comprising: (a-i) about 5% by weight of the aficamten, or pharmaceutically acceptable salt thereof; (a-ii-1) about 54% by weight of mannitol; (a-ii-2) about 20% by weight of microcrystalline cellulose; (a-iii) about 2% by weight of hydroxypropyl cellulose; (a-iv) about 3% by weight of croscarmellose sodium; and (a-v) about 2% by weight of sodium lauryl sulfate; and (b) an extra-granular component comprising: (b-i) about 11% by weight of microcrystalline cellulose; (b-ii) about 2% by weight of croscarmellose sodium; and (b-iii) about 1% by weight of extra-granular lubricant. In some embodiments, the extra-granular lubricant is magnesium stearate. In some embodiments, the total core weight is about 50 mg, about 70 mg, about 100 mg, about 150 mg, about 200 mg, or about 400 mg. In some embodiments, the total core weight is about 50 mg, about 100 mg, about 150 mg, or about 200 mg. In some embodiments, the total core weight is about 50 mg. In some embodiments, the total core weight is about 70 mg. In some embodiments, the total core weight is about 100 mg. In some embodiments, the total core weight is about 150 mg. In some embodiments, the total core weight is about 200 mg. In some embodiments, the total core weight is about 400 mg.

In some embodiments, the tablet comprises a coating layer comprising a coating agent. In some embodiments, the coating layer surrounds the total core of the tablet. In some embodiments, the coating agent is selected from the group consisting of Opadry QX White 21A180025, Opadry I, and Opadry II. In some embodiments, the coating agent is Opadry QX White 21A180025. In some embodiments, the tablet comprises about 0.5% to about 10% of the total core weight of coating agent. In some embodiments, the tablet comprises about 1% to about 5% of the total core weight of coating agent. In some embodiments, the tablet comprises about 2% to about 4% of the total core weight of coating agent. In some embodiments, the tablet comprises about 3% of the total core weight of coating agent. In some embodiments, the tablet comprises about 0.5% to about 10% of the total core weight of Opadry QX White 21A180025. In some embodiments, the tablet comprises about 1% to about 5% of the total core weight of Opadry QX White 21A180025. In some embodiments, the tablet comprises about 2% to about 4% of the total core weight of Opadry QX White 21A180025. In some embodiments, the tablet comprises about 3% of the total core weight of Opadry QX White 21A180025.

In some embodiments, the amount of aficamten (which may exist in various forms) in a formulation, e.g., a tablet, is about a daily dose as described herein, e.g., of about 5 mg, about 10 mg, about 15 mg, or about 20 mg of aficamten. In some embodiments, the amount of aficamten (which may exist in various forms) in a formulation (e.g., a tablet) is about half of a daily dose as described herein. In some embodiments, the amount of aficamten in a formulation (e.g., a tablet) is about 2.5 mg, about 5 mg, about 7.5 mg, or about 10 mg of aficamten.

In some embodiments, aficamten exists in its free base form in various formulations, e.g., tablets. In some embodiments, a method, e.g., a method described in an Example, uses such tablets.

Liquid pharmaceutically administrable compositions can, for example, be prepared by dissolving, dispersing, or suspending etc. a compound disclosed and/or described herein and optional pharmaceutical additives in a carrier (e.g., water, saline, aqueous dextrose, glycerol, glycols, ethanol or the like) to form a solution or suspension. Injectables can be prepared in conventional forms, either as liquid solutions or suspensions, as emulsions, or in solid forms suitable for dissolution or suspension in liquid prior to injection. The percentage of the compound contained in such parenteral compositions depends, for example, on the physical nature of the compound, the activity of the compound and the needs of the subject. However, percentages of active ingredient of 0.01% to 10% in solution are employable, and may be higher if the composition is a solid which will be subsequently diluted to another concentration. In some embodiments, the composition will comprise from about 0.2 to 2% of a compound disclosed and/or described herein in solution.

Pharmaceutical compositions of the compounds disclosed and/or described herein may also be administered to the respiratory tract as an aerosol or solution for a nebulizer, or as a microfine powder for insufflation, alone or in combination with an inert carrier such as lactose. In such a case, the particles of the pharmaceutical composition may have diameters of less than 50 microns, or in some embodiments, less than 10 microns. Pharmaceutical composition of the compounds disclosed and/or described herein may also be administered transdermally, e.g., in the form of a patch, a cream, an ointment or other formulation suitable for administration to the skin.

In addition, pharmaceutical compositions can include a compound disclosed and/or described herein and one or more additional medicinal agents, pharmaceutical agents, adjuvants, and the like. Suitable medicinal and pharmaceutical agents include those described herein.

### Polymorphs

In some embodiments, aficamten is in a polymorph form of (R)—N-(5-(5-ethyl-1,2,4-oxadiazol-3-yl)-2,3-dihydro-1H-inden-1-yl)-1-methyl-1H-pyrazole-4-carboxamide. Suitable polymorphs of aficamten include those disclosed in and may be prepared according to WO 2021/011807 and U.S. Patent Publication US-2022-0315571-A1, which are incorporated by reference herein in its entirety. The polymorphs may have properties such as bioavailability and stability under certain conditions that are suitable for medical or pharmaceutical uses. Variations in the crystal structure of a pharmaceutical drug substance may affect the dissolution rate (which may affect bioavailability, etc.), manufacturability (e.g., case of handling, case of purification, ability to consistently prepare doses of known strength, etc.) and stability (e.g., thermal stability, shelf life (including resistance to degradation), etc.) of a pharmaceutical drug product. Such variations may affect the methods of preparation or formulation of pharmaceutical compositions in different dosage or delivery forms, such as solid oral dosage forms including tablets and capsules. Compared to other forms such as non-crystalline or amorphous forms, polymorphs may provide desired or suitable hygroscopicity, particle size control, dissolution rate, solubility, purity, physical and chemical stability, manufacturability, yield, reproducibility, and/or process control. Thus, polymorphs of (R)—N-(5-(5-ethyl-1,2,4-oxadiazol-3-yl)-2,3-dihydro-1H-inden-1-yl)-1-methyl-1H-pyrazole-4-carboxamide may provide advantages of improving the manufacturing process of an active agent or the stability or storability of a drug product form of the active agent, or having suitable bioavailability and/or stability as an active agent. In some embodiments, the aficamten in a formulation, e.g., a tablet, is one of polymorphic Form I, Form II, Form III, Form IV, Form V, or Form VI, or a combination thereof. In some embodiments, the aficamten is one of polymorphic Form I, Form II, Form III, Form IV, Form V, or Form VI. In some embodiments, the aficamten is polymorphic Form I or Form IV. In some embodiments, the aficamten is polymorphic Form I. In some embodiments, the aficamten is polymorphic Form IV.

### Form I

In some embodiments, the aficamten is polymorphic Form I of (R)—N-(5-(5-ethyl-1,2,4-oxadiazol-3-yl)-2,3-dihydro-1H-inden-1-yl)-1-methyl-1H-pyrazole-4-carboxamide. Angles 2-theta and relative peak intensities that may be observed for Form I using XRPD are shown in Table P-1. In some embodiments, Form I has an XRPD pattern substantially as shown in FIG. 1A of WO 2021/011807 and U.S. Patent Publication US-2022-0315571-A1.

TABLE P-1

| Angle (°2θ) | d value (Å) | Intensity (counts) |
|---|---|---|
| 3.7 | 23.6 | 55228.97 |
| 11.2 | 7.9 | 126123.6 |
| 12.9 | 6.9 | 94231.09 |
| 13.5 | 6.6 | 12945.13 |
| 14.4 | 6.2 | 22543.29 |
| 14.9 | 6.0 | 1982.81 |
| 16.6 | 5.3 | 12426.88 |
| 17.8 | 5.0 | 3641.86 |
| 18.6 | 4.8 | 15100.54 |
| 21.6 | 4.1 | 10927.77 |
| 22.2 | 4.0 | 10386.49 |
| 22.4 | 4.0 | 29345.32 |
| 22.8 | 3.9 | 4094.45 |
| 23.2 | 3.8 | 9276.26 |
| 23.9 | 3.7 | 12944.35 |
| 24.4 | 3.7 | 8288.4 |
| 24.7 | 3.6 | 14182.35 |
| 25.0 | 3.6 | 16601.84 |
| 25.8 | 3.4 | 11350.3 |
| 26.1 | 3.4 | 14448.7 |
| 28.6 | 3.1 | 3088.71 |
| 29.0 | 3.1 | 4033.18 |
| 29.4 | 3.0 | 2451.42 |
| 29.9 | 3.0 | 3631.24 |
| 30.6 | 2.9 | 4172.12 |
| 33.8 | 2.6 | 3752.58 |
| 36.1 | 2.5 | 543.33 |
| 36.8 | 2.4 | 405.69 |
| 37.8 | 2.4 | 482.5 |
| 39.8 | 2.3 | 2685.52 |

In some embodiments, polymorphic Form I has an XRPD pattern displaying at least two, at least three, at least four, at least five, at least six, at least seven, at least eight, at least nine, or at least ten of the peaks at angles 2-theta with the greatest intensity in the XRPD pattern substantially as shown in FIG. 1A of WO 2021/011807 and U.S. Patent Publication US-2022-0315571-A1 or as provided in Table P-1. It should be understood that relative intensities can vary depending on a number of factors, including sample preparation, mounting, and the instrument and analytical procedure and settings used to obtain the spectrum. Relative peak intensities and peak assignments can vary within experimental error. In some embodiments, peak assignments listed herein, including for polymorphic Form I, can vary by about ±0.6 degrees, ±0.4 degrees, ±0.2 degrees, or ±0.1 degrees 2-theta.

In some embodiments, polymorphic Form I has an XRPD pattern comprising peaks at angles 2-theta of 3.7±0.2, 11.2±0.2, 12.9±0.2, 13.5±0.2, 14.4±0.2, 14.9±0.2, 16.6±0.2, 17.8±0.2, 18.6±0.2, 21.6±0.2, 22.2±0.2, 22.4±0.2, 22.8±0.2, 23.2±0.2, 23.9±0.2, 24.4±0.2, 24.7±0.2, 25.0±0.2, 25.8±0.2, 26.1±0.2, 28.6±0.2, 29.0±0.2, 29.4±0.2, 29.9±0.2, 30.6±0.2, 33.8±0.2, 36.1±0.2, 36.8±0.2, 37.8±0.2, and 39.8±0.2 degrees. In some embodiments, polymorphic Form I has an XRPD pattern comprising peaks at angles 2-theta of 3.7±0.2, 11.2±0.2, 12.9±0.2, 13.5±0.2, 14.4±0.2, 18.6±0.2, 22.4±0.2, 24.7±0.2, 25.0±0.2, and 26.1±0.2 degrees. In some embodiments, polymorphic Form I has an XRPD pattern comprising peaks at angles 2-theta of 3.7±0.2, 11.2±0.2, 12.9±0.2, 14.4±0.2, and 22.4±0.2 degrees. It is to be understood that additional peaks in the XRPD pattern other than those shown in FIG. 1A of WO 2021/011807 and U.S. Patent Publication US-2022-0315571-A1 or as provided in Table P-1 may be observed, for instance, due to the presence of impurities, solvent, or other polymorphs or amorphic forms present in the test sample.

In some embodiments, Form I has a differential DSC graph substantially as shown in FIG. 1B of WO 2021/011807 and U.S. Patent Publication US-2022-0315571-A1. In some embodiments, Form I is characterized as having an endotherm onset at about 199° C. as determined by DSC. In some embodiments, Form I is characterized as having an endotherm onset at 199±2° C. (e.g., 199±1.9° C., 199±1.8° C., 199±1.7° C., 199±1.6° C., 199±1.5° C., 199±1.4° C., 199±1.3° C., 192±1.2° C., 199±1, 199±0.9° C., 199±0.8° C., 199±0.7° C., 199±0.6° C., 199±0.5° C., 199±0.4° C., 199±0.3° C., 199±0.2° C., or 199±0.1° C.) as determined by DSC.

In some embodiments, Form I has a TGA graph substantially as shown in FIG. 1B of WO 2021/011807 and U.S. Patent Publication US-2022-0315571-A1.

In some embodiments, Form I has a DVS graph substantially as shown in FIG. 1C of WO 2021/011807 and U.S. Patent Publication US-2022-0315571-A1.

In some embodiments of Form I, at least one, at least two, at least three, at least four, at least five, or all of the following (a)-(f) apply:
(a) Form I has an XRPD pattern comprising peaks at angles 2-theta of 3.7±0.2, 11.2±0.2, 12.9±0.2, 14.4±0.2, and 22.4±0.2 degrees; an XRPD pattern comprising peaks at angles 2-theta of 3.7±0.2, 11.2±0.2, 12.9±0.2, 13.5±0.2, 14.4±0.2, 18.6±0.2, 22.4±0.2, 24.7±0.2, 25.0±0.2, and 26.1±0.2 degrees; or an XRPD pattern comprising peaks at angles 2-theta of 3.7±0.2, 11.2±0.2, 12.9±0.2, 13.5±0.2, 14.4±0.2, 14.9±0.2, 16.6±0.2, 17.8±0.2, 18.6±0.2, 21.6±0.2, 22.2±0.2, 22.4±0.2, 22.8±0.2, 23.2±0.2, 23.9±0.2, 24.4±0.2, 24.7±0.2, 25.0±0.2, 25.8±0.2, 26.1±0.2, 28.6±0.2, 29.0±0.2, 29.4±0.2, 29.9±0.2, 30.6±0.2, 33.8±0.2, 36.1±0.2, 36.8±0.2, 37.8±0.2, and 39.8±0.2 degrees;
(b) Form I has an XRPD pattern substantially as shown in FIG. 1A of WO 2021/011807 and U.S. Patent Publication US-2022-0315571-A1;
(c) Form I has a DSC graph substantially as shown in FIG. 1B of WO 2021/011807 and U.S. Patent Publication US-2022-0315571-A1;
(d) Form I is characterized as having an endotherm onset at about 199° C. as determined by DSC;
(e) Form I has a TGA graph substantially as shown in FIG. 1B of WO 2021/011807 and U.S. Patent Publication US-2022-0315571-A1; and
(f) Form I has a DVS graph substantially as shown in FIG. 1C of WO 2021/011807 and U.S. Patent Publication US-2022-0315571-A1.

### Form II

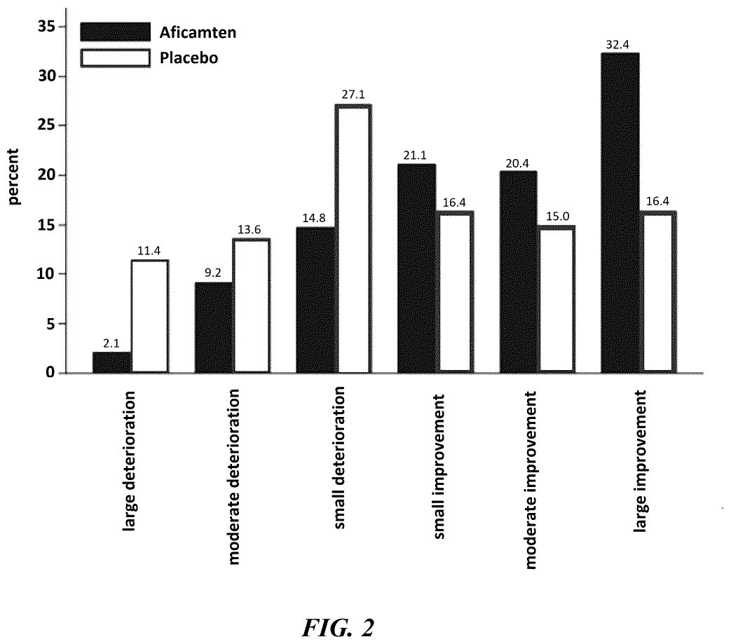

In some embodiments, the aficamten is polymorphic Form II of (R)—N-(5-(5-ethyl-1,2,4-oxadiazol-3-yl)-2,3-dihydro-1H-inden-1-yl)-1-methyl-1H-pyrazole-4-carboxamide. Angles 2-theta and relative peak intensities that may be observed for Form II using XRPD are shown in Table P-2. In some embodiments, Form II has an XRPD pattern substantially as shown in FIG. 2A of WO 2021/011807 and U.S. Patent Publication US-2022-0315571-A1.

TABLE P-2

| Angle (°2θ) | d value (Å) | Intensity (counts) |
|---|---|---|
| 3.7 | 23.6 | 46289.09 |
| 7.4 | 12.0 | 2626.2 |
| 9.8 | 9.0 | 13093.89 |
| 11.1 | 7.9 | 15067.72 |
| 12.8 | 6.9 | 24560.2 |
| 13.5 | 6.6 | 4597.35 |
| 14.4 | 6.2 | 4359.38 |
| 14.7 | 6.0 | 12272.37 |
| 16.1 | 5.5 | 12841.63 |
| 17.0 | 5.2 | 8725.7 |
| 18.5 | 4.8 | 11802.76 |
| 20.4 | 4.4 | 16037.66 |
| 21.6 | 4.1 | 4907.93 |
| 22.3 | 4.0 | 10307.6 |
| 23.3 | 3.8 | 8940.83 |
| 24.0 | 3.7 | 6680.21 |
| 24.3 | 3.7 | 7959.39 |
| 24.8 | 3.6 | 6336.24 |
| 25.8 | 3.4 | 4019.98 |
| 27.4 | 3.3 | 1872.66 |
| 28.8 | 3.1 | 1783 |
| 29.5 | 3.0 | 1203.02 |
| 30.5 | 2.9 | 1080.7 |

In some embodiments, polymorphic Form II has an XRPD pattern displaying at least two, at least three, at least four, at least five, at least six, at least seven, at least eight, at least nine, or at least ten of the peaks at angles 2-theta with the greatest intensity in the XRPD pattern substantially as shown in FIG. 2A of WO 2021/011807 and U.S. Patent Publication US-2022-0315571-A1 or as provided in Table P-2. It should be understood that relative intensities can vary depending on a number of factors, including sample preparation, mounting, and the instrument and analytical procedure and settings used to obtain the spectrum. Relative peak intensities and peak assignments can vary within experimental error. In some embodiments, peak assignments listed herein, including for polymorphic Form II, can vary by about ±0.6 degrees, ±0.4 degrees, ±0.2 degrees, or ±0.1 degrees 2-theta.

In some embodiments, polymorphic Form II has an XRPD pattern comprising peaks at angles 2-theta of 3.7±0.2, 7.4±0.2, 9.8±0.2, 11.1±0.2, 12.8±0.2, 13.5±0.2, 14.4±0.2, 14.7±0.2, 16.1±0.2, 17.0±0.2, 18.5±0.2, 20.4±0.2, 21.6±0.2, 22.3±0.2, 23.3±0.2, 24.0±0.2, 24.3±0.2, 24.8±0.2, 25.8±0.2, 27.4±0.2, 28.8±0.2, 29.5±0.2, and 30.5±0.2 degrees. In some embodiments, polymorphic Form II has an XRPD pattern comprising peaks at angles 2-theta of 3.7±0.2, 9.8±0.2, 11.1±0.2, 12.8±0.2, 14.7±0.2, 16.1±0.2, 18.5±0.2, 20.4±0.2, 22.3±0.2, and 23.3±0.2 degrees. In some embodiments, polymorphic Form II has an XRPD pattern comprising peaks at angles 2-theta of 3.7±0.2, 9.8±0.2, 11.1±0.2, 12.8±0.2, and 20.4±0.2 degrees. It is to be understood that additional peaks in the XRPD pattern other than those shown in FIG. 2A of WO 2021/011807 and U.S. Patent Publication US-2022-0315571-A1 or as provided in Table P-2 may be observed, for instance, due to the presence of impurities, solvent, or other polymorphs or amorphic forms present in the test sample.

In some embodiments, Form II has a DSC graph substantially as shown in FIG. 2B of WO 2021/011807 and U.S. Patent Publication US-2022-0315571-A1. In some embodiments, Form II is characterized as having an endotherm onset at about 199° C. as determined by DSC. In some embodiments, Form II is characterized as having an endotherm onset at about 199±2° C. (e.g., 199±1.9° C., 199±1.8° C., 199±1.7° C., 199±1.6° C., 199±1.5° C., 199±1.4° C., 199±1.3° C., 199±1.2° C., 199±1, 199±0.9° C., 199±0.8° C., 199±0.7° C., 199±0.6° C., 199±0.5° C., 199±0.4° C., 199±0.3° C., 199±0.2° C., or 199±0.1° C.) as determined by DSC.

In some embodiments, Form II has a TGA graph substantially as shown in FIG. 2B of WO 2021/011807 and U.S. Patent Publication US-2022-0315571-A1.

In some embodiments of Form II, at least one, at least two, at least three, at least four, or all of the following (a)-(e) apply:
(a) Form II has an XRPD pattern comprising peaks at angles 2-theta of 3.7±0.2, 9.8±0.2, 11.1±0.2, 12.8±0.2, and 20.4±0.2 degrees; an XRPD pattern comprising peaks at angles 2-theta of 3.7±0.2, 9.8±0.2, 11.1±0.2, 12.8±0.2, 14.7±0.2, 16.1±0.2, 18.5±0.2, 20.4±0.2, 22.3±0.2, and 23.3±0.2 degrees; or an XRPD pattern comprising peaks at angles 2-theta of 3.7±0.2, 7.4±0.2, 9.8±0.2, 11.1±0.2, 12.8±0.2, 13.5±0.2, 14.4±0.2, 14.7±0.2, 16.1±0.2, 17.0±0.2, 18.5±0.2, 20.4±0.2, 21.6±0.2, 22.3±0.2, 23.3±0.2, 24.0±0.2, 24.3±0.2, 24.8±0.2, 25.8±0.2, 27.4±0.2, 28.8±0.2, 29.5±0.2, and 30.5±0.2 degrees;
(b) Form II has an XRPD pattern substantially as shown in FIG. 2A of WO 2021/011807 and U.S. Patent Publication US-2022-0315571-A1;
(c) Form II has a DSC graph substantially as shown in FIG. 2B of WO 2021/011807 and U.S. Patent Publication US-2022-0315571-A1;
(d) Form II is characterized as having a melting endotherm onset at about 199° C. as determined by DSC; and
(e) Form II has a TGA graph substantially as shown in FIG. 2B of WO 2021/011807 and U.S. Patent Publication US-2022-0315571-A1.

### Form III

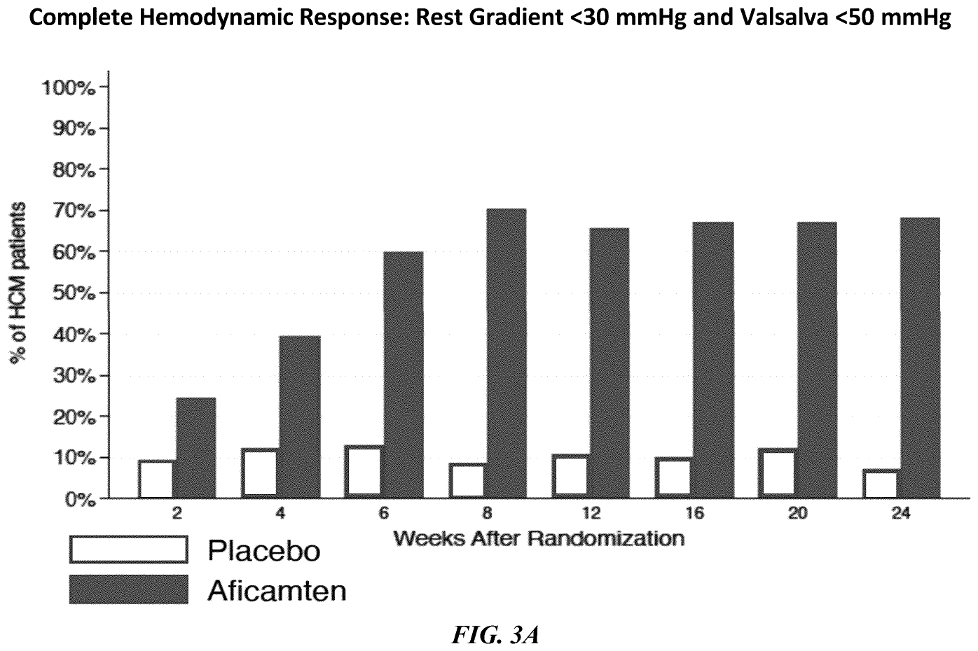

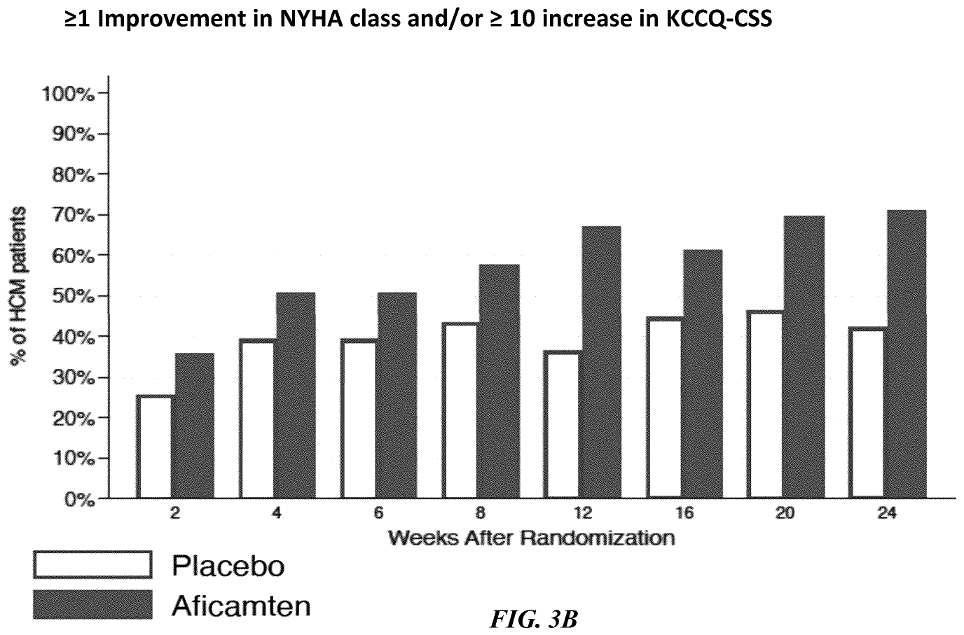

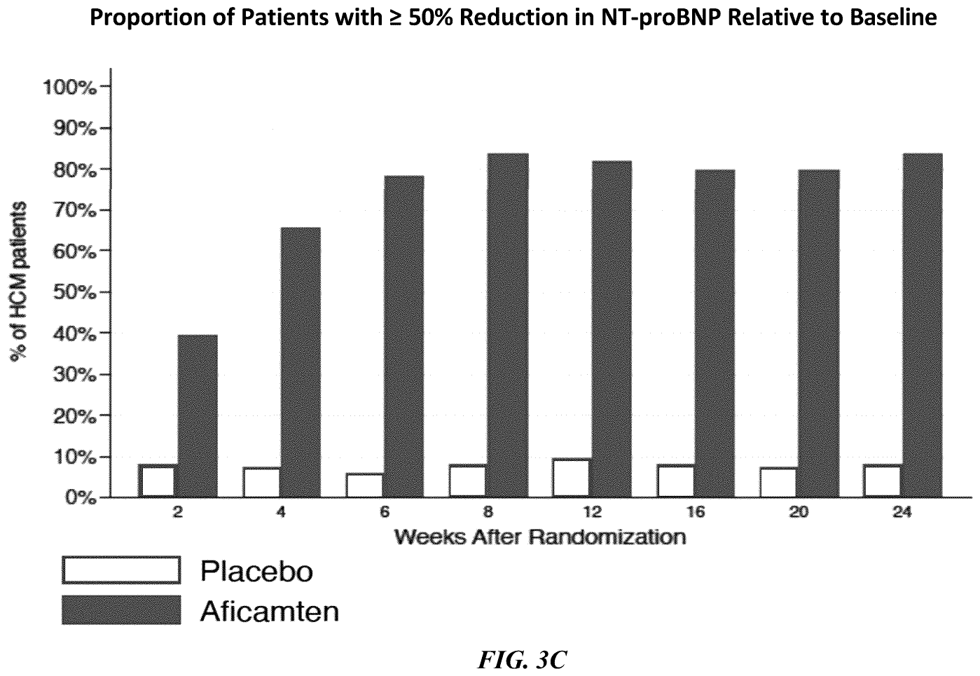

In some embodiments, the aficamten is polymorphic Form III of (R)—N-(5-(5-ethyl-1,2,4-oxadiazol-3-yl)-2,3-dihydro-1H-inden-1-yl)-1-methyl-1H-pyrazole-4-carboxamide. Angles 2-theta and relative peak intensities that may be observed for a mixture of Forms I and III using XRPD are shown in Table P-3. In some embodiments, a mixture of Forms I and III has an XRPD pattern substantially as shown in FIG. 3A of WO 2021/011807 and U.S. Patent Publication US-2022-0315571-A1.

TABLE P-3

| Angle (°2θ) | d value (Å) | Intensity (counts) |
|---|---|---|
| 3.6 | 24.3 | 4294.42 |
| 7.2 | 12.2 | 1591.53 |
| 9.6 | 9.2 | 7874.85 |
| 10.9 | 8.1 | 14071.97 |
| 11.1 | 7.9 | 4718.33 |
| 12.6 | 7.0 | 3488.46 |
| 12.8 | 6.9 | 11281.44 |
| 13.1 | 6.8 | 2684.87 |
| 13.5 | 6.5 | 1648.15 |
| 14.5 | 6.1 | 7367.71 |
| 15.8 | 5.6 | 35234.75 |
| 16.6 | 5.3 | 3504.83 |
| 18.1 | 4.9 | 41037.79 |
| 18.9 | 4.7 | 1062.55 |
| 19.2 | 4.6 | 6379.73 |
| 20.2 | 4.4 | 16176.19 |
| 20.3 | 4.4 | 17371.66 |
| 21.9 | 4.1 | 5995.19 |
| 22.2 | 4.0 | 6813.28 |
| 22.7 | 3.9 | 994.33 |
| 23.1 | 3.8 | 8769.11 |
| 23.9 | 3.7 | 16157.73 |
| 24.5 | 3.6 | 4099.43 |
| 24.9 | 3.6 | 927.84 |
| 25.5 | 3.5 | 5405.35 |
| 25.8 | 3.4 | 1470.89 |
| 26.1 | 3.4 | 2594.53 |
| 26.3 | 3.4 | 4109.63 |
| 27.1 | 3.3 | 6802.97 |
| 27.5 | 3.2 | 2786.18 |
| 28.6 | 3.1 | 2999.86 |
| 29.0 | 3.1 | 3257.84 |
| 29.9 | 3.0 | 1863.20 |
| 31.2 | 2.9 | 1010.52 |
| 31.9 | 2.8 | 1185.03 |
| 34.4 | 2.6 | 417.85 |
| 36.7 | 2.4 | 364.18 |
| 37.6 | 2.4 | 414.02 |
| 38.4 | 2.3 | 560.53 |

In some embodiments, polymorphic Form III has an XRPD pattern comprising peaks at angles 2-theta of 9.6±0.2, 10.9±0.2, 15.8±0.2, and 18.1±0.2 degrees. In some embodiments, polymorphic Form III has an XRPD pattern comprising peaks at angles 2-theta of 9.6±0.2, 10.9±0.2, 14.5±0.2, 15.8±0.2, and 18.1±0.2 degrees. In some embodiments, polymorphic Form III has an XRPD pattern comprising peaks at angles 2-theta of 9.6±0.2, 10.9±0.2, 14.5±0.2, 15.8±0.2, 18.1±0.2, and 20.2±0.2 degrees. It should be understood that relative intensities can vary depending on a number of factors, including sample preparation, mounting, and the instrument and analytical procedure and settings used to obtain the spectrum. Relative peak intensities and peak assignments can vary within experimental error. In some embodiments, peak assignments listed herein, including for polymorphic Form III, can vary by about ±0.6 degrees, ±0.4 degrees, ±0.2 degrees, or ±0.1 degrees 2-theta.

In some embodiments, a mixture of polymorphic Forms I and III has a DSC graph substantially as shown in FIG. 3B of WO 2021/011807 and U.S. Patent Publication US-2022-0315571-A1.

In some embodiments, a mixture of polymorphic Forms I and III has a TGA graph substantially as shown in FIG. 3B of WO 2021/011807 and U.S. Patent Publication US-2022-0315571-A1.

### Form IV

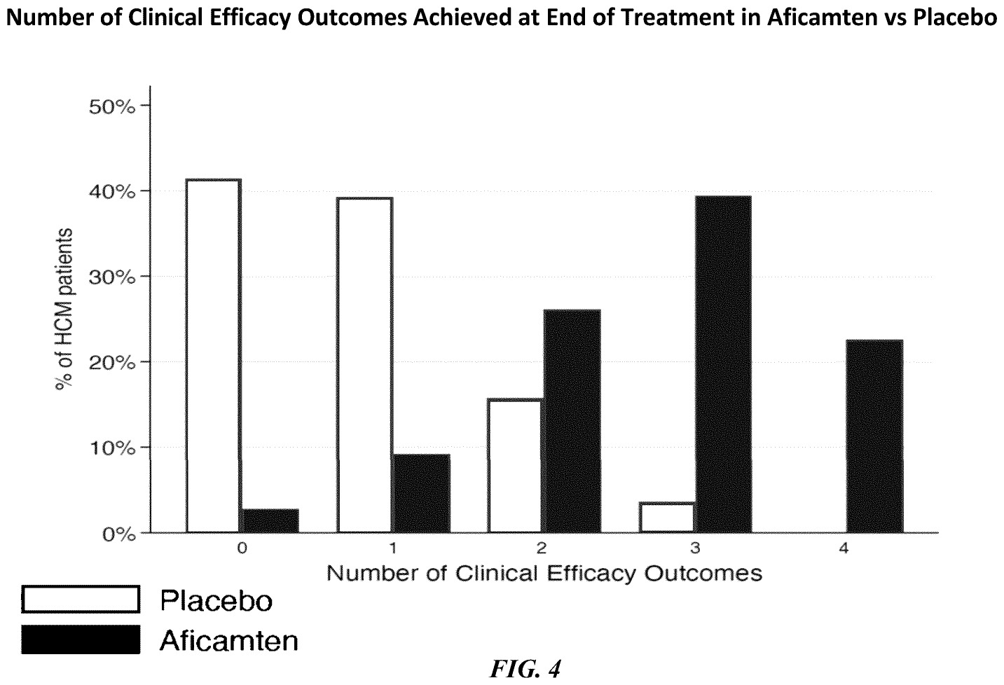

In some embodiments, the aficamten is polymorphic Form IV of (R)—N-(5-(5-ethyl-1,2,4-oxadiazol-3-yl)-2,3-dihydro-1H-inden-1-yl)-1-methyl-1H-pyrazole-4-carboxamide. Angles 2-theta and relative peak intensities that may be observed for Form IV using XRPD are shown in Table P-4. In some embodiments, Form IV has an XRPD pattern substantially as shown in FIG. 4A of WO 2021/011807 and U.S. Patent Publication US-2022-0315571-A1.

TABLE P-4

| Angle (°2θ) | d value (Å) | Intensity (counts) |
|---|---|---|
| 3.7 | 23.6 | 20546.07 |
| 7.7 | 11.5 | 316.33 |
| 11.1 | 7.9 | 50282.89 |
| 12.4 | 7.1 | 9463.20 |
| 12.8 | 6.9 | 128952.37 |
| 13.5 | 6.6 | 73004.53 |
| 14.3 | 6.2 | 1107.15 |
| 15.5 | 5.7 | 1601.21 |
| 16.6 | 5.3 | 4393.72 |
| 17.9 | 4.9 | 18738.68 |
| 18.5 | 4.8 | 10243.00 |
| 18.6 | 4.8 | 5141.69 |
| 19.1 | 4.6 | 807.92 |
| 19.9 | 4.4 | 1686.97 |
| 20.9 | 4.2 | 14781.60 |
| 21.5 | 4.1 | 11233.06 |
| 21.6 | 4.1 | 8729.09 |
| 21.9 | 4.1 | 29321.27 |
| 22.3 | 4.0 | 16474.92 |
| 22.4 | 4.0 | 12987.42 |
| 22.8 | 3.9 | 35633.79 |
| 23.1 | 3.8 | 21970.49 |
| 23.5 | 3.8 | 24153.01 |
| 23.9 | 3.7 | 6592.59 |
| 24.4 | 3.6 | 42430.12 |
| 24.8 | 3.6 | 27600.62 |
| 25.0 | 3.6 | 18197.26 |
| 25.3 | 3.5 | 17487.34 |
| 25.8 | 3.4 | 20166.11 |
| 26.2 | 3.4 | 9283.12 |
| 27.1 | 3.3 | 1860.56 |
| 27.4 | 3.3 | 3534.99 |
| 28.0 | 3.2 | 2537.61 |
| 28.6 | 3.1 | 13746.55 |
| 29.0 | 3.1 | 1991.34 |
| 30.0 | 3.0 | 11236.64 |
| 30.5 | 2.9 | 5185.28 |
| 30.8 | 2.9 | 7657.41 |
| 31.0 | 2.9 | 2753.63 |
| 31.4 | 2.8 | 5359.89 |
| 33.8 | 2.6 | 1085.12 |
| 35.0 | 2.6 | 1833.47 |
| 35.7 | 2.5 | 1218.65 |
| 36.1 | 2.5 | 496.93 |
| 36.7 | 2.4 | 1373.07 |
| 37.9 | 2.4 | 2127.34 |
| 38.1 | 2.4 | 883.05 |
| 39.8 | 2.3 | 1200.46 |

In some embodiments, polymorphic Form IV has an XRPD pattern displaying at least two, at least three, at least four, at least five, at least six, at least seven, at least eight, at least nine, or at least ten of the peaks at angles 2-theta with the greatest intensity in the XRPD pattern substantially as shown in FIG. 4A of WO 2021/011807 and U.S. Patent Publication US-2022-0315571-A1 or as provided in Table P-4. It should be understood that relative intensities can vary depending on a number of factors, including sample preparation, mounting, and the instrument and analytical procedure and settings used to obtain the spectrum. Relative peak intensities and peak assignments can vary within experimental error. In some embodiments, peak assignments listed herein, including for polymorphic Form IV, can vary by about ±0.6 degrees, ±0.4 degrees, ±0.2 degrees, or ±0.1 degrees 2-theta.

In some embodiments, polymorphic Form IV has an XRPD pattern comprising peaks at angles 2-theta of 3.7±0.2, 7.7±0.2, 11.1±0.2, 12.4±0.2, 12.8±0.2, 13.5±0.2, 14.3±0.2, 15.5±0.2, 16.6±0.2, 17.9±0.2, 18.5±0.2, 18.6±0.2, 19.1±0.2, 19.9±0.2, 20.9±0.2, 21.5±0.2, 21.6±0.2, 21.9±0.2, 22.3±0.2, 22.4±0.2, 22.8±0.2, 23.1±0.2, 23.5±0.2, 23.9±0.2, 24.4±0.2, 24.8±0.2, 25.0±0.2, 25.3±0.2, 25.8±0.2, 26.2±0.2, 27.1±0.2, 27.4±0.2, 28.0±0.2, 28.6±0.2, 29.0±0.2, 30.0±0.2, 30.5±0.2, 30.8±0.2, 31.0±0.2, 31.4±0.2, 33.8±0.2, 35.0±0.2, 35.7±0.2, 36.1±0.2, 36.7±0.2, 37.9±0.2, 38.1±0.2, 39.8±0.2 degrees. In some embodiments, polymorphic Form IV has an XRPD pattern comprising peaks at angles 2-theta of 3.7±0.2, 11.1±0.2, 12.8±0.2, 13.5±0.2, 21.9±0.2, 22.8±0.2, 23.1±0.2, 23.5±0.2, 24.4±0.2, and 24.8±0.2 degrees. In some embodiments, polymorphic Form IV has an XRPD pattern comprising peaks at angles 2-theta of 11.1±0.2, 12.8±0.2, 13.5±0.2, 22.8±0.2, and 24.4±0.2 degrees. It is to be understood that additional peaks in the XRPD pattern other than those shown in FIG. 4A of WO 2021/011807 and U.S. Patent Publication US-2022-0315571-A1 or as provided in Table P-4 may be observed, for instance, due to the presence of impurities, solvent, or other polymorphs or amorphic forms present in the test sample.

In some embodiments, Form IV has a DSC graph substantially as shown in FIG. 4B of WO 2021/011807 and U.S. Patent Publication US-2022-0315571-A1. In some embodiments, Form IV is characterized as having an endotherm onset at about 200° C. as determined by DSC. In some embodiments, Form IV is characterized as having a melting endotherm onset at about 200±2° C. (e.g., 200±1.9° C., 200±1.8° C., 200±1.7° C., 200±1.6° C., 200±1.5° C., 200±1.4° C., 200±1.3° C., 200±1.2° C., 200±1, 200±0.9° C., 200±0.8° C., 200±0.7° C., 200±0.6° C., 200±0.5° C., 200±0.4° C., 200±0.3° C., 200±0.2° C., or 200±0.1° C.) as determined by DSC.

In some embodiments, Form IV has a TGA graph substantially as shown in FIG. 4B of WO 2021/011807 and U.S. Patent Publication US-2022-0315571-A1.

In some embodiments of Form IV, at least one, at least two, at least three, at least four, or all of the following (a)-(e) apply:
(a) Form IV has an XRPD pattern comprising peaks at angles 2-theta of 11.1±0.2, 12.8±0.2, 13.5±0.2, 22.8±0.2, and 24.4±0.2 degrees; an XRPD pattern comprising peaks at angles 2-theta of 3.7±0.2, 11.1±0.2, 12.8±0.2, 13.5±0.2, 21.9±0.2, 22.8±0.2, 23.1±0.2, 23.5±0.2, 24.4±0.2, and 24.8±0.2 degrees; or an XRPD pattern comprising peaks at angles 2-theta of 3.7±0.2, 7.7±0.2, 11.1±0.2, 12.4±0.2, 12.8±0.2, 13.5±0.2, 14.3±0.2, 15.5±0.2, 16.6±0.2, 17.9±0.2, 18.5±0.2, 18.6±0.2, 19.1±0.2, 19.9±0.2, 20.9±0.2, 21.5±0.2, 21.6±0.2, 21.9±0.2, 22.3±0.2, 22.4±0.2, 22.8±0.2, 23.1±0.2, 23.5±0.2, 23.9±0.2, 24.4±0.2, 24.8±0.2, 25.0±0.2, 25.3±0.2, 25.8±0.2, 26.2±0.2, 27.1±0.2, 27.4±0.2, 28.0±0.2, 28.6±0.2, 29.0±0.2, 30.0±0.2, 30.5±0.2, 30.8±0.2, 31.0±0.2, 31.4±0.2, 33.8±0.2, 35.0±0.2, 35.7±0.2, 36.1±0.2, 36.7±0.2, 37.9±0.2, 38.1±0.2, 39.8±0.2 degrees;
(b) Form IV has an XRPD pattern substantially as shown in FIG. 4A of WO 2021/011807 and U.S. Patent Publication US-2022-0315571-A1;
(c) Form IV has a DSC graph substantially as shown in FIG. 4B of WO 2021/011807 and U.S. Patent Publication US-2022-0315571-A1;
(d) Form IV is characterized as having a melting endotherm onset at about 200° C. as determined by DSC; and
(e) Form IV has a TGA graph substantially as shown in FIG. 4B of WO 2021/011807 and U.S. Patent Publication US-2022-0315571-A1.

### Form V

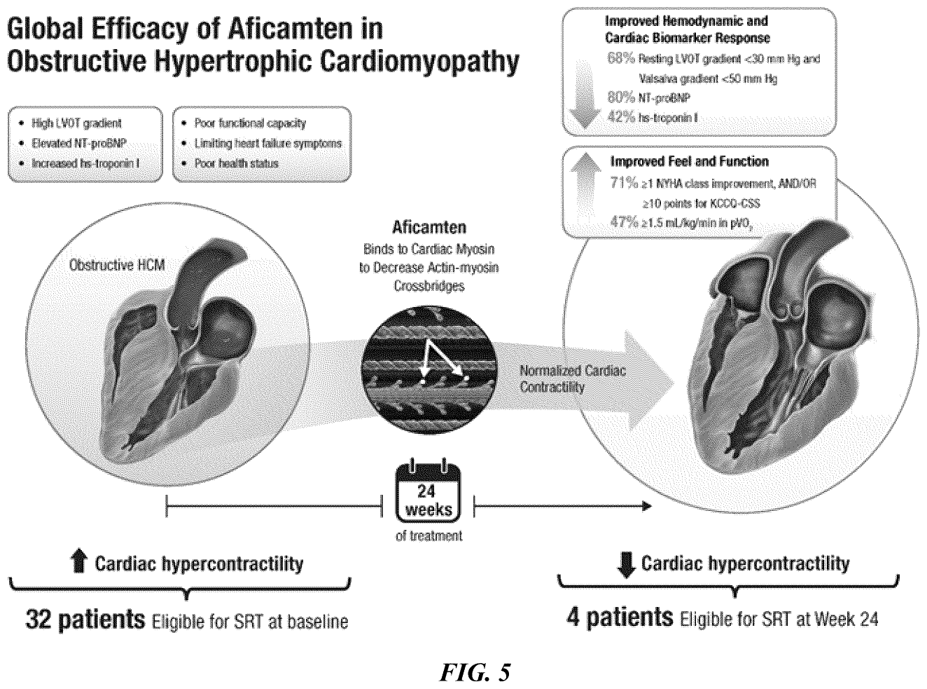

In some embodiments, the aficamten is polymorphic Form V of (R)—N-(5-(5-ethyl-1,2,4-oxadiazol-3-yl)-2,3-dihydro-1H-inden-1-yl)-1-methyl-1H-pyrazole-4-carboxamide. Angles 2-theta and relative peak intensities that may be observed for Form V using XRPD are shown in Table P-5. In some embodiments, Form V has an XRPD pattern substantially as shown in FIG. 5 of WO 2021/011807 and U.S. Patent Publication US-2022-0315571-A1.

TABLE P-5

| Angle (°2θ) | d value (Å) | Intensity (counts) |
|---|---|---|
| 5.7 | 15.4 | 920.22 |
| 8.3 | 10.7 | 902.83 |
| 11.5 | 7.7 | 9978.6 |
| 13.8 | 6.4 | 370.79 |
| 15.5 | 5.7 | 100.26 |
| 15.8 | 5.6 | 290.17 |
| 16.3 | 5.4 | 3772.89 |
| 16.6 | 5.4 | 397.64 |
| 17.2 | 5.1 | 500.92 |
| 17.8 | 5.0 | 473.82 |
| 18.5 | 4.8 | 237.44 |
| 18.9 | 4.7 | 388.87 |
| 19.1 | 4.6 | 2061.82 |
| 19.8 | 4.5 | 391.52 |
| 20.0 | 4.4 | 2661.48 |
| 20.2 | 4.4 | 1338.2 |
| 20.7 | 4.3 | 650.6 |
| 21.2 | 4.2 | 3071.64 |
| 21.6 | 4.1 | 295.42 |
| 23.0 | 3.9 | 446.39 |
| 23.1 | 3.9 | 165.78 |
| 23.3 | 3.8 | 1093.75 |
| 24.0 | 3.7 | 1807.48 |
| 24.2 | 3.7 | 176.77 |
| 24.3 | 3.7 | 472.09 |
| 24.6 | 3.6 | 216.92 |
| 24.7 | 3.6 | 3905.17 |
| 25.2 | 3.5 | 138.38 |
| 25.6 | 3.5 | 2004.45 |
| 26.7 | 3.3 | 1751.94 |
| 27.1 | 3.3 | 53.39 |
| 27.3 | 3.3 | 55.93 |
| 27.5 | 3.2 | 25.62 |
| 27.9 | 3.2 | 217.7 |
| 28.1 | 3.2 | 1093.17 |
| 28.4 | 3.1 | 215.32 |
| 28.9 | 3.1 | 222.86 |
| 29.2 | 3.1 | 615.11 |
| 29.7 | 3.0 | 856.96 |
| 29.8 | 3.0 | 361.62 |
| 29.9 | 3.0 | 544.6 |
| 30.4 | 2.9 | 248.45 |
| 30.6 | 2.9 | 181.85 |
| 31.1 | 2.9 | 481.57 |
| 31.3 | 2.9 | 57.68 |
| 31.5 | 2.8 | 312.95 |
| 32.0 | 2.8 | 174.91 |
| 32.9 | 2.7 | 101.41 |
| 33.0 | 2.7 | 61.9 |
| 33.2 | 2.7 | 71.42 |
| 33.5 | 2.7 | 99.57 |
| 34.4 | 2.6 | 162.66 |
| 34.6 | 2.6 | 42.98 |
| 34.9 | 2.6 | 15.79 |
| 35.3 | 2.5 | 33.86 |
| 35.7 | 2.5 | 241.09 |
| 36.0 | 2.5 | 67.72 |
| 36.2 | 2.5 | 17.68 |
| 36.5 | 2.5 | 12.09 |
| 36.6 | 2.5 | 100.18 |
| 37.0 | 2.4 | 39.38 |
| 37.1 | 2.4 | 63.03 |
| 37.5 | 2.4 | 49.61 |
| 37.8 | 2.4 | 59.59 |
| 37.9 | 2.4 | 68.66 |
| 38.3 | 2.4 | 18.63 |
| 38.4 | 2.3 | 28.07 |
| 38.7 | 2.3 | 21.55 |
| 38.8 | 2.3 | 52.54 |
| 39.3 | 2.3 | 31.1 |
| 39.4 | 2.3 | 123.61 |
| 39.6 | 2.3 | 51.44 |
| 39.9 | 2.3 | 74.51 |

In some embodiments, polymorphic Form V has an XRPD pattern displaying at least two, at least three, at least four, at least five, at least six, at least seven, at least eight, at least nine, or at least ten of the peaks at angles 2-theta with the greatest intensity in the XRPD pattern substantially as shown in FIG. 5 of WO 2021/011807 and U.S. Patent Publication US-2022-0315571-A1 or as provided in Table P-5. It should be understood that relative intensities can vary depending on a number of factors, including sample preparation, mounting, and the instrument and analytical procedure and settings used to obtain the spectrum. Relative peak intensities and peak assignments can vary within experimental error. In some embodiments, peak assignments listed herein, including for polymorphic Form V, can vary by about ±0.6 degrees, ±0.4 degrees, ±0.2 degrees, or ±0.1 degrees 2-theta.

In some embodiments, polymorphic Form V has an XRPD pattern comprising peaks at angles 2-theta of 5.7±0.2, 8.3±0.2, 11.5±0.2, 13.8±0.2, 15.5±0.2, 15.8±0.2, 16.3±0.2, 16.6±0.2, 17.2±0.2, 17.8±0.2, 18.5±0.2, 18.9±0.2, 19.1±0.2, 19.8±0.2, 20.0±0.2, 20.2±0.2, 20.7±0.2, 21.2±0.2, 21.6±0.2, 23.0±0.2, 23.1±0.2, 23.3±0.2, 24.0±0.2, 24.2±0.2, 24.3±0.2, 24.6±0.2, 24.7±0.2, 25.2±0.2, 25.6±0.2, 26.7±0.2, 27.1±0.2, 27.3±0.2, 27.5±0.2, 27.9±0.2, 28.1±0.2, 28.4±0.2, 28.9±0.2, 29.2±0.2, 29.7±0.2, 29.8±0.2, 29.9±0.2, 30.4±0.2, 30.6±0.2, 31.1±0.2, 31.3±0.2, 31.5±0.2, 32.0±0.2, 32.9±0.2, 33.0±0.2, 33.2±0.2, 33.5±0.2, 34.4±0.2, 34.6±0.2, 34.9±0.2, 35.3±0.2, 35.7±0.2, 36.0±0.2, 36.2±0.2, 36.5±0.2, 36.6±0.2, 37.0±0.2, 37.1±0.2, 37.5±0.2, 37.8±0.2, 37.9±0.2, 38.3±0.2, 38.4±0.2, 38.7±0.2, 38.8±0.2, 39.3±0.2, 39.4±0.2, 39.6±0.2, and 39.9±0.2 degrees. In some embodiments, polymorphic Form V has an XRPD pattern comprising peaks at angles 2-theta of 5.7±0.2, 8.3±0.2, 11.5±0.2, 16.3±0.2, 17.2±0.2, 19.1±0.2, 20.0±0.2, 20.2±0.2, 20.7±0.2, 21.2±0.2, 23.3±0.2, 24.0±0.2, 24.7±0.2, 25.6±0.2, 26.7±0.2, 28.1±0.2, 29.2±0.2, 29.7±0.2, 29.9±0.2, and 31.1±0.2 degrees. In some embodiments, polymorphic Form V has an XRPD pattern comprising peaks at angles 2-theta of 11.5±0.2, 16.3±0.2, 19.1±0.2, 20.0±0.2, 20.2±0.2, 21.2±0.2, 24.0±0.2, 24.7±0.2, 25.6±0.2, and 26.7±0.2 degrees. In some embodiments, polymorphic Form V has an XRPD pattern comprising peaks at angles 2-theta of 11.5±0.2, 16.3±0.2, 20.0±0.2, 21.2±0.2, and 24.7±0.2 degrees. It is to be understood that additional peaks in the XRPD pattern other than those shown in FIG. 5 of WO 2021/011807 and U.S. Patent Publication US-2022-0315571-A1 or as provided in Table P-5 may be observed, for instance, due to the presence of impurities, solvent, or other polymorphs or amorphic forms present in the test sample.

In some embodiments of Form V, at least one or both of the following (a)-(b) apply:
(a) Form V has an XRPD pattern comprising peaks at angles 2-theta of 11.5±0.2, 16.3±0.2, 20.0±0.2, 21.2±0.2, and 24.7±0.2 degrees; an XRPD pattern comprising peaks at angles 2-theta of 11.5±0.2, 16.3±0.2, 19.1±0.2, 20.0±0.2, 20.2±0.2, 21.2±0.2, 24.0±0.2, 24.7±0.2, 25.6±0.2, and 26.7±0.2 degrees; or an XRPD pattern comprising peaks at angles 2-theta of 5.7±0.2, 8.3±0.2, 11.5±0.2, 16.3±0.2, 17.2±0.2, 19.1±0.2, 20.0±0.2, 20.2±0.2, 20.7±0.2, 21.2±0.2, 23.3±0.2, 24.0±0.2, 24.7±0.2, 25.6±0.2, 26.7±0.2, 28.1±0.2, 29.2±0.2, 29.7±0.2, 29.9±0.2, and 31.1±0.2 degrees; and
(b) Form V has an XRPD pattern substantially as shown in FIG. 5 of WO 2021/011807 and U.S. Patent Publication US-2022-0315571-A1.

### Form VI

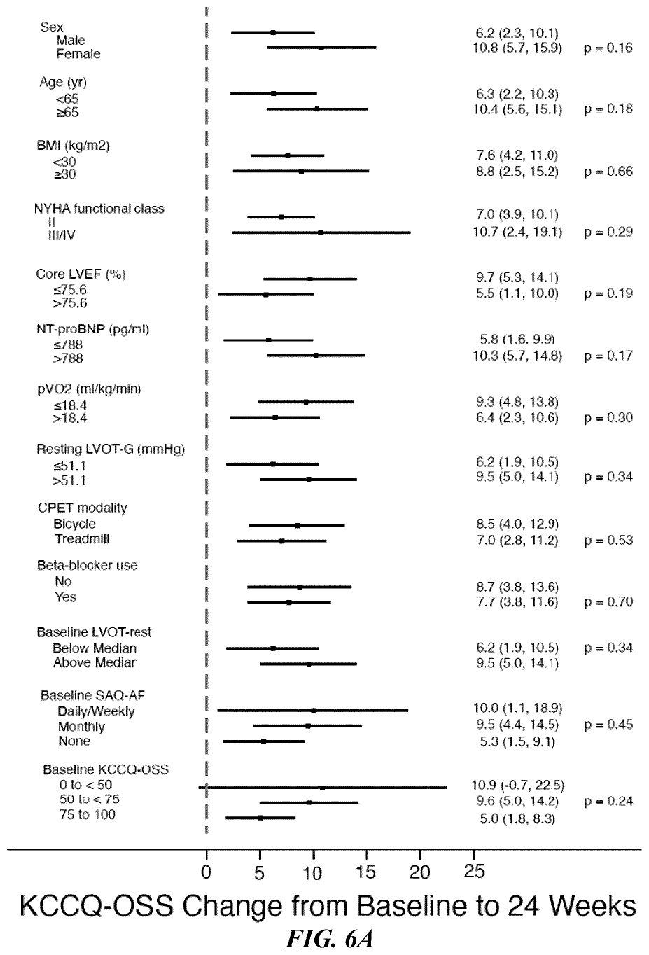

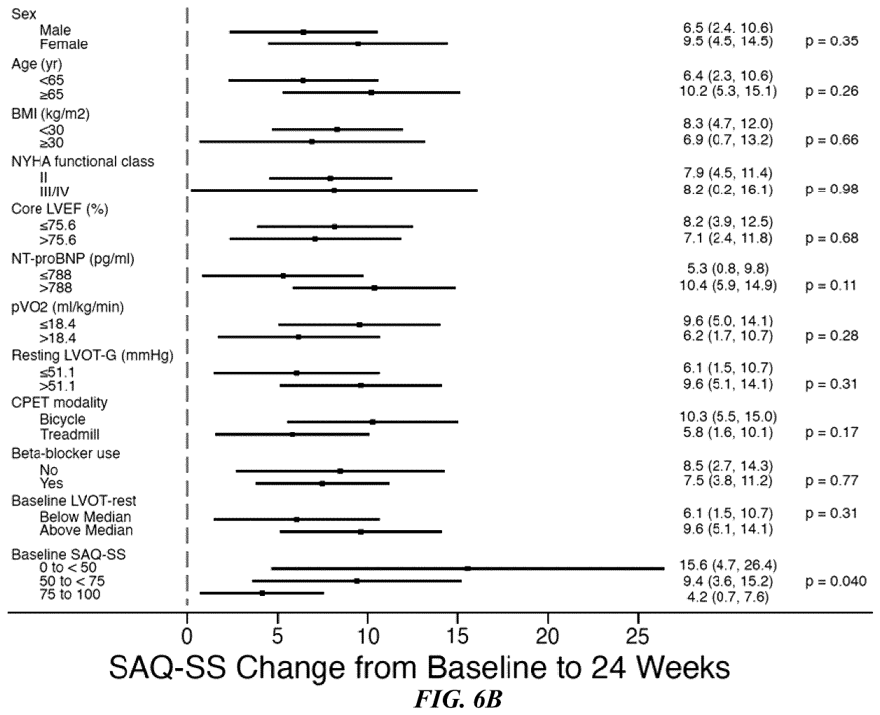

In some embodiments, the aficamten is polymorphic Form VI of (R)—N-(5-(5-ethyl-1,2,4-oxadiazol-3-yl)-2,3-dihydro-1H-inden-1-yl)-1-methyl-1H-pyrazole-4-carboxamide. Angles 2-theta and relative peak intensities that may be observed for Form VI using XRPD are shown in Table P-6. In some embodiments, Form VI has an XRPD pattern substantially as shown in FIG. 6A of WO 2021/011807 and U.S. Patent Publication US-2022-0315571-A1.

TABLE P-6

| Angle (°2θ) | d value (Å) | Intensity (counts) |
|---|---|---|
| 3.0 | 29.4 | 54.91 |
| 5.0 | 17.7 | 127.50 |
| 5.4 | 16.3 | 453.97 |
| 5.9 | 14.9 | 587.10 |
| 7.2 | 12.3 | 125.69 |
| 8.1 | 10.9 | 567.03 |
| 8.9 | 10.0 | 345.88 |
| 9.6 | 9.2 | 432.70 |
| 9.9 | 8.9 | 79.69 |
| 10.6 | 8.3 | 788.69 |
| 12.1 | 7.3 | 797.99 |
| 13.3 | 6.6 | 159.40 |
| 14.0 | 6.3 | 476.49 |
| 14.4 | 6.1 | 77.78 |
| 14.7 | 6.0 | 220.91 |
| 15.0 | 5.9 | 593.87 |
| 15.4 | 5.8 | 46.97 |
| 16.1 | 5.5 | 1350.47 |
| 16.5 | 5.4 | 388.67 |
| 17.8 | 5.0 | 805.27 |
| 18.9 | 4.7 | 55.59 |
| 19.0 | 4.7 | 100.12 |
| 19.2 | 4.6 | 53.05 |
| 19.6 | 4.5 | 24.26 |
| 20.0 | 4.4 | 98.96 |
| 20.3 | 4.4 | 55.64 |
| 20.7 | 4.3 | 145.05 |
| 21.1 | 4.2 | 226.01 |
| 21.9 | 4.1 | 246.53 |
| 22.6 | 3.9 | 115.35 |
| 22.9 | 3.9 | 78.78 |
| 23.6 | 3.8 | 399.26 |
| 23.6 | 3.8 | 373.80 |
| 23.8 | 3.7 | 58.05 |
| 24.4 | 3.6 | 146.88 |
| 24.8 | 3.6 | 348.39 |
| 25.5 | 3.5 | 226.63 |
| 26.4 | 3.4 | 42.15 |
| 26.7 | 3.3 | 137.53 |
| 27.3 | 3.3 | 112.85 |
| 27.6 | 3.2 | 120.57 |
| 28.2 | 3.2 | 55.34 |
| 28.5 | 3.1 | 60.37 |
| 29.0 | 3.1 | 26.44 |
| 29.6 | 3.0 | 70.01 |
| 29.9 | 3.0 | 60.95 |
| 30.4 | 2.9 | 49.00 |
| 30.9 | 2.9 | 14.91 |
| 31.6 | 2.8 | 12.97 |
| 32.2 | 2.8 | 38.90 |
| 32.6 | 2.7 | 40.95 |
| 33.1 | 2.7 | 16.26 |
| 33.3 | 2.7 | 16.56 |
| 34.5 | 2.6 | 10.35 |
| 35.0 | 2.6 | 16.35 |
| 35.5 | 2.5 | 36.10 |
| 38.5 | 2.3 | 20.00 |

In some embodiments, polymorphic Form VI has an XRPD pattern displaying at least two, at least three, at least four, at least five, at least six, at least seven, at least eight, at least nine, or at least ten of the peaks at angles 2-theta with the greatest intensity in the XRPD pattern substantially as shown in FIG. 6A of WO 2021/011807 and U.S. Patent Publication US-2022-0315571-A1 or as provided in Table P-6. It should be understood that relative intensities can vary depending on a number of factors, including sample preparation, mounting, and the instrument and analytical procedure and settings used to obtain the spectrum. Relative peak intensities and peak assignments can vary within experimental error. In some embodiments, peak assignments listed herein, including for polymorphic Form VI, can vary by about ±0.6 degrees, ±0.4 degrees, ±0.2 degrees, or ±0.1 degrees 2-theta.

In some embodiments, polymorphic Form VI has an XRPD pattern comprising peaks at angles 2-theta of 3.0±0.2, 5.0±0.2, 5.4±0.2, 5.9±0.2, 7.2±0.2, 8.1±0.2, 8.9±0.2, 9.6±0.2, 9.9±0.2, 10.6±0.2, 12.1±0.2, 13.3±0.2, 14.0±0.2, 14.4±0.2, 14.7±0.2, 15.0±0.2, 15.4±0.2, 16.1±0.2, 16.5±0.2, 17.8±0.2, 18.9±0.2, 19.0±0.2, 19.2±0.2, 19.6±0.2, 20.0±0.2, 20.3±0.2, 20.7±0.2, 21.1±0.2, 21.9±0.2, 22.6±0.2, 22.9±0.2, 23.6±0.2, 23.8±0.2, 24.4±0.2, 24.8±0.2, 25.5±0.2, 26.4±0.2, 26.7±0.2, 27.3±0.2, 27.6±0.2, 28.2±0.2, 28.5±0.2, 29.0±0.2, 29.6±0.2, 29.9±0.2, 30.4±0.2, 30.9±0.2, 31.6±0.2, 32.2±0.2, 32.6±0.2, 33.1±0.2, 33.3±0.2, 34.5±0.2, 35.0±0.2, 35.5±0.2, and 38.5±0.2 degrees. In some embodiments, polymorphic Form VI has an XRPD pattern comprising peaks at angles 2-theta of 5.4±0.2, 5.9±0.2, 8.1±0.2, 9.6±0.2, 10.6±0.2, 12.1±0.2, 14.0±0.2, 15.0±0.2, 16.1±0.2, and 17.8±0.2 degrees. In some embodiments, polymorphic Form VI has an XRPD pattern comprising peaks at angles 2-theta of 10.6±0.2, 12.1±0.2, 15.0±0.2, 16.1±0.2, and 17.8±0.2 degrees. It is to be understood that additional peaks in the XRPD pattern other than those shown in FIG. 6A of WO 2021/011807 and U.S. Patent Publication US-2022-0315571-A1 or as provided in Table P-6 may be observed, for instance, due to the presence of impurities, solvent, or other polymorphs or amorphic forms present in the test sample.

In some embodiments, Form VI has a TGA graph substantially as shown in FIG. 6B of WO 2021/011807 and U.S. Patent Publication US-2022-0315571-A1 or a TGA graph substantially as shown in FIG. 6C of WO 2021/011807 and U.S. Patent Publication US-2022-0315571-A1. In some embodiments, Form VI exhibits a weight loss of about 2%±0.5% between 25° C. and 200° C. as determined by TGA.

In some embodiments, Form VI has a DSC graph substantially as shown in FIG. 6D of WO 2021/011807 and U.S. Patent Publication US-2022-0315571-A1 or a DSC graph substantially as shown in FIG. 6E of WO 2021/011807 and U.S. Patent Publication US-2022-0315571-A1. In some embodiments, Form VI is characterized as having a melting endotherm onset at about 200±2° C. (e.g., 200±1.9° C., 200±1.8° C., 200±1.7° C., 200±1.6° C., 200±1.5° C., 200±1.4° C., 200±1.3° C., 200±1.2° C., 200±1, 200±0.9° C., 200±0.8° C., 200±0.7° C., 200±0.6° C., 200±0.5° C., 200±0.4° C., 200±0.3° C., 200±0.2° C., or 200±0.1° C.) as determined by DSC. In some embodiments, Form VI is characterized as having an endotherm onset at about 200±2° C. (e.g., 200±1.9° C., 200±1.8° C., 200±1.7° C., 200±1.6° C., 200±1.5° C., 200±1.4° C., 200±1.3° C., 200±1.2° C., 200±1, 200±0.9° C., 200±0.8° C., 200±0.7° C., 200±0.6° C., 200±0.5° C., 200±0.4° C., 200±0.3° C., 200±0.2° C., or 200±0.1° C.), an exotherm onset at about 115±2° C. (e.g., 115±1.9° C., 115±1.8° C., 115±1.7° C., 115±1.6° C., 115±1.5° C., 115±1.4° C., 115±1.3° C., 115±1.2° C., 115±1, 115±0.9° C., 115±0.8° C., 115±0.7° C., 115±0.6° C., 115±0.5° C., 115±0.4° C., 115±0.3° C., 115±0.2° C., or 115±0.1° C.), or an endotherm onset at about 41±2° C. (e.g., 41±1.9° C., 41±1.8° C., 41±1.7° C., 41±1.6° C., 41±1.5° C., 41±1.4° C., 41±1.3° C., 41±1.2° C., 41±1, 41±0.9° C., 41±0.8° C., 41±0.7° C., 41±0.6° C., 41±0.5° C., 41±0.4° C., 41±0.3° C., 41±0.2° C., or 41±0.1° C.), or any combinations thereof.

In some embodiments of Form VI, at least one, at least two, at least three, at least four, at least five, at least six or all of the following (a)-(g) apply:
(a) Form VI has an XRPD pattern comprising peaks at angles 2-theta of 10.6±0.2, 12.1±0.2, 15.0±0.2, 16.1±0.2, and 17.8±0.2 degrees; an XRPD pattern comprising peaks at angles 2-theta of 5.4±0.2, 5.9±0.2, 8.1±0.2, 9.6±0.2, 10.6±0.2, 12.1±0.2, 14.0±0.2, 15.0±0.2, 16.1±0.2, and 17.8±0.2 degrees; or an XRPD pattern comprising peaks at angles 2-theta of 3.0±0.2, 5.0±0.2, 5.4±0.2, 5.9±0.2, 7.2±0.2, 8.1±0.2, 8.9±0.2, 9.6±0.2, 9.9±0.2, 10.6±0.2, 12.1±0.2, 13.3±0.2, 14.0±0.2, 14.4±0.2, 14.7±0.2, 15.0±0.2, 15.4±0.2, 16.1±0.2, 16.5±0.2, 17.8±0.2, 18.9±0.2, 19.0±0.2, 19.2±0.2, 19.6±0.2, 20.0±0.2, 20.3±0.2, 20.7±0.2, 21.1±0.2, 21.9±0.2, 22.6±0.2, 22.9±0.2, 23.6±0.2, 23.8±0.2, 24.4±0.2, 24.8±0.2, 25.5±0.2, 26.4±0.2, 26.7±0.2, 27.3±0.2, 27.6±0.2, 28.2±0.2, 28.5±0.2, 29.0±0.2, 29.6±0.2, 29.9±0.2, 30.4±0.2, 30.9±0.2, 31.6±0.2, 32.2±0.2, 32.6±0.2, 33.1±0.2, 33.3±0.2, 34.5±0.2, 35.0±0.2, 35.5±0.2, and 38.5±0.2 degrees;
(b) Form VI has an XRPD pattern substantially as shown in FIG. 6A of WO 2021/011807 and U.S. Patent Publication US-2022-0315571-A1;
(c) Form VI has a TGA graph substantially as shown in FIG. 6B of WO 2021/011807 and U.S. Patent Publication US-2022-0315571-A1 or FIG. 6C of WO 2021/011807 and U.S. Patent Publication US-2022-0315571-A1.
(d) Form VI has a weight loss of about 2%±0.5% between 25° C. and 200° C. as determined by TGA;
(e) Form VI has a DSC graph substantially as shown in FIG. 6D of WO 2021/011807 and U.S. Patent Publication US-2022-0315571-A1 or FIG. 6E of WO 2021/011807 and U.S. Patent Publication US-2022-0315571-A1; and
(f) Form IV is characterized as having a melting endotherm onset at about 200° C. as determined by DSC; and
(g) Form VI is characterized as having an endotherm onset at about 200° C., an exotherm onset at about 115° C., or an endotherm onset at about 41° C., or any combination thereof, as determined by DSC.

### Kits

Also provided are articles of manufacture and kits containing any of the compounds or pharmaceutical compositions provided herein. The article of manufacture may comprise a container with a label. Suitable containers include, for example, bottles, vials, and test tubes. The containers may be formed from a variety of materials such as glass or plastic. The container may hold a pharmaceutical composition provided herein. The label on the container may indicate that the pharmaceutical composition is used for preventing, treating, or suppressing a condition described herein, and may also indicate directions for either in vivo or in vitro use.

In one aspect, provided herein are kits containing a compound or composition described herein and instructions for use. The kits may contain instructions for use in the treatment of a heart disease in an individual or subject in need thereof. A kit may additionally contain any materials or equipment that may be used in the administration of the compound or composition, such as vials, syringes, or IV bags. A kit may also contain sterile packaging.

In some embodiments, the present disclosure provides methods for manufacturing a medicament comprising aficamten, comprising:
manufacturing a first collection of tablets the amounts of aficamten (which may exist in various forms) in which are about, or are about half of, a first daily dose;
manufacturing a second collection of tablets the amounts of aficamten (which may exist in various forms) in which are about, or are about half of, a second daily dose;
optionally manufacturing a third collection of tablets the amounts of aficamten (which may exist in various forms) in which are about, or are about half of, a third daily dose; and
optionally manufacturing a fourth collection of tablets the amounts of aficamten (which may exist in various forms) in which are about, or are about half of, a fourth daily dose.

In some embodiments, a method comprises manufacturing a third collection of tablets the amounts of aficamten (which may exist in various forms) in which are about, or are about half of, a third daily dose. In some embodiments, a method comprises manufacturing a fourth collection of tablets the amounts of aficamten (which may exist in various forms) in which are about, or are about half of, a fourth daily dose.

In some embodiments, the present disclosure provides methods for manufacturing medicament comprising aficamten, comprising:
manufacturing a first collection of tablets the amounts of aficamten (which may exist in various forms) in which are about a first daily dose;
manufacturing a second collection of tablets the amounts of aficamten (which may exist in various forms) in which are about a second daily dose;
optionally manufacturing a third collection of tablets the amounts of aficamten (which may exist in various forms) in which are about a third daily dose; and
optionally manufacturing a fourth collection of tablets the amounts of aficamten (which may exist in various forms) in which are about a fourth daily dose.

In some embodiments, a method comprises manufacturing a third collection of tablets the amounts of aficamten (which may exist in various forms) in which are about a third daily dose. In some embodiments, a method comprises manufacturing a fourth collection of tablets the amounts of aficamten (which may exist in various forms) in which are about a fourth daily dose.

Various first, second, third and fourth daily doses are described herein. In some embodiments, a daily dose is about 5 mg of aficamten. In some embodiments, a first daily dose is about 5 mg of aficamten. In some embodiments, a daily dose is about 10 mg of aficamten. In some embodiments, a daily dose is about 15 mg of aficamten. In some embodiments, a daily dose is about 20 mg of aficamten. In some embodiments, tablets with at least two different daily doses are manufactured. In some embodiments, tablets the amounts of aficamten (which may exist in various forms) in which are about 15 mg are manufactured. In some embodiments, tablets the amounts of aficamten (which may exist in various forms) in which are about 2.5 mg are manufactured. In some embodiments, tablets the amounts of aficamten (which may exist in various forms) in which are about 7.5 mg are manufactured. Those skilled in the art will appreciate that in addition to aficamten, tablets may comprise various other components as described herein, e.g., filler, binder, disintegrant, surfactant, lubricant, etc. In some embodiments, tablets are film-coated.

### EXAMPLES

The application may be better understood by reference to the following non-limiting examples, which are provided as exemplary embodiments of the application. The following examples are presented in order to more fully illustrate embodiments and should in no way be construed, however, as limiting the broad scope of the application. While certain embodiments of the present application have been shown and described herein, it will be obvious that such embodiments are provided by way of example only. Numerous variations, changes, and substitutions may occur to those skilled in the art without departing from the spirit and scope of the invention. It should be understood that various alternatives to the embodiments described herein may be employed in practicing the methods described herein.

Abbreviations: AE=adverse event; CMI=cardiac myosin inhibitor; CGI=Clinical Global Impression; CPET=cardiopulmonary exercise testing; c′=early diastolic mitral annular velocity; E/e′=ratio between early mitral inflow velocity and early diastolic mitral annular velocity; EQ-5D-5L=EuroQol 5-dimension 5-level instrument; hs-cTnI or hsTnI=high sensitivity cardiac troponin I; HCM=hypertrophic cardiomyopathy; KCCQ=Kansas City Cardiomyopathy Questionnaire; KCCQ-CSS=KCCQ-Clinical Summary Score; KCCQ-OSS=KCCQ-Overall Summary Score; LA=left atrium/atrial; LAVI=left atrial volume index; LVEF=left ventricular ejection fraction; LV GCS=left ventricular global circumferential strain; LV GLS=left ventricular global longitudinal strain; LVMI=left ventricular mass index; MRI=magnetic resonance imaging; LVOT=left ventricular outflow tract; nHCM=non-obstructive hypertrophic cardiomyopathy; NT-proBNP=N-terminal prohormone brain natriuretic peptide; NYHA=New York Heart Association; oHCM=obstructive hypertrophic cardiomyopathy; PGI-C=Patient Global Impression of Change; PK=pharmacokinetic(s); PRO=patient-reported outcome; pVO₂=peak oxygen uptake; SAQ-7=Seattle Angina Questionnaire-7; SBP=systolic blood pressure; VAT=ventilatory anaerobic threshold; VE/VCO₂=minute ventilation/carbon dioxide production.

### Example 1

The first-in-human study of aficamten (also referred to as aficamten) was undertaken to evaluate its safety, pharmacokinetic, and pharmacodynamic profile, including effects of food or a CYP2D6 poor metabolizer (CYP2D6-PM) phenotype and was described as Example 1 in WO 2023/288324 and in U.S. Patent Publication US-2023-0058927-A1, the disclosures of which are incorporated by reference herein.

### Example 2

A multi-center, randomized, placebo-controlled, double-blind, dose finding phase 2 clinical trial of aficamten in patients with symptomatic non-obstructive HCM (nHCM) or HCM with MVO was conducted and described as Example 2 in WO 2024/020468 and in U.S. Patent Publication US-2024-0091203-A1, the disclosures of which are incorporated by reference herein.

### Example 3

An open-label extension clinical trial of aficamten in patients with symptomatic nHCM or HCM with MVO is described as Example 3 in WO 2024/020468 and in U.S. Patent Publication US-2024-0091203-A1, the disclosures of which are incorporated by reference herein. The treatment duration is anticipated to be multiple years. The primary objective of the trial is to determine the safety and tolerability of aficamten over a 5-year period.

### Example 4

A Phase 3, multi-center, randomized, double-blind, placebo-controlled trial in participants with symptomatic nHCM was described as Example 4 in WO 2024/020468 and in U.S. Patent Publication US-2024-0091203-A1, the disclosures of which are incorporated by reference herein.

### Example 5

A multi-center, randomized, placebo-controlled, double-blind, dose finding phase 2 clinical trial of aficamten in patients with symptomatic obstructive HCM (oHCM) was conducted and described as Example 2 in WO 2024/031016 and in U.S. Patent Publication US-2024-0115554-A1, the disclosures of which are incorporated by reference herein.

### Example 6a

An open-label extension clinical trial of aficamten in patients with symptomatic oHCM was initiated and was described as Example 3a in WO 2024/031016 and in U.S. Patent Publication US-2024-0115554-A1, the disclosures of which are incorporated by reference herein. The primary objective of the trial was to determine the safety and tolerability of aficamten over a 5 year period.

### Example 6b

An open-label extension clinical trial of aficamten in patients with symptomatic oHCM was initiated and was described as Example 3b in WO 2024/031016 and in U.S. Patent Publication US-2024-0115554-A1, the disclosures of which are incorporated by reference herein. The treatment duration is anticipated to be multiple years. The primary objective of the trial is to determine the safety and tolerability of aficamten over a 5-year period.

Safety and efficacy of aficamten in patients with nonobstructive hypertrophic cardiomyopathy (nHCM). Thirty-four patients were enrolled (mean age±SD 57.2±15.3 years, 62% women). At baseline, patients were symptomatic (41% NYHA class III) with median (IQR) NT-proBNP and hs-cTnI of 1190 μg/mL (735, 1735) and 24.9 ng/L (9.7, 46.4), respectively. By the end of the titration period (week 6), 3 (8.8%), 3 (8.8%), 6 (17.6%) and 22 (64.7%) patients received doses of 5, 10, 15, and 20 mg daily, respectively. At baseline, LVEF (mean±SD) was 70%±6.1; over 36-weeks, there was a modest reduction of 4.3%±5.2 (p<0.0001). NYHA class improved by ≥1 class in 27 (79.4%) patients, including 20 (58.8%) were asymptomatic (NYHA class I) by week 36. The mean KCCQ-CSS improved by 13.8±12.52 points relative to baseline, with 24/31 (77.4%) patients reporting improvements of ≥5 points. NT-proBNP levels were reduced by week 36 compared with baseline (median [IQR] change; −665.5 pg/mL [−1244.0, −232.0], (p<0.0001) while hs-cTnI was unchanged (−2.7 ng/L [−11.3, 1.6], p=ns). Over the study period, aficamten appeared to be well tolerated and there were no drug discontinuations due to adverse events (AE). LVEF<50% occurred in 2 (5.9%) patients, one following pulmonary vein isolation and one associated with atrial fibrillation. There were no permanent discontinuations.

In this 36-week analysis of symptomatic patients with nHCM from FOREST-HCM (NCT04848506), aficamten was well tolerated and associated with reduced heart failure symptoms, improved functional class, and decreased cardiac biomarkers. Most patients achieved the highest available dose while maintaining normal LVEF.

Safety and Outcomes of Standard of Care Medications Withdrawal in Patients with Obstructive Hypertrophic Cardiomyopathy (oHCM) Treated with Aficamten in FOREST-HCM Trial. Standard of care (SoC) medications for the treatment of obstructive hypertrophic cardiomyopathy (oHCM) are currently recommended as first-line therapy. There are, however, off-target side-effects that often render them poorly tolerated.

Patients were classified as receiving SoC therapy (both first line and second line) if treated with at least one drug (beta-blocker [BB], non-dihydropyridine calcium channel blocker [CCB], and/or disopyramide). Patients were eligible for protocol-guided SoC withdrawal after receiving a stable dose of aficamten for ≥4-weeks and after Week 12 of the study, at the discretion of the site investigator. Successful SoC withdrawal was defined as at least a 50% dose-reduction in one medication relative to baseline. All patients were also required to have ≥1 clinical, echocardiographic and biomarker assessment after attempted withdrawal. Adverse events (AE) were prospectively evaluated 1-2 weeks after any SoC withdrawal. A cohort of patients receiving aficamten who did not undergo SoC withdrawal was used as a comparator.

Interim results show that, of the 145 oHCM patients followed for at least 24 weeks (mean age 60.5±13.22 years, 44.8% female, 42% NYHA Class III) 136 (93.8%) were receiving at least one SoC therapy (BB (84[61.8%]), CCB (15[11.0%]), BB+CCB (9[6.6%]), BB+Disopyramide (23[16.9%]), CCB+Disopyramide (2[1.5%]) and BB+CCB+Disopyramide (3[2.2%]). Of the 68 (50%) who underwent withdrawal attempt, 63 (92.6%) achieved successful withdrawal, with 42 (61.8%) patients completely discontinuing at least one SoC medication, and 30 (44.1%) of those achieving aficamten monotherapy (9[13.2%] monotherapy patients later restarted a SoC medication). Independent of attempted withdrawal, patients maintained relief of obstruction, improvement in symptoms and health status, and low cardiac biomarkers (Table 1). In the group with successful SoC withdrawal, no clinically significant differences were observed between pre and post SoC withdrawal attempt (Table 1). Overall, there was a modest reduction in left ventricular ejection fraction (LVEF) of −3.5%±6.0 from baseline, also with no differences between groups. There were no serious AEs attributed to SoC withdrawal and treatment emergent AEs were comparable between the two groups.

Patients with oHCM receiving SoC medications in FOREST-HCM frequently underwent successful withdrawal of these medications while on aficamten treatment without meaningfully negatively impacting hemodynamics, symptoms or cardiac biomarkers compared to pre-withdrawal, nor compared with patients who did not attempt SoC withdrawal.

TABLE 1
Key Parameters by SoC Withdrawal Group

| Successful SoC Withdrawal Group | | |
|---|---|---|
| Pre SoC | Post SoC | Change |
| withdrawal | withdrawal | (95% CI, p-value) |

Biomarkers

| | | | |
|---|---|---|---|
| NT-proBNP | 330.0 | 250.0 | −68.0 |
| (pg/mL) median | (131.0, 711.0) | (78.0, 485.0) | (−123.4, −12.6) |
| [Q1, Q3] | | | 0.02 |
| hs-cardiac-TaI | 5.8 | 5.8 | 0.0 |
| (ng/L) median | (3.5, 16.1) | (3.5, 10.6) | (−0.9, 0.9) |
| [Q1, Q3] | | | 1.00 |

Hemodynamics

| | | | |
|---|---|---|---|
| LVEF* (%), | 64.8 (5.8) | 65.5 (6.4) | 0.6 |
| Mean (SD) | | | (−0.7, 2.0), |
| | | | 0.34 |
| LVOT-G*, Rest | 13.2 (11.0) | 16.6 (13.7) | 3.4 |
| (mmHg), Mean | | | (−0.4, 7.2), |
| (SD) | | | 0.08 |
| LVOT-G*, | 27.9 (22.4) | 35.6 (29.1) | 7.7 (0.8, 14.6), |
| Valsalva | | | 0.03 |
| (mmHg), Mean | | | |
| (SD) | | | |

Symptoms

| NYHA class, | I: 23 (41.1%) | I: 26 (46.4%) | | |
|---|---|---|---|---|
| n(%) | II: 25(44.6%) | II: 26 (46,4%) | | |
| | III: 8(14.3%) | III: 4 (7.1%) | | |
| | IV: 0 | IV: 0 | | |
| KCCQ CSS, | 80.2 (17.20) | 83.8 (18.18) | 3.5 (−0.5, 7.5) | |
| Mean (SD) | | | 0.08 | |
| | No SoC Withdrawal Group | | | |
| | | | | Δ change |
| | | | | between |
| | | | | cohorts at |
| | | | | Week 24 |
| | | | Change | (95% CI, |
| | Pre | Post | (95% CI, | P-value |
| | (Week 12) | (Week 24) | p-value) | ** ) |

Biomarkers

| | | | | |
|---|---|---|---|---|
| NT-proBNP | 191.5 | 158.5 | −3.0 | −65.0 |
| (pg/mL) median | (77.0, 534.0) | (76.0, | (−24.3, | (−124.7, |
| [Q1, Q3] | | 362.0) | 18.3) | −5.3) |
| | | | 0.78 | 0.03 |
| hs-cardiac-TnI | 7.6 | 6.3 | −0.5 | 0.5 |
| (ng/L) median | (4.0, 15.9) | (3.5, 12.8) | (−1.5, 0.5) | (−0.8, |
| [Q1, Q3] | | | 0.33 | 1.8) |
| | | | | 0.46 |

Hemodynamics

| | | | | |
|---|---|---|---|---|
| LVEF* (%), | 65.3 (4.9) | 64.4 (5.3) | −0.9 | 1.5 |
| Mean (SD) | | | (−2.2, 0.4) | (−0.3, |
| | | | 0.17 | 3.3) |
| | | | | 0.10 |
| LVOT-G*, Rest | 19.5 (17.6) | 14.0 (12.6) | −5.5 | 8.9 (3.7, |
| (mmHg), Mean | | | (−9.0, −1.9) | 14.0) |
| (SD) | | | 0.003 | 0.0009 |
| LVOT-G*, | 42.2 (26.0) | 32.2 (25.2) | −10.0 | 17.7 (8.6, |
| Valsalva | | | (−16.2, | 26.8) |
| (mmHg), Mean | | | −3.9) | 0.0002 |
| (SD) | | | 0.002 | |

Symptoms

| | | | | |
|---|---|---|---|---|
| NYHA class, | I: 34 (53.19%) | I: 35 (54.7%) | | |
| n(%) | II: 27 42.2%) | II: 27 42.2%) | | |
| | III: 3 (4,7%) | III: 2 (3.1%) | | |
| | IV: 0 | IV: 0 | | |
| KCCQ CSS, | 87.3 (13.12) | 88.5 (12.55) | 1.2 (−1.0, | 2.3 (−2.1, |
| Mean (SD) | | | 3.5), | 6.7), |
| | | | 0.28 | 0.30 |

*site read echo results
** P value comparing change in the SoC successful withdrawal cohort vs change in controls (no successful SoC withdrawal).

### Example 7

A Phase 3, multi-center, randomized, double-blind, active-comparator trial in participants with symptomatic oHCM and elevated LVOT-G was described as Example 4 in WO 2024/031016 and in U.S. Patent Publication US-2024-0115554-A1, the disclosures of which are incorporated by reference herein.

### Example 8

The following example describes a phase 3, multi-center, randomized, double-blind, placebo-controlled trial to evaluate the efficacy and safety of aficamten in adults with symptomatic hypertrophic cardiomyopathy and left ventricular outflow tract obstruction. This trial evaluated the effects of treatment with aficamten over a 24-week period on cardiopulmonary exercise capacity and health status in patients with symptomatic oHCM. This trial was intended to establish the efficacy and safety of aficamten with respect to improvements in exercise capacity and patient symptoms, as well as reduction in left ventricular outflow tract gradient (LVOT-G) in patients with oHCM.

This was a Phase 3 randomized, placebo-controlled, double-blind, multi-center trial in patients with symptomatic oHCM. Approximately 270 eligible patients were to be randomized in a 1:1 ratio to receive aficamten or placebo. Doses of 5, 10, 15, or 20 mg or matching placebo were administered in an escalating manner using echocardiography to guide dose titration. Randomization was stratified by use of beta blockers and CPET exercise modality.

The trial comprised three periods. The screening period was up to 6 weeks in duration. The double-blind placebo-controlled treatment period lasted 24 weeks. Following the final dose of aficamten, there was a 4-week safety follow-up period. Aficamten was administered orally once daily. During the initial six weeks of the treatment period, aficamten doses were individually titrated at Weeks 2, 4, and 6 using echocardiography. Dose escalation at the Weeks 2, 4, and 6 visits occurred only if a patient had a post-Valsalva LVOT-G≥30 mmHg and a biplane LVEF≥55%. An echocardiogram was performed at each subsequent visit during the trial and the dose down-titrated if necessary. The primary endpoint of pVO₂ was measured by CPET at screening and at end of treatment (Week 24). If applicable, patients continued taking background HCM medications consistent with regional clinical practice guidelines during the trial.

A CMR imaging sub-study was open to approximately 40 patients who consented to participate.

Genetics sub-study: Patients who consented had DNA analyzed using whole-genome sequencing, whole-exome sequencing, next-generation sequencing, and/or other methods to identify genetic variants.

The main mitigation strategy was facilitated by an individualized dose titration scheme based on each patient's PD response to aficamten with application of pre-specified echocardiographic criteria, including LVEF thresholds for dose escalation, down-titration, and drug discontinuation.

Patients enrolled in this trial were required to have an LVEF≥60% prior to randomization, as confirmed by the central echocardiography laboratory. A low starting dose of 5 mg and a maximum dose of 20 mg were chosen as these were found to be well-tolerated in the Phase 2 study (CY 6021) of patients with oHCM and effective at reducing the LVOT-G without adversely impacting overall LVEF. Dose escalation was performed on an individualized basis only if the following criteria were met: both post-Valsalva LVOT-G≥30 mmHg and biplane LVEF≥55%. Importantly, in contrast to CY 6021, the lower limit of LVEF for dose escalations was increased from 50% to 55% to provide a safety margin from the threshold of LVEF (<50%) that would trigger dose reduction. If the LVEF was <50% at any time, the dose of aficamten was down-titrated, and if the LVEF is <40% at any time, aficamten was temporarily interrupted.

The primary objective of this trial was to evaluate the effect of aficamten on exercise capacity in patients with symptomatic oHCM. The indicated endpoint was a change in peak oxygen uptake (pVO₂) by cardiopulmonary exercise testing (CPET) from baseline to Week 24.

A secondary objective of this trial was to evaluate the effect of aficamten on patient health status, as determined by changes in the Kansas City Cardiomyopathy Questionnaire-Clinical Summary Score (KCCQ-CSS) from baseline to Week 12 and Week 24.

A further secondary objective of this trial was to evaluate the effect of aficamten on New York Heart Association (NYHA) Functional Classification, as determined by the proportion of patients with ≥1 class improvement in NYHA Functional Class from baseline to Week 12 and Week 24.

A further secondary objective was to evaluate the effect of aficamten on post-Valsalva left ventricular outflow tract gradients (LVOT-G), as determined by change in post-Valsalva LVOT-G from baseline to Week 12 and Week 24, and the proportion of patients with post-Valsalva LVOT-G<30 mmHg at Week 12 and Week 24.

A further secondary objective was to evaluate the effect of aficamten on exercise capacity, as determined by change in total workload during CPET from baseline to Week 24.

To evaluate the safety and tolerability profile of aficamten in patients with symptomatic oHCM, the following were recorded: (1) incidence of reported major adverse cardiac events (cardiovascular [CV] death, cardiac arrest, non-fatal stroke, non-fatal myocardial infarction, CV hospitalization); (2) incidence of new onset persistent atrial fibrillation; (3) incidence of appropriate implantable cardiac defibrillator (ICD) discharges and aborted sudden cardiac death; (4) incidence of left ventricular ejection fraction (LVEF)<50%; and (5) incidence of treatment emergent adverse events.

An exploratory objective of this trial was to evaluate the effect of aficamten on exercise capacity and functional class, as determined by comparing with baseline, the number of patients at Week 24 achieving either (1) change from baseline of ≥1.5 mL/kg/min in pVO₂ AND ≥1 class improvement in NYHA Functional Class; or (2) Change from baseline of ≥3.0 mL/kg/min in pVO₂ AND no worsening of NYHA Functional Class.

A further exploratory objective of this trial was to evaluate the effect of aficamten on patient response over time, as determined by (1) proportion of patients with improvement in KCCQ-CSS>5 points at Weeks 12 and 24; (2) proportion of patients with resting LVOT-G<30 mmHg, post-Valsalva LVOT-G<50 mmHg, and NYHA Functional Class I at Weeks 12 and 24; and (3) proportion of patients with resting LVOT-G<30 mmHg, post-Valsalva LVOT-G<50 mmHg, and >1 class improvement in NYHA Functional Class at Weeks 12 and 24.

A further exploratory objective of this trial was to evaluate the effect of aficamten on other CPET parameters, as determined by changes from baseline to Week 24 in: (1) ventilator efficiency (VE/VCO₂ slope); (2) circulatory power (VO₂× systolic BP); and (3) ventilator anaerobic threshold (VAT).

A further exploratory objective of this trial was to evaluate the effect of aficamten on health status and health-related quality of life as measured by PRO questionnaire, as determine by changes from baseline to Week 24 in individual responses to the EuroQol 5-dimension 5-level instrument (EQ-5D-5L).

A further exploratory objective of this trial was to evaluate the effect of aficamten on cardiac function and structure, as determined by change from baseline to Week 24 in echocardiographic measurements of cardiac structure and of systolic function including: LVEF, left ventricular end-systolic and end-diastolic volumes (LVESV and LVEDV, respectively), and left atrial volume.

A further exploratory objective of this trial was to evaluate the effect of aficamten on biomarker levels, as determined by changes from baseline values in NT-pro-BNP, hs-cardiac-TnI and other biomarkers through Week 24.

A further exploratory objective of this trial was to evaluate the effect of aficamten on left ventricular mass, function, and structure by cardiac magnetic resonance (CMR) imaging, as determined by changes from baseline to Week 24 in CMR measurements of left ventricular (LV) mass index, LVEF, septal and free wall thickness, left arterial volume index, LVESV, and LVEDV.

A further exploratory objective of this trial was to assess the pharmacokinetics of aficamten and its metabolites, as determined by pharmacokinetic parameters through Week 24.

Overall Design. This was a Phase 3, randomized, placebo-controlled, double-blind, multi-center trial in patients with symptomatic oHCM. Approximately 270 eligible patients were randomized in a 1:1 ratio to receive aficamten or placebo. Randomization was stratified by use of beta blockers (yes or no) and CPET exercise modality (treadmill or bicycle) and implemented in the Interactive Web Response System (IWRS). A cap on the number of patients taking beta blockers did not exceed approximately 70% of total enrollment. The number of patients with persistent atrial fibrillation at screening was also capped at approximately 15%, and the number of patients using the bicycle CPET exercise modality will be capped at approximately 50% as well.

Aficamten was administered orally once daily with or without food. During the initial six weeks of the treatment period, aficamten doses were individually titrated at Weeks 2, 4, and 6 using echocardiography. Dose escalation at Weeks 2, 4, and 6 occurred only if a patient had a post-Valsalva LVOT-G≥30 mmHg and a biplane LVEF≥55%. Echocardiograms were performed at each subsequent visit during the trial and the dose down titrated if necessary. The primary endpoint of pVO₂ was measured by CPET at screening and at end of treatment (Week 24). If applicable, patients continued taking background HCM medications consistent with regional clinical practice guidelines during the trial.

All patients were followed according to the Schedule of Activities (SoA) (see trial design, FIG. 1) from randomization through the date of their final visit irrespective of whether the patient continued to receive aficamten, unless the patient had discontinued prematurely from the trial or withdrawn consent. An early discontinuation visit was performed for patients that discontinue prematurely from the trial.

This trial was designed to provide data supporting the clinical efficacy and safety of aficamten in patients with symptomatic oHCM and an LVOT-G≥50 mmHg post-Valsalva. Reduction of the LVOT-G was expected to correlate with improvement in the patients' symptoms, health status and exercise capacity. Since patient characteristics vayy substantially in this disease, individualized dose titration to a PD response (reduction of the post-Valsalva LVOT-G to <30 mmHg with preservation of LVEF≥55%) was being employed to maximize efficacy and safety. The eligibility criteria were designed to enable enrollment of a patient population representative of the general population of patients with oHCM while ensuring the safety of the patients in this trial. A placebo control and double-blinded approach were being employed in this trial to avoid bias in data collection, including the safety assessments and PD measures that comprised the primary and secondary endpoints.

Patients were eligible to be included in the trial only if all the following criteria applied: (1) Able to comprehend and willing to sign an ICF and willing to comply with all trial procedures and restrictions for the duration specified in the Schedule of Activities. (2) Males and females between 18 and 85 years of age, inclusive, at screening. (3) Body mass index <35 kg/m². (4) Diagnosed with HCM per the following criteria: (a) has LV hypertrophy and non-dilated LV chamber in the absence of other cardiac disease, and (b) has an end-diastolic LV wall thickness as measured by the echocardiography core laboratory of: ≥15 mm in one or more myocardial segments OR ≥13 mm in one or more wall segments and a known-disease-causing gene mutation or positive family history of HCM. (5) Had resting LVOT-G≥30 mmHg and post-Valsalva LVOT-G≥50 mmHg during screening as determined by the echocardiography core laboratory. (6) LVEF≥60% at screening was determined by the echocardiography core laboratory. (7) New York Heart Association (NYHA) Functional Class II or III at screening. (8) Hemoglobin ≥10 g/dL at screening. (9) Respiratory exchange ratio (RER) ≥1.05 and pVO2<80% predicted on the screening CPET per the core laboratory. (10) Patients on beta blockers, verapamil, or diltiazem should have been on a stable regimen for >6 weeks prior to randomization and anticipate remaining on the same medication regimen during the trial. (11) Male patients were eligible to participate if they agree to the following during the trial and for at least 4 weeks after the last dose of aficamten: (a) Refrain from donating sperm, plus (b) either (i) be abstinent from heterosexual intercourse as their preferred and usual lifestyle (abstinent on a long term and persistent basis) and agree to remain abstinent, or (ii) must agree to use a male condom and, when his female partner is a woman of childbearing potential, have his female partner use a highly effective method of contraception. (12) a female patient was eligible to participate if she was not pregnant, breastfeeding or planning to donate eggs, and at least one of the following conditions applied: (a) is not a woman of childbearing potential (WOCBP), or is a WOCBP and using a highly effective method of contraception and male partner agrees to use a condom, during the trial and for at least 4 weeks after the last dose of aficamten, and (b) a WOCBP must have a negative pregnancy test (urine or serum as required by local regulations) at Day 1, prior to the first dose of study aficamten. (13) Able to complete all screening procedures.

Patients were excluded from the trial if any of the following criteria apply: (1) Significant valvular heart disease (per investigator judgment), including moderate-severe valvular aortic stenosis and/or regurgitation, or moderate-severe mitral regurgitation not due to systolic anterior motion of the mitral valve. (2) Documented history of current obstructive coronary artery disease (>70% stenosis in one or more epicardial coronary arteries) or documented history of myocardial infarction. (3) Known or suspected infiltrative, genetic or storage disorder causing cardiac hypertrophy that mimics oHCM (e.g., Noonan syndrome, Fabry disease, amyloidosis). (4) Prior treatment with cardiotoxic agents such as doxorubicin or similar. (5) History of LV systolic dysfunction (LVEF<45%) or stress cardiomyopathy at any time during their clinical course. (6) Has any ECG abnormality considered by the investigator to pose a risk to patient safety (e.g., second degree atrioventricular block type II). (7) Documented paroxysmal atrial fibrillation during the screening period. (8) Paroxysmal or permanent atrial fibrillation requiring rhythm restoring treatment (eg, direct-current cardioversion, atrial fibrillation ablation procedure, or antiarrhythmic therapy) ≤6 months prior to screening. (This exclusion does not apply if atrial fibrillation has been treated with anticoagulation and adequately rate-controlled for >6 months.) (9) History of syncope or sustained ventricular tachyarrhythmia with exercise within 6 months prior to screening. (10) ICD placement within 3 months prior to screening or planned ICD placement during the trial. (11) History of appropriate ICD discharge for life-threatening ventricular arrhythmia within 6 months prior to screening. (12) Has been treated with septal reduction therapy (surgical myectomy or percutaneous alcohol septal ablation) or has plans for either treatment during the trial period. (13) Inability to exercise on a treadmill or bicycle (e.g., orthopedic limitations). (14) Documented room air oxygen saturation reading <90% at screening. (15) Hepatic impairment defined by a total bilirubin (TBL)≥1.5× the upper limit of normal (ULN), or alanine aminotransferase (ALT) or aspartate aminotransferase (AST)≥3×ULN at screening. Patients with documented Gilbert syndrome and TBL≥1.5×ULN due to unconjugated hyperbilirubinemia, without other hepatic impairment, are permitted. (16) Recipient of a major organ transplant (eg, heart, lung, liver, bone marrow, renal) or anticipated transplantation within 12 months from randomization. (17) History or evidence of any other clinically significant disorder, malignancy, active infection, other condition, or disease that, in the opinion of the investigator or the Medical Monitor, would pose a risk to patient safety or interfere with the trial evaluation, procedures, or completion. (18) Estimated glomerular filtration rate (cGFR)<30 mL/min/1.73 m2 (by the modified Modification of Diet in Renal Disease equation) at screening. (19) Concurrently participating in another investigational device or drug trial or received an investigational device or drug <1 month (or 5 half-lives for drugs, whichever is longer) prior to screening. Other investigational procedures while participating in this trial were not permitted. (20) Has received prior treatment with aficamten or mavacamten. (21) Any known hypersensitivity to excipients in study drug tablets (e.g., mannitol, microcrystalline cellulose, croscarmellose, hydroxypropyl cellulose, sodium lauryl sulfate, magnesium stearate, Opadry QX White 21A180025).

Exclusion Criteria for CMR sub-study included (1) inability to tolerate CMR, (2) had an ICD, or (3) had a cardiac pacemaker.

Dose modifications and scheduled dose titrations. Patients randomized to aficamten received up to four escalating doses of aficamten over the initial 6 weeks of the trial as outlined in Table 2 and FIG. 1. Patients receiving aficamten started at a dose of 5 mg once daily (Dose 1) and escalated through doses of 10, 15, and 20 mg once daily if they continued to meet the escalation criteria or stopped at their current dose when escalation criteria were not met.

TABLE 2
Echocardiogram Criteria for Scheduled Dose Titrations

| | | Post-Valsalva | |
|---|---|---|---|
| Biplane LVEF | | LVOT-G | Action |
| <50% | | | Reduce Dose^{a} |
| ≥50%-55% | | | No Dose Change |
| ≥55% | and | <30 mmHg | No Dose Change |
| ≥55% | and | ≥30 mmHg | Increase Dose |

^{a}Once a patient′s aficamten dose was down titrated, no further escalation was permitted. If LVEF <50% on 5 mg, the patient received placebo.

After randomization, each patient received Dose 1 (5 mg) once daily for two weeks. At the Week 2 visit, the patient had an echocardiogram 2 hours following administration of their dose of aficamten. Patients up-titrated to Dose 2 (10 mg) if the following conditions were met on echocardiography: Post-Valsalva LVOT-G≥30 mmHg, and the biplane LVEF≥55%. Otherwise, the patient remained on Dose 1, except that if LVEF was <50% at Week 2, the IWRS assigned the patient to placebo.

After two more weeks on the assigned dose, at the Week 4 visit each patient had an echocardiogram 2 hours following administration of their dose of aficamten. Patients up-titrated to the next higher dose if the following conditions were met on echocardiography: Post-Valsalva LVOT-G≥30 mmHg, and the biplane LVEF≥55%. Otherwise, the patient remained on the same dose, except that if LVEF is <50% at Week 4, the IWRS assigned the patient to the prior dose level or to placebo if the patient was on Dose 1.

After 2 more weeks on the assigned dose, at the Week 6 visit each patient had an echocardiogram 2 hours following administration of their dose of aficamten. Patients up-titrated to the next higher dose if the following conditions were met on echocardiography: post-Valsalva LVOT-G≥30 mmHg, and the biplane LVEF≥55%. Otherwise, the patient remained on the same dose, except that if LVEF is <50% at Week 6, the IWRS assigned the patient to the prior dose level or to placebo if the patient was on Dose 1.

After two additional weeks on the assigned dose, at the Week 8 visit each patient had an echocardiogram 2 hours following administration of their dose of aficamten to ensure the LVEF is ≥50%. If the LVEF is <50% at Week 8, the IWRS assigned the patient to the next lower dose or to placebo if the patient was on Dose 1.

After Week 6, no further dose escalations occurred. During the course of the study, for safety reasons, dose reductions would occur at scheduled or unscheduled visits. Dose reductions is determined by the IWRS system based on echocardiography results. After Week 8, dose reductions were based on echocardiogram results from the initial scheduled or unscheduled visits. If the LVEF was <50%, then the IWRS assigned the patient to the next lower dose or to placebo if the patient was on Dose 1. The IWRS did not further reduce the dose for at least seven days after the previous reduction.

Cardiopulmonary Exercise Testing (CPET). All patients underwent CPET with gas-exchange analysis and the methodology will be standardized across all participating sites, as described in the CPET manual. Testing included continuous ECG monitoring by trained personnel and was performed in an area that was equipped for cardiopulmonary resuscitation. Treadmill was the preferred modality for exercise testing. For CPET laboratories that did not perform treadmill testing, cycle ergometry was an acceptable alternative. Exercise protocols for both modalities were provided in the CPET manual. Patients had to use the same testing modality for all exercise tests during the trial. Whenever possible, CPET was administered by the same trial personnel using the same equipment and performed after the other trial procedures on that visit day (including echocardiogram, KCCQ, EQ-5D-5L, CGI, PGI-C, NYHA class, SAQ-7, vital signs, ECG, blood sampling, IP administration). Patients naïve to exercise protocols were familiarized with the technique during screening.

All CPET testing was symptom-limited and patients were strongly encouraged to achieve maximal exertion and an RER ≥1.05. The reason(s) for termination of sub-maximal exercise tests were documented. A test was identified as being maximal effort if the RER is ≥1.05.

The Week 24 CPET was performed at approximately the same time of day (eg, morning, mid-day, afternoon) as the baseline CPET at screening, at a consistent time after the last dose of beta blocker and IP. Whenever possible, patients performed exercise testing between three and ten hours after taking beta blocking agents.

If a life-threatening arrhythmia, early ischemia, severe hypotension or other serious finding was identified by the investigator during CPET, the patient was asked to stop the exercise test, and his/her physicians notified of the results. If the patient was performing the screening test, s/he was not randomized to the trial. Enrolled patients who had a non-life-threatening event or finding that stopped the test could resume testing when it was safe to do so and after appropriate treatment, per the investigator.

Echocardiography. Echocardiography was done during screening and prior to dosing on Day 1. Echocardiography was also performed 2 hours after dosing in the clinic on Weeks 2, 4, 6, 8, 12, 16, 20, 24, and 28.

Certified sonographers performed echocardiography using standard high-quality, high-fidelity machines. Whenever possible, the same sonographer performed all studies for a single patient. Echocardiograms were performed after the patient had been resting in a supine position for at least 10 minutes and in accordance with the echocardiography manual. Instructions for the performance of the Valsalva maneuver and imaging the LVOT-G were also included in the echocardiography manual.

When echocardiograms were scheduled at the same time as blood draws, vital signs, and/or ECGs, the order of evaluation was vital signs, ECGs, blood draw and echocardiogram. The blood draw was obtained at the scheduled time point and the echocardiograms followed.

Echocardiographic parameters measured at least included the left ventricular parameters (resting left ventricular outflow tract pressure gradient (LVOT-G), post-Valsalva LVOT-G, LVEF, LVFS, global longitudinal strain (GLS), left ventricular end diastolic diameter (LVEDD), left ventricular end diastolic volume (LVEDV), left ventricular end systolic diameter (LVESD), left ventricular end systolic volume (LVESV), left ventricular cardiac output (LVCO), LV Stroke Volume, LVOT velocity time integral (VTI), interventricular septum thickness (IVST), isovolumic contraction time (IVCT), IVRT, E/E, ratio (septal and lateral), and left atrial volume (LAV)) in addition to right heart function metrics detailed in the echocardiography protocol.

Unscheduled echocardiograms could be obtained when clinically indicated, for example to assess an AE or follow-up a clinically significant change in a prior echocardiogram, as determined by the investigator. Results were interpreted by the unblinded Echo Cardiologist at the investigational site.

All echocardiograms (including unscheduled) were sent to the core laboratory for interpretation. On-site interpretation of LVEF and LVOT-G were used for dose escalation and reduction decisions via IWRS. The core laboratory quantification of the echocardiograms was used for all statistical analyses.

Baseline echocardiography demonstrated findings consistent with oHCM with a mean maximum LV wall thickness of 21±3 mm with greater interventricular septal wall thickness compared to the inferolateral wall (Table 3). Abnormal mean measures of diastolic function included reduced e′ velocities, elevated E/e′, and mildly dilated LA. LV dimensions were within normal range with generally hyperdynamic LVEF (75%±6). At baseline, the majority of patients (91% [228/250], missing data n=32) demonstrated mitral valve systolic anterior motion (SAM). LV GLS was mildly reduced, while LV GCS was normal to increased. Measures of RV systolic function (TAPSE, RV s′ velocity) were within normal range (Table 3).

TABLE 3
Baseline and Change in Echocardiographic Parameters

| | Placebo | Aficamten |
|---|---|---|
| | (n = 140) | (n = 142) |
| Echo variable | Baseline | Baseline |
| LVOT Gradients | | |
| LVOT Gradient, rest, mmHg | 55 ± 32 | 55 ± 27.0 |
| LVOT Gradient, Valsalva, mmHg | 83 ± 33 | 83 ± 32 |
| LV Structure | | |
| Max wall thickness, mm | 21.0 ± 3.0 | 20.7 ± 3.0 |
| Interventricular septal wall, mm | 19.4 ± 3.3 | 18.9 ± 2.9 |
| Inferolateral wall, mm | 13.2 ± 2.9 | 12.5 ± 2.6 |
| LV mass index, g/m2 | 134.6 ± 36.6 | 129.6 ± 31.0 |
| LV end diastolic dimension, mm | 38.8 ± 5.9 | 39.4 ± 5.1 |
| LV end systolic dimension, mm | 21.7 ± 4.1 | 21.9 ± 3.8 |
| LV end diastolic volume index, ml/m2 | 36.0 ± 9.2 | 35.9 ± 7.8 |
| LV end systolic volume index, ml/m2 | 9.1 ± 3.8 | 9.1 ± 2.9 |
| LV Systolic Function | | |
| LV Ejection Fraction, % | 75 ± 6 | 75 ± 6 |
| LV Fractional Shortening, % | 44 ± 8 | 45 ± 8 |
| LV global longitudinal strain*, % | 15.3 ± 3.3 | 15.4 ± 3.1 |
| LV global circumferential strain*, % | 33.4 ± 8.1 | 33.0 ± 7.1 |
| RV Systolic Function | | |
| TAPSE, mm | 21 ± 4 | 21 ± 4 |
| RV s′ velocity, cm/s | 13.2 ± 2.4 | 12.7 ± 2.5 |
| LV Diastolic Function | | |
| LA volume index, ml/m2 | 40.9 ± 15.1 | 40.1 ± 12.7 |
| LA width, mm | 42 ± .6 | 42 ± 6 |
| Peak E wave velocity, cm/s | 87.4 ± 32.0 | 82.2 ± 24.5 |
| Peak A wave velocity, cm/s | 83.6 ± 29.9 | 81.7 ± 27.8 |
| Lateral e′ velocity, cm/s | 6.1 ± 2.2 | 6.0 ± 2.0 |
| Septal e′ velocity, cm/s | 4.6 ± 1.6 | 4.6 ± 1.4 |
| Lateral E/e′ | 15.9 ± 7.8 | 15.4 ± 7.3 |
| Septal E/e′ | 20.5 ± 9.3 | 19.5 ± 8.4 |
| | Treatment (Week 24) | |
| | Placebo-corrected | |
| | Treatment | |
| | Difference | |
| Echo variable | (95% CI) | P value^{†} |
| LVOT Gradients | | |
| LVOT Gradient, rest, mmHg | −40 (−46, −34) | <0.001 |
| LVOT Gradient, Valsalva, mmHg | −50 (−57, −44) | <0.001 |
| LV Structure | | |
| Max wall thickness, mm | −1.2 (−1.8, −0.6) | <0.001 |
| Interventricular septal wall, mm | −1.0 (−1.6, −0.3) | 0.003 |
| Inferolateral wall, mm | −0.8 (−1.3, −0.3) | 0.003 |
| LV mass index, g/m2 | −12.2 (−18.0, −6.5) | <0.001 |
| LV end diastolic dimension, mm | −0.3 (−1.1, 0.5) | 0.5 |
| LV end systolic dimension, mm | +1.6 (0.7, 2.4) | 0.001 |
| LV end diastolic volume index, ml/m2 | −0.2 (−1.5, 1.2) | 0.81 |
| LV end systolic volume index, ml/m2 | +1.7 (1.0, 2.4) | <0.001 |
| LV Systolic Function | | |
| LV Ejection Fraction, % | −5 (−6, −3) | <0.001 |
| LV Fractional Shortening, % | −4 (−7, −2) | <0.001 |
| LV global longitudinal strain*, % | −0.4 (−0.9, 0.1) | 0.13 |
| LV global circumferential strain*, % | −3.7 (−5.6, −1.8) | <0.001 |
| RV Systolic Function | | |
| TAPSE, mm | −2.1 (−3.2,−1.1) | <0.001 |
| RV s′ velocity, cm/s | −1.4(−2.0, −0.9) | <0.001 |
| LV Diastolic Function | | |
| LA volume index, ml/m2 | −3.8 (−5.5, −2.2) | <0.001 |
| LA width, mm | −2.7 (−3.8, −1.7) | <0.001 |
| Peak E wave velocity, cm/s | −7.5 (−11.7, −3.2) | 0.001 |
| Peak A wave velocity, cm/s | −2.4 (−6.3, 1.5) | 0.22 |
| Lateral e′ velocity, cm/s | 1.2 (0.7, 1.6) | <0.001 |
| Septal e′ velocity, cm/s | 0.5 (0.2, 0.8) | 0.001 |
| Lateral E/e′ | −3.9 (−5.0, −2.8) | <0.001 |
| Septal E/e′ | −3.6 (−4.8, −2.5) | <0.001 |
| | Washout (Week 28) | |
| | Placebo-corrected | |
| | Treatment | |
| | Difference | |
| Echo variable | (95% CI) | P value^{†} |
| LVOT Gradients | | |
| LVOT Gradient, rest, mmHg | −1 (−6, 7) | 0.83 |
| LVOT Gradient, Valsalva, mmHg | −1 (−8, 6) | 0.85 |
| LV Structure | | |
| Max wall thickness, mm | — | |
| Interventricular septal wall, mm | −0.3 (−1.0, 0.4) | 0.38 |
| Inferolateral wall, mm | −0.2 (−0.8, 0.3) | 0.43 |
| LV mass index, g/m2 | −3.7 (−10.2, 2.8) | 0.27 |
| LV end diastolic dimension, mm | −0.7 (−1.6, 0.2) | 0.14 |
| LV end systolic dimension, mm | −0.2 (−1.1, 0.8) | 0.72 |
| LV end diastolic volume index, ml/m2 | −1.2 (−2.5, 0.1) | 0.07 |
| LV end systolic volume index, ml/m2 | −0.1 (−0.7, 0.5) | 0.74 |
| LV Systolic Function | | |
| LV Ejection Fraction, % | −1 (−2, 1) | 0.21 |
| LV Fractional Shortening, % | −1 (−3, 2) | 0.62 |
| LV global longitudinal strain*, % | −0.1 (−0.6, 0.5) | 0.74 |
| LV global circumferential strain*, % | −0.8 (−2.5, 0.9) | 0.36 |
| RV Systolic Function | | |
| TAPSE, mm | +0.8 (−0.3, 1.9) | 0.16 |
| RV s′ velocity, cm/s | +0.3 (−0.2, 0.9) | 0.28 |
| LV Diastolic Function | | |
| LA volume index, ml/m2 | −3.0 (−4.9, −1.0) | 0.003 |
| LA width, mm | −0.8 (−1.8, 0.3) | 0.14 |
| Peak E wave velocity, cm/s | −1.6 (−5.8, 2.7) | 0.47 |
| Peak A wave velocity, cm/s | 3.2 (−0.5, 6.9) | 0.09 |
| Lateral e′ velocity, cm/s | −0.2 (−0.6, 0.3) | 0.45 |
| Septal e′ velocity, cm/s | −0.0 (−0.3, 0.2) | 0.96 |
| Lateral E/e′ | −0.2 (−1.6, 1.2) | 0.78 |
| Septal E/e′ | −0.6 (−1.7, 0.6) | 0.35 |

Baseline values are presented as mean±SD. Placebo corrected differences are presented as mean±95% CI and adjusted for treatment and stratification by beta-blockers and exercise mode (bicycle vs treadmill). Max wall thickness was not measured at week 28.
*Absolute strain values are presented.
^{†}P value for placebo-corrected treatment difference compared to baseline
e′=Early diastolic mitral annular velocity, E wave=Early mitral inflow velocity, E/e′=ratio between early mitral inflow velocity and early diastolic mitral annular velocity, LA=Left atrium, LV=Left ventricle, LVOT=Left ventricular outflow tract, RV=Right ventricle, s′=Peak systolic annular velocity, TAPSE=Tricuspid annular plane systolic excursion, TR=Tricuspid regurgitation.

In addition to significant improvements in resting and Valsalva LVOT gradients (−40 mmHg, −50 mmHg, respectively), aficamten treatment for 24 weeks resulted in a decrease in LA volume index (−3.8 ml/m2) and improvements in lateral and septal e′ velocities (+1.2 cm/s, +0.5 cm/s, respectively) and lateral and septal E/e′ (−3.9, −3.6, respectively) (all p<0.001, Table 3). LV end systolic volume increased (+1.7 ml/m2) while all wall thickness measurements decreased (all p≤0.003) (Table 3). Aficamten resulted in a mild decrease in LVEF [−4.8% (95% CI, −6.4 to −3.3), p<0.001] and absolute LV GCS [−3.7 (1.8, 5.6), p<0.001], with GCS approaching normal range, while LV GLS was not changed (Table 3). There was also a mild decrease in RV systolic function (TAPSE and RV s′); however, these measures remained within normal range. Several measures, including LVEF, LVOT gradients, and E/e′ returned to baseline following washout. Aficamten resulted in complete resolution of mitral valve SAM in approximately half of patients [52% (61/117), missing data n=17] with 82% (50/61) of this group reverting to baseline mitral valve SAM after washout.

Relationship Between Changes in pVO₂ and Other End Points with Changes in Echocardiographic Measures. Among those treated with aficamten, improvement in pVO₂ (per 1 ml/kg/min) over 24 weeks was associated with improvement in LA volume index (−0.46 ml/m2), peak A velocity (−1.17 cm/s), lateral e′ velocity (+0.16 cm/s), lateral E/e′ (−0.29), and septal E/e′ (−0.26) (all p<0.05). A reduction in NT-proBNP (per-log 2 change or 50% reduction) was significantly associated with reduction in resting and Valsalva LVOT gradients (−5.6 mmHg, −7.4 mmHg, respectively), reduction in maximum wall thickness and interventricular septal wall thickness (−0.50 mm, −0.06 mm, respectively), increase in lateral and septal e′ velocities (+0.53 cm/s, +0.26 cm/s, respectively), decrease in lateral and septal E/e′ (−1.1, −1.0, respectively), and increase in absolute LV GLS (0.43%) (all p<0.05). Improvement in KCCQ-CSS (per 5-point increase) was significantly associated with reduction in resting and Valsalva LVOT gradients (−2.7 mmHg, −3.8 mmHg, respectively), reduction in inferolateral wall thickness (−0.19 mm), and increase in LV end systolic dimension (+0.34 mm) (all p<0.05). A reduction in hsTnI (per-log 2 change or 50% reduction) was significantly associated with reduction in the Valsalva LVOT gradient (−6.6 mmHg), reduction in maximum wall thickness and interventricular septal wall thickness (−1.2 mm, −1.0 mm, respectively), and increase in septal e′ velocity (+0.39 cm/s) (all p<0.05). Changes in pVO₂, NT-proBNP, KCCQ-CSS, and hsTnI were not significantly associated with change in LV ejection fraction.

Cardiac Magnetic Resonance. A CMR imaging sub-study assessed the effects of administration of aficamten dosing on cardiac morphology, function, and fibrosis in approximately 40 oHCM patients who were eligible and consented to participate. CMR was performed during screening period and Week 24. Patients with EGFR <30 mL/min/1.73 m₂ or an allergy to gadolinium may have had a non-contrast CMR.

Baseline Characteristics: 282 patients were enrolled. Patient characteristics at baseline are shown in Table 5. The mean (SD) age was 59.1 (12.94) years, 40.1% were female, and 22% were non-white. Baseline New York Heart Association functional class was Class II for 203 patients (72%), Class III for 67 (23.8%), and Class IV for 1 patient (0.4%). More than half the patients (172; 68%) were taking beta blockers. The mean baseline pVO2 was 18.5 (SD 4.5) mL/kg/min or 57.1% of predicted maximum, and the mean Kansas City Cardiomyopathy

Questionnaire Clinical Symptom Score was 74.7 (SD 18.1). Geometric mean high-sensitivity troponin I was 16.9 (7.7, 27.2) ng/L. Left ventricular ejection fraction, LVOT-G, and N-terminal pro brain natriuretic peptide were blinded. Patient characteristics at baseline of the Non-CMR and CMR cohorts are shown in Table 4.

TABLE 4
Baseline Characteristics

| | Non-CMR Cohort | CMR Cohort |
|---|---|---|
| Baseline Characteristics | n = 225 | n = 57 |
| Age, y | 59.3 ± 13.5 | 58.5 ± 10.8 |
| Female sex, n (%) | 95 (42.2) | 20 (35.1) |
| Non-White race , n (%) | 55 (24.4) | 4 (7.1) |
| Known HCM-causing | 41 (18.2) | 8 (14.0) |
| gene mutation, n (%) | | |
| Positive family history of | 60 (26.7) | 15 (26.3) |
| HCM, n (%) | | |
| Time since diagnosis, | 4.0 (1.4, 9.0) | 4.4 (1.4, 6.7) |
| median (IQR), y | | |
| Beta-blocker, n (%) | 138 (61.3) | 35 (61.4) |
| Non-dihydropyridine calcium | 65 (28.9) | 16 (28.1) |
| channel blocker | | |
| Disopyramide | 31 (13.8) | 5 (8.8) |
| History of atrial | 37 (16.4) | 7 (12.3) |
| fibrillation, n (%) | | |
| NYHA FC ≥ III, n (%) | 50 (22.2) | 18 (31.6) |
| NT-proBNP, median (IQR), | 808 (346, 1828) | 655 (358, 1146) |
| pg/mL | | |
| Cardiopulmonary exercise | | |
| testing | | |
| % predicted pVO₂, % | 56.2 ± 12.1 | 59.4 ± 10.5 |
| pVO₂, mL/kg/min | 18.2 ± 4.5 | 19.5 ± 4.2 |

CPET End Points. We looked at whether aficamten would improve a novel measure of integrated exercise performance that combines complementary measures previously independently related to oHCM prognosis, and that incorporates both submaximal and maximal exercise capacity. A primary outcome was the change from baseline to week 24 in integrated exercise performance, defined as the 2-component Z-score of pVO2 and ventilatory efficiency (VE/VCO2 slope). Response rates for achieving clinically meaningful thresholds for change in pVO2 and correlations with clinical measures of treatment effect (health status, echocardiographic, cardiac biomarkers) were also assessed.

The primary analysis was the change from baseline to Week 24 in integrated exercise performance, normalized to a composite of Z-scores for pVO₂ and VE/VCO₂ slope to capture physiologic responses to maximal and submaximal exercise. The Z-score was derived by reversing the directionality of VE/VCO₂ slope such that increases in both Z-score components indicate benefit and equal weights were used for each Z-score component. Secondary end points included assessments for changes from baseline to week 24 in CPET-derived measures during 2 phases of exercise performance: (1) peak exercise (pVO₂, peak workload, peak metabolic equivalents [METS], peak circulatory power [VO₂× systolic blood pressure], exercise duration, peak respiratory exchange ratio [RER], heart rate reserve, peak heart rate, O₂ pulse at peak exercise, proportionate pulse pressure); and (2) during submaximal exercise (pre-anaerobic threshold VE/VCO₂ slope, VE/VCO₂ slope throughout exercise, ventilatory power, ventilatory anaerobic threshold, and aerobic efficiency).

TABLE 5
Baseline Characteristics

| | N (%) or Mean (SD) |
|---|---|
| Baseline Characteristics | except where indicated |
| Age, years | 59.1 (13) |
| Female | 115 (40.1) |
| Race/ethnicity: | |
| White | 220 (78) |
| Black | 3 (1.1) |
| Asian | 53 (18.8) |
| Hispanic | 9 (3.2) |
| Region: | |
| United States | 94 (33.3) |
| China | 46 (16.3) |
| Europe and Israel | 142 (50.4) |
| Vital signs: | |
| Weight (kg) | 82.0 (15.7) |
| Body mass index (kg/m²) | 28.2 (3.7) |
| Systolic blood pressure (mmHg) | 125.7 (16.2) |
| Diastolic blood pressure (mmHg) | 74.4 (10.8) |
| Heart rate (bpm) | 65.4 (11.2) |
| HCM history: | |
| History of known HCM-causing gene | 46 (16.3) |
| mutation | |
| Positive family history of HCM | 70 (24.8) |
| Time since initial HCM diagnosis | 5.9 (IQR 1.5-8.5) |
| (median), years | |
| HCM medical therapies: | |
| Beta blocker (%) | 172 (67.5) |
| Non-dihydropyridine calcium channel | 67 (23.8) |
| blocker (%) | |
| Disopyramide (%) | 36 (12.8) |
| HCM symptoms: | |
| KCCQ-CSS | 74.6 (18.1) |
| NYHA class II/III/IV (%) | 203 (72)/67 (23.8)/1 (0.4) |
| | [11 (3.9) not reported] |
| SRT guideline eligible* | 67 (23.8) |
| Comorbidities: | |
| Hypertension** | 130 (46.1) |
| Diabetes^{†} | 24 (8.5) |
| Permanent atrial fibrillation (%) | 1 (0.4) |
| Paroxysmal atrial fibrillation (%) | 39 (13.8) |
| CPET metrics: | |
| Treadmill (%) | 154 (54.6) |
| pVO₂ (mg/kg/min) | 18.5 (4.5) |
| pVO₂ percent of predicted maximum | 57.2 (12.0) |
| (%)^{††} | |
| Total workload (Watts) | 122.8 (41.2) |
| Biomarker: | |
| hs-cTnI (ng/L) geometric mean | 16.9 (7.7, 27.2) |

*NYHA Class III and any LVOTO ≥50 mmHg
**combines “hypertension” and “essential hypertension”
^{†}combines “type 2 diabetes mellitus”, “type 1 diabetes mellitus”, and “diabetes mellitus”
^{††}Fletcher et al, Circulation, 1995;91:580-615
bpm = beats per minute;
KCCQ-CSS = Kansas City Cardiomyopathy Questionnaire Clinical Symptom Score;
NYHA = New York Heart Association;
SRT = septal reduction therapy;
pVO₂ = peak oxygen uptake;
hs-cTnI = high-sensitivity cardiac troponin-I

Results. Among 282 randomized patients (mean age 59.1±12.9 years, 115 female [40.8%]), 263 (93.0%) had a core-lab validated CPETs at baseline and week 24. Integrated composite exercise performance improved in the aficamten group (Z-score +0.17±0.51) from baseline to week 24, while the placebo group deteriorated (Z-score −0.19±0.45) yielding a placebo-corrected improvement of +0.35 (95% CI+0.25, +0.46; p<0.001). Aficamten treatment demonstrated significant improvements in total workload, circulatory power, exercise duration, heart rate reserve, peak heart rate, ventilatory efficiency, ventilatory power and anaerobic threshold. In the aficamten group, large improvements (≥3.0 mL/kg/min) in pVO₂ were more common than large reductions (≥3.0 mL/kg/min) in pVO₂ (32% and 2%, respectively), compared to placebo (16% and 11%, respectively). Improvements in pVO₂ and VE/VCO₂ slope were significantly correlated with improvements in symptom burden, hemodynamics, cardiac structure, and cardiac biomarkers. This prespecified analysis of SEQUOIA-HCM found that aficamten treatment improved a broad range of exercise performance measures.

Patient Population. 282 eligible patients were randomized to aficamten or placebo. Additional baseline characteristics are shown in Table 6.

TABLE 6
Additional Baseline Characteristics

| | Aficamten | Placebo |
|---|---|---|
| Characteristic | (N = 142) | (N = 140) |
| Cardiac Biomarkers | | |
| Median NT-proBNP (IQR)—pg/ml | 818 (377-1630) | 692 (335-1795) |
| Median hs-cTnl (IQR)—ng/l | 12.9 (7.6-33.6) | 11.5 (7.7-25.0) |
| Echocardiographic parameters | | |
| Valsalva outflow tract | 83 (32) | 83 (33) |
| gradient—mm Hg | | |
| Resting LV outflow tract | 55 (27) | 55 (32) |
| gradient—mm Hg | | |
| LVEF—% | 75 (5.5) | 75 (6.3) |
| LAVI—ml/m² | 40.1 (12.7) | 40.9 (15.1) |
| Maximal wall thickness—cm | 2.1 (0.3) | 2.1 (0.3) |

Percentages may not total 100 because of rounding. Value in parenthesis represents the SD unless otherwise specified, hs-cTnI high-sensitivity cardiac troponin I, IQR interquartile range, NT-proBNP N-terminal pro-B-type natriuretic peptide, and LVEF Left ventricular ejection fraction, LAVI Left atrial volume index.

The mean (SD) age was 59.1 (12.9) years, 115 (40.8%) of the participants were female, and 59 (20.9%) were non-White. Background HCM therapy included 173 (61.3%) receiving beta blockers, 81 (28.7%) non-dihydropyridine calcium-channel blockers, 36 (12.8%) disopyramide, and 41 (14.5%) none. The mean (SD) LVEF was 75% (5.5), 214 (75.9%) patients were NYHA functional class II, and the mean baseline KCCQ-clinical summary score (KCCQ-CSS) was 74.7 (18.0). The median high-sensitivity cardiac troponin I (hs-cTnI) and NT-proBNP concentrations were 12.1 pg/mL (IQR, 7.7-27.3) and 788 ng/L (IQR, 346-1699) respectively. Exercise capacity was reduced as evidenced by the baseline mean pVO₂ of 18.5 (4.5) mL/kg/min representing 56.9 (11.8) percent of age- and sex-predicted pVO₂.

Patient Disposition. At least 1 dose of study medication was received by all randomized patients. At week 8, after completion of the titration phase, aficamten doses were 5 (3.6%), 18 (12.9%), 49 (35%), and 68 (48.6%) for 5 mg, 10 mg, 15 mg, and 20 mg dose levels respectively. Of those randomized, 263 (93.0%) patients completed both baseline and week 24 CPETs that were deemed physiologically interpretable and valid by the core-lab. Of the nineteen (7%, 9 aficamten and 10 placebo) patients who did not have a core-lab interpretable week 24 CPET available for analysis, 6 (2.1%, 3 aficamten and 3 placebo) early terminated from the study prior to week 24 CPET, and 13 (4.6%, 6 aficamten and 7 placebo) were determined by the core-lab to have an invalid week 24 CPET (3 did not follow the CPET manual of operations and 10 were technical failures).

Primary and Secondary End Points. The change from baseline to week 24 in the integrated composite for exercise performance demonstrated improvement in the aficamten group (Z-score +0.17±0.51), compared to a deterioration in the placebo group (Z-score −0.19±0.45), yielding a significant placebo-corrected increase (Z-score +0.35; 95% CI+0.25, +0.46; p<0.001) (Table 7).

TABLE 7
CPET Parameters by Treatment Assignment

Aficamten

| | | | Week | Abs. diff. |
|---|---|---|---|---|
| Variable | n | Baseline | 24 | (SD)^{a} |
| Integrated 2 component | 133 | −0.01 ± | 0.16 ± | 0.17 ± |
| Z-score metric | | 0.82 | 0.76 | 0.51 |
| pVO₂, mL/kg/min^{c} | 133 | 18.4 ± | 20.2 ± | 1.8 ± |
| | | 4.5 | 5.2 | 3.1 |
| Peak workload, watt^{d} | 134 | 120 ± | 134 ± | 14 ± |
| | | 40 | 50 | 27 |
| Peak METs, mL/kg/min | 133 | 5.3 ± | 5.8 ± | 0.51 ± |
| | | 1.3 | 1.5 | 0.89 |
| Peak circulatory power, | 133 | 3013 ± | 3550 ± | 537 ± |
| mmHg*mL/min/kg | | 924 | 1140 | 995 |
| Exercise duration, min | 122 | 11.2 ± | 12.4 ± | 1.2 ± |
| | | 3.0 | 3.9 | 2.1 |
| Peak respiratory | 135 | 1.19 ± | 1.20 ± | 0.01 ± |
| exchange ratio^{e} | | 0.10 | 0.11 | 0.10 |
| Heart rate reserve | 135 | 59 ± | 66 ± | 7 ± |
| | | 18 | 22 | 15 |
| Peak heart rate, | 135 | 128 ± | 135 ± | 8 ± |
| beats/min | | 20 | 23 | 14 |
| O₂ pulse at peak | 133 | 0.14 ± | 0.15 ± | 0.00 ± |
| exercise, mL/beat | | 0.03 | 0.03 | 0.02 |
| Proportionate pulse | 129 | 0.49 ± | 0.51 ± | 0.02 ± |
| pressure (peak) | | 0.11 | 0.12 | 0.14 |
| Proportionate pulse | 129 | 0.39 ± | 0.37 ± | −0.02 ± |
| pressure (rest) | | 0.10 | 0.09 | 0.10 |
| Ventilatory efficiency | 135 | 29.2 ± | 27.4 ± | −1.9 ± |
| pre-anaerobic | | 5.4 | 4.4 | 4.7 |
| thresholdf | | | | |
| Ventilatory efficiency | 135 | 33.2 ± | 30.9 ± | −2.2 ± |
| throughout exercise^{f} | | 6.4 | 5.7 | 4.0 |
| Ventilatory power, | 129 | 5.1 ± | 5.9 ± | 0.8 ± |
| mmHg | | 1.5 | 1.6 | 1.3 |
| Ventilatory anaerobic | 133 | 898 ± | 958 ± | 60 ± |
| threshold, mL^{g} | | 266 | 276 | 107 |
| Aerobic efficiency, | 133 | 8.3 ± | 8.6 ± | 0.3 ± |
| mL/min/watt^{h} | | 2.5 | 2.5 | 1.8 |

| | Placebo | | | | Adjusted | |
|---|---|---|---|---|---|---|
| | | | Week | Abs. diff. | difference | |
| Variable | n | Baseline | 24 | (SD)^{a} | (95% CI)^{b} | p-value |
| Integrated 2 component | 130 | 0.02 ± | −0.17 ± | −0.19 ± | 0.35 | p < 0.001 |
| Z-score metric | | 0.75 | 0.74 | 0.45 | (0.25, 0.46) | |
| pVO², mL/kg/min^{c} | 130 | 18.6 ± | 18.6 ± | 0.0 ± | 1.7 | p < 0.001 |
| | | 4.6 | 4.7 | 2.7 | (1.0, 2.4) | |
| Peak workload, watt^{d} | 129 | 126 ± | 127 ± | 1 ± | 12 | p < 0.001 |
| | | 43 | 44 | 21 | (6, 18) | |
| Peak METs, mL/kg/min | 130 | 5.3 ± | 5.3 ± | 0.00 ± | 0.49 | p < 0.001 |
| | | 1.3 | 1.3 | 0.78 | (0.29, 0.69) | |
| Peak circulatory power, | 125 | 3160 ± | 3074 ± | −86 ± | 586 | p < 0.001 |
| mmHg*mL/min/kg | | 1136 | 1152 | 731 | (379, 793) | |
| Exercise duration, min | 122 | 11.5 ± | 11.7 ± | 0.1 ± | 1.0 | p < 0.001 |
| | | 3.0 | 3.2 | 1.5 | (0.5, 1.4) | |
| Peak respiratory | 130 | 1.18 ± | 1.19 ± | 0.01 ± | 0.00 | p = 0.84 |
| exchange ratio^{e} | | 0.09 | 0.10 | 0.10 | (−0.02, 0.02) | |
| Heart rate reserve | 129 | 57 ± | 59 ± | 1 ± | 6 | p < 0.001 |
| | | 19 | 20 | 10 | (3, 9) | |
| Peak heart rate, | 130 | 128 ± | 127 ± | −1 ± | 9 | p < 0.001 |
| beats/min | | 23 | 23 | 12 | (6, 12) | |
| O₂ pulse at peak | 130 | 0.15 ± | 0.15 ± | 0.00 ± | 0.003 | p = 0.21 |
| exercise, mL/beat | | 0.03 | 0.03 | 0.02 | (−0.002, 0.008) | |
| Proportionate pulse | 124 | 0.50 ± | 0.50 ± | −0.00 ± | 0.01 | p = 0.29 |
| pressure (peak) | | 0.10 | 0.11 | 0.10 | (−0.01, 0.04) | |
| Proportionate pulse | 124 | 0.39 ± | 0.38 ± | −0.01 ± | −0.01 | p = 0.33 |
| pressure (rest) | | 0.10 | 0.10 | 0.11 | (−0.03, 0.01) | |
| Ventilatory efficiency | 129 | 29.1 ± | 28.8 ± | −0.3 ± | −1.5 | p = 0.002 |
| pre-anaerobic | | 4.7 | 5.6 | 4.2 | (−2.5, −0.6) | |
| threshold^{f} | | | | | | |
| Ventilatory efficiency | 130 | 32.9 ± | 32.9 ± | 0.1 ± | −2.3 | p < 0.001 |
| throughout exercisef | | 6.0 | 6.4 | 3.7 | (−3.2, −1.4) | |
| Ventilatory power, | 125 | 5.2 ± | 5.1 ± | −0.1 ± | 0.9 | p < 0.001 |
| mmHg | | 1.6 | 1.5 | 1.0 | (0.6, 1.1) | |
| Ventilatory anaerobic | 129 | 931 ± | 927 ± | −3 ± | 59 | p < 0.001 |
| threshold, mL^{g} | | 261 | 257 | 108 | (33, 85) | |
| Aerobic efficiency, | 128 | 8.2 ± | 8.2 ± | 0.1 ± | 0.2 | p = 0.22 |
| mL/min/watt^{h} | | 2.3 | 2.4 | 1.7 | (−0.2, 0.6) | |

Abbreviations: Abs. diff., absolute difference; MET, metabolic equivalent; pVO₂, peak oxygen uptake; VCO₂, carbon dioxide output; VE, minute ventilation; VO₂, oxygen uptake.
^{a}The absolute difference corresponds to the change from baseline to week 24.
^{b}The adjusted difference corresponds to the least-squares mean treatment difference.
^{c}pVO₂ measures the peak rate of oxygen uptake during maximum incremental workload exercise on a treadmill or cycle ergometer. Higher values reflect greater cardiorespiratory fitness and greater ability to perform physical activity. A change of 1 mL/kg/min is considered clinically significant.
^{d}Workload measures work performed during exercise (on a cycle or treadmill) in watts.
^{e}Respiratory exchange ratio measures volume of carbon dioxide exhaled divided by the amount of oxygen uptake (VCO₂/VO₂). When anaerobic metabolism takes place during maximum effort exercise, buffering of lactic acid results in CO₂ formation in excess of VO₂. Hence, a VCO₂/VO₂ of 1.05 or greater is an indicator of maximum volitional effort exercise.
^{f}Ventilatory efficiencey is measured as the slope of minute ventilation divided by volume of carbon dioxide exhaled during exercise testing (VE/VCO₂). Ventilatory efficiency is a valid measure during submaximal exercise (ie, without maximal exercise effort). A slope greater than 34 represents inefficient ventilation and higher risk of morbidity and mortality.
^{g}Ventilatory anaerobic threshold is a marker of exercise intensity, measured at the inflection point at which the shift occurs from aerobic to anaerobic metabolism, leading to increased carbon dioxide production (VCO₂). Determined by V-slope method.
^{h}Aerobic efficiency (ΔVO₂/ΔWork) describes the relationship of O₂ utilization to the amount of work performed. A normal VO₂-work rate relationship during the incremental ramp portion of CPET is 10 ± 1.5mL/min/Watt.

Aficamten improved peak exercise performance as measured by the total workload, circulatory power, exercise duration, heart rate reserve, and peak heart rate (Table 7). Aficamten also improved sub-maximal exercise performance as measured by ventilatory efficiency, increased ventilatory power, and increased ventilatory anaerobic threshold.

Responder Analysis. Large pVO₂ improvements (≥3.0 mL/kg/min) were more frequent with aficamten treatment compared with placebo (32% versus 16% respectively), and large pVO₂ deteriorations (≤−3.0 mL/kg/min) were less frequent aficamten treatment versus placebo (2% versus 11% respectively) (FIG. 2). The odds ratios for achieving any improvement (OR 3.09, 95% CI 1.88 to 5.09), moderate to large improvements ≥1.5 mL/kg/min (OR 2.44, 95% CI 1.50, 3.96), and large improvements ≥3.0 mL/kg/min (OR 2.44, 95% CI 1.38, 4.29) all favor aficamten treatment and correspond with a number needed to treat of 3.8, 4.7 and 6.3 patients (Table 8).

TABLE 8
Responder analyses of categorical change in peak oxygen
vuptake with aficamten ersus placebo at 24 weeks

| | | | OR | Risk Difference | |
|---|---|---|---|---|---|
| Outcome | Placebo | Aficamten | (95% CI) | (95% CI) | NNT |
| Any | 67 | 105 | 3.09 | +26% | 3.8 |
| Improvement | (47.9%) | (73.9%) | (1.88, 5.09) | (+15%, −37%) | |
| (small/ | | | | | |
| moderate/ | | | | | |
| large) | | | | | |
| Moderate/ | 44 | 75 | 2.44 | +21% | 4.7 |
| Large | (31.4%) | (52.8%) | (1.50, 3.96) | (+10%, −33%) | |
| Improvement | | | | | |
| Large | 23 | 46 | 2.44 | +15.% | 6.3 |
| Improvement | (16.4%) | (32.4%) | (1.38, 4.29) | (+6%, +26%) | |
| Any | 73 | 37 | 0.32 | −26% | −3.8 |
| Deterioration | (52.1%) | (26.1%) | (0.20, 0.53) | (−37%, −15%) | |
| (small/ | | | | | |
| moderate/ | | | | | |
| large) | | | | | |

Data represented as proportion of patients experiencing small (0 to <1.5), moderate (≥1.5, <3), or large (≥3.0) deteriorations or improvements in pVO₂: (mL/kg/min) per treatment group. Abbreviations: CI, confidence interval; NNT, number needed to treat; OR, odds ratio; pVO₂, peak oxygen uptake.

Clinical Correlations. Univariate analyses revealed significant associations between improvements in pVO₂ and improvements in KCCQ-CSS, NYHA functional class, septal E/e,′ resting and Valsalva LVOT-G, NT-proBNP, and hs-cTnI (Table 9).

TABLE 9
Univariate Correlations Between Change in Components of
Integrated Exercise Response Z-score and Changes in other Measures

| | | Overall | | |
|---|---|---|---|---|
| | | N | Correlation | P-value |
| | Clinical Variable | | | |
| Change in | KCCQ-CSS | 262 | 0.206 | p < 0.001 |
| pVO₂ vs. | NYHA functional class | 263 | −0.173 | p = 0.005 |
| | log-transformed NT-proBNP | 254 | −0.328 | p < 0.001 |
| | log-transformed hs-cTnI | 246 | −0.141 | p = 0.028 |
| | Resting LVOT-G | 258 | −0.162 | p = 0.009 |
| | Valsalva LVOT-G | 258 | −0.135 | p = 0.030 |
| | LAVI | 253 | −0.094 | p = 0.14 |
| | Septal E/e′ | 251 | −0.244 | p < 0.001 |
| | Mitral regurgitation | 255 | −0.104 | p = 0.10 |
| | Exercise Test Variable | | | |
| | Workload | 262 | 0.517 | p < 0.001 |
| | Exercise duration | 243 | 0.518 | p < 0.001 |
| | Peak respiratory exchange ratio | 263 | 0.000 | p = 1.00 |
| | Peak systolic blood pressure | 252 | 0.290 | p < 0.001 |
| | Ventilatory efficiency throughout | 263 | −0.227 | p < 0.001 |
| | exercise | | | |
| | Ventilatory efficiency pre- | 262 | −0.285 | p < 0.001 |
| | anaerobic threshold | | | |
| | Anaerobic threshold | 262 | 0.750 | p < 0.001 |
| | Clinical Variable | | | |
| Change in | KCCQ-CSS | 264 | −0.158 | p = 0.010 |
| VE/VCO₂ | NYHA functional class | 265 | 0.284 | p < 0.001 |
| slope vs. | log-transformed NT-proBNP | 256 | 0.312 | p < 0.001 |
| | log-transformed hs-cTnI | 248 | 0.074 | p = 0.24 |
| | Resting LVOT-G | 260 | 0.244 | p < 0.001 |
| | Valsalva LVOT-G | 260 | 0.222 | p < 0.001 |
| | LAVI | 255 | 0.136 | p = 0.030 |
| | Septal E/e′ | 253 | 0.105 | p = 0.09 |
| | Mitral regurgitation | 257 | −0.103 | p = 0.10 |
| | Exercise Variable | | | |
| | Workload | 263 | −0.050 | p = 0.42 |
| | Exercise duration | 244 | −0.097 | p = 0.13 |
| | Peak respiratory exchange ratio | 265 | −0.035 | p = 0.57 |
| | Peak systolic blood pressure | 254 | −0.079 | p = 0.21 |
| | Ventilatory efficiency throughout | 265 | 1.000 | p < 0.001 |
| | exercise | | | |
| | Ventilatory efficiency pre- | 264 | 0.505 | p < 0.001 |
| | anaerobic threshold | | | |
| | Anaerobic threshold | 262 | −0.234 | p < 0.001 |

Abbreviations: hs-cTnI, high-sensitivity cardiac troponin I; KCCQ-CSS, Kansas City Cardiomyopathy Questionnaire-Clinical Summary Score; LAVI, left atrial volume index; LVOT-G, left ventricular outflow tract gradient; NT-proBNP, N-terminal pro-B-type natriuretic peptide; NYHA, New York Heart Association; VO₂, oxygen uptake.

Sequential regression multivariate analyses revealed that changes in NT-proBNP accounted for the greatest amount of variance in change in pVO₂, while changes in E/e′ and KCCQ-CSS also significantly added to the observed variance (Table 10).

TABLE 10
Multivariate regression model to evaluate independent
degree of variance explained in change in pVO₂ from
baseline to week 24 by clinical variables

| | Multivariate Regression Model | | Change |
|---|---|---|---|
| | Covariates | R² | in R² |
| Variance | 1) pVO₂ | 0.037 | N/A |
| Explained in | 2) model 1 + log-transformed NT-proBNP | 0.200 | 0.163* |
| Change in | 3) model 2 + E/e′ | 0.215 | 0.015* |
| pVO₂ | 4) model 3 + KCCQ-CSS | 0.239 | 0.024* |
| | 5) model 4 + NYHA functional class | 0.253 | 0.014 |
| | 6) model 5 + resting LVOT-G | 0.260 | 0.007 |
| | 7) model 6 + log-transformed hs-cTnI | 0.269 | 0.009 |
| | 8) model 7 + Valsalva LVOT-G | 0.283 | 0.014 |
| | 9) model 8 + mitral regurgitation | 0.290 | 0.008 |
| | 10) model 9 + LAVI | 0.309 | 0.019 |
| Variance | 1) VE/VCO2 | 0.094 | N/A |
| Explained in | 2) model 1 + log transform NTpro-BNP | 0.167 | 0.073* |
| Change in | 3) model 2 + NYHA Class | 0.181 | 0.014 |
| VE/VCO₂ | 4) model 3 + LVOT during Rest | 0.197 | 0.016 |
| slope | 5) model4 + LVOT during Valsalva | 0.200 | 0.003 |
| | 6) model 5 + KCCQ-CSS | 0.201 | 0.002 |
| | 7) model 6 + LA Volume Index | 0.224 | 0.023* |
| | 8) model 7 + E/e′ | 0.237 | 0.012 |
| | 9) model 8 + Mitral Regurgitation | 0.273 | 0.036* |

Abbreviations: hs-cTnI, high-sensitivity cardiac troponin I; KCCQ-CSS, Kansas City Cardiomyopathy Questionnaire Clinical Summary Score; LAVI, left atrial volume index; LVOT-G, left ventricular outflow tract gradient; NT-proBNP, N-terminal pro-B-type natriuretic peptide; NYHA, New York Heart Association; pVO₂, peak oxygen uptake. * indicates a significance level with p<0.05 for incremental variance explained in change in pVO₂ or change in VE/VCO₂ slope from baseline to week 24.

In univariate analysis, improvements in VE/VCO₂ slope were associated with changes in many of the same clinical measures as change in pVO₂. However, change in VE/VCO₂ slope demonstrated higher correlation with changes in LVOT gradients (rest and Valsalva) and changes in LAVI, and lower correlation with changes in E/e′ and hs-cTnI. In the multivariate analysis, changes in NT-proBNP, LAVI, and mitral regurgitation significantly explained variance in VE/VCO₂ slope (Table 10). In addition to distinct correlates for pVO₂ and VE/VCO₂ slope, we observed only a modest relationship between the change in pVO₂ and the change in VE/VCO₂ slope (correlation coefficient 0.23) highlighting the orthogonal nature of the two variables selected for our 2-component Z-score.

We developed a novel metric that is relevant to daily physical activity and that integrates both maximal and submaximal exercise performance by CPET, and demonstrated that this measure is significantly improved with aficamten versus placebo treatment in oHCM patients, and that the benefits appear to go well beyond a simple increase in pVO₂. Importantly, there is only a modest relationship between changes in pVO₂ and VE/VCO₂ (correlation coefficient <0.25), but a stronger correlation of both variables with changes in NT-proBNP. This finding is supportive of the concept that both pVO₂ and VE/VCO₂ embedded in the 2-component Z-score are complementary.

In our categorical threshold analyses, aficamten patients were twice as likely to achieve a large pVO₂ increase (≥3.0 mL/kg/min) and more than 5 times less likely to experience worsening than those on placebo (FIG. 2), with <4 patients requiring treatment with aficamten to achieve exercise capacity improvement versus placebo. These findings were particularly noteworthy, given that improvements in pVO₂ of this magnitude and consistency are rarely seen with previous medical therapies for individuals with either HCM or heart failure.

CPET metrics that are both cardio-specific (VE/VCO₂ slope, circulatory power, heart rate reserve, peak heart rate) as well as those that are highly integrated with whole body physiology (ie, pVO₂) (Table 7 and Table 9) improved in the setting of aficamten treatment. By expanding the range of CPET parameters evaluated, we gained a more holistic understanding of the physiological responses to exercise in patients with oHCM. For certain conditions like heart failure with preserved ejection fraction, the determinants of pVO₂ extend beyond cardiac output to include factors such as peripheral abnormalities (ie, impaired peripheral oxygen extraction by muscles), making improvement in exercise capacity harder to achieve with cardiospecific pharmacologic interventions. The observation that aficamten improved both heart rate reserve and peak heart rate is particularly interesting.

Our comprehensive prespecified analysis of CPET metrics, including a novel integrated exercise performance metric, showed improved exercise response patterns in oHCM patients treated with aficamten. Improvements in exercise physiology correlated with improvements in cardiac structure and function extending beyond reduction in LVOT, while also relating to change in symptom burden. Our findings indicate that aficamten affected both submaximal and maximal exercise performance, spanning the array of experiences and activities that are important to patients with symptomatic oHCM.

Beta Blocker Co-administration. 61.3% of patients were taking beta blockers in addition to aficamten. The LSM difference in pVO2 (mL/kg/min, 95% CI) was 1.6 (0.7 to 2.5) in patients who were also on beta blockers and 1.9 (0.8 to 3.1) in patients who were not on beta blockers. This represents a nonsignificant difference and shows that aficamten's effect on pVO2 was similar with and without background beta blocker use. Change in Exercise Duration as measured by CPET (Tables 11A and 11B) and in peak metabolic equivalents (peak METs) as measured by CPET (Tables 12A and 12B) showed a significant improvement for patients receiving aficamten which was not significantly diminished by the use of beta blockers. This result contrasts with mavacamten (EXPLORER-HCM), which showed attenuation of benefit, such as pVO2, exercise duration (also called “peak exercise time”), and peak METs, in patients receiving beta blockers (see European Journal of Heart Failure (2023), 25, 260-270).

TABLE 11A
Change in Exercise Duration (minutes) on CPET from
Baseline to Week 24-Baseline Beta Blocker Use: Yes

| Visit | Aficamten | Placebo | Overall |
|---|---|---|---|
| Statistics | (N = 86) | (N = 87) | (N = 173) |
| Baseline | | | |
| n | 76 | 85 | 161 |
| Mean (SD) | 11.4 (2.55) | 11.3 (2.99) | 11.4 (2.78) |
| Median | 11.1 | 11.3 | 11.3 |
| Q1, Q3 | 9.4, 13.7 | 9.2, 13.4 | 9.3, 13.6 |
| Min, Max | 6, 17 | 5, 19 | 5, 19 |
| Week 24 | | | |
| n | 72 | 78 | 150 |
| Mean (SD) | 12.5 (3.61) | 11.5 (3.35) | 12.0 (3.50) |
| Median | 12.4 | 11.6 | 11.8 |
| Q1, Q3 | 9.9, 14.9 | 8.9, 13.7 | 9.5, 14.2 |
| Min, Max | 7, 26 | 5, 20 | 5, 26 |
| Change from Baseline-Week 24 | | | |
| n | 72 | 78 | 150 |
| Mean (SD) | 1.1 (1.98) | 0.1 (1.66) | 0.6 (1.87) |
| Median | 0.5 | −0.1 | 0.2 |
| Q1, Q3 | −0.2, 1.8 | −0.8, 1.0 | −0.5, 1.2 |
| Min, Max | −3, 10 | −4, 8 | −4, 10 |
| LS Mean (SE) | 1.0 (0.22) | 0.1 (0.21) | |
| LS Mean 95% CI | (0.6, 1.4) | (−0.3, 0.5) | |
| LS Mean Diff vs. Placebo (SE) | 0.9 (0.30) | | |
| LS Mean Diff vs. Placebo 95% CI | (0.3, 1.5) | | |
| p-value | 0.003 | | |

TABLE 11B
Change in Exercise Duration (minutes) on CPET from Baseline
to Week 24-Baseline Beta Blocker Use: No

| Visit | Aficamten | Placebo | Overall |
|---|---|---|---|
| Statistics | (N = 56) | (N = 53) | (N = 109) |
| Baseline | | | |
| n | 53 | 47 | 100 |
| Mean (SD) | 11.0 (3.52) | 11.8 (2.94) | 11.3 (3.27) |
| Median | 10.3 | 11.4 | 10.9 |
| Q1, Q3 | 8.1, 13.3 | 9.5, 13.7 | 8.5, 13.6 |
| Min, Max | 6, 20 | 6, 19 | 6,20 |
| Week 24 | | | |
| n | 50 | 44 | 94 |
| Mean (SD) | 12.2 (4.39) | 11.9 (3.04) | 12.1 (3.80) |
| Median | 11.3 | 11.9 | 11.5 |
| Q1, Q3 | 8.8, 14.2 | 9.9, 14.2 | 9.3, 14.2 |
| Min, Max | 6, 23 | 6, 18 | 6, 23 |
| Change from Baseline-Week 24 | | | |
| n | 50 | 44 | 94 |
| Mean (SD) | 1.3 (2.34) | 0.1 (1.24) | 0.8 (1.98) |
| Median | 1.1 | 0.0 | 0.5 |
| Q1, Q3 | −0.1, 2.0 | −0.8, 1.0 | −0.4, 1.5 |
| Min, Max | −3, 13 | −2, 3 | −3, 13 |
| LS Mean (SE) | 1.2 (0.26) | 0.1 (0.28) | |
| LS Mean 95% CI | (0.7, 1.7) | (−0.4, 0.7) | |
| LS Mean Diff vs. Placebo (SE) | 1.1 (0.38) | | |
| LS Mean Diff vs. Placebo 95% CI | (0.3, 1.8) | | |
| p-value | 0.004 | | |
| Subgroup by Treatment | 0.683 | | |
| Interaction p-value | | | |

TABLE 12A
Change in peak METs (pVO₂/3.5 (mL/kg/min)) from Baseline on CPET
Parameter to Week 24-Baseline Beta Blocker Use: Yes

| Visit | Aficamten | Placebo | Overall |
|---|---|---|---|
| Statistics | (N = 86) | (N = 87) | (N = 173) |
| Baseline | | | |
| n | 86 | 87 | 173 |
| Mean (SD) | 5.24 (1.251) | 5.20 (1.308) | 5.22 (1.276) |
| Median | 5.28 | 5.20 | 5.27 |
| Q1, Q3 | 4.38, 6.03 | 4.30, 5.89 | 4.30, 5.96 |
| Min, Max | 2.5, 8.3 | 2.6, 9.2 | 2.5, 9.2 |
| Week 24 | | | |
| n | 80 | 80 | 160 |
| Mean (SD) | 5.70 (1.497) | 5.18 (1.386) | 5.44 (1.461) |
| Median | 5.64 | 5.12 | 5.46 |
| Q1, Q3 | 4.71, 6.65 | 4.23, 6.15 | 4.41, 6.34 |
| Min, Max | 2.6, 11.0 | 2.5, 9.0 | 2.5, 11.0 |
| Change from | | | |
| Baseline-Week 24 | | | |
| n | 80 | 80 | 160 |
| Mean (SD) | 0.41 (0.781) | −0.02 (0.688) | 0.20 (0.764) |
| Median | 0.31 | −0.10 | 0.10 |
| Q1, Q3 | −0.11, 0.83 | −0.40, 0.40 | −0.25, 0.64 |
| Min, Max | −1.0, 3.7 | −1.6, 2.1 | −1.6, 3.7 |
| LS Mean (SE) | 0.40 (0.093) | −0.06 (0.092) | |
| LS Mean 95% CI | (0.22, 0.58) | (−0.24, 0.12) | |
| LS Mean Diff vs. | 0.46 (0.130) | | |
| Placebo (SE) | | | |
| LS Mean Diff vs. | (0.20, 0.71) | | |
| Placebo 95% CI | | | |
| p-value | <0.001 | | |

TABLE 12B
Change in peak METs (pVO₂/3.5 (mL/kg/min)) from Baseline on CPET
Parameter to Week 24-Baseline Beta Blocker Use: No

| Visit | Aficamten | Placebo | Overall |
|---|---|---|---|
| Statistics | (N = 56) | (N = 53) | (N = 109) |
| Baseline | | | |
| n | 56 | 53 | 109 |
| Mean (SD) | 5.27 (1.314) | 5.49 (1.244) | 5.38 (1.279) |
| Median | 5.11 | 5.63 | 5.21 |
| Q1, Q3 | 4.19, 5.99 | 4.51, 6.41 | 4.29, 6.24 |
| Min, Max | 3.3, 8.8 | 3.1, 7.9 | 3.1, 8.8 |
| Week 24 | | | |
| n | 53 | 50 | 103 |
| Mean (SD) | 5.89 (1.481) | 5.52 (1.270) | 5.71 (1.389) |
| Median | 5.66 | 5.40 | 5.49 |
| Q1, Q3 | 5.00, 7.06 | 4.57, 6.26 | 4.74, 6.60 |
| Min, Max | 2.7, 9.6 | 3.0, 10.1 | 2.7, 10.1 |
| Change from Baseline-Week 24 | | | |
| n | 53 | 50 | 103 |
| Mean (SD) | 0.65 (1.029) | 0.03 (0.914) | 0.35 (1.019) |
| Median | 0.47 | −0.12 | 0.33 |
| Q1, Q3 | 0.08, 1.09 | −0.69, 0.51 | −0.24, 0.79 |
| Min, Max | −1.6, 4.1 | −2.1, 2.8 | −2.1, 4.1 |
| LS Mean (SE) | 0.62 (0.113) | 0.06 (0.117) | |
| LS Mean 95% CI | (0.40, 0.84) | (−0.17, 0.29) | |
| LS Mean Diff vs. Placebo (SE) | 0.55 (0.162) | | |
| LS Mean Diff vs. | (0.24, 0.87) | | |
| Placebo 95% CI | | | |
| p-value | <0.001 | | |
| Subgroup by Treatment | 0.633 | | |
| Interaction p-value | | | |

Secondary Analysis of the Results of the SEQUOIA-HCM Study. In a sub-analysis of the results of Example 8 (i.e, the results of the SEQUOIA-HCM Study) we sought to comprehensively characterize the broad clinical impact of aficamten with respect to responder analyses across a number of clinically relevant, patient-centric outcome domains.

For each clinical efficacy outcome, week 24 data was used when available. For patients with missing data, the last available observation (week 8 to week 20) was used. Patients with no available data on or after week 8 were classified as not meeting the efficacy criteria. Patients were then classified according to the number of efficacy outcome definitions that they met. Baseline characteristics were summarized using means and standard deviations or median and interquartile for continuous normal and skewed variables, as well as counts and percentages for categorical variables. These characteristics were compared across groups using trend tests (linear regression, chi-squared tests of trend, and Cuzick's non-parametric trend tests). Proportions of patients meeting each efficacy outcome definition were compared between treatment groups using chi-squared tests, and linear regression was used to assess the treatment effect of aficamten vs placebo on change in efficacy outcomes while also adjusting for baseline value, CPET modality at baseline and beta blocker use at baseline. No adjustments were made for multiple comparisons. Two-sided p-values less than 0.05 were considered statistically significant.

At 24 weeks, compared to placebo, patients on aficamten showed significant improvement in limiting symptoms (71% vs. 42%), proportion with complete hemodynamic response (68% vs. 7%), enhanced exercise capacity (47% vs. 24%) and a decrease of ≥50% in NT-proBNP (84% vs. 8%) (P≤0.002 vs. placebo for all). An improvement in ≥1 of these outcome measures was achieved in 97% of patients on aficamten, including 23% who achieved all 4. Among those eligible for SRT, 88% on aficamten were no longer eligible at 24 weeks, compared to 52% on placebo (P=0.002).

LV outflow tract gradients. At week 24, a complete hemodynamic response was achieved in 97 (68%) patients receiving aficamten and 10 (7%) patients on placebo (P<0.001) (FIG. 3A). As early as week 8, following completion of dose titration and achievement of steady state dose, a complete hemodynamic response was achieved in 100 (70%) of patients receiving aficamten and 12 (9%) on placebo (P<0.001). As early as week 2, rest and Valsalva gradients were reduced 31% and 25% respectively in patients on aficamten compared to baseline. In addition, over the treatment period, magnitude of gradient reduction was greater in patients treated with aficamten with rest and Valsalva LV outflow tract gradients decreased from 55±27 mmHg to 20±17 mmHg (placebo corrected difference-40 mmHg, 95% CI: −46, −34; P<0.001) and 83±32 mmHg to 35±25 mmHg, respectively (placebo-corrected difference-50 mmHg 95% CI: −57, −44; p<0.001).

Heart failure symptoms (NYHA class and/or KCCQ-CSS). At week 24, a significantly greater proportion in the aficamten group vs placebo experienced an improvement of ≥1 NYHA functional class and/or a ≥10 point increase in KCCQ-CSS (101[71%] vs. 59[42%]; P<0.001) (FIG. 3B). Over the treatment period, the proportion of patients on aficamten with an improvement in heart failure symptoms increased significantly from 51 (36%) at week 2 to 82 (58%) at week 8 and 101 (71%) at week 24 (P<0.001). Of the 101 aficamten symptomatic responders, 18 (18%) achieved a ≥10 point improvement in KCCQ-CSS score with no improvement in NYHA class, 31 (31%) had a ≤10 point improvement in KCCQ but a ≥1 improvement in NYHA functional class. The remaining 52 (51%) patients experienced an improvement in both NYHA and KCCQ (vs. 13[9%] on placebo; P<0.001).

With respect to NYHA functional class alone, among the 142 patients treated with aficamten, 55 (39%) improved from class II to I, 15 (11%) from class III to I, 13 (9%) from class III to II, 54 (38%) had no change in NYHA class, and 3 (2%) worsened from class II to class III (2 patients withdrew early). With respect to KCCQ-CSS, 18 (13%) of the 142 patients on aficamten experienced a clinically relevant, but small, improvement of ≥5 to <10, 39 (27%) a moderate to large improvement of ≥10 to <20, and 31 (22%) a very large change of ≥20; 52 (37%) had no change or a decrease in KCCQ-CCS score (<5 points).

Exercise capacity. An improvement in exercise capacity with a pVO₂ of ≥1.5 mL/kg/min occurred in 66 (47%) patients receiving aficamten and in 34 (24%) receiving placebo (P <0.001). The proportion of patients who achieved pVO₂ of ≥1.5 mL/kg/min and ≥1 improvement in NYHA class at Week 24 was greater in patients on aficamten vs. placebo (31% vs. 6%; P<0.001) as was the proportion with ≥3.0 mL/kg/min² and no worsening in NYHA (26% vs. 9%; P <0.001) or either of these outcomes (42% vs. 14%, p<0.001) (Table 13). The responder endpoint of pVO₂≥1.5 mL/kg/min and >1 improvement in NYHA class or pVO₂≥3.0 mL/kg/min² and no worsening in NYHA was met by 42.3% of patients on aficamten and 13.6% of patients on placebo, for a difference vs. placebo of 28.7% (95% CI: 18.8, 38.6, p<0.001) and a number needed to treat (NNT) of 3.

TABLE 13
Change with aficamten in exercise capacity by pVO₂ and
limiting symptoms with NYHA functional class

| | Aficamten | Placebo |
|---|---|---|
| | (N = 142) | (N = 140) |
| >1.5 mL/kg/min increase in pVO2 | 60 (42) | 19 (14) |
| and ≥1 NYHA class improvement | | |
| or ≥3.0 mL/kg/min increase in | | |
| pVO2 and no worsening of | | |
| NYHA class, n (%) | | |
| ≥1.5 mL/kg/min increase in | 44 (31) | 9 (6) |
| pVO2 and ≥1 NYHA | | |
| class improvement, n (%) | | |
| ≥3.0 mL/kg/min increase in pVO2 and | 37 (26) | 13 (9) |
| no worsening of NYHA class, n (%) | | |
| ≥3.0 mL/kg/min increase in | 21 (15) | 3 (2) |
| pVO2 and ≥1 NYHA | | |
| class improvement, n (%) | | |
| Common Rate Difference (vs. Placebo) | 29 | |
| 95% CI Common Rate Difference | (19, 39) | |
| p-value | <0.0001 | |

Cardiac biomarkers. Aficamten treatment led to an immediate and significant reduction in both NT-proBNP and hs-cTnI, with substantial lowering of both biomarkers by week 2. Notably, by week 8 the lowering of both biomarkers from aficamten treatment was nearly maximal; NT-proBNP was reduced by 79% (95% CI 83%, 76%, p<0.001) and hs-cTnI by 41% (95% CI 49%, 32%, p<0.001). Biomarker lowering persisted to week 24 when the relative reduction from baseline was-80% (95% CI-83%, −77%, p<0.001) in NT-proBNP and −43% (−49%, 36%, p<0.001) in hs-cTnI. Compared to baseline, the proportion of patients in the aficamten group with a 50% relative reduction in NT-proBNP and hs-troponin concentrations at week 24 was significantly greater than in the placebo group (84% vs. 8% (FIG. 3C) and 31% vs. 4%, respectively; P<0.001). Serum levels of NT-proBNP decreased from baseline to week 24 from 735 (604, 894) ng/L to 143 (120, 171) ng/L in patients receiving aficamten and remained unchanged from 710 (587, 859) ng/L to 717 (596, 863) ng/L in those receiving placebo, while hs-troponin decreased from 17.1 (14.1, 20.8) ng/L to 9.5 (8.0, 11.3) ng/L with aficamten and remained unchanged from 16.6 (13.3, 20.7) to 16.5 (13.7, 19.9) ng/L in placebo (P<0.001 for both comparisons compared to placebo). Following completion of the treatment period, cessation of aficamten resulted in concentrations of both NT-proBNP and hs-cTnI returning to baseline values.

Both biomarkers were found to be inversely correlated with change in pVO2 and health status (with increase in KCCQ-CSS), and directly correlated with change in Valsalva LVOT-G, maximal LV wall thickness, and E/e′. NT-proBNP change by 24 weeks was directly correlated with change in left atrial volume indexed (LAVi), while hs-cTnI was not.

Mediation analyses of the primary endpoint of pVO2 change suggested that NT-proBNP reduction over the 24-week treatment period strongly correlated with effect of aficamten on the primary endpoint, reflecting 101% (41%, 195%) of aficamten's effect on pVO2. In contrast hs-cTnI reduction reflected 3% (−18%, 30%) of aficamten's effect on pVO2.

Changes in NT-proBNP and hs-cTnI concentration by 24 weeks were associated with improvements in cardiac structure and function extending beyond reduction in LVOT-G. Importantly, change in NT-proBNP strongly reflected the impact of aficamten on improved exercise capacity. In fact, the change in NT-proBNP by 24 weeks was an accurate surrogate for change in pVO2.

Eligibility for Septal Reduction Therapy (SRT). At study entry, 61 (22%) patients with oHCM were eligible for SRT based on guideline criteria (LV outflow gradient ≥50 mmHg at rest or Valsalva and NYHA class III or IV), of which 32 (23%) were assigned to aficamten and 29 (20%) to placebo. By week 12, 32 (100%) patients treated with aficamten were no longer guideline-eligible for SRT vs. 7 (24%) patients treated with placebo (P<0.001). At week 24, 28 (88%) patients treated with aficamten were no longer guideline-eligible for SRT vs. 15 (52%) on placebo (P<0.002).

Global Clinical Response to Aficamten. At week 24 of treatment, 138 patients (97%) receiving aficamten achieved ≥1 of the prespecified clinical response measures vs. 82 (59%) of the placebo patients (p<0.001). Within the aficamten treatment arm, 32 (23%) improved in all 4 measures, 56 (39%) in 3, 37 (26%) in 2 and 13 (9%) achieved one, while in the placebo arm no patient achieved all 4 measures, 5 (4%) achieved 3, 22 (16%) achieved 2 and 55 (39%) achieved one (FIG. 4 and FIG. 5). A categorical reduction of ≤50% in NT-proBNP was the most common outcome measure achieved with aficamten treatment (84%), followed by improvement in heart failure symptoms (71%) and hemodynamic response (68%).

At the end of the treatment period, aficamten was associated with a complete hemodynamic response (rest and Valsalva gradient <30 mmHg and <50 mmHg, respectively) in two thirds of patients. Further, close to half of patients treated with aficamten resolved their symptoms completely (i.e., become NYHA class I) by the end of the treatment period.

Aficamten substantially improved overall performance of the integrated CPET Z-score by a placebo-adjusted difference of 0.35 (95% CI, 0.25, 0.46; p<0.001). Additionally, of patients treated with aficamten, 72.2% experienced an improvement in pVO2, compared to 43.8% of patients treated with placebo. Among the patients treated with aficamten, 27.8% had a large improvement (≥3.0 mL/kg/min) in pVO2, 21.8% had a moderate improvement (≥1.5 to <3.0 mL/kg/min) and 22.6% had a small improvement (0 to <1.5). Furthermore, enhanced exercise performance was shown to be correlated with improvements in clinically important measures including Kansas City Cardiomyopathy Questionnaire Clinical Summary Score (KCCQ-CSS) (p=0.001), New York Heart Association (NYHA) Functional Class (p<0.001), resting LVOT-G (p=0.003), Valsalva LVOT-G (p=0.001), NT-proBNP (p<0.001) and high-sensitivity cardiac troponin I (p=0.010). Importantly, these correlations demonstrate that the therapeutic effects of aficamten manifest broadly and are interrelated.

Patients with Hypertrophic Obstructive Cardiomyopathy and Very Severe Left Ventricular Outflow Tract Obstruction. Patients enrolled in SEQUOIA-HCM (the study of Example 8), the pivotal phase 3 controlled trial of aficamten in adults with symptomatic oHCM, were grouped according to baseline Valsalva LVOT-G (severe obstruction [<100 mmHg] versus very severe obstruction [≥100 mmHg]). The prespecified primary endpoint was the proportional change from baseline to Week 24 in rest and Valsalva LVOT-G in severe versus very severe obstruction groups. Secondary endpoints included change in symptoms, cardiac biomarkers, and exercise capacity by group (Table 14). Of the 282 patients randomized, 209 patients had severe obstruction, and 73 had very severe obstruction. The baseline characteristics of each subgroup are shown in Table 14. Compared to baseline, there was a profound reduction in resting and provoked obstruction in both groups. By week 24, the proportional reductions (95% CI) from baseline in the severely obstructed cohort were 69% (74 to 63) and 64% (69 to 56), and 73% (81 to 63) and 71% (79 to 61) in the very severe obstruction cohort, but there was no between-group difference (p>0.05). All secondary endpoints (symptom burden, cardiac biomarkers, and exercise capacity) demonstrated significant improvements relative to baseline within each group, but no between-group differences (Table 14). The post-titration doses by group were similar, with 81% and 88% in each cohort receiving the 15 mg and 20 mg dose strengths (p=0.21). There were no instances of per protocol down-titration in the very severe obstruction group, and treatment emergent adverse events (TEAE's) were balanced between groups (any TEAE 77 [71%] in severe versus 28 [85%] in very severe obstruction; p=0.60).

Independent of the severity of baseline obstruction, patients treated with aficamten in SEQUOIA-HCM showed significant improvements in proportional hemodynamic response, symptom relief, exercise capacity, and reduction in cardiac biomarkers independent of the severity of baseline obstruction. Additionally, aficamten treatment appeared safe between hemodynamic subgroups.

TABLE 14
Changes in endpoints from Baseline to Week 24 by provoked LVOT-G severity:
Severe (<100 mmHg) versus Very Severe (≥100 mmHg)

| | Baseline Valsalva LVOT-G <100 mmHg | | | | |
|---|---|---|---|---|---|
| | Aficamten | | Placebo | | |
| | (N = 109) | | (N = 100) | | |
| | | Mean (SD) | | Mean (SD) | |
| | | Change | | Change | |
| | | from | | from | Adjusted diff |
| | n (%) | Baseline | n (%) | Baseline | (95% CI) |
| LVOT-G - Rest, | 106 | −28 ± 25 | 96 | 9 ± 31 | −35 |
| mmHg | (97%) | | (96%) | | (−41,−28) |
| Proportional | 106 | 0.34 | 96 | 1.19 | −69 |
| LVOT-G - Rest | (97%) | (0.29, 0.40) | (96%) | (1.03, 1.39) | (−74, −63) |
| (95% CI), % | | | | | |
| LVOT-G - | 106 | −38 ± 31 | 96 | 8 ± 31 | −43 |
| Valsalva, mmHg | (97%) | | (96%) | | (−50, −36) |
| Proportional | 106 | 0.39 | 96 | 1.09 | −64 |
| LVOT-G - | (97%) | (0.33, 0.45) | (96%) | (0.98, 1.22) | (−69, −56) |
| Valsalva | | | | | |
| (95% CI), % | | | | | |
| Proportion of | 79 | N/A | 9 | N/A | +63.7 |
| patients with | (73%) | | (9%) | | (+53.3, +74.1) |
| rest LVOT- | | | | | |
| G <30 mmHg & | | | | | |
| Valsalva LVOT- | | | | | |
| G <50 mmHg | | | | | |
| (95% CI), % | | | | | |
| Change in | 107 | 12 | 97 | 4 | +8 |
| KCCQ CSS | (98%) | (13) | (97%) | (12) | (+5, +12) |
| NYHA FC | 67 | N/A | 23 | N/A | +38.2 |
| improvement | (62%) | | (23%) | | (+25.6, +50.8) |
| (Rate difference | | | | | |
| 95% CI), % | | | | | |
| NT-proBNP | 103 | 0.20 | 95 | 0.98 | −79 |
| (95% CI), % | (94%) | (0.17, 0.24) | (95%) | (0.89, 1.09) | (−82, −74) |
| hs-cTpI | 103 | 0.57 | 89 | 1.03 | −45 |
| (95% CI), % | (94%) | (0.52, 0.64) | (89%) | (0.92, 1.15) | (−52, −37) |
| Peak VO₂, | 102 | 1.8 ± 3.2 | 93 | 0.2 ± 2.9 | +1.6 |
| mL/kg/min) | (94%) | | (93%) | | (+0.8, +2.5) |

| | Baseline Valsalva LVOT-G ≥100mmHg | | | | | |
|---|---|---|---|---|---|---|
| | Aficamten (N=33) | | Placebo (N=40) | | | |
| | | Mean (SD) | | Mean (SD) | | |
| | | Change | | Change | | |
| | | from | | from | Adjusted diff | Interaction |
| | n (%) | Baseline | n (%) | Baseline | (95% CI) | P-value |
| LVOT-G - Rest, | 31 | −60 ± 29 | 38 | −10 ± 40 | −52 | 0.009 |
| mmHg | (94%) | | (95%) | | (−66, −38) | |
| Proportional | 31 | 0.23 | 38 | 0.84 | −73 | 0.42 |
| LVOT-G - Rest | (94%) | (0.17, 0.30) | (95%) | (0.68, 1.05) | (−81,−63) | |
| (95% CI), % | | | | | | |
| LVOT-G - | 31 | −82 ± 38 | 38 | −14 ± 39 | −68 | 0.002 |
| Valsalva, mmHg | (94%) | | (95%) | | (−84, −52) | |
| Proportional | 31 | 0.25 | 38 | 0.84 | −71 | 0.21 |
| LVOT-G - | (94%) | (0.18, 0.34) | (95%) | (0.72, 0.97) | (−79, −61) | |
| Valsalva | | | | | | |
| (95% CI), % | | | | | | |
| Proportion of | 17 | N/A | 1 (3%) | N/A | +51.2 | 0.17 |
| patients with | (52%) | | | | (+34.1, +68.2) | |
| rest LVOT- | | | | | | |
| G <30 mmHg & | | | | | | |
| Valsalva LVOT- | | | | | | |
| G <50 mmHg | | | | | | |
| (95% CI), % | | | | | | |
| Change in | 31 | 9 | 40 | 7 | +4 | 0.19 |
| KCCQ CSS | (94%) | (13) | (100%) | (10) | (−1, +9) | |
| NYHA FC | 16 | N/A | 11 | N/A | +22.4 | 0.20 |
| improvement | (49%) | | (28%) | | (−0.7, +45.6) | |
| (Rate difference | | | | | | |
| 95% CI), % | | | | | | |
| NT-proBNP | 30 | 0.16 | 38 | 1.00 | −85 | 0.07 |
| (95% CI), % | (91%) | (0.12, 0.21) | (95%) | (0.85, 1.18) | (−89, −81) | |
| hs-cTpI | 30 | 0.58 | 37 | 0.95 | −34 | 0.24 |
| (95% CI), % | (91%) | (0.48, 0.71) | (93%) | (0.79, 1.14) | (−48, −18) | |
| Peak VO₂, | 31 | 1.6 ± 2.8 | 37 | −0.6 ± 2.3 | +1.8 | 0.79 |
| mL/kg/min) | (94%) | | (93%) | | (+0.6, +2.9) | |

SD, standard deviation; CI, confidence interval; LVOT-G, left ventricular outflow tract gradient; KCCQ-CSS, Kansas City Cardiomyopathy Questionnaire Clinical Summary Score; NYHA FC, New York Heart Association Class functional class; NT-proBNP, n-terminal pro-brain natriuretic peptide; hs-cTnI, high sensitivity cardiac troponin I; VO₂, oxygen uptake

Improved exercise capacity, disease-specific quality-of-life (QoL), and hemodynamics in patients with obstructive hypertrophic cardiomyopathy (oHCM). Participants underwent blinded dose escalation over 6 weeks and were treated for 24 weeks followed by 4-week washout. EQ-5D-5L index (ranges from 0 to 1) and the Visual Analogue Scale (VAS; from 0 to 100) was measured at baseline and weeks 0, 2, 4, 8, 12, 16, 20, 24, with higher scores indicating better QoL. Aficamten treatment effect on EQ-5D-5L was estimated using linear regression (specific study visits) and mixed effects regression (overall treatment effect) using all follow-up data through week 24, with model adjustments for baseline EQ-5D-5L score and randomization stratification variables.

Mean age (n=282) was 59.1±12.9 years (40.8% women; 79.1% white). Aficamten baseline EQ-5D-5L index and VAS scores were 0.80±0.19 and 71.2±17.9 (placebo: 0.78±0.20; 68.9±18.6), respectively. Aficamten improved the EQ-5D-5L index by 0.026 (95% CI: −0.001, 0.052; p=0.056) and the VAS by 2.8 points (95% CI: 0.8, 4.8; p=0.006) versus placebo with significant differences as early as 8 weeks after treatment initiation (p=0.005). After treatment withdrawal at 24 weeks, QoL worsened in aficamten-treated patients compared with placebo (Index, p<0.001; VAS, p=0.003), despite other clinical parameters returning to baseline. Aficamten yielded early, sustained, and significant improvement in overall QOL

compared with placebo. Withdrawal of aficamten was associated with the loss of treatment benefits and perhaps an increased intolerance of return to baseline symptoms. These data support a role of aficamten in improving QoL among patients with oHCM.

Effect of Aficamten on Cardiac Structure and Function in Patients with Obstructive Hypertrophic Cardiomyopathy. For some patients with HCM, adverse remodeling of cardiac structure and function occur over time and are associated with increased risk of adverse events, including heart failure, sudden death and atrial fibrillation. Eligible participants enrolled in SEQUOIA-HCM (the study of Example 8), were offered enrolment in the cardiovascular magnetic resonance (CMR) substudy with CMR performed at baseline and at Week 24. Image analysis was performed in a blinded fashion by a core laboratory.

Of the 282 randomized HCM patients, 57 (20%) participated in the CMR substudy, and of those, 50 patients (88%) completed both baseline and Week 24 CMR. Baseline characteristics (mean age 58.5±10.8 years, 35% female) were similar to the overall study population (mean age 59.3±13.5, 42% female). Twenty-one of the 50 patients received aficamten and 29 received placebo. Consistent with the overall study population, patients enrolled in the CMR substudy who received aficamten treatment demonstrated improvements relative to placebo (A least squares mean; 95% CI) in echocardiographic resting LVOT-G (−46 mm Hg; −68, −23; p=0.0002) and Valsalva LVOT-G (−50 mm Hg; −73, −27; p<0.0001), and NT-proBNP (−85%, −92%, −72%, p<0.0001). CMR substudy patients receiving aficamten demonstrated significant reductions relative to placebo (Δ least squares mean, 95% CI) in LV mass index (−15 g/m²; −25, −6; p=0.001); maximal LV wall thickness (−2.1 mm; −3.1, −1.1; p<0.001), left atrial volume index (−13 mL/m²; −19, −7; p<0.001) and native Tl relaxation time (−37 ms; −69, −5; p-0.026) while there were no significant change in LV end-diastolic and end-systolic volumes. In patients treated with aficamten, there was no change in LV replacement fibrosis (late gadolinium enhancement [LGE] mass −0.7 g; −2.9, 1.6; p=0.54), or LV extracellular volume fraction [ECV] (0.7%; −2.2, 3.6; p=0.61) (Table 15). When comparing CMR (n=57) to non-CMR patients (n=225), the treatment effect observed on cardiac structure and function using CMR was consistent directionally, but generally larger than observed with echocardiography (Table 16). In particular, echocardiography appeared to underestimate aficamten's treatment effect on left atrial volume index as compared to CMR.

In this CMR substudy of SEQUOIA-HCM, aficamten treatment for 24 weeks resulted in favorable cardiac remodeling.

TABLE 15
Change in key parameters and cardiac magnetic resonance measures from baseline to week 24
in patients enrolled in the SEQUOIA-HCM CMR Substudy who completed
both baseline and follow-up CMR

| | Aficamten | |
|---|---|---|
| | N = 21 | |
| Variables | Baseline | Week 24 |
| Echo LVOT Gradient | 55 ± 32 | 21 ± 18 |
| at Rest (mm Hg) | | |
| Echo LVOT Gradient | 85 ± 45 | 35 ± 26 |
| with Valsalva (mm Hg) | | |
| NT-proBNP (pg/mL), | 708 | 79 |
| median (IQR) | (440, 1160) | (38, 207) |
| Peak Oxygen | 20.1 ± 4.4 | 21.1 ± 4.7 |
| Consumption | | |
| (pVO₂; mL/kg/min) | | |
| NYHA Class (%) | II/III | I/II/III |
| | (67%/33%) | (57%/29%/14%) |

Cardiac Magnetic Resonance Measures

| LV Mass Index (g/m²) | 113 ± 33 | 103 ± 28 | | |
|---|---|---|---|---|
| LV Maximal Wall | 19.3 ± 4.3 | 17.8 ± 4.5 | | |
| Thickness (mm) | | | | |
| LV Ejection Fraction | 77.4 ± 5.6 | 73.8 ± 5.9 | | |
| (%) | | | | |
| LV End Diastolic | 67 ± 15 | 60 ± 11 | | |
| Volume Index (mL/m²) | | | | |
| LV End Systolic | 15.3 ± 5.9 | 16.0 ± 5.1 | | |
| Volume Index (mL/m²) | | | | |
| LA Volume Index | 68 ± 19 | 52 ± 14 | | |
| (mL/m²) | | | | |
| Mitral Regurgitation | 24 | 13 | | |
| Fraction (%) | [15, 47] | [7, 20] | | |
| Mitral Regurgitation | 22 | 12 | | |
| Volume (mL) | [13, 56] | [5, 25] | | |
| LGE Mass (g) | 3.2 | 1.5 | | |
| | [0.5, 5.7] | [1.0, 3.4] | | |
| LGE (% of LV mass) | 2.0 | 1.4 | | |
| | [0.3, 3.8] | [0.7, 2.2] | | |
| LV Native T1 (ms) | 1066 ± 121 | 1021 ± 93 | | |
| LV Extracellular | 26.7 ± 2.7 | 27.4 ± 7.1 | | |
| Volume Fraction (%) | | | | |
| LV extracellular | 31 ± 10 | 29 ± 12 | | |
| volume mass index | | | | |
| (%/g/m²) | | | | |
| Absolute myocyte mass | 84 ± 25 | 75 ± 22 | | |
| index (g/m²) | | | | |
| | | | Treatment | |
| | Placebo N = 29 | | Effect (95% | |
| Variables | Baseline | Week 24 | CI | p-value |
| Echo LVOT Gradient | 51 ± 34 | 62 ± 42 | −46 | 0.0002 |
| at Rest (mm Hg) | | | (−68, −23) | |
| Echo LVOT Gradient | 84 ± 35 | 83 ± 41 | −50 | <0.0001 |
| with Valsalva (mm Hg) | | | (−73, −27) | |
| NT-proBNP (pg/mL), | 556 | 539 | −85% | <0.0001 |
| median (IQR) | (289, 892) | (269, 946) | (−92%, −72%) | |
| Peak Oxygen | 19.8 ± 3.6 | 19.8 ± 4.1 | 1.0 | 0.21 |
| Consumption | | | (−0.57, 2.58) | |
| (pVO₂; mL/kg/min) | | | | |
| NYHA Class (%) | II/III | I/II/III | 36% | 0.0135* |
| | (72%/28%) | (21%/59%/21%) | (9%, 62%) | |

Cardiac Magnetic Resonance Measures

| | | | | |
|---|---|---|---|---|
| LV Mass Index (g/m²) | 110 ± 27 | 116 ± 33 | −15 | <0.001 |
| | | | (−25, −6) | |
| LV Maximal Wall | 19.9 ± 4.4 | 20.4 ± 4.1 | −2.1 | <0.001 |
| Thickness (mm) | | | (−3.1, −1.1) | |
| LV Ejection Fraction | 79.0 ± 5.6 | 77.3 ± 7.2 | −2.6 | 0.12 |
| (%) | | | (−5.9, 0.7) | |
| LV End Diastolic | 67 ± 15 | 61 ± 15 | −1 | 0.79 |
| Volume Index (mL/m²) | | | (−6, 5) | |
| LV End Systolic | 14.4 ± 6.4 | 14.1 ± 6.8 | 1.4 | 0.30 |
| Volume Index (mL/m²) | | | (−1.3, 4.1) | |
| LA Volume Index | 61 ± 16 | 60 ± 15 | −13 | <0.001 |
| (mL/m²) | | | (−19, −7) | |
| Mitral Regurgitation | 30 | 21 | −6 | 0.22 |
| Fraction (%) | [18, 41] | [14, 26] | (−16, 4) | |
| Mitral Regurgitation | 28 | 19 | −7 | 0.13 |
| Volume (mL) | [18, 50] | [11,25] | (−16, 2) | |
| LGE Mass (g) | 4.0 | 3.3 | −0.7 (−2.9, 1.6) | 0.54 |
| | [0.4, 9.1] | [0.9, 6.5] | | |
| LGE (% of LV mass) | 2.5 | 1.7 | −0.4 (−2.1, 1.2) | 0.60 |
| | [0.7, 7.4] | [0.6, 3.8] | | |
| LV Native T1 (ms) | 1044 ± 116 | 1045 ± 114 | −37 (−69, −5) | 0.026 |
| LV Extracellular | 28.5 ± 4.7 | 28.1 ± 3.1 | 0.7 (−2.2, 3.6) | 0.61 |
| Volume Fraction (%) | | | | |
| LV extracellular | 30.7 ± 9.4 | 33 ± 11 | −3.9 (−7.0, | 0.014 |
| volume mass index | | | −0.9) | |
| (%/g/m²) | | | | |
| Absolute myocyte mass | 77 ± 20 | 84 ± 23 | −14 (−23, −4) | 0.004 |
| index (g/m²) | | | | |

*Treatment Effect ≥1 NYHA Class Improvement. Data shown as mean ± SD unless otherwise specified.
CMR, cardiac magnetic resonance;
LA, left atrial;
LGE, late gadolinium enhancement;
LV, left ventricular;
LVOT, left ventricular outflow tract gradient;
NT-proBNP, N-terminal pro b-type natriuretic peptide;
NYHA, New York Heart Association.

TABLE 16
Comparative treatment effect between CMR measurements
and echocardiographic measurements in the CMR substudy and in
the non-CMR cohort of SEQUOIA-HCM

| | | CMR Measurements | | |
|---|---|---|---|---|
| | | CMR Cohort | | |
| | | N = 57 | | |
| | | | | Treatment |
| | Study | Aficamten | Placebo | Effect |
| | Visit | N = 25 | N = 32 | (95% CI) |
| LV Mass | Baseline | 113 ± 33 | 110 ± 27 | −15 |
| Index (g/m²) | Week 24 | 103 ± 28 | 116 ± 33 | (−25, −6) |
| | | | | p < 0.001 |
| LV Maximal | Baseline | 19.3 ± 4.3 | 19.9 ± 4.4 | −2.1 |
| Wall | | | | |
| Thickness | Week 24 | 17.8 ± 4.5 | 20.4 ± 4.1 | (−3.1, −1.1) |
| (mm) | | | | p < 0.001 |
| LV Ejection | Baseline | 77.4 ± 5.6 | 79.0 ± 5.6 | −2.6 |
| Fraction (%) | Week 24 | 73.8 ± 5.9 | 77.3 ± 7.2 | (−5.9, 0.7) |
| | | | | p = 0.12 |
| LA Volume | Baseline | 68 ± 19 | 61 ± 16 | −13 |
| Index | | | | |
| (mL/m²) | Week 24 | 52 ± 14 | 60 ± 15 | (−19, −7)* |
| | | | | p < 0.001 |
| NT-proBNP, | Baseline | 708 | 644 | −85 |
| median (IQR) | | (447,1160) | (310,1121) | (−89, −78) |
| (pg/mL) | Week 24 | 81 | 594 | p < 0.001 |
| | | (40,207) | (269,1815) | |
| Peak oxygen | Baseline | 19.8 ± 4.7 | 19.4 ± 3.9 | 1.2 |
| consumption, | | | | |
| pVO₂, | Week 24 | 21.0 ± 4.9 | 19.4 ± 4.1 | (−0.3, 2.6) |
| mL/kg/min | | | | p = 0.11 |
| | Baseline | N/A | N/A | |
| Improvement | Week 24 | 15 (60.0) | 11 (34.4) | OR 3.00 |
| in NYHA by | | | | (0.99, |
| ≥1 FC, n (%) | | | | 9.10) |
| | | | | p = 0.05 |
| | | Echo Measurements^{†} | | |
| | | CMR Cohort | | |
| | | N = 57 | | |
| | | | | Treatment |
| | Study | Aficamten | Placebo | Effect |
| | Visit | N = 25 | N = 32 | (95% CI) |
| LVOT | Baseline | 55 ± 30 | 51 ± 34 | −38 |
| gradient at | Week 24 | 25 ± 25 | 63 ± 41 | (−56, −20) |
| rest | | | | p < 0.001 |
| (mmHg) | | | | |
| LVOT | Baseline | 84 ± 42 | 84 ± 35 | −47 |
| gradient | | | | |
| with | Week 24 | 38 ± 30 | 85 ± 40 | (−65, −30) |
| Valsalva | | | | p < 0.001 |
| (mmHg) | | | | |
| LV Mass | Baseline | 126 ± 22 | 129 ± 37 | −10 |
| Index (g/m2) | Week 24 | 120 ± 32 | 131 ± 30 | (−24, 3) |
| | | | | p = 0.13 |
| LV | Baseline | 20.6 ± 2.7 | 20.6 ± 2.6 | −1.5 |
| Maximal | | | | |
| Wall | | | | |
| Thickness | Week 24 | 18.7 ± 3.1 | 20.0 ± 2.6 | (−2.7, −0.2) |
| (mm) | | | | p = 0.027 |
| | Baseline | 74.3 ± 4.7 | 75.7 ± 4.7 | |
| LV Ejection | Week 24 | 68.2 ± 7.8 | 72.2 ± 5.5 | −3.6 |
| Fraction | | | | (−7.1, −0.2) |
| (%) | | | | p = 0.040 |
| LA Volume | Baseline | 40 ± 11 | 38 ± 11 | −1 |
| Index | Week 24 | 39 ± 10 | 39 ± 10 | (−5, 2) |
| (mL/m²) | | | | p = 0.53 |
| | | Echo Measurements^{†} | | |
| | | Non-CMR Cohort | | |
| | | N = 225 | | |
| | | | | Treatment |
| | Study | Aficamten | Placebo | Effect |
| | Visit | N = 117 | N = 108 | (95% CI) |
| LV Mass | Baseline | 131 ± 33 | 138 ± 36 | −13 |
| Index (g/m²) | Week 24 | 126 ± 33 | 145 ± 40 | (−20, −7) |
| | | | | p < 0.001 |
| LV | Baseline | 20.7 ± 3.0 | 21.1 ± 3.1 | −1.2 |
| Maximal | Week 24 | 19.2 ± 3.2 | 20.6 ± 3.1 | (−1.9, −0.5) |
| Wall | | | | p < 0.001 |
| Thickness | | | | |
| (mm) | | | | |
| LV Ejection | Baseline | 74.8 ± 5.6 | 74.6 ± 6.5 | −5.3 |
| Fraction | Week 24 | 67.8 ± 7.4 | 73.0 ± 6.8 | (−7.0, −3.5) |
| (%) | | | | p < 0.001 |
| LA Volume | Baseline | 40 ± 13 | 40 ± 14 | −5 |
| Index | | | | |
| (mL/m²) | Week 24 | 37 ± 11 | 42 ± 12 | (−6, −3) |
| | | | | p < 0.001 |

*p < 0.05 for comparison with echo measurement in CMR cohort.
^{†}no significant interactions between CMR cohort and non-CMR cohort using echo measurements. Data shown as mean ± SD unless specified otherwise.
CMR, cardiac magnetic resonance;
LA, left atrial;
LV, left ventricular.

Effect of Aficamten on Patient-Reported Health Status in Obstructive Hypertrophic Cardiomyopathy. Participants were randomized to aficamten or placebo with standard-of-care therapy for 24 weeks followed by a 4-week washout period. The Kansas City Cardiomyopathy Questionnaire (KCCQ) and Seattle Angina Questionnaire (SAQ) were administered at each study visit. Changes in mean KCCQ Overall Summary score (KCCQ-OSS) and SAQ Summary score (SAQ-SS) from baseline to week 24 were compared by treatment group using linear regression, adjusting for randomization strata. Proportions of patients with clinically important changes were compared.

Baseline mean±SD KCCQ-OSS (69±20 vs. 67±19) and SAQ-SS (72±21 vs. 72±18) were similar in the aficamten (n=142) and placebo (n=140) groups. Aficamten was associated with greater 24-week improvements in KCCQ-OSS (13.3±16.3 vs 6.1±12.6, p<0.001) and SAQ-SS (11.6±17.4 vs 3.8±14.4, p<0.001) than placebo irrespective of baseline patient characteristics. Many more aficamten-treated patients had very large improvements (≥20 points) in their KCCQ-OSS and SAQ-SS (29.7% vs. 12.4%; NNT=5.8; and 31.2% vs. 13.9%; NNT=5.8, respectively). The benefits of aficamten on health status rapidly receded following drug washout from week 24 to 28.

Aficamten markedly improved the health status and angina symptoms of patients with symptomatic oHCM during treatment with loss of these benefits after drug withdrawal. When treatment was withdrawn between weeks 24 and 28, a decline in the health status of both treatment groups was observed, with much larger decrements in those who had been treated with aficamten. Compared with week 24, those in the aficamten arm experienced greater decreases in their KCCQ-OSS (−16.2±19.0 vs −3.0±9.6) and SAQ-OSS (−17.4±21.4 vs. −2.5±13.3) scores (both p-values <0.001).

Subgroup Analyses. FIGS. 6A and 6B show the treatment benefit of aficamten across subgroups for the KCCQ-OS and SAQ-SS, respectively. No heterogeneity of treatment benefit was observed by participants' demographics (i.e., sex, age, BMI), echocardiographic characteristics (i.e., LVEF and LVOT-G at rest), exercise capacity (i.e., pVO2), NTpro-BNP levels, randomization strata (beta-blocker use at baseline, cardiopulmonary exercise test modality), or baseline KCCQ-OSS (all interaction p-values >0.05). Further, the baseline frequency of chest pain did not appear to impact treatment benefit. However, there was a signal that those with poor to fair overall angina-related health status (SAQ-SS<50) attained greater benefit from aficamten (greater change with aficamten in SAQ-SS=15.6 points 95% CI: 4.7, 26.4) than those with good to excellent baseline angina-related health status (SAQ-SS-75-100, interaction p-value=0.04), although the latter still benefited from treatment (SAQ-SS improvement with aficamten=4.2 (95% CI: 0.7, 7.6).

Modifiability of Post-Exercise Oxygen Uptake Recovery (VO₂Rec) Patterns: A Substudy of the SEQUOIA-Hypertrophic Cardiomyopathy Randomized Trial.

Patients were randomized 1:1 to aficamten or placebo for 24 weeks with a primary endpoint of pVO₂. For this analysis, VO₂Rec delay was measured as the time taken after exercise cessation for VO₂ to decline by >0%, 12.5% (t_{12.5%}), 25%, or 50% of pVO₂. Change in t_{12.5%} from baseline (BL) to week 24 (W24) was the primary endpoint. Response rates for achieving clinically meaningful threshold (>15 seconds) for change in t_{12.5%}, and correlations with changes in cardiac function (echo/cardiac biomarkers) were assessed.

Among 282 randomized patients (mean age 59.1±12.9 years, 115 female [41%]), 263 (93%) had valid CPETs at BL and W24 with VO₂Rec values as shown (Table 17). At W24, t_{12.5%} improved by 18% (11 to 27%) in the aficamten group (p<0.001), compared to 9% improvement in pVO₂ (p<0.001). No change in the 2 measures was observed in the placebo arm. Those on aficamten were more likely to improve t_{12.5%} by ≥15 seconds (OR 3.68 95% CI 1.9-6.9, NNT=4.8) than those on placebo. Reductions in t_{12.5%} correlated with reductions in NTproBNP, troponinT, and LVOT gradient and improvements in NYHA class and KCCQ scores (all p<0.005).

This study established that VO₂Rec is modifiable in response to an effective cardio-specific intervention and accelerates proportionate to improvements in resting cardiac function with aficamten treatment of oHCM.

TABLE 17
Comparative treatment effect on VO₂Rec

AFICAMTEN

| | | | | Absolute |
|---|---|---|---|---|
| Variable | n | Baseline | Week 24 | Δ (SD) |
| Peak VO₂ | 133 | 18.4 ± | 20.2 ± | 1.8 ± |
| (mL/kg/min) | | 4.5 | 5.2 | 3.1 |
| VO₂ recovery delay | 134 | 19 ± | 15 ± | −4 ± |
| (sec) | | 20 | 18 | 19 |
| VO₂ recovery 12.5% | 126 | 45 ± | 38 ± | −7 ± |
| (sec) | | 20 | 18 | 19 |
| VO₂ recovery 25% | 123 | 66 ± | 60 ± | −6 ± |
| (sec) | | 21 | 19 | 18 |
| VO₂ recovery 50% | 117 | 115 ± | 107 ± | −8 ± |
| (sec) | | 32 | 32 | 27 |

| | PLACEBO | | | | | |
|---|---|---|---|---|---|---|
| | | | | Absolute | Adjusted Δ | |
| Variable | n | Baseline | Week 24 | Δ (SD) | (95% CI) | p-value |
| Peak VO₂ | 130 | 18.6 ± | 18.6 ± | 0.0 ± | 1.7 | p < 0.001 |
| (mL/kg/min) | | 4.6 | 4.7 | 2.7 | (1.0, 2.4) | |
| VO₂ recovery delay | 129 | 17 ± | 18 ± | 1 ± | −4 | p = 0.047 |
| (sec) | | 19 | 19 | 19 | (−8, −0) | |
| VO₂ recovery 12.5% | 127 | 45 ± | 46 ± | 1 ± | −8 | p < 0.001 |
| (sec) | | 22 | 23 | 16 | (−12, −5) | |
| VO₂ recovery 25% | 126 | 70 ± | 70 ± | −0 ± | −7 | p < 0.001 |
| (sec) | | 27 | 28 | 17 | (−11, −3) | |
| VO₂ recovery 50% | 116 | 116 ± | 116 ± | 0 ± | −8 | p = 0.010 |
| (sec) | | 38 | 36 | 26 | (−15, −2) | |

pVO₂, peak oxygen uptake; The absolute difference corresponds to the change from baseline to week 24. The adjusted difference corresponds to the LSM treatment difference.

Global Remodelling Changes with Aficamten in Patients with Obstructive Hypertrophic Cardiomyopathy: A Secondary Analysis of the SEQUOIA-HCM trial.

SEQUOIA-HCM recruited patients with symptomatic oHCM (New York Heart Association [NYHA]≥class 2, baseline pVO₂ of ≤90% of predicted, respiratory exchange ratio [RER] of ≥1.05). Patients were randomized 1:1 to aficamten (5-20 mg daily) or placebo, and doses were adjusted during the first 6 weeks to achieve relief from obstruction (LVOT-G<30 mmHg) while maintaining left ventricular ejection fraction (LVEF) ≥50%, for 24 weeks. In this secondary analysis, remodelling response was assessed across five domains including: 1) left atrial volume index (LAVI) graded as normal (16-34 mL/m²), mild (35-41 mL/m²), moderate (42-48 mL/m²), severe enlargement (>48 mL/m²); 2) presence of left ventricular hypertrophy (LVH) with strain pattern on electrocardiogram (EKG) assessed by core lab, 3) Hyperdynamic (≥72) LVEF by core lab; 4)N-terminal brain natriuretic peptide (NT-proBNP) improvement of ≥50%, and; and 5) decrease of maximal wall thickness (MWT) ≥1.5 mm.

Individual remodelling domains demonstrated significant improvement for aficamten vs placebo as follows: improvement by one category in LAVI among the patients with abnormal LAVI at baseline (>34 mL/m²) at baseline (44/94 (46.8%) vs 17/91 (18.7%) placebo, p<0.001), reduction in prevalence of LVH with strain pattern on EKG (27 (19.0%) vs 6 (4.3%) placebo, p<0.001), resolution of hyperdynamic LVEF (50 (35.2%) aficamten vs 29 (20.7%) placebo, p=0.007), improvement in MWT (68 (47.9%) vs 43 (30.7%) placebo, p=0.003), and decrease by ≥50% in NT-proBNP (116 (81.7%) vs 10 (7.1%) placebo, p<0.001). Overall, of 142 aficamten treated patients, 119 (84%) had beneficial remodelling in at least one of five domains vs 55 (39%) of 140 patients on placebo (p<0.001), yielding a number needed to treat to achieve beneficial remodelling of 2.2.

Treatment with aficamten resulted in beneficial global remodelling changes in multiple domains encompassing cardiac structure and function by echocardiogram, EKG, and cardiac biomarkers.

### Example 9

Polymorphic Form I, Form II, Form III, Form IV, Form V, and Form VI of aficamten were characterized by various analytical techniques, including XRPD, DSC, TGA, and DVS, as described in WO 2021/011807 and U.S. Patent Publication US-2022-0315571-A1, the disclosures of which are incorporated by reference herein.

1. A method of improving a heart rate dependent CPET (cardiopulmonary exercise testing) parameter in a patient in need thereof, comprising administering to the patient a therapeutically effective amount of aficamten 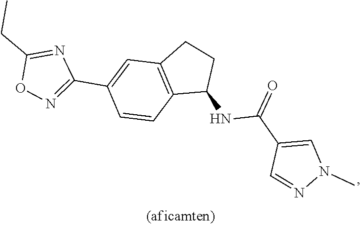 [SMILES: CCc1nc(-c2ccc3c(c2)CC[C@H]3NC(=O)c2cnn(C)c2)no1]
or a pharmaceutically acceptable salt thereof, and
wherein the patient is concurrently undergoing a treatment with a beta blocker.

2. (canceled)

3. The method of claim 1,
wherein the improvement in the heart rate dependent CPET (cardiopulmonary exercise testing) parameter is not substantially diminished by the treatment with the beta blocker.

4-7. (canceled)

8. A method of treating a patient in need thereof, comprising administering to the patient a therapeutically effective amount of aficamten 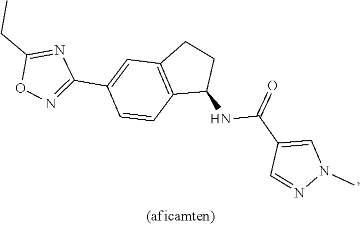 [SMILES: CCc1nc(-c2ccc3c(c2)CC[C@H]3NC(=O)c2cnn(C)c2)no1]
or a pharmaceutically acceptable salt thereof, and a beta blocker, wherein
(a) the co-administration of the beta blocker with aficamten, or a pharmaceutically acceptable salt thereof, does not cause chronotropic incompetence in the patient, and/or:
(b) the co-administration of the beta blocker with aficamten, or a pharmaceutically acceptable salt thereof, does not impact the ability of the patient's heart to increase its rate commensurate with increased activity or demand, and/or
(c) the patient's heart maintains its ability to increase its rate commensurate with increased activity or demand compared to administration of aficamten, or a pharmaceutically acceptable salt thereof, without a beta blocker.

9-17. (canceled)

18. The method of claim 1, wherein the heart rate dependent CPET (cardiopulmonary exercise testing) parameter is peak metabolic equivalents (METs).

19. (canceled)

20. The method of claim 18,
wherein the improvement in peak metabolic equivalents (METs) is not substantially diminished by the treatment with the beta blocker.

21-26. (canceled)

27. The method of claim 1, wherein the heart rate dependent CPET (cardiopulmonary exercise testing) parameter is exercise duration.

28. (canceled)

29. The method of claim 27,
wherein the improvement in exercise duration is not substantially diminished by the treatment with the beta blocker.

30-35. (canceled)

36. A method of improving one or more of integrated 2 component Z-score metric, pVO₂, peak workload, peak METs, peak circulatory power, exercise duration, peak respiratory exchange ratio, heart rate reserve, peak heart rate, O₂ pulse at peak exercise, peak proportionate pulse pressure, proportionate pulse pressure at rest, ventilatory efficiency pre-anaerobic threshold, ventilatory efficiency throughout exercise, ventilatory power, ventilatory anaerobic threshold, and aerobic efficiency, in a patient in need thereof, comprising administering to the patient a therapeutically effective amount of aficamten 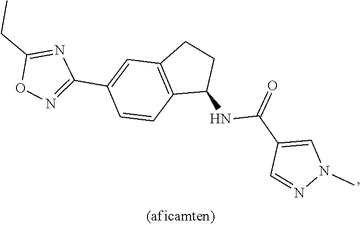 [SMILES: CCc1nc(-c2ccc3c(c2)CC[C@H]3NC(=O)c2cnn(C)c2)no1]
or a pharmaceutically acceptable salt thereof, and
wherein the patient is concurrently undergoing a treatment with a beta blocker.

37. (canceled)

38. The method of claim 36,
wherein the improvement in the one or more of integrated 2 component Z-score metric, pVO₂, peak workload, peak METs, peak circulatory power, exercise duration, peak respiratory exchange ratio, heart rate reserve, peak heart rate, O₂ pulse at peak exercise, peak proportionate pulse pressure, proportionate pulse pressure at rest, ventilatory efficiency pre-anaerobic threshold, ventilatory efficiency throughout exercise, ventilatory power, ventilatory anaerobic threshold, and aerobic efficiency, is not substantially diminished by the treatment with the beta blocker.

39-43. (canceled)

44. The method of claim 1, wherein the heart rate dependent CPET (cardiopulmonary exercise testing) parameter is peak oxygen uptake (pVO₂).

45. (canceled)

46. The method of claim 44,
wherein the improvement in peak oxygen uptake (pVO₂) is not substantially diminished by the treatment with the beta blocker.

47-57. (canceled)

58. A method of
(i) improving a heart rate dependent CPET (cardiopulmonary exercise testing) parameter in a patient in need thereof, or
(ii) accelerating oxygen uptake recovery (VO₂Rec) in a patient in need thereof, or
(iii) reducing or resolving left ventricular hypertrophy (LVH) in a patient in need thereof, or
(iv) decreasing maximal wall thickness (MWT) in a patient in need thereof, or
(v) reducing chest pain in a patient in need thereof, or
(vi) treating oHCM in a patient in need thereof, wherein the patient had a SAQ-SS score of less than 50 prior to beginning administration of aficamten;
comprising administering to the patient a therapeutically effective amount of aficamten 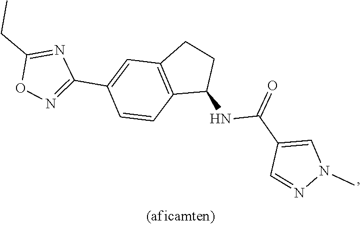 [SMILES: CCc1nc(-c2ccc3c(c2)CC[C@H]3NC(=O)c2cnn(C)c2)no1]
or a pharmaceutically acceptable salt thereof.

59-75. (canceled)

76. A method of treating a disease or condition in a patient in need thereof, comprising administering to the patient a therapeutically effective amount of aficamten 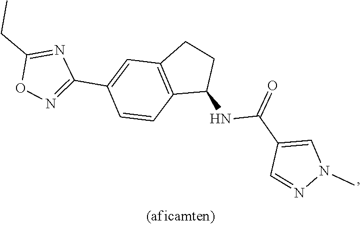 [SMILES: CCc1nc(-c2ccc3c(c2)CC[C@H]3NC(=O)c2cnn(C)c2)no1]
or a pharmaceutically acceptable salt thereof, and optionally a beta blocker;
wherein the disease or condition is selected from the group consisting of:
(a) hypertrophic cardiomyopathy (HCM);
(b) a heart disease selected from the group consisting of diastolic dysfunction, primary or secondary restrictive cardiomyopathy, myocardial infarction, and angina pectoris, left ventricular outflow tract obstruction, hypertensive heart disease, congenital heart disease, cardiac ischemia, coronary heart disease, diabetic heart disease, congestive heart failure, right heart failure, cardiorenal syndrome, and infiltrative cardiomyopathy; and
(c) a heart disease that is or is related to one or more conditions selected from the group consisting of cardiac senescence, diastolic dysfunction due to aging, left ventricular hypertrophy and concentric left ventricular remodeling;
and
wherein the method improves one or more parameter in the patient wherein each of the one or more parameter is selected from the group consisting of:
(1) heart rate reserve;
(2) peak metabolic equivalents (METs);
(3) exercise duration;
(4) peak oxygen uptake (pVO₂); and
(5) one or more of integrated 2 component Z-score metric, peak workload, peak circulatory power, peak respiratory exchange ratio, peak heart rate, O₂ pulse at peak exercise, peak proportionate pulse pressure, proportionate pulse pressure at rest, ventilatory efficiency pre-anaerobic threshold, ventilatory efficiency throughout exercise, ventilatory power, ventilatory anaerobic threshold, and aerobic efficiency in the patient.

77-139. (canceled)

140. A method of treating a patient in need thereof, comprising administering to the patient a therapeutically effective amount of aficamten 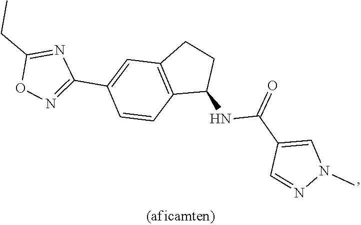 [SMILES: CCc1nc(-c2ccc3c(c2)CC[C@H]3NC(=O)c2cnn(C)c2)no1]
or a pharmaceutically acceptable salt thereof,
wherein the method improves one or more parameter in the patient wherein each of the one or more parameter is selected from the group consisting of: improvement in mitral valve SAM, improvement in peak A velocity, complete resolution of mitral valve SAM, increase in LV end systolic volume index, increase in LV end systolic volume, increase in Lateral e′ velocity, increase in Septal e′ velocity, increase in LV end systolic dimension, increase in absolute LV GLS (left ventricular global longitudinal strain), decrease in LA volume index, decrease in wall thickness, decrease in maximum wall thickness, decrease in interventricular septal wall thickness, decrease in interventricular septal wall, decrease in inferolateral wall thickness, decrease in LV mass index, decrease in LV global circumferential strain, decrease in TAPSE, decrease in RV s′ velocity, decrease in LA width, decrease in lateral E/e′, decrease in septal E/e′, decrease in peak E wave velocity, and decrease in LV GCS (left ventricular global circumferential strain).

141-144. (canceled)
